(19) United States
(12) Patent Application Publication
(10) Pub. No.: US 2025/0197360 A1
(43) Pub. Date: Jun. 19, 2025

(54) NOVEL DIAZEPINES THAT TARGET YELLOW FEVER VIRUS NON-STRUCTURAL 4B (NS4B) PROTEIN AND THEIR METHOD OF USE

(71) Applicant: BARUCH S. BLUMBERG INSTITUTE, Doylestown, PA (US)

(72) Inventors: Yanming DU, Cheshire, CT (US); Jinhong CHANG, Chalfont, PA (US); Ju-Tao GUO, Lansdale, PA (US)

(21) Appl. No.: 18/845,256

(22) PCT Filed: Mar. 10, 2023
(86) PCT No.: PCT/US2023/014957

§ 371 Date: Sep. 9, 2024

Related U.S. Application Data
(60) Provisional application No. 63/318,538, filed on Mar. 10, 2022.

(51) Int. Cl. C07D 243/24; A61K 31/5513; A61P 31/14; C07D 243/14; C07D 401/04; C07D 401/12; C07D 403/04; C07D 403/06; C07D 403/12; C07D 405/04; C07D 405/12; C07D 409/12

(52) U.S. Cl. CPC C07D 243/24; A61K 31/5513; A61P 31/14; C07D 243/14; C07D 401/04; C07D 401/12; C07D 403/04; C07D 403/06; C07D 403/12; C07D 405/04; C07D 405/12; C07D 409/12

(57) ABSTRACT

Pharmaceutical compositions of the invention comprise diazepines derivatives having a disease-modifying action in the treatment of diseases associated with biological effect that include disease state, and any disease type/class involving biological effect.

### CROSS-REFERENCE TO RELATED APPLICATION

This application claims the benefit of priority from U.S. Provisional Application No. 63/318,538 filed Mar. 10, 2022, the contents of which are hereby incorporated herein by reference in their entirety.

### STATEMENT REGARDING FEDERALLY SPONSORED RESEARCH

The invention was made with government support under grant number R01AI134732 awarded by the National Institutes of Health. The government has certain rights in the invention.

### FIELD OF THE DISCLOSURE

The present invention describes compounds that target Yellow Fever virus (YFV) non-structural 4B (NS4B) protein, useful for the treatment of viral infections and related conditions. The present invention further describes a novel chemotype useful for the prevention of Yellow fever viral infections.

### BACKGROUND

Yellow fever is an acute viral hemorrhagic disease which threatens approximately one billion people living in tropical areas of Africa, Central and South America. Although a highly effective yellow fever vaccine has been available for more than seven decades, the low vaccination rate fails to prevent outbreaks in at-risk regions. It has been estimated that up to 1.7 million YFV infections occur in Africa each year, resulting in 29,000 to 60,000 deaths, which outnumber the estimated death toll of global dengue virus infection. Once an outbreak starts, vaccination is less effective, and other intervention strategies, such as antiviral therapies, are needed. However, there are no specific antiviral therapeutics to treat the life-threatening disease, and development of antiviral agents that inhibit YFV replication is a major medical need.

Viral non-structural 4B (NS4B) protein is a nonenzymatic integral membrane protein. Flaviviral NS4B proteins participate in viral RNA replication and evasion of host innate immune response. The critical roles of this protein in viral replication and pathogenesis are well illustrated by the fact that many cell culture and animal adaptive mutations map to NS4B. The exact functional involvement of flavivirus NS4B in the viral replication cycle and the molecular mechanism to evade host innate immunity remain to be determined. Prior studies have already demonstrated that NS4B dimerization and interaction with other viral nonstructural proteins, including NS1, NS2A, NS3, and NS4A, as well as many host cellular proteins, are required for its function in forming viral RNA replication organelles (ROs) and antagonism of the immune response. Thus, direct binding to NS4B and/or blocking the NS4B protein interaction with other viral and host cellular proteins is a viable method of restricting viral replication. Further, compounds capable of interfering with or blocking the interaction of viral NSB4 with other viral nonstructural proteins, including NS1, NS2A, NS3, and NS4A, as well as many host cellular proteins are useful for the treatment and prevention of flavivirus infection. Flavivirus infection treatable with compounds that target NSB4 to block or interfere with the interaction of viral NSB4 with other viral nonstructural proteins, including NS1, NS2A, NS3, and NS4A, as well as host cellular proteins include Yellow Fever virus, Dengue virus, and Japanese Encephalitis virus.

### SUMMARY

The present invention is directed toward novel diazepine, compounds of formula (I),

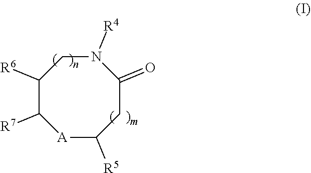

Including enantiomers, diasteromers, hydrates, solvates, pharmaceutically acceptable salts, isotopic analogs, prodrugs and complexes thereof, wherein:
A is selected from the groups consisting of

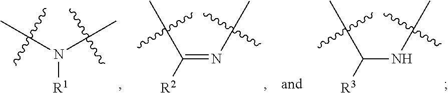

When A is

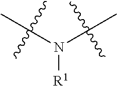

m is 0;
When A is

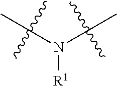

n is selected from 0 and 1;

R¹ is selected from the group consisting of C3-7 cycloalkyl, C3-C7 branched alkyls; C1-C6 haloalkyls, C3-C7 branched haloalkyl, optionally substituted aryl and optionally substituted heteroaryl;
When A is

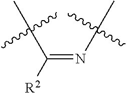

m is selected from 0 and 1;
When A is

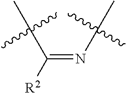

n is selected from 0 and 1;
R² is selected from consisting of C3-C8 cycloalkyl, C4-C10 bicyclic alkyl, C3-C7 branched alkyls, C1-C6 haloalkyls, C3-C7 branched haloalkyl, optionally substituted aryl, and optionally substituted heteroaryl;
When A is

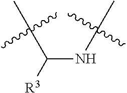

m is selected from 0 and 1;
When A is

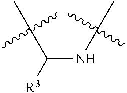

n is selected from 0 and 1;
n is selected from consisting of C3-C8 cycloalkyl; C3-C7 branched alkyls, C1-C6 haloalkyls, C3-C7 branched haloalkyl, optionally substituted aryl, and optionally substituted heteroaryl;
R⁴ is selected from the groups consisting of

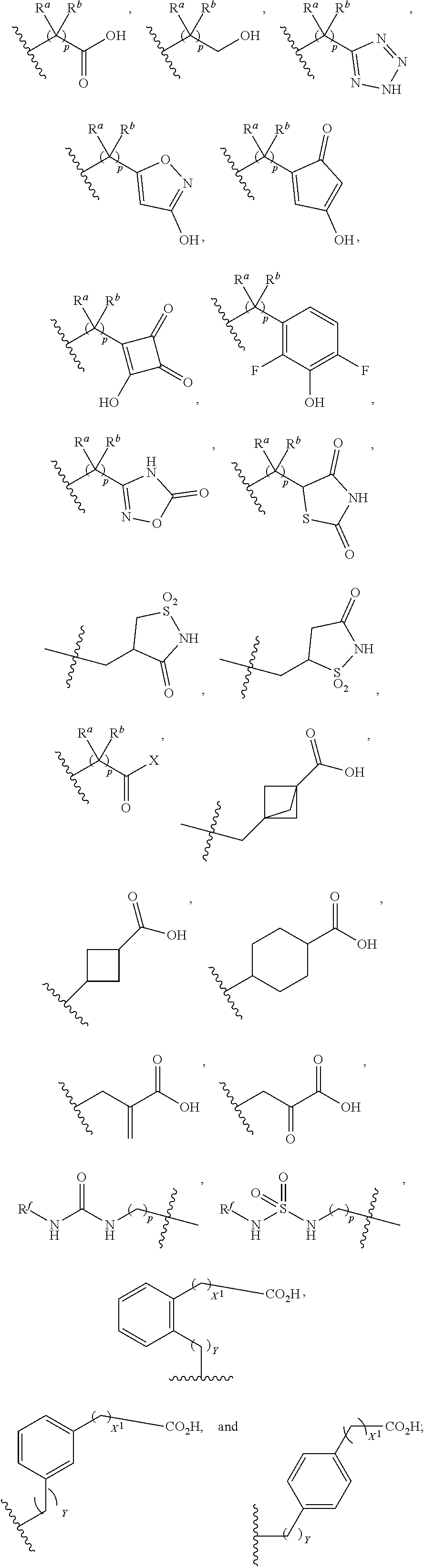

p is 1,2,3,4 or 5;
X¹ is 0, 1, 2, or 3;
Y is 0, 1, 2, 3, or 4;
When R⁴ is

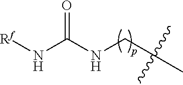

p is not 1;
When R⁴ is

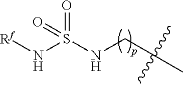

p is not 1;
X is selected from the group consisting of

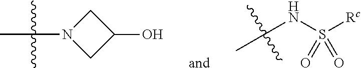

R^{a} is selected from the group consisting of H, C1-C6 alkyl, C3-C6 cycloalkyl, halogan;
R^{b} is selected from the group consisting of H, C1-C6 alkyl, C3-C6 cycloalkyl, halogen;
R^{c} is selected from the group consisting of C1-C12 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, NR^{d}R^{e}, optionally substituted benzyl, optionally substituted CH₂heteroaryl, optionally substituted aryl, optionally substituted heteroaryl, and C3-C6 cycloalkyl ring optionally containing an oxygen,
R^{a} and R^{b} can be taken together to form a C3-C6 cycloalkyl ring optionally containing an oxygen;
R^{d} is selected from the group consisting of hydrogen, C1-C6 alkyl, C3-C6 cycloalyl, optionally aromatic ring, and optionally substituted heteroaromatic ring;
R^{e} is selected from the group consisting of hydrogen, C1-C6 alkyl, C3-C6 cycloalyl, optionally aromatic ring, and optionally substituted heteroaromatic ring;
R^{f} is selected from the group consisting of hydrogen, C1-C12 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, optionally substituted benzyl, optionally substituted CH₂heteroaryl, optionally substituted aryl, optionally substituted heteroaryl, and C3-C6 cycloalkyl ring optionally containing an oxygen;
R⁵ is selected from the groups consisting of C2-C8 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, optionally substituted benzyl, optionally substituted CH₂heteroaryl, CH₂OR^{5a}, CH(CH₃)OR^{5a}, C(R^{5e})₂OR^{5a}, C(R^{5c})₂OR^{5a}, CH₂SR^{5a}, CH₂CH₂SCH₃, CH₂CH₂SO₂CH₃, CH₂CH₂CH₂NR^{5c} R^{5d}, CH₂COR^{5b}, CH₂CH₂COR^{1b}, (CH₂)_{z}(C3-C7cycloakyl), and C3-C6 cycloalkyl ring optionally containing an oxygen;
z is 1, 2, 3, 4, 5, or 6;
R^{5a} is selected from the group consisting of hydrogen, C1-C6 alkyl, C3-C7 branched alkyl, optionally substituted phenyl, optionally substituted benzyl, and optionally substituted CH₂CH₂Ar;
R^{5b} is selected from the group consisting of OH, C1-C6 alkoxy, and NH₂;
R^{5c} and R^{5d} are each independently is selected from the group consisting of hydrogen and optionally substituted C1-C6 alkyl;
R^{5c} is C1-C4 alkyl;
R⁶ and R⁷ are hydrogen;
Or
R⁶ and R⁷ are taken together with the atoms to which they are bound to form a 6 membered aromatic ring that is substituted with at least one moiety that is selected from the group consisting of halogen,
C1-C6 haloalkyl, cyano, C1-C6 alkyl,

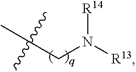

C3-C8 cycloalkyl, C3-C8 heterocycle, aryl, and heteroaryl;
Or
R⁶ and R⁷ are taken together with the atoms to which they are bound to form a substituted heteroaromatic ring containing 5 to 6 members containing 1 to 2 nitrogens that is substituted with at least one moiety that is selected from the group consisting of halogen, C1-C6 haloalkyl, cyano, C1-C6 alkoxy,

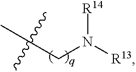

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;

The compounds of the present invention include compounds having formula (II):

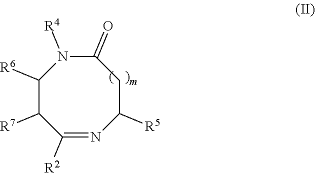

Including enantiomers, diasteromers, hydrates, solvates, pharmaceutically acceptable salts, prodrugs and complexes thereof.

The compounds of the present invention include compounds having formula (III):

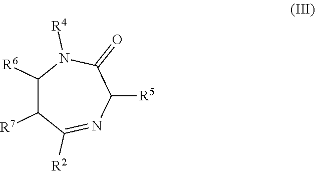

Including enantiomers, diasteromers, hydrates, solvates, pharmaceutically acceptable salts, prodrugs and complexes thereof.

The compounds of the present invention include compounds having formula (IV):

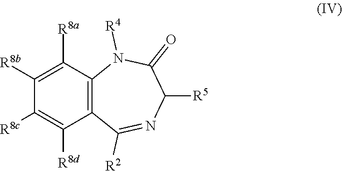

Including enantiomers, diasteromers, hydrates, solvates, pharmaceutically acceptable salts, prodrugs and complexes thereof wherein:
R^{8a} is selected from the group consisting of hydrogen, halogen, C1-C6 haloalkyl, cyano, C1-C6 alkyl, C1-C6 alkoxy,

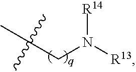

C3-C8 cycloalkyl, C3-C8 heterocycle, aryl, and heteroaryl;
R³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{5b} is selected from the group consisting of hydrogen, halogen, C1-C6 haloalkyl, cyano, C1-C6 alkyl,
C1-C6 alkoxy,

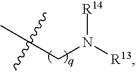

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{8c} is selected from the group consisting of halogen, C1-C6 haloalkyl, cyano, C1-C6 alkyl, C1-C6 alkoxy,

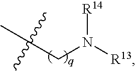

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{8d} is selected from the group consisting of hydrogen, halogen, C1-C6 haloalkyl, cyano, C1-C6 alkyl,
C1-C6 alkoxy,

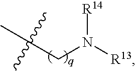

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
At least two of R^{8a}, R^{8b}, and R^{8d} are hydrogen;
The compounds of the present invention include compounds having formula (V):

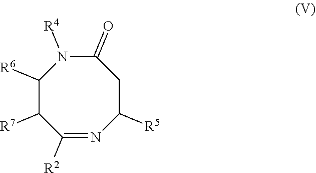

Including enantiomers, diasteromers, hydrates, solvates, pharmaceutically acceptable salts, prodrugs and complexes thereof.

The compounds of the present invention include compounds having formula (VI):

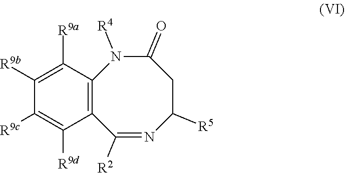

Including enantiomers, diasteromers, hydrates, solvates, pharmaceutically acceptable salts, prodrugs and complexes thereof wherein:
R^{9a} is selected from the group consisting of hydrogen, halogen, C1-C6 haloalkyl, cyano, C1-C6 alkyl, C1-C6 alkoxy,

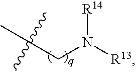

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{9b} is selected from the group consisting of hydrogen, halogen, C1-C6 haloalkyl, cyano, C1-C6 alkyl,
C1-C6 alkoxy,

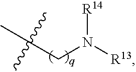

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{9c} is selected from the group consisting of halogen, C1-C6 haloalkyl, cyano, C1-C6 alkyl, C1-C6 alkoxy,

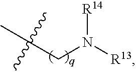

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{9d} is selected from the group consisting of hydrogen, halogen, C1-C6 haloalkyl, cyano, C1-C6 alkyl,
C1-C6 alkoxy,

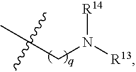

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{9a}, R^{9b}, and R^{9d} are hydrogen;

The compounds of the present invention include compounds having formula (VII):

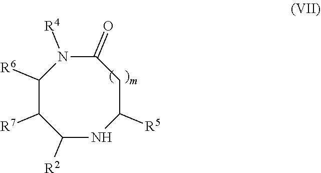

Including enantiomers, diasteromers, hydrates, solvates, pharmaceutically acceptable salts, prodrugs and complexes thereof.

The compounds of the present invention include compounds having formula (VIII):

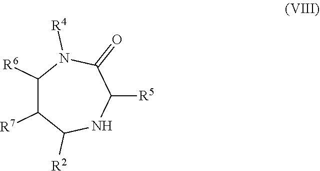

Including enantiomers, diasteromers, hydrates, solvates, pharmaceutically acceptable salts, prodrugs and complexes thereof.

The compounds of the present invention include compounds having formula (IX):

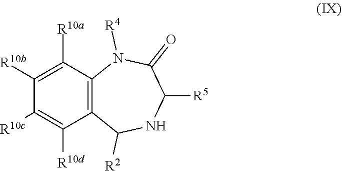

Including enantiomers, diasteromers, hydrates, solvates, pharmaceutically acceptable salts, prodrugs and complexes thereof wherein
R^{10a} is selected from the group consisting of hydrogen, halogen, C1-C6 haloalkyl, cyano, C1-C6 alkyl, C1-C6 alkoxy,

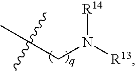

C3-C8 cycloalkyl, C3-C8 heterocycle, aryl, and heteroaryl;
R¹³ is selected from the group consisting of hydrogen and C1-C6 alkyl;
R⁴ is selected from the group consisting of hydrogen and C1-C6 alkyl;
q is 0, 1, 2, 3, 4, 5, or 6;
R^{10b} is selected from the group consisting of hydrogen, halogen, C1-C6 haloalkyl, cyano, C1-C6 alkyl, C1-C6 alkoxy,

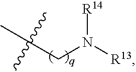

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{10c} is selected from the group consisting of halogen, C1-C6 haloalkyl, cyano, C1-C6 alkyl, C1-C6 alkoxy,

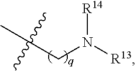

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{10d} is selected from the group consisting of hydrogen, halogen, C1-C6 haloalkyl, cyano, C1-C6 alkyl, C1-C6 alkoxy,

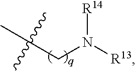

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{10a}, R^{10c}, and R^{10d} are hydrogen;
The compounds of the present invention include compounds having formula (X):

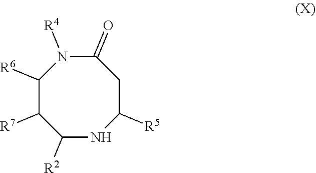

Including enantiomers, diasteromers, hydrates, solvates, pharmaceutically acceptable salts, prodrugs and complexes thereof.

The compounds of the present invention include compounds having formula (XI):

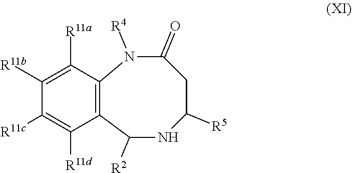

Including enantiomers, diasteromers, hydrates, solvates, pharmaceutically acceptable salts, prodrugs and complexes thereof wherein;
R^{10a} is selected from the group consisting of hydrogen, halogen, C1-C6 haloalkyl, cyano, C1-C6 alkyl, C1-C6 alkoxy,

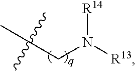

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{1b} is selected from the group consisting of hydrogen, halogens, C1-C6 haloalkyl, cyano, C1-C6

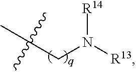

alkyl, C1-C6 alkoxy, C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{11c} is selected from the group consisting of halogen, C1-C6 haloalkyl, cyano, C1-C6 alkyl, C1-C6 alkoxy,

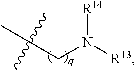

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{11d} is selected from the group consisting of hydrogen, halogen, C1-C6 haloalkyl, cyano, C1-C6 alkyl, C1-C6 alkoxy,

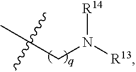

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{11a}, R^{11b}, and R^{11d} are hydrogen;

The compounds of the present invention include compounds having formula (XII):

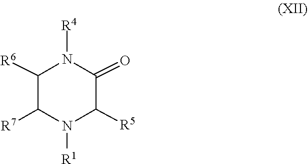

Including enantiomers, diasteromers, hydrates, solvates, pharmaceutically acceptable salts, prodrugs and complexes thereof.

The compounds of the present invention include compounds having formula (XIII):

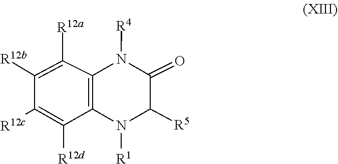

Including enantiomers, diasteromers, hydrates, solvates, pharmaceutically acceptable salts, prodrugs and complexes thereof wherein
R^{12a} is selected from the group consisting of hydrogen, halogen, C1-C6 haloalkyl, cyano, C1-C6 alkyl, C1-C6 alkoxy,

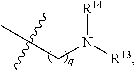

C3-C8 cycloalkyl, C3-C8 heterocycle, aryl, and heteroaryl;
R³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{12b} is selected from the group consisting of hydrogen, halogen, C1-C6 haloalkyl, cyano, C1-C6 alkyl, C1-C6 alkoxy,

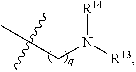

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R¹²° is selected from the group consisting of halogens, C1-C6 haloalkyls, cyano, C1-C6 alkyl, C1-C6 alkoxy,

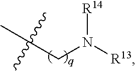

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{12d} is selected from the group consisting of hydrogen, halogen, C1-C6 haloalkyl, cyano, C1-C6 alkyl, C1-C6 alkoxy,

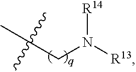

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{12a}, R^{12b}, and R^{12d} are hydrogen;

The compounds of the present invention include compounds having formula (XIV):

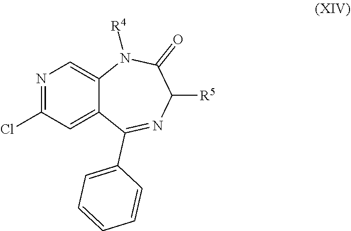

Including enantiomers, diasteromers, hydrates, solvates, pharmaceutically acceptable salts, prodrugs and complexes thereof.

The compounds of the present invention include compounds having formula (XV):

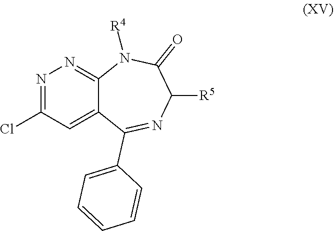

Including enantiomers, diasteromers, hydrates, solvates, pharmaceutically acceptable salts, prodrugs and complexes thereof.

The compounds of the present invention include compounds having formula (XVI):

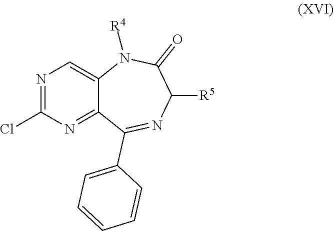

Including enantiomers, diasteromers, hydrates, solvates, pharmaceutically acceptable salts, prodrugs and complexes thereof.

The compounds of the present invention include compounds having formula (XVII):

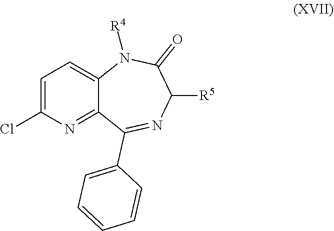

Including enantiomers, diasteromers, hydrates, solvates, pharmaceutically acceptable salts, prodrugs and complexes thereof.

The compounds of the present invention include compounds having formula (XVIII):

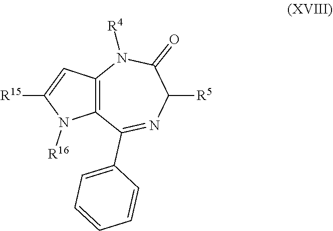

Including enantiomers, diasteromers, hydrates, solvates, pharmaceutically acceptable salts, prodrugs and complexes thereof wherein
R¹⁵ is selected from the group consisting of C1-C6 alkyl, C3-C6 cycloalkyl, and C1-C6 haloalkyl;
R¹⁶ is selected from the group consisting of C1-C6 alkyl, C3-C6 cycloalkyl, and C1-C6 haloalkyl;

The compounds of the present invention include compounds having formula (XIX):

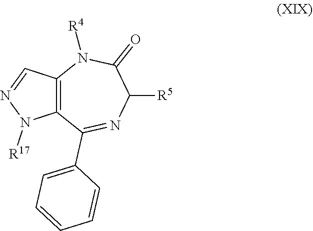

Including enantiomers, diasteromers, hydrates, solvates, pharmaceutically acceptable salts, prodrugs and complexes thereof wherein
R¹⁷ is selected from the group consisting of C1-C6 alkyl, C3-C6 cycloalkyl, and C1-C6 haloalkyl;

The compounds of the present invention include compounds having formula (XX):

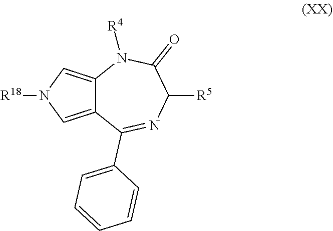

Including enantiomers, diasteromers, hydrates, solvates, pharmaceutically acceptable salts, prodrugs and complexes thereof wherein R¹⁸ is selected from the group consisting of C1-C6 alkyl, C3-C6 cycloalkyl, and C1-C6 haloalkyl;
The compounds of the present invention include compounds having formula (XXI):

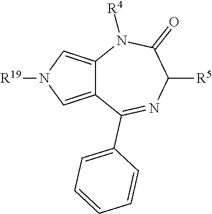

Including enantiomers, diasteromers, hydrates, solvates, pharmaceutically acceptable salts, prodrugs and complexes thereof wherein
R¹⁹ is selected from the group consisting of C1-C6 alkyl, C3-C6 cycloalkyl, and C1-C6 haloalkyl;
The following compounds are explicitly excluded from the scope of the invention:

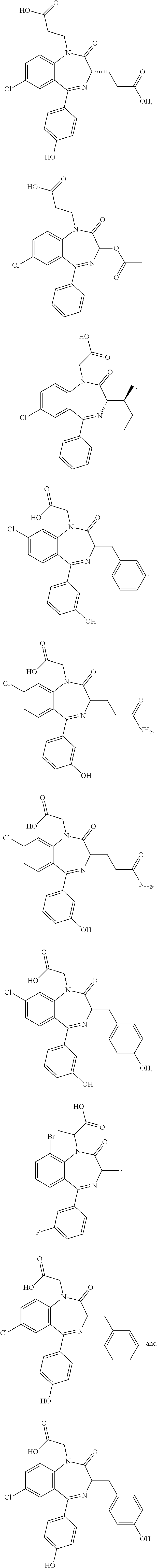

The present invention further relates to compositions comprising: an effective amount of one or more compounds according to the present invention and an excipient.

The present invention also relates to a method for treating or preventing Yellow Fever virus infection, said method comprising administering to a subject an effective amount of a compound or composition according to the present invention.

The present invention yet further relates to a method for treating or preventing Yellow Fever Virus infection, wherein said method comprises administering to a subject a composition comprising an effective amount of one or more compounds according to the present invention and an excipient.

The present invention also relates to a method for treating or preventing disease or conditions associated with Yellow Fever virus infection. Said methods comprise administering to a subject an effective amount of a compound or composition according to the present invention.

The present invention yet further relates to a method for treating or preventing disease or conditions associated with Yellow Fever virus infection, wherein said method comprises administering to a subject a composition comprising an effective amount of one or more compounds according to the present invention and an excipient.

The present invention also relates to a method for treating or preventing Dengue virus infection, said method comprising administering to a subject an effective amount of a compound or composition according to the present invention.

The present invention yet further relates to a method for treating or preventing Dengue virus infection, wherein said method comprises administering to a subject a composition comprising an effective amount of one or more compounds according to the present invention and an excipient.

The present invention also relates to a method for treating or preventing disease or conditions associated with Dengue virus infection. Said methods comprise administering to a subject an effective amount of a compound or composition according to the present invention.

The present invention yet further relates to a method for treating or preventing disease or conditions associated with Dengue virus infection, wherein said method comprises administering to a subject a composition comprising an effective amount of one or more compounds according to the present invention and an excipient.

The present invention also relates to a method for treating or preventing Japanese Encephalitis virus infection, said method comprising administering to a subject an effective amount of a compound or composition according to the present invention.

The present invention yet further relates to a method for treating or preventing Japanese Encephalitis virus infection, wherein said method comprises administering to a subject a composition comprising an effective amount of one or more compounds according to the present invention and an excipient.

The present invention also relates to a method for treating or preventing disease or conditions associated with Japanese Encephalitis virus infection. Said methods comprise administering to a subject an effective amount of a compound or composition according to the present invention.

The present invention yet further relates to a method for treating or preventing disease or conditions associated with Japanese Encephalitis virus infection, wherein said method comprises administering to a subject a composition comprising an effective amount of one or more compounds according to the present invention and an excipient.

The present invention further relates to a process for preparing the diazepines of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION

The diazepines of the present invention are capable of treating and preventing YFV infection through targeting viral NS4B and its interaction with other viral nonstructural proteins, including NS1, NS2A, NS3, NS4A, and host cellular proteins. It has been discovered that blocking the NS4B protein interaction with other viral including NS1, NS2A, NS3, NS4A, and host cellular proteins is a viable method of restricting viral replication. Further, compounds capable of interfering with or blocking the interaction of viral NS4B with other viral nonstructural proteins, including NS1, NS2A, NS3, and NS4A, as well as host cellular proteins are useful for the treatment and prevention of flavivirus infection. Flavivirus infection treatable with compounds that target NS4B to block or interfere with the interaction of viral NS4B with other viral nonstructural proteins, including NS1, NS2A, NS3, and NS4A, as well as host cellular proteins include Yellow Fever virus, Dengue virus, and Japanese Encephalitis virus.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group-alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl(e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl(valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]iimidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl(e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C1-C5 heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

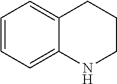

is, for the purposes of the present invention, considered a heterocyclic unit. 6,7-Dihydro-5H-cyclopentapyrimidine having the formula:

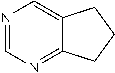

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

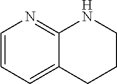

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR^{x}, —SR^{x}, —N(R^{x})₂, —NR^{x}C(O)R^{x}, —SO₂R^{x}, —SO₂OR^{x}, —SO₂N(R^{x})₂, —C(O)R^{x}, —C(O)OR^{x}, —C(O)N(R^{x})₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R^{x}; wherein R^{x}, at each occurrence, independently is hydrogen, —OR^{x+1}, —SR^{x+1}, —C(O)R^{x+1}, —C(O)OR^{x+1}, —C(O)N(R^{x+1})₂, —SO₂R^{x+1}, —S(O)₂OR^{x+1}, —N(R^{x+1})₂, —NR^{x+1}-C(O)R^{x+1}, C₁₋₆ alkyl, C₁₋₆ haloalkyl, C₂₋₈ alkenyl, C₂₋₈ alkynyl, cycloalkyl(e.g., C₃₋₆ cycloalkyl), aryl, heterocycle, or heteroaryl, or two R^{x} units taken together with the atom(s) to which they are bound form an optionally substituted carbocycle or heterocycle wherein said carbocycle or heterocycle has 3 to 7 ring atoms; wherein R^{x+1}, at each occurrence, independently is hydrogen, C₁₋₆ alkyl, C₁₋₆ haloalkyl, C₂₋₈ alkenyl, C₂₋₈ alkynyl, cycloalkyl(e.g., C₃₋₆ cycloalkyl), aryl, heterocycle, or heteroaryl, or two R^{x+1} units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR^{x+2}; for example, —OH, —OCH₃, —OCH₂CH₃, —OCH₂CH₂CH₃;
ii) —C(O)R^{x+2}; for example, —COCH₃, —COCH₂CH₃, —COCH₂CH₂CH₃;
iii) —C(O)OR^{x+2}; for example, —CO₂CH₃, —CO₂CH₂CH₃, —CO₂CH₂CH₂CH₃;
iv) —C(O)N(R¹²)₂; for example, —CONH₂, —CONHCH₃, —CON(CH₃)₂;
v) —N(R^{x12})₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R^{x+2}; for example, —SO₂H; —SO₂CH₃; —SO₂C₆H₅;
ix) C₁-C₆ linear, branched, or cyclic alkyl;
x) Cyano
xi) Nitro;
xii) N(R^{x+2})C(O)R^{x+2}
xiii) Oxo (═O);
xiv) Heterocycle; and
xv) Heteroaryl.

wherein each R^{x+2} is independently hydrogen, optionally substituted C1-C6 linear or branched alkyl(e.g. , optionally substituted C1-C4 linear or branched alkyl), or optionally substituted C₃-C₆ cycloalkyl(e.g optionally substituted C₃-C₄ cycloalkyl); or two R^{x+2} units can be taken together to form a ring comprising 3-7 ring atoms. In certain aspects, each R^{x+2} is independently hydrogen, C1-C6 linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the Biological effect agent described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, malcic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R^{x+1})₂, each R^{x+1} may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

The diazepines of the present invention

The compounds of the present invention are diazepines, and include all enantiomeric and diastereomeric forms and pharmaceutically accepted salts thereof having the formula (I):

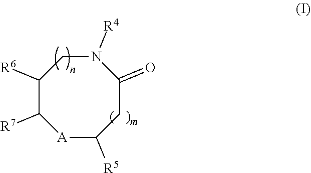

Including enantiomers, diasteromers, hydrates, solvates, pharmaceutically acceptable salts, isotopic analogs, prodrugs and complexes thereof, wherein:
A is selected from the groups consisting of

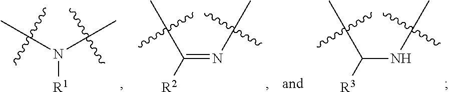

When A is

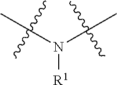

m is 0;
When A is

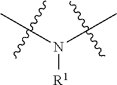

n is selected from 0 and 1;

R¹ is selected from the group consisting of 3-7 cycloalkyl, C3-C7 branched alkyls; C1-C6 haloalkyls, C3-C7 branched haloalkyl, optionally substituted aryl and optionally substituted heteroaryl;

When A is

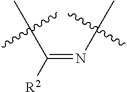

m is selected from 0 and 1;

When A is

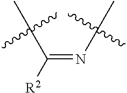

n is selected from 0 and 1;
R² is selected from consisting of C3-C8 cycloalkyl, C4-C10 bicyclic alkyl, C3-C7 branched alkyls; C1-C6 haloalkyls, C3-C7 branched haloalkyl, optionally substituted aryl, and optionally substituted heteroaryl;

When A is

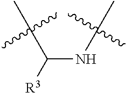

m is selected from 0 and 1;

When A is

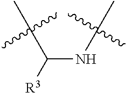

n is selected from 0 and 1;
R³ is selected from consisting of C3-C8 cycloalkyl, C3-C7 branched alkyls; C1-C6 haloalkyls, C3-C7 branched haloalkyl, optionally substituted aryl, and optionally substituted heteroaryl;
R⁴ is selected from the groups consisting of

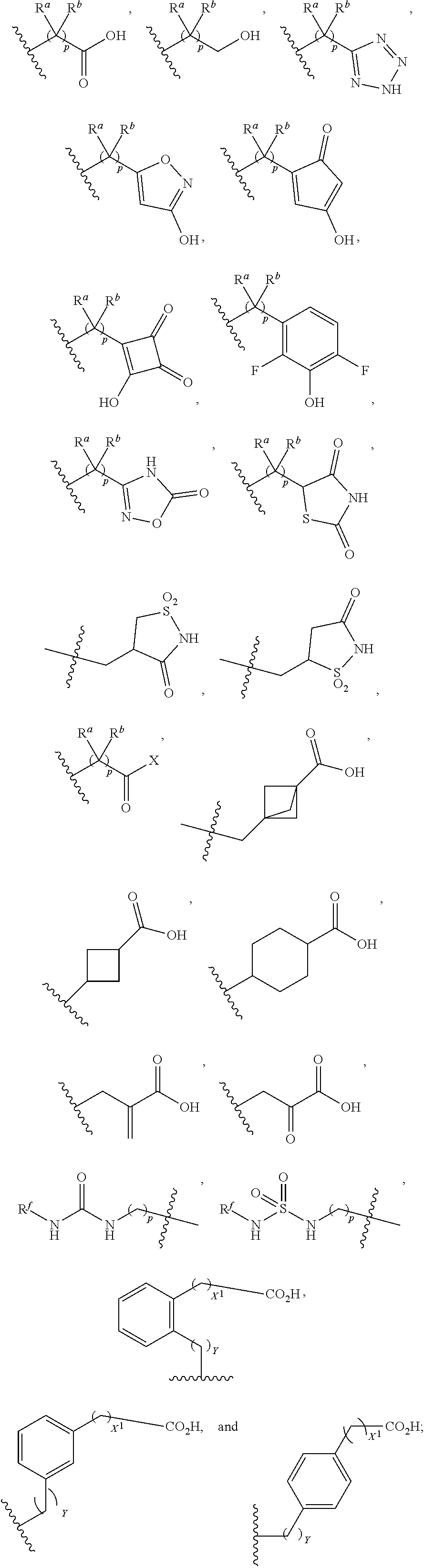

p is 1, 2, 3, 4, or 5;
When R⁴ is

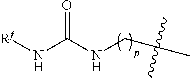

p is not 1;
When R⁴ is

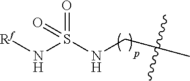

p is not 1;
X¹ is 0, 1, 2, or 3;
Y is 0, 1, 2, 3, or 4;
X is selected from the group consisting of

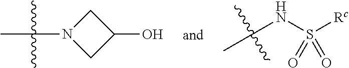

R^{a} is selected from the group consisting of H, C1-C6 alkyl, C3-C6 cycloalkyl, halide;
R^{b} is selected from the group consisting of H, C1-C6 alkyl, C3-C6 cycloalkyl, halide;
R^{c} is selected from the group consisting of C1-C12 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, NR^{d}R^{e}, optionally substituted benzyl, optionally substituted CH₂heteroaryl, optionally substituted aryl, and optionally substituted heteroaryl, and C3-C6 cycloalkyl ring optionally containing an oxygen;
R^{a} and R^{b} can be taken together to form a C3-C6 cycloalkyl ring optionally containing an oxygen;
R^{d} is selected from the group consisting of hydrogen, C1-C6 alkyl, C3-C6 cycloalyl, optionally aromatic ring, and optionally substituted heteroaromatic ring;
R^{f} is selected from the group consisting of hydrogen, C1-C6 alkyl, C3-C6 cycloalyl, optionally aromatic ring, and optionally substituted heteroaromatic ring;
R^{f} is selected from the group consisting of hydrogen, C1-C12 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, optionally substituted benzyl, optionally substituted CH₂heteroaryl, optionally substituted aryl, optionally substituted heteroaryl, and C3-C6 cycloalkyl ring optionally containing an oxygen:
R⁵ is selected from the groups consisting of C2-C8 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, optionally substituted benzyl, optionally substituted CH₂heteroaryl, CH₂OR^{5a}, CH(CH₃)OR^{5a}, C(R^{5e})₂OR^{5a}, C(R^{5e})₂OR^{5a}, CH₂SR^{5a}, CH₂CH₂SCH₃, CH₂CH₂SO₂CH₃, CH₂CH₂CH₂NR^{5c}R^{5d}, CH₂COR^{5b}, CH₂CH₂COR^{5b}, (CH₂)_{z}(C3-C7cycloakyl), and C3-C6 cycloalkyl ring optionally containing an oxygen;
z is 1, 2, 3, 4, 5, or 6;
R^{5a} is selected from the group consisting of hydrogen, C1-C6 alkyl, C3-C7 branched alkyl, optionally substituted phenyl, optionally substituted benzyl, and optionally substituted CH₂CH₂Ar;
R^{5b} is selected from the group consisting of OH, C1-C6 alkoxy, and NH₂;
R^{5c} and R^{5d} are each independently is selected from the group consisting of hydrogen and optionally substituted C1-C6 alkyl;
R^{5e} is C1-C4 alkyl;
R⁶ and R⁷ are hydrogen;
Or
R⁶ and R⁷ are taken together with the atoms to which they are bound to form a 6 membered aromatic ring that is substituted with at least one moiety that is selected from the group consisting of halogen,
C1-C6 haloalkyl cyano, C1-C6 alkyl, C1-C6 alkoxy,

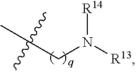

C3-C8 cycloalkyl, C3-C8 heterocycle, aryl, and heteroaryl;
Or
R⁶ and R⁷ are taken together with the atoms to which they are bound to form a substituted heteroaromatic ring containing 5 to 6 members containing 1 to 2 nitrogens that is substituted with at least one moiety that is selected from the group consisting of halogen, C1-C6 haloalkyl, cyano, C1-C6 alkoxy,

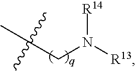

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;

The compounds of the present invention include compounds having formula (II):

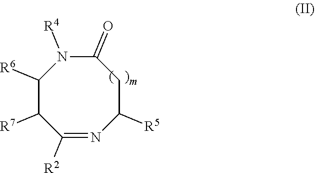

Including enantiomers, diasteromers, hydrates, solvates, pharmaceutically acceptable salts, prodrugs and complexes thereof.

The compounds of the present invention include compounds having formula (III):

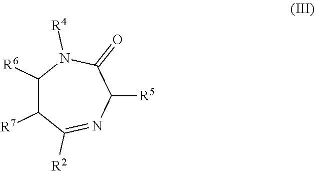

Including enantiomers, diasteromers, hydrates, solvates, pharmaceutically acceptable salts, prodrugs and complexes thereof.

The compounds of the present invention include compounds having formula (IV):

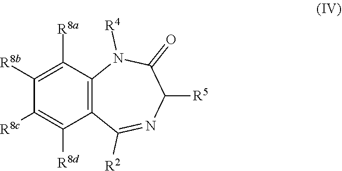

Including enantiomers, diasteromers, hydrates, solvates, pharmaceutically acceptable salts, prodrugs and complexes thereof wherein:
R^{8a} is selected from the group consisting of hydrogen halogen, C1-C6 haloalkyl, cyano, C1-C6 alkyl, C₁-C₆ alkoxy,

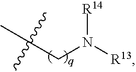

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{8b} is selected from the group consisting of hydrogen, halogen, C1-C6 haloalkyl, cyano, C1-C6 alkyl, C1-C6 alkoxy,

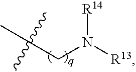

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{8c} is selected from the group consisting of halogen, C1-C6 haloalkyl, cyano, C1-C6 alkyl, C1-C6 alkoxy,

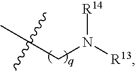

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{8c} is selected from the group consisting of hydrogen, halogen, C1-C6 haloalkyl, cyano, C1-C6 alkyl, C1-C6 alkoxy,

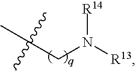

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{8a}, R^{8b}, and R^{8d} are hydrogen;

The compounds of the present invention include compounds having formula (V):

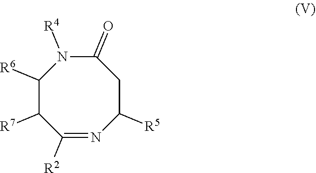

Including enantiomers, diasteromers, hydrates, solvates, pharmaceutically acceptable salts, prodrugs and complexes thereof.

The compounds of the present invention include compounds having formula (VI):

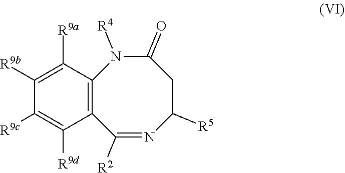

Including enantiomers, diasteromers, hydrates, solvates, pharmaceutically acceptable salts, prodrugs and complexes thereof wherein:
R^{9a} is selected from the group consisting of hydrogen, halogen, C1-C6 haloalkyl, cyano, C1-C6 alkyl, C₁-C₆ alkoxy,

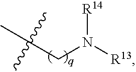

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{9b} is selected from the group consisting of hydrogen, halogen, C1-C6 haloalkyl, cyano, C1-C6 alkyl, C₁-C₆ alkoxy,

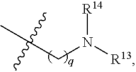

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{9c} is selected from the group consisting of halogen, C1-C6 haloalkyl, cyano, C1-C6 alkyl, C1-C6 alkoxy

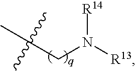

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{9d} is selected from the group consisting of hydrogen, halogen, C1-C6 haloalkyl, cyano, C1-C6 alkyl, C₁-C₆ alkoxy,

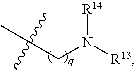

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{9a}, R^{9b}, and R^{9d} are hydrogen;
The compounds of the present invention include compounds having formula (VII):

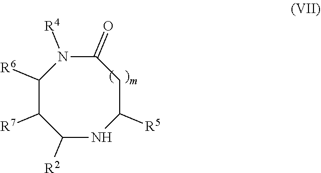

Including enantiomers, diasteromers, hydrates, solvates, pharmaceutically acceptable salts, prodrugs and complexes thereof.

The compounds of the present invention include compounds having formula (VIII):

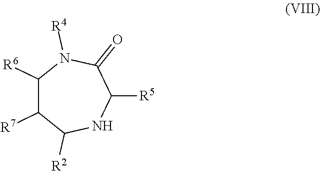

Including enantiomers, diasteromers, hydrates, solvates, pharmaceutically acceptable salts, prodrugs and complexes thereof.

The compounds of the present invention include compounds having formula (IX):

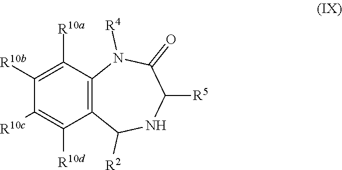

Including enantiomers, diasteromers, hydrates, solvates, pharmaceutically acceptable salts, prodrugs and complexes thereof wherein
R^{10a} is selected from the group consisting of hydrogen, halogen, C1-C6 haloalkyl, cyano, C1-C6 alkyl, C1-C6 alkoxy,

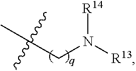

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{10b} is selected from the group consisting of hydrogen, halogen, C1-C6 haloalkyl, cyano, C1-C6 alkyl, C1-C6 alkoxy,

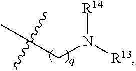

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R¹⁰ is selected from the group consisting of halogen, C1-C6 haloalkyl, cyano, C1-C6 alkyl, C1-C6 alkoxy

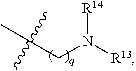

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{10d} is selected from the group consisting of hydrogen, halogen, C1-C6 haloalkyl, cyano, C1-C6 alkyl, C1-C6 alkoxy,

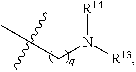

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{10a}, R^{10b}, and R^{10d} are hydrogen;
The compounds of the present invention include compounds having formula (X):

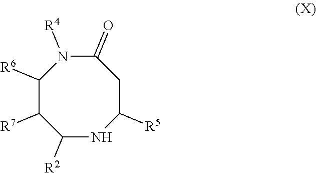

Including enantiomers, diasteromers, hydrates, solvates, pharmaceutically acceptable salts, prodrugs and complexes thereof.

The compounds of the present invention include compounds having formula (XI):

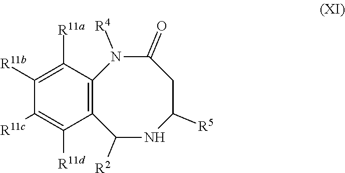

Including enantiomers, diasteromers, hydrates, solvates, pharmaceutically acceptable salts, prodrugs and complexes thereof wherein;
R^{11a} is selected from the group consisting of hydrogen, halogen, C1-C6 haloalkyl, cyano, C1-C6 alkyl, C1-C6 alkoxy,

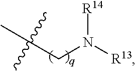

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{1b} is selected from the group consisting of hydrogen, halogens, C1-C6 haloalkyl, cyano, C1-C6 alkyl, C1-C6 alkoxy,

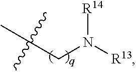

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{11c} is selected from the group consisting of halogen, C1-C6 haloalkyl, cyano, C1-C6 alkyl, C1-C6 alkoxy,

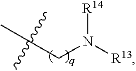

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{11d} is selected from the group consisting of hydrogen, halogen, C1-C6 haloalkyl, cyano, C1-C6 alkyl, C1-C6 alkoxy,

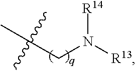

C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
At least two of R^{11a}, R^{11b}, and R^{11d} are hydrogen;

The compounds of the present invention include compounds having formula (XII):

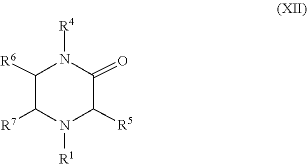

Including enantiomers, diasteromers, hydrates, solvates, pharmaceutically acceptable salts, prodrugs and complexes thereof.

The compounds of the present invention include compounds having formula (XIII):

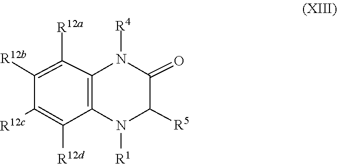

Including enantiomers, diasteromers, hydrates, solvates, pharmaceutically acceptable salts, prodrugs and complexes thereof wherein
R^{12a} is selected from the group consisting of hydrogen, halogen, C1-C6 haloalkyl, cyano, C1-C6 alkyl, C1-C6 alkoxy,

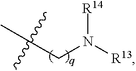

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{12d} is selected from the group consisting of hydrogen, halogen, C1-C6 haloalkyl, cyano, C1-C6 alkyl, C1-C6 alkoxy,

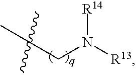

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{12c} is selected from the group consisting of halogens, C1-C6 haloalkyls, cyano, C1-C6 alkyl, C1-C6 alkoxy,

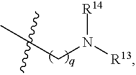

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6;
R^{12a} is selected from the group consisting of hydrogen, halogen, C1-C6 haloalkyl, cyano, C1-C6 alkyl, C1-C6 alkoxy,

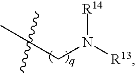

C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
At least two of R^{12a}, R^{12b}, and R^{12a} are hydrogen;

The compounds of the present invention include compounds having formula (XIV):

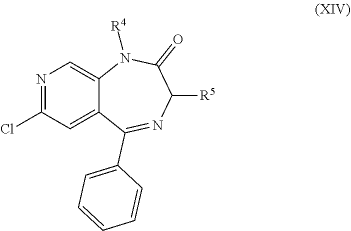

Including enantiomers, diasteromers, hydrates, solvates, pharmaceutically acceptable salts, prodrugs and complexes thereof.

The compounds of the present invention include compounds having formula (XV):

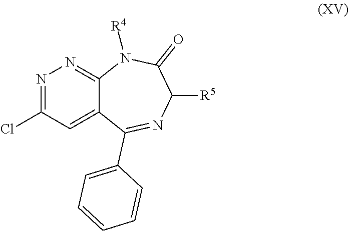

Including enantiomers, diasteromers, hydrates, solvates, pharmaceutically acceptable salts, prodrugs and complexes thereof.

The compounds of the present invention include compounds having formula (XVI):

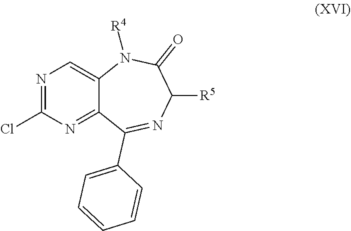

Including enantiomers, diasteromers, hydrates, solvates, pharmaceutically acceptable salts, prodrugs and complexes thereof.

The compounds of the present invention include compounds having formula (XVII):

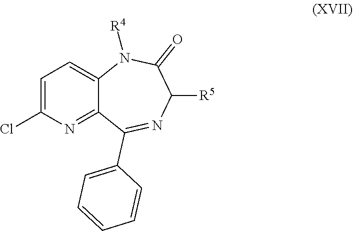

Including enantiomers, diasteromers, hydrates, solvates, pharmaceutically acceptable salts, prodrugs and complexes thereof.

The compounds of the present invention include compounds having formula (XVIII):

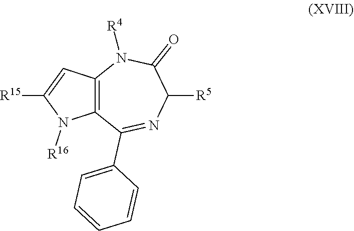

Including enantiomers, diasteromers, hydrates, solvates, pharmaceutically acceptable salts, prodrugs and complexes thereof wherein
R¹⁵ is selected from the group consisting of C1-C6 alkyl, C3-C6 cycloalkyl, and C1-C6 haloalkyl;
R¹⁶ is selected from the group consisting of C1-C6 alkyl, C3-C6 cycloalkyl, and C1-C6 haloalkyl;

The compounds of the present invention include compounds having formula (XIX):

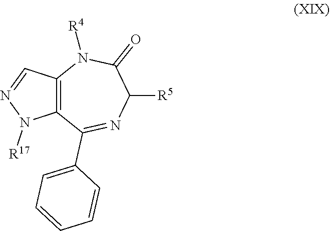

Including enantiomers, diasteromers, hydrates, solvates, pharmaceutically acceptable salts, prodrugs and complexes thereof wherein
R¹⁷ is selected from the group consisting of C1-C6 alkyl, C3-C6 cycloalkyl, and C1-C6 haloalkyl;

The compounds of the present invention include compounds having formula (XX):

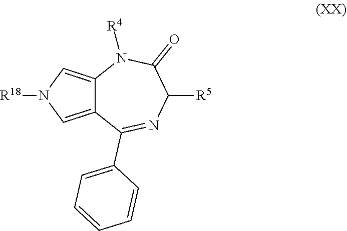

Including enantiomers, diasteromers, hydrates, solvates, pharmaceutically acceptable salts, prodrugs and complexes thereof wherein
R¹⁸ is selected from the group consisting of C1-C6 alkyl, C3-C6 cycloalkyl, and C1-C6 haloalkyl;

The compounds of the present invention include compounds having formula (XXI):

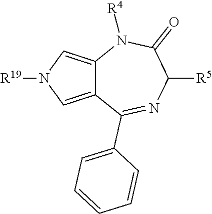

Including enantiomers, diasteromers, hydrates, solvates, pharmaceutically acceptable salts, prodrugs and complexes thereof wherein
R¹⁹ is selected from the group consisting of C1-C6 alkyl, C3-C6 cycloalkyl, and C1-C6 haloalkyl;

The following compounds are explicitly excluded from the scope of the invention:

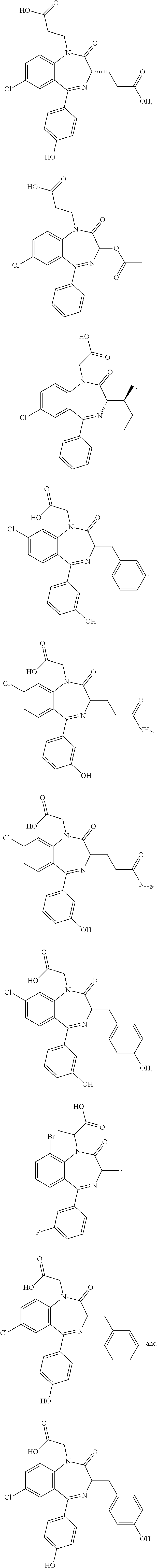

In some embodiments A is

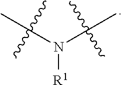

In some embodiments A is

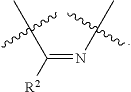

In son embodiments A is

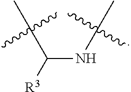

In some embodiments m is 0.

In some embodiments m is 1.

In some embodiments n is 0.

In some embodiments n is 1.

In some embodiments R¹ is C3-7 cycloalkyl.

In some embodiments R¹ is C3-C7 branched alkyls.

In some embodiments R¹ is C1-C6 haloalkyls.

In some embodiments R^{t} is C3-C7 branched haloalkyl.

In some embodiments R¹ is optionally substituted aryl.

In some embodiments R¹ is optionally substituted heteroaryl.

In some embodiments R² is C3-C8 cycloalkyl.

In some embodiments R² is C4-C10 bicyclic alkyl

In some embodiments R² is C3-C7 branched alkyls.

In some embodiments R² is C1-C6 haloalkyls.

In some embodiments R² is C3-C7 branched haloalkyl.

In some embodiments R² is optionally substituted aryl.

In some embodiments R² is optionally substituted heteroaryl.

In some embodiments R³ is C3-C8 cycloalkyl.

In some embodiments R³ is C3-C7 branched alkyls.

In some embodiments R³ is C1-C6 haloalkyls.

In some embodiments R³ is C3-C7 branched haloalkyl.

In some embodiments R³ is optionally substituted aryl.

In some embodiments R³ is optionally substituted heteroaryl.

In some embodiments R⁴ is

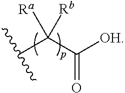

In some embodiments R⁴ is

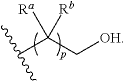

In some embodiments R⁴ is

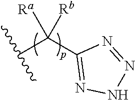

In some embodiments R⁴ is

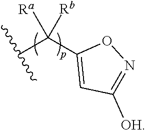

In some embodiments R⁴ is

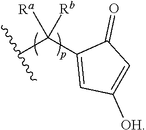

In some embodiments R⁴ is

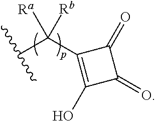

In some embodiments R⁴ is

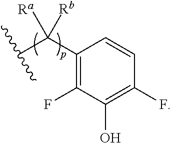

In some embodiments R⁴ is

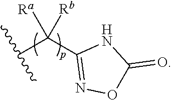

In some embodiments R⁴ is

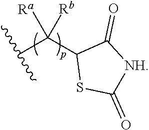

In some embodiments R⁴ is

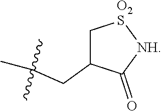 [SMILES: *CC1CS(=O)(=O)NC1=O]

In some embodiments R⁴ is

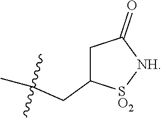 [SMILES: *CC1CC(=O)NS1(=O)=O]

In some embodiments R⁴ is

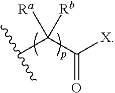

In some embodiments R⁴ is

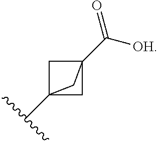 [SMILES: *C12CC(C(=O)O)(C1)C2]

In some embodiments R⁴ is

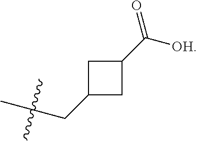 [SMILES: *CC1CC(C(=O)O)C1]

In some embodiments R⁴ is

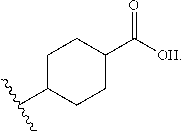 [SMILES: *C1CCC(C(=O)O)CC1]

In some embodiments R⁴ is

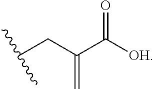 [SMILES: *CC(=C)C(=O)O]

In some embodiments R⁴ is

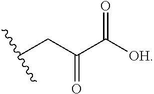 [SMILES: *CC(=O)C(=O)O]

In some embodiments R⁴ is

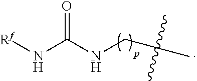

In some embodiments R⁴ is

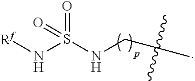

In some embodiments R⁴ is

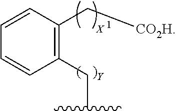

In some embodiments R⁴ is

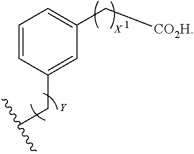

In some embodiments R⁴ is

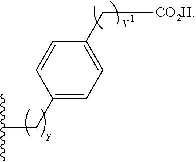

In some embodiments X¹ is 0.

In some embodiments X¹ is 1.

In some embodiments X¹ is 2.

In some embodiments X¹ is 3.

In some embodiments p is 1.

In some embodiments p is 2.

In some embodiments p is 3.

In some embodiments p is 4.

In some embodiments p is 5.

In some embodiments Y is 0.

In some embodiments Y is 1.

In some embodiments Y is 2.

In some embodiments Y is 3.

In some embodiments Y is 4.

In some embodiments X is

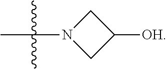 [SMILES: *N1CC(O)C1]

In some embodiments X is

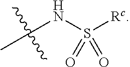

In some embodiments R^{a} is H.

In some embodiments R^{a} is C1-C6 alkyl.

In some embodiments R^{a} is C3-C6 cycloalkyl

In some embodiments R^{a} is a halogen.

In some embodiments R^{b} is H.

In some embodiments R^{b} is C1-C6 alkyl.

In some embodiments R^{b} is C3-C6 cycloalkyl

In some embodiments R^{b} is a halogen.

In some embodiments R^{c} is C1-C12 alkyl.

In some embodiments R^{c} is C3-C11 branched alkyl.

In some embodiments R^{c} is C3-C8 cycloalkyl.

In some embodiments R^{c} is C5-C15 bicycloalkyls.

In some embodiments R^{c} is CH₂F.

In some embodiments R^{c} is CHF₂.

In some embodiments R^{c} is CF₃.

In some embodiments R^{c} is C2-C9 haloalkyls.

In some embodiments R^{c} is C3-C9 branched haloalkyl.

In some embodiments R^{c} is optionally substituted benzyl.

In some embodiments R^{c} is optionally substituted CH₂heteroaryl.

In some embodiments R^{c} is optionally substituted aryl.

In some embodiments R^{c} is optionally substituted heteroaryl.

In some embodiments R^{c} is a 3 membered cycloalkyl ring.

In some embodiments R^{c} is a 4 membered cycloalkyl ring.

In some embodiments R^{c} is a 5 membered cycloalkyl ring.

In some embodiments R^{c} is a 6 membered cycloalkyl ring.

In some embodiments R^{a} is a 3 membered cycloalkyl ring containing an oxygen.

In some embodiments R^{c} is a 4 membered cycloalkyl ring containing an oxygen.

In some embodiments R^{c} is a 5 membered cycloalkyl ring containing an oxygen.

In some embodiments R^{c} is a 6 membered cycloalkyl ring containing an oxygen.

In some embodiments R^{a} and R^{b} are taken together to form a 3 membered cycloalkyl ring.

In some embodiments R^{a} and R^{b} are taken together to form a 4 membered cycloalkyl ring.

In some embodiments R^{a} and R^{b} are taken together to form a 5 membered cycloalkyl ring.

In some embodiments R^{a} and R^{b} are taken together to form a 6 membered cycloalkyl ring.

In some embodiments R^{a} and R^{b} are taken together to form a 3 membered cycloalkyl ring containing an oxygen.

In some embodiments R^{a} and R^{b} are taken together to form a 4 membered cycloalkyl ring containing an oxygen.

In some embodiments R^{a} and R^{b} are taken together to form a 5 membered cycloalkyl ring containing an oxygen.

In some embodiments R^{a} and R^{b} are taken together to form a 6 membered cycloalkyl ring containing an oxygen.

In some embodiments R^{d} is hydrogen.

In some embodiments R^{d} is C1-C6 alkyl.

In some embodiments R^{d} is C3-C6 cycloalyl.

In some embodiments R^{d} is optionally aromatic ring.

In some embodiments R^{d} is optionally substituted heteroaromatic ring.

In some embodiments R^{c} is hydrogen.

In some embodiments R^{e} is C1-C6 alkyl.

In some embodiments R^{c} is C3-C6 cycloalyl.

In some embodiments R^{c} is optionally aromatic ring.

In some embodiments R^{c} is optionally substituted heteroaromatic ring.

In some embodiments R^{a} is hydrogen.

In some embodiments R^{a} is C1-C12 alkyl.

In some embodiments R^{a} is C3-C11 branched alkyl.

In some embodiments R^{e} is C3-C8 cycloalkyl.

In some embodiments R^{f} is C5-C15 bicycloalkyls.

In some embodiments R^{a} is CH₂F.

In some embodiments R^{a} is CHF₂.

In some embodiments R^{a} is CF₃.

In some embodiments R^{a} is C2-C9 haloalkyls.

In some embodiments R^{e} is C3-C9 branched haloalkyl.

In some embodiments R^{f} is optionally substituted benzyl,

In some embodiments R^{f} is optionally substituted CH₂heteroaryl.

In some embodiments R^{f} is optionally substituted aryl.

In some embodiments R^{f} is optionally substituted heteroaryl.

In some embodiments R^{f} is C3 cycloalkyl ring.

In some embodiments R^{a} is C4 cycloalkyl ring.

In some embodiments R^{a} is C5 cycloalkyl ring.

In some embodiments R^{a} is C6 cycloalkyl ring.

In some embodiments R^{a} is C3 cycloalkyl ring containing an oxygen.

In some embodiments R^{a} is C4 cycloalkyl ring containing an oxygen.

In some embodiments R^{a} is C5 cycloalkyl ring containing an oxygen.

In some embodiments R^{a} is C6 cycloalkyl ring containing an oxygen.

In some embodiments R⁵ is C2-C8 alkyl.

In some embodiments R⁵ is C3-C11 branched alkyl.

In some embodiments R⁵ is C3-C8 cycloalkyl.

In some embodiments R⁵ is C5-C15 bicycloalkyls.

In some embodiments R⁵ is CH₂F/

In some embodiments R⁵ is CHF₂.

In some embodiments R⁵ is CF₃.

In some embodiments R⁵ is C2-C9 haloalkyls.

In some embodiments R⁵ is C3-C9 branched haloalkyl.

In some embodiments R⁵ is optionally substituted benzyl.

In some embodiments R⁵ is optionally substituted CH₂heteroaryl.

In some embodiments R⁵ is CH₂OR^{5a}

In some embodiments R⁵ is CH(CH₃)OR^{5a}

In some embodiments R⁵ is C(R^{5e})₂OR^{5a}.

In some embodiments R⁵ is C(R^{5e})₂OR^{5a}

In some embodiments R⁵ is CH₂SR^{5a}

In some embodiments R⁵ is CH₂CH₂SCH₃.

In some embodiments R⁵ is CH₂CH₂SO₂CH₃.

In some embodiments R⁵ is CH₂CH₂CH₂NR^{5c} R^{5d}.

In some embodiments R⁵ is CH₂COR^{5b}.

In some embodiments R⁵ is CH₂CH₂COR^{5b}.

In some embodiments R⁵ is (CH₂)_{z}(C3-C7cycloakyl).

In some embodiments z is 0.

In some embodiments z is 1.

In some embodiments z is 2.

In some embodiments z is 3.

In some embodiments z is 4.

In some embodiments z is 5.

In some embodiments z is 6.

In some embodiments R⁵ C₃-C₆ cycloalkyl ring optionally containing an oxygen

In some embodiments R^{5a} is hydrogen.

In some embodiments R^{5a} is C1-C6 alkyl.

In some embodiments R^{5a} is C3-C7 branched alkyl,

In some embodiments R^{5a} is optionally substituted phenyl.

In some embodiments R^{5a} is optionally substituted benzyl.

In some embodiments R^{5a} is optionally substituted CH₂CH₂Ar.

In some embodiments R^{1b} is OH.

In some embodiments R^{1b} is C1-C6 alkoxy.

In some embodiments R^{1b} is NH₂.

In some embodiments R^{1c} is hydrogen.

In some embodiments R^{1c} is optionally substituted C1-C6 alkyl.

In some embodiments Rid is hydrogen.

In some embodiments R^{5d} is optionally substituted C1-C6 alkyl.

In some embodiments R^{5e} is C1-C4 alkyl.

In some embodiments R⁷ is hydrogen.

In some embodiments R⁷ is hydrogen.

In some embodiments R⁶ and R⁷are taken together with the atoms to which they are bound to form a 6 membered aromatic ring that is substituted with at least one moiety that is selected from the group consisting of halogen, C1-C6 haloalkyl, cyano, C1-C6 alkyl, C1-C6 alkoxy,

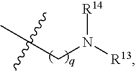

C3-C8 cycloalkyl, C3-C8 heterocycle, aryl, and heteroaryl.

In some embodiments R⁶ and R⁷ are taken together with the atoms to which they are bound to form a substituted heteroaromatic ring containing 5 members containing one nitrogen that is substituted with at least one moiety that is selected from the group consisting of halogen, C1-C6 haloalkyl, cyano, C1-C6 alkoxy,

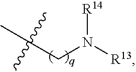

C3-C8 cycloalkyl, C3-C8 heterocycle, aryl, and heteroaryl.

In some embodiments R⁶ and R⁷ are taken together with the atoms to which they are bound to form a substituted heteroaromatic ring containing 5 members containing two nitrogens that is substituted with at least one moiety that is selected from the group consisting of halogen, C1-C6 haloalkyl, cyano, C1-C6 alkoxy,

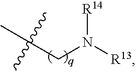

C3-C8 cycloalkyl, C3-C8 heterocycle, aryl, and heteroaryl.

In some embodiments R⁶ and R⁷ are taken together with the atoms to which they are bound to form a substituted heteroaromatic ring containing 6 members containing 1 nitrogen that is substituted with at least one moiety that is selected from the group consisting of halogen, C1-C6 haloalkyl, cyano, C1-C6 alkoxy,

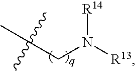

C3-C8 cycloalkyl, C3-C8 heterocycle, aryl, and heteroaryl.

In some embodiments R⁶ and R⁷ are taken together with the atoms to which they are bound to form a substituted heteroaromatic ring containing 6 members containing 2 nitrogens that is substituted with at least one moiety that is selected from the group consisting of halogen, C1-C6 haloalkyl, cyano, C1-C6 alkoxy,

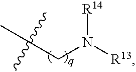

C3-C8 cycloalkyl, C3-C8 heterocycle, aryl, and heteroaryl.

In some embodiments R^{8a} is hydrogen.

In some embodiments R^{8a} is halogen.

In some embodiments R^{8a} is C1-C6 haloalkyl.

In some embodiments R^{8a} is cyano.

In some embodiments R^{8a} is C1-C6 alkyl.

In some embodiments R^{8a} is C1-C6 alkoxy.

In some embodiments R^{8a} is

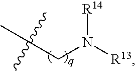

In some embodiments R^{8a} is C3-C8 cycloalkyl.

In some embodiments R^{8a} is C3-C8 heterocycle, aryl.

In some embodiments R^{8a} is heteroaryl.

In some embodiments R^{8b} is hydrogen.

In some embodiments R^{8b} is halogen.

In some embodiments R^{8b} is C1-C6 haloalky.

In some embodiments R^{8b} is, cyano.

In some embodiments R^{8b} is C1-C6 alkyl.

In some embodiments R^{8b} is C1-C6 alkoxy.

In some embodiments R^{8b} is

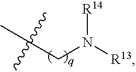

In some embodiments R^{8b} is C3-C8 cycloalkyl.

In some embodiments R^{8b} is C3-C8 heterocycle.

In some embodiments R^{8b} is aryls.

In some embodiments R^{8b} is heteroaryl.

In some embodiments R^{8c} is halogen.

In some embodiments R^{8c} is C1-C6 haloalkyl.

In some embodiments R^{8c} is cyano.

In some embodiments R^{8c} is C1-C6 alkyl.

In some embodiments R^{8c} is C1-C6 alkoxy.

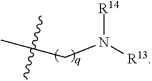

In some embodiments R^{8c} is q

In some embodiments R^{8c} is C3-C8 cycloalkyl.

In some embodiments R^{8c} is C3-C8 heterocycle

In some embodiments R^{8c} is aryl.

In some embodiments R^{8c} is heteroaryl.

In some embodiments R^{8d} is hydrogen.

In some embodiments R^{8d} is halogen.

In some embodiments R^{8d} is C1-C6 haloalkyl.

In some embodiments R^{8d} is cyano.

In some embodiments R^{8d} is C1-C6 alkyl.

In some embodiments R^{8d} is C1-C6 alkoxy.

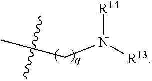

In some embodiments R^{8d} is q

In some embodiments R^{8d} is C3-C8 cycloalkyl.

In some embodiments R^{8d} is C3-C8 heterocycle.

In some embodiments R^{5d} is aryl.

In some embodiments R^{8d} is heteroaryl.

In some embodiments R^{9a} is hydrogen.

In some embodiments R^{9a} is halogen.

In some embodiments R^{9a} is C1-C6 haloalkyl.

In some embodiments R^{9a} is cyano.

In some embodiments R^{9a} is C1-C6 alkyl.

In some embodiments R^{9a} is C1-C6 alkoxy.

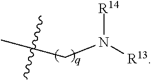

In some embodiments R^{9a} is q

In some embodiments R^{9a} is C3-C8 cycloalkyl.

In some embodiments R^{9a} is C3-C8 heterocycle.

In some embodiments R^{9a} is aryl.

In some embodiments R^{9a} is heteroaryl.

In some embodiments R^{9b} is hydrogen.

In some embodiments R^{9b} is halogen.

In some embodiments R^{9b} is C1-C6 haloalkyl.

In some embodiments R^{9b} is cyano.

In some embodiments R^{9b} is C1-C6 alkyl.

In some embodiments R^{9b} is C1-C6 alkoxy.

In some embodiments R^{9b} is

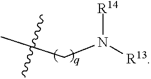

In some embodiments R^{9b} is C3-C8 cycloalkyl.

In some embodiments R^{9b} is C3-C8 heterocycle.

In some embodiments R^{9b} is aryl.

In some embodiments R^{9b} is heteroaryl.

In some embodiments R^{9b} is hydrogen.

In some embodiments R^{9b} is halogen.

In some embodiments R^{9b} is C1-C6 haloalkyl.

In some embodiments R^{9b} is cyano.

In some embodiments R^{9b} is C1-C6 alkyl.

In some embodiments R^{9b} is C1-C6 alkoxy.

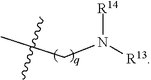

In some embodiments R^{9b} is q

In some embodiments R^{9b} is C3-C8 cycloalkyl.

In some embodiments R^{9b} is C3-C8 heterocycle.

In some embodiments R^{9b} is aryl.

In some embodiments R^{9b} is heteroaryl.

In some embodiments R^{9c} is halogen.

In some embodiments R^{9c} is C1-C6 haloalkyl.

In some embodiments R^{9c} is cyano.

In some embodiments R^{9c} is C1-C6 alkyl.

In some embodiments R^{9c} is C1-C6 alkoxy.

In some embodiments R^{9c} is

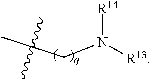

In some embodiments R^{9c} is C3-C8 cycloalkyl.

In some embodiments R^{9c} is C3-C8 heterocycle

In some embodiments R^{9c} is aryl.

In some embodiments R^{9c} is heteroaryl.

In some embodiments R^{9d} is hydrogen.

In some embodiments R^{9d} is halogen.

In some embodiments R^{9d} is C1-C6 haloalkyl.

In some embodiments R^{9d} is cyano.

In some embodiments R^{9d} is C1-C6 alkyl.

In some embodiments R^{9d} is C1-C6 alkoxy.

In some embodiments R^{9d} is

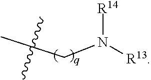

In some embodiments R^{9d} is C3-C8 cycloalkyl.

In some embodiments R^{9d} is C3-C8 heterocycle.

In some embodiments R^{9d} is aryl.

In some embodiments R^{9d} is heteroaryl.

In some embodiments R^{10a} is hydrogen.

In some embodiments R^{10a} is halogen.

In some embodiments R^{10a} is C1-C6 haloalkyl.

In some embodiments R^{10a} is cyano.

In some embodiments R^{10a} is C1-C6 alkyl.

In some embodiments R^{10a} is C1-C6 alkoxy.

In some embodiments R^{10a} is

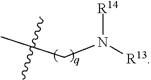

In some embodiments R^{10a} is C3-C8 cycloalkyl.

In some embodiments R^{10a} is C3-C8 heterocycle.

In some embodiments R^{10a} is aryl.

In some embodiments R^{10a} is heteroaryl.

In some embodiments R^{10b} is hydrogen.

In some embodiments R^{10b} is halogen.

In some embodiments R^{10c} is C1-C6 haloalkyl.

In some embodiments R^{10c} is cyano.

In some embodiments R^{10b} is C1-C6 alkyl.

In some embodiments R^{10b} is C1-C6 alkoxy.

In some embodiments R^{10b} is

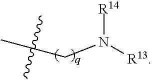

In some embodiments R^{10b} is C3-C8 cycloalkyl.

In some embodiments R^{10b} is C3-C8 heterocycle.

In some embodiments R^{10b} is aryl.

In some embodiments R^{10b} is heteroaryl.

In some embodiments R^{10c} is halogen.

In some embodiments R^{10c} is C1-C6 haloalkyl.

In some embodiments R^{10c} is cyano.

In some embodiments R^{10c} is C1-C6 alkyl.

In some embodiments R^{10c} is C1-C6 alkoxy.

In some embodiments R^{10c} is,

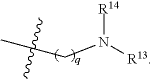

In some embodiments R^{10c} is C3-C8 cycloalkyl.

In some embodiments R^{10c} is C3-C8 heterocycle.

In some embodiments R^{10c} is aryl.

In some embodiments R^{10c} is heteroaryl.

In some embodiments R^{10d} is hydrogen.

In some embodiments R^{10d} is halogen.

In some embodiments R^{10d} is C1-C6 haloalkyl.

In some embodiments R^{10d} is cyano.

In some embodiments R^{10d} is C1-C6 alkyl.

In some embodiments R^{10d} is C1-C6 alkoxy.

In some embodiments R^{10d} is

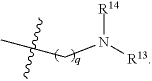

In some embodiments R^{10d} is C3-C8 cycloalkyl.

In some embodiments R^{10d} is C3-C8 heterocycle.

In some embodiments R^{10d} is aryl.

In some embodiments R^{10d} is heteroaryl.

In some embodiments R^{11a} is hydrogen.

In some embodiments R^{11a} is halogen.

In some embodiments R^{11a} is C1-C6 haloalkyl.

In some embodiments R^{11a} is cyano.

In some embodiments R^{11a} is C1-C6 alkyl.

In some embodiments R^{11a} is C1-C6 alkoxy.

In some embodiments R^{11a} is

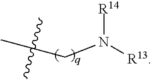

In some embodiments R^{11a} is C3-C8 cycloalkyl.

In some embodiments R^{11a} is C3-C8 heterocycle.

In some embodiments R^{11a} is aryl.

In some embodiments R^{11a} is heteroaromatic ring.

In some embodiments R^{11a} is hydrogen.

In some embodiments R^{1b} is halogens.

In some embodiments R^{1b} is C1-C6 haloalkyl.

In some embodiments R^{11b} is cyano.

In some embodiments R^{11b} is C1-C6 alkyl.

In some embodiments R^{11b} is C1-C6 alkoxy

In some embodiments R^{11b} is

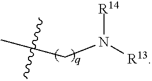

In some embodiments R^{11b} is C3-C8 cycloalkyl.

In some embodiments R^{11b} is C3-C8 heterocycle.

In some embodiments R^{11b} is aryl.

In some embodiments R^{11b} is heteroaromatic ring.

In some embodiments R^{11c} is halogen.

In some embodiments R^{11c} is C1-C6 haloalkyl.

In some embodiments R^{11c} is cyano.

In some embodiments R^{11c} is C1-C6 alkyl.

In some embodiments R^{11c} is C1-C6 alkoxy.

In some embodiments R^{11c} is

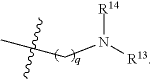

In some embodiments R^{11c} is C3-C8 cycloalkyl.

In some embodiments R^{11c} is C3-C8 heterocycle.

In some embodiments R^{11c} is aryl.

In some embodiments R^{11c} is heteroaromatic ring.

In some embodiments R^{11d} is hydrogen.

In some embodiments R^{11d} is halogen

In some embodiments R^{11d} is C1-C6 haloalkyl.

In some embodiments R^{11d} is cyano.

In some embodiments R^{11d} is C1-C6 alkyl.

In some embodiments R^{11d} is C1-C6 alkoxy.

In some embodiments R^{11d} is

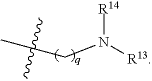

In some embodiments R^{11d} is C3-C8 cycloalkyl.

In some embodiments R^{11d} is C3-C8 heterocycle.

In some embodiments R^{11d} is aryl.

In some embodiments R^{8d} is heteroaromatic ring.

In some embodiments R^{12a} is hydrogen.

In some embodiments R^{12a} is halogen.

In some embodiments R^{12a} is C1-C6 haloalkyl.

In some embodiments R^{12a} is cyano.

In some embodiments R^{12a} is C1-C6 alkyl.

In some embodiments R^{12a} is C1-C6 alkoxy.

In some embodiments R^{12a} is

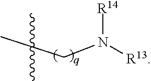

In some embodiments R^{12a} is C3-C8 cycloalkyl.

In some embodiments R^{12a} is C3-C8 heterocycle.

In some embodiments R^{12a} is aryl.

In some embodiments R^{12a} is heteroaryl.

In some embodiments R^{12a} is hydrogen.

In some embodiments R^{12a} is halogen.

In some embodiments R^{12b} is C1-C6 haloalkyl.

In some embodiments R^{12b} is cyano.

In some embodiments R^{12b} is C1-C6 alkyl.

In some embodiments R^{12b} is C1-C6 alkoxy.

In some embodiments R^{12b} is

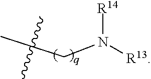

In some embodiments R^{12b} is C3-C8 cycloalkyl.

In some embodiments R^{12d} is C3-C8 heterocycle.

In some embodiments R^{12d} is aryl.

In some embodiments R^{12d} is heteroaryl.

In some embodiments R^{12c} is halogen.

In some embodiments R^{12c} is C1-C6 haloalkyl.

In some embodiments R^{12c} is cyano.

In some embodiments R^{12c} is C1-C6 alkyl.

In some embodiments R^{12c} is C1-C6 alkoxy.

In some embodiments R^{12c} is

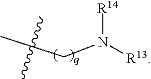

In some embodiments R^{12c} is C3-C8 cycloalkyl.

In some embodiments R^{12c} is C3-C8 heterocycle.

In some embodiments R^{12c} is aryl.

In some embodiments R^{12c} is heteroaryl.

In some embodiments R^{12d} is hydrogen.

In some embodiments R^{12d} is halogen.

In some embodiments R^{12d} is C1-C6 haloalkyl.

In some embodiments R^{12d} is cyano.

In some embodiments R^{12d} is C1-C6 alkyl.

In some embodiments R^{12d} is C1-C6 alkoxy.

In some embodiments R^{12d} is

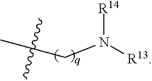

In some embodiments R^{12d} is C3-C8 cycloalkyl.

In some embodiments R^{12d} is C3-C8 heterocycle.

In some embodiments R^{12d} is aryl.

In some embodiments R^{12d} is heteroaryl.

In some embodiments R¹³ is hydrogen.

In some embodiments R¹³ is C1-C6 alkyl.

In some embodiments R¹⁴ is hydrogen.

In some embodiments R¹⁴ is C1-C6 alkyl.

In some embodiments R¹⁵ is C1-C6 alkyl.

In some embodiments R¹⁵ is C3-C6 cycloalkyl.

In some embodiments R¹⁵ is C1-C6 haloalkyl.

In some embodiments R¹⁶ is C1-C6 alkyl.

In some embodiments R¹⁶ is C3-C6 cycloalkyl.

In some embodiments R¹⁶ is C1-C6 haloalkyl.

In some embodiments R¹⁷ is C1-C6 alkyl.

In some embodiments R¹⁷ is C3-C6 cycloalkyl.

In some embodiments R¹⁷ is C1-C6 haloalkyl.

In some embodiments R¹⁸ is C1-C6 alkyl.

In some embodiments R¹⁸ is C3-C6 cycloalkyl.

In some embodiments R¹⁸ is C1-C6 haloalkyl.

In some embodiments R¹⁹ is C1-C6 alkyl.

In some embodiments R¹⁹ is C3-C6 cycloalkyl.

In some embodiments R¹⁹ is C1-C6 haloalkyl.

In some embodiments q is 1.

In some embodiments q is 2.

In some embodiments q is 3.

In some embodiments q is 4.

In some embodiments q is 5.

In some embodiments q is 6.

For the purposes of demonstrating the manner in which the compounds of the present invention are named and referred to herein, the compound having the formula:

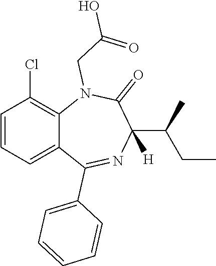 [SMILES: CC[C@H](C)[C@@H]1N=C(c2ccccc2)c2cccc(Cl)c2N(CC(=O)O)C1=O]

has the chemical name 2-((S)-3-((S)-sec-butyl)-9-chloro-2-oxo-5-phenyl-2,3-dihydro-1H-benzo[e][1,4]diazepin-1-yl)acetic acid.

For the purposes of demonstrating the manner in which the compounds of the present invention are named and referred to herein, the compound having the formula:

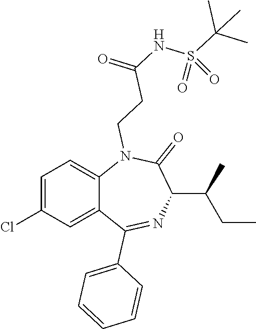 [SMILES: CC[C@H](C)[C@@H]1N=C(c2ccccc2)c2cc(Cl)ccc2N(CCC(=O)NS(=O)(=O)C(C)(C)C)C1=O]

has the chemical name 3-((S)-3-((S)-sec-butyl)-7-chloro-2-oxo-5-phenyl-2,3-dihydro-1H-benzo[e][1,4]diazepin-1-yl)-N-(N,N-dimethylsulfamoyl)propanamide.

For the purposes of the present invention, a compound depicted by the racemic formula, for example:

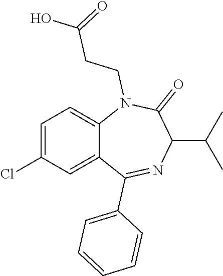 [SMILES: CC(C)C1N=C(c2ccccc2)c2cc(Cl)ccc2N(CCC(=O)O)C1=O]

will stand equally well for either of the two enantiomers having the formula:

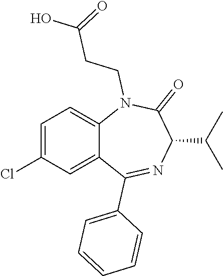 [SMILES: CC(C)[C@@H]1N=C(c2ccccc2)c2cc(Cl)ccc2N(CCC(=O)O)C1=O]

or the formula:

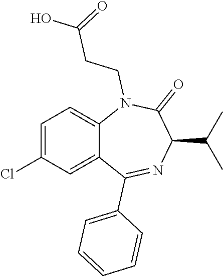 [SMILES: CC(C)[C@H]1N=C(c2ccccc2)c2cc(Cl)ccc2N(CCC(=O)O)C1=O]

or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### Process

The present invention further relates to a process for preparing the Biological effect agents of the present invention.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes.

### General Synthetic Schemes for Preparation of Compounds.

The reagents used in the preparation of the compounds of this invention can be either commercially obtained or can be prepared by standard procedures described in the literature. In accordance with this invention, compounds in the genus may be produced by one of the following reaction schemes.

Compounds of formula (I) may be prepared according to the process outlined in schemes 1-36.

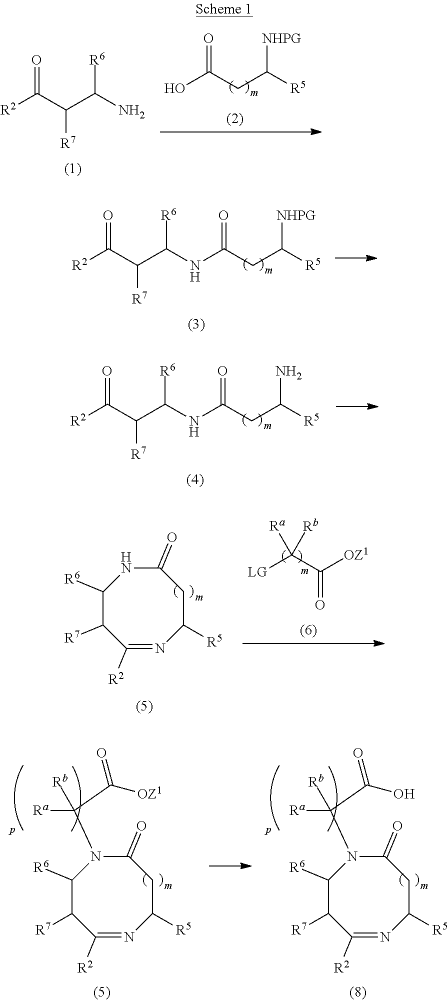

Accordingly, a suitably substituted compound of the formula (1), a known compound or compound prepared by known methods, is reacted with a compound of the formula (2) wherein PG is a protecting group such as tert-butyloxycarbonyl, benzylcarbonyl, fluorenylmethoxycarbonyl, and the like, in the presence of coupling agent such as dicyclohexylcarbodiimide, 1-ethyl-3-(3-dimethylaminopropyl)carbodiimide, and the like in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (3). A compound of the formula (3) is reacted with an acid such trifluoroacetic acid, acetic acid, formic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally with heating, optionally with microwave irradiation to provide a compound of the formula (4). Alternatively, a compound of the formula (3) is reacted with a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (4). Alternatively, a compound of the formula (3) is reacted with hydrogen in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (4). A compound of the formula (4) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (5). A compound of the formula (5) is reacted with a compound of the formula (6) wherein LG is a leaving group such as chlorine, bromine, iodine, tosylate, mesylate, and the like and Z¹ is a C₁-C₆ alkyl, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (7). Alternatively, a compound of the formula (5) is reacted with a compound of the formula (6) wherein LG is a leaving group such as chlorine, bromine, iodine, tosylate, mesylate, and the like and Z¹ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (7). A compound of the formula (7) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (8).

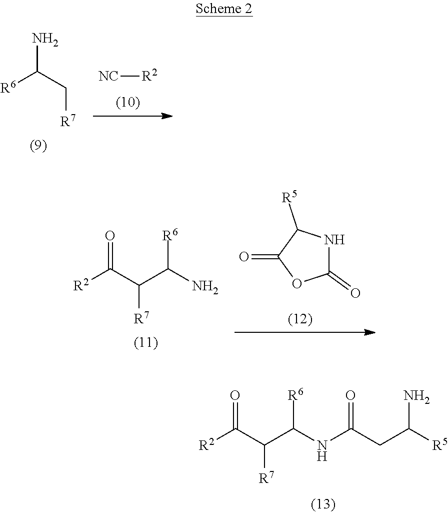

A compound of the formula (9), a known compound or compound prepared by known methods, is reacted with a compound of the formula (10), a known compound or a compound prepared by known methods, in the presence of boron trichloride, in the presence of aluminum trichloride, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (11). A compound of the formula (11) is reacted with a compound of the formula (12) in the presence of an acid such as trifluoroacetic acid, acetic acid, formic acid, hydrochloric acid, sulfuric acid, and the like, in a solvent such as toluene, benzene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (13).

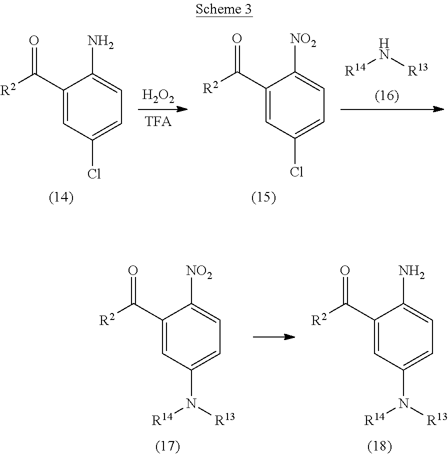

A compound of the formula (14), a known compound or compound prepared by known methods, is reacted with aqueous hydrogen peroxide optionally in the presence of a solvent such as methanol, ethanol, isopropanol, 1,4-dioxane, acetonitrile, 1,2-dimethoxyethane, and the like, optionally with heating, optionally with microwave irradiation to provide a compound of the formula (14). A compound of the formula (15) is reacted with a compound of the formula (16), a known compound or a compound prepared by known methods, in the presence of a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (17). A compound of the formula (17) is reacted with hydrogen in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (18).

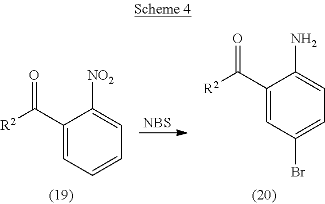

A compound of the formula (19), a own compound or compound prepared by known methods, is reacted with N-bromosuccinimide in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (20).

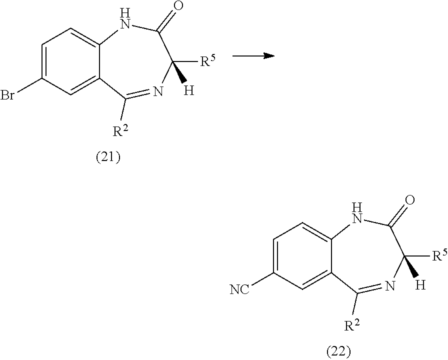

A compound of the formula (21), is reacted with a cyanide salt such as copper cyanide, sodium cyanide, potassium cyanide, lithium cyanide, and the like, in the presence of a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally with heating, optionally with microwave irradiation to provide a compound of the formula (22).

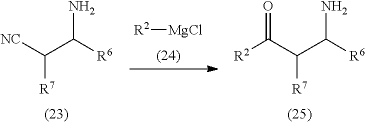

A compound of the formula (23), a known compound or compound prepared by known methods, is reacted with a compound of the formula (24), a known compound or a compound prepared by known methods, in the presence of a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally with heating, optionally with microwave irradiation to provide a compound of the formula (25).

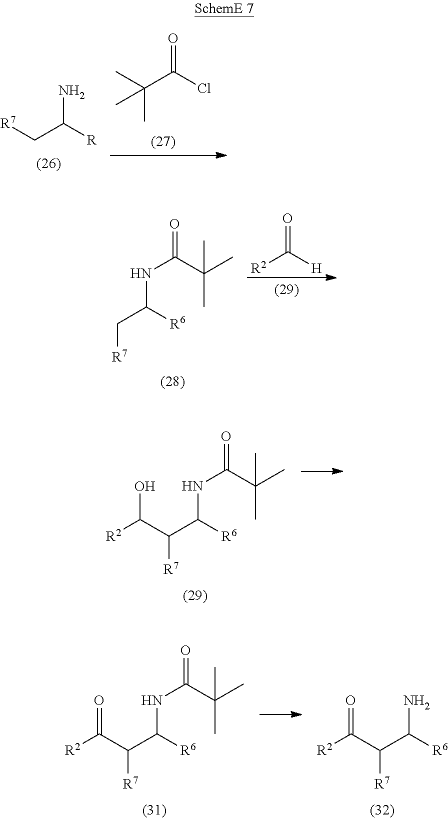

A compound of the formula (26), a known compound or compound prepared by known methods, is reacted with a compound of the formula (27) in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (27). A compound of the formula (28) is reacted with a compound of the formula (29), a known compound or a compound prepared by known methods, in the presence of a base such as n-butyl lithium, sec-butyl lithium, tert-butyl lithium and the like, in the presence of a solvent such as methylene chloride, 1,2-dichloroethane, tetrahydrofuran, 1,4-dioxane, and the like, optionally with heating, optionally with microwave irradiation to provide a compound of the formula (30). A compound of the formula (30) is reacted with Dess Martin periodinane in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (31). A compound of the formula (31) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (32).

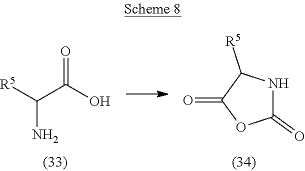

A compound of the formula (33), a known compound or compound prepared by known methods, is reacted with phosgene in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (34). Alternatively, a compound of the formula (33), a known compound or compound prepared by known methods, is reacted with triphosgene in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (34).

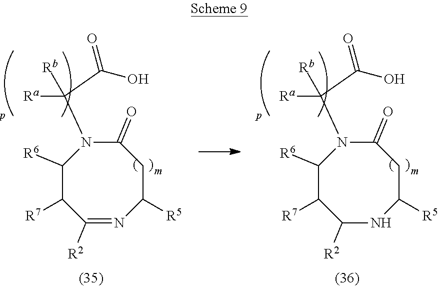

A compound of the formula (35) is reacted with a reducing agent such as sodium cyanoborohydride, lithium cyanoborohydride, sodium borohydride, lithium borohydride, Sodium triacetoxyborohydride, lithium triacetoxyborohydride, and the like, optionally in the presence of an acid such as trifluoroacetic acid, acetic acid, formic acid, hydrochloric acid, sulfuric acid, and the like, in a solvent such as methanol, ethanol, tetrahydrofuran, 1,4-dioxane, acetonitrile, 1,2-dimethoxyethane, and the like, optionally with heating, optionally with microwave irradiation to provide a compound of the formula (36).

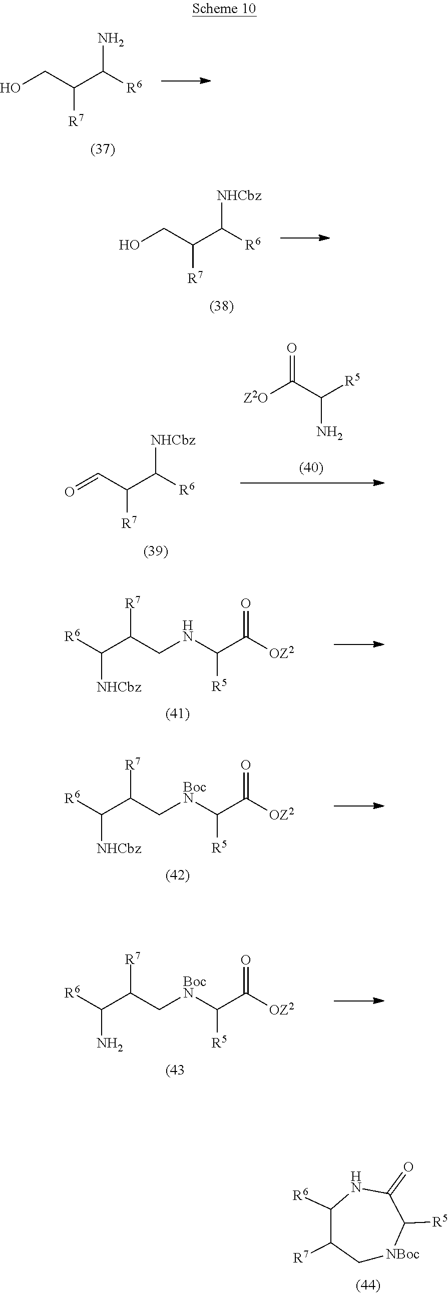

A compound of the formula (37), a known compound or compound prepared by known methods, is reacted benzyl chloroformate, optionally in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (38). A compound of the formula (38) is reacted with Dess Martin periodinane in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (39). A compound of the formula (39) is reacted with a of the formula (40), a known compound or a compound prepared by known methods wherein Z² is C1-C6 alkyl, in the presence of a reducing agent such as sodium cyanoborohydride, lithium cyanoborohydride, sodium borohydride, lithium borohydride, sodium triacetoxyborohydride, lithium triacetoxyborohydride, and the like, optionally in the presence of an acid such as trifluoroacetic acid, acetic acid, formic acid, hydrochloric acid, sulfuric acid, and the like, in a solvent such as methanol, ethanol, tetrahydrofuran, 1,4-dioxane, acetonitrile, 1,2-dimethoxyethane, and the like, optionally with heating, optionally with microwave irradiation to provide a compound of the formula (41). A compound of the formula (41) is reacted with di-tert-butyl dicarbonate in the presence of a base such as triethylamine, N,N-diisopropylethylamine, pyridine, 2,6-dimethylpyridine, and the like, in the presence of a solvent such as N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (42). A compound of the formula (42) is reacted with hydrogen in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (43). A compound of the formula (43) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (44).

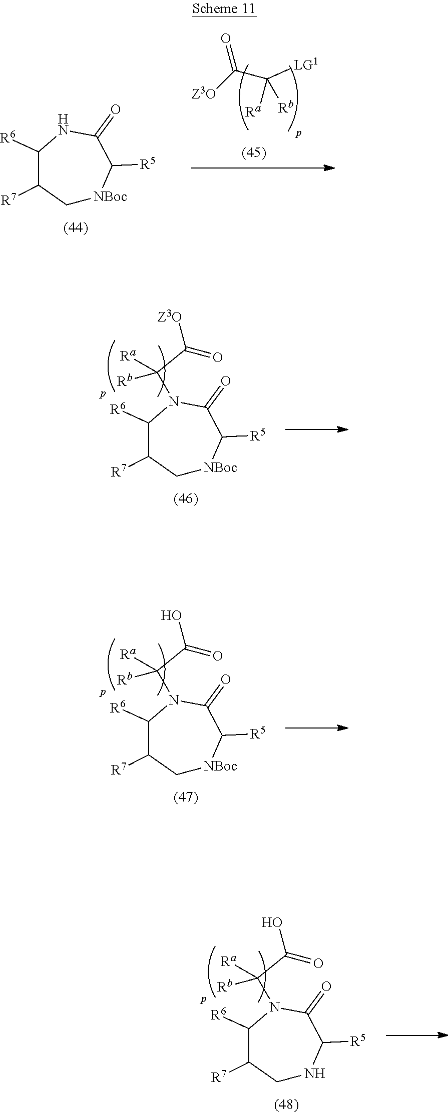

A compound of the formula (44) is reacted with compound of the formula (45), a known compound or compound prepared by known methods in which Z³ is C1-C6 alkyl and LG¹ is a leaving group such as chlorine, bromine, iodine, tosylate, mesylate, and the like, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (46). A compound of the formula (46) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (47). A compound of the formula (47) is reacted with an acid such as trifluoroacetic acid, acetic acid, formic acid, hydrochloric acid, sulfuric acid, and the like, in a solvent such as methanol, ethanol, tetrahydrofuran, 1,4-dioxane, acetonitrile, 1,2-dimethoxyethane, and the like, optionally with heating, optionally with microwave irradiation to provide a compound of the formula (48).

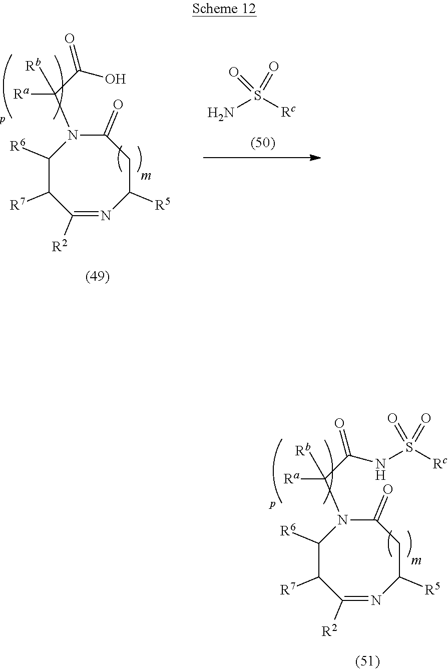

A compound of the formula (49) is reacted with compound of the formula (50), a known compound or compound prepared by known methods in the presence of coupling agent such as dicyclohexylcarbodiimide, 1-ethyl-3-(3-dimethylaminopropyl)carbodiimide, and the like in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (51).

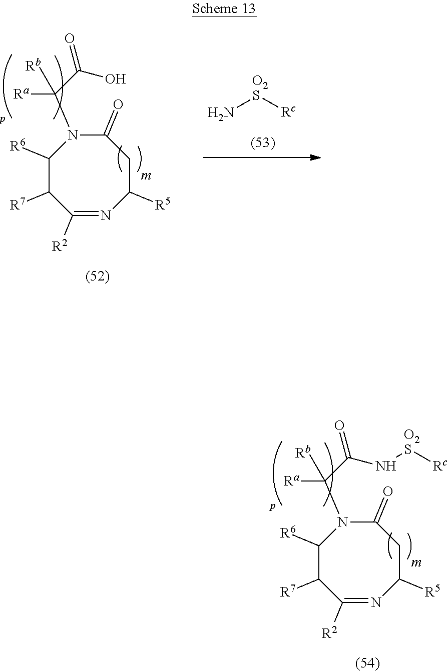

A compound of the formula (52) is reacted with compound of the formula (53), a known compound or compound prepared by known methods in the presence of coupling agent such as dicyclohexylcarbodiimide, 1-ethyl-3-(3-dimethylaminopropyl)carbodiimide, optionally in the presence of hydroxybenzotriazole, optionally in the presence of a base such as triethylamine, N,N-diisopropylethylamine, pyridine, 2,6-dimethylpyridine, and the like, in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (54).

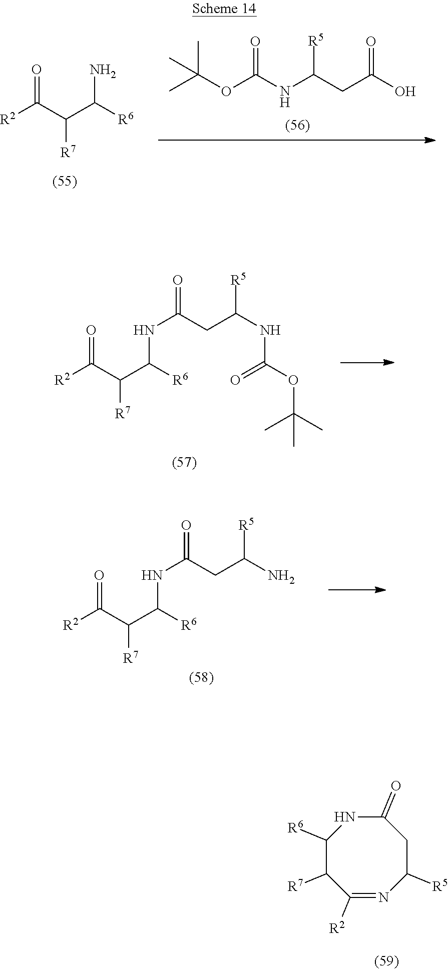

A compound of the formula (55), a known compound or a compound prepared by known methods, is reacted with a compound of the formula (56), a known compound or a compound prepared by known methods, in the presence of coupling agent such as dicyclohexylcarbodiimide, 1-ethyl-3-(3-dimethylaminopropyl)carbodiimide, and the like in the presence of a solvent such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (57). A compound of the formula (57) is then reacted with an acid such trifluoroacetic acid, acetic acid, formic acid, hydrochloric acid, sulfuric acid, and the like, in a solvent such as methylene chloride, 1,2-dichloroethane, tetrahydrofuran, 1,4-dioxane, methanol, ethanol, N,N-dimethylformamide, dimethylsulfoxide, N,N-dimethylacetamide, and the like, optionally with heating, optionally with microwave irradiation to provide a compound of the formula (58). A compound of the formula (58) reacted in a solvent such as methanol, ethanol, isopropanol, 2-butanol, tetrahydrofuran, 1,4-dioxane, N,N-dimethylformamide, dimethylsulfoxide, N,N-dimethylacetamide, and the like, optionally with heating, optionally with microwave irradiation to provide a compound of the formula (59).

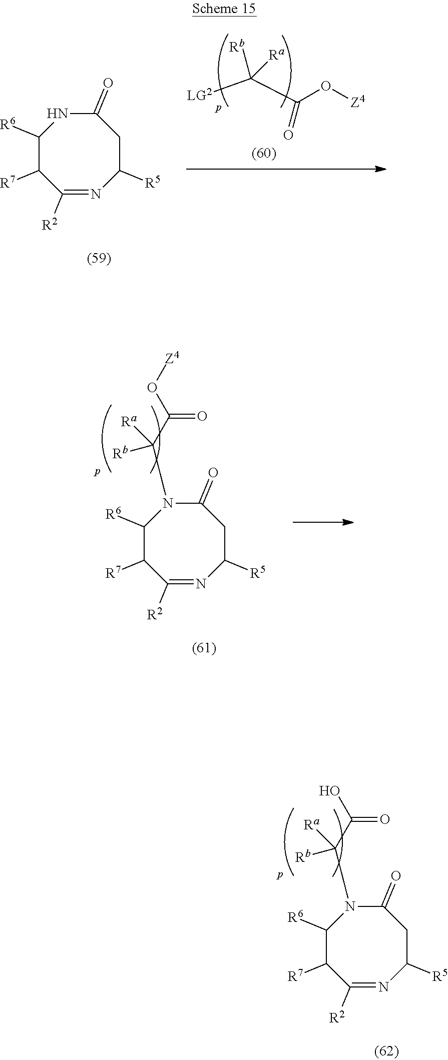

A compound of the formula (59) is reacted with a compound of the formula (60), a known compound or a compound prepared by known methods in which Z⁴ is C1-C6 alkyl and LG² is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylamnonium bromide, optionally with heating, optionally with microwave irradiation to provide a compound of the formula (61). Alternatively, a compound of the formula (59) is reacted with a compound of the formula (60) wherein LG² is a leaving group such as chlorine, bromine, iodine, tosylate, mesylate, and the like and Z⁴ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (61). A compound of the formula (61) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (62).

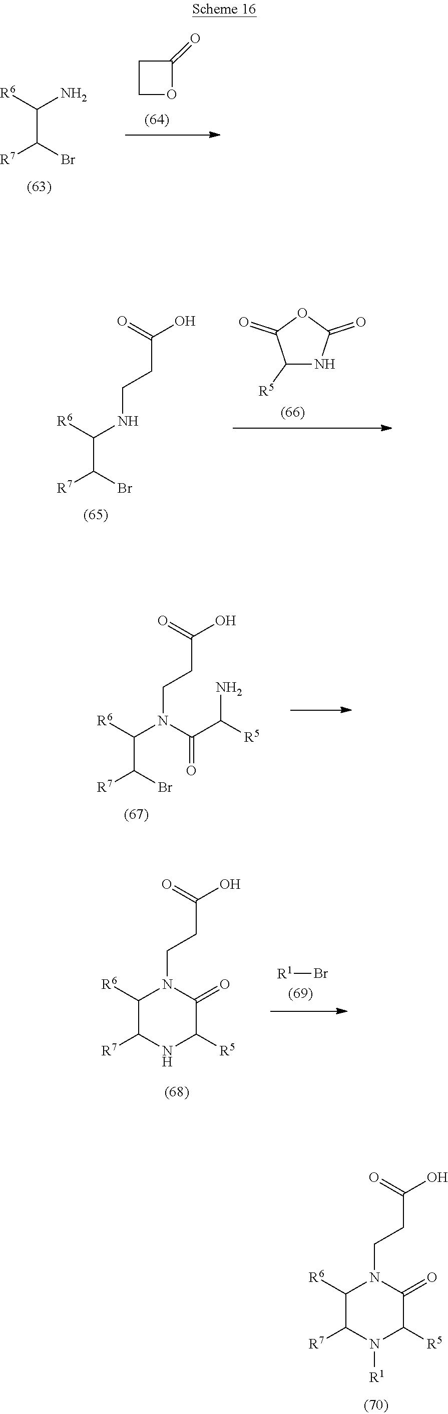

A compound of the formula (63), a known compound or a compound prepared by known methods, is reacted with a compound of the formula (64) in the presence of a solvent such as such as methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (65). A compound of the formula (65) is reacted with a compound of the formula (66) in the presence of an acid such as trifluoroacetic acid, acetic acid, formic acid, hydrochloric acid, sulfuric acid, and the like, in a solvent such as toluene, benzene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (67). Alternatively, a compound of the formula (65) is reacted with a compound of the formula (66) in the presence of a base such as sodium carbonate, lithium carbonate, potassium carbonate, cesium carbonate, sodium hydroxide, lithium hydroxide, potassium hydroxide, cesium hydroxide and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (67). A compound of the formula (67) is reacted with tri-tert-butylphosphine tetrafluoroborate in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a base such as sodium carbonate, lithium carbonate, potassium carbonate, cesium carbonate, sodium hydroxide, lithium hydroxide, potassium hydroxide, cesium hydroxide and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (68). A compound of the formula (68) is reacted with a compound of the formula (69), a known compound or a compound prepared by known methods, in the presence of a palladium catalyst palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (70).

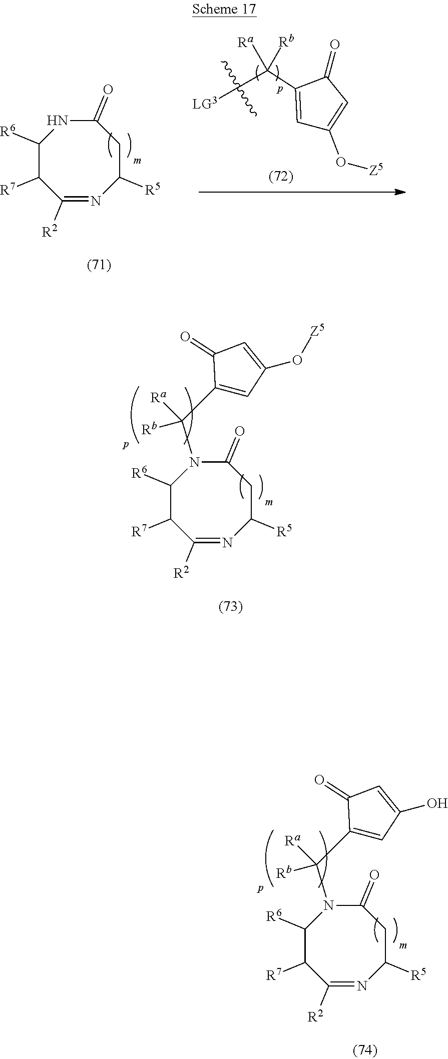

A compound of the formula (71) is reacted with a compound of the formula (72), a known compound or a compound prepared by known methods in which Z⁵ is C1-C6 alkyl and LG³ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (73). Alternatively, a compound of the formula (71) is reacted with a compound of the formula (72) wherein LG³ is a leaving group such as chlorine, bromine, iodine, tosylate, mesylate, and the like and Z⁵ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (73). A compound of the formula (73) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (74).

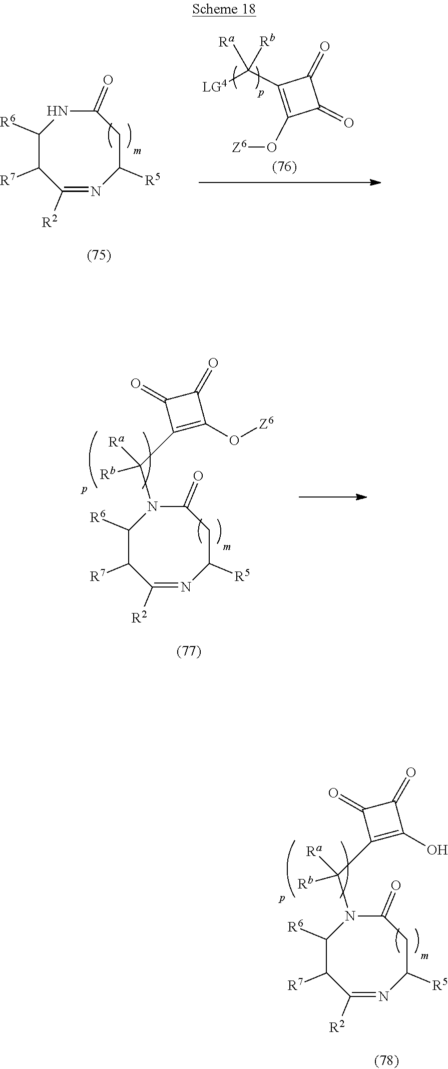

A compound of the formula (75) is reacted with a compound of the formula (76), a known compound or a compound prepared by known methods in which Z⁶ is C1-C6 alkyl and LG⁴ is a leaving group such as chlorine, bromine, iodine, tosylate, mesylate, and the like, optionally in the presence of a base such as triethylainine, N,N-diisopropylethylanine, pyridine, 2,6-dimethylpyridine, and the like, optionally in the presence of benzyltributylammonium bromide, optionally with heating, optionally with microwave irradiation to provide a compound of the formula (77). Alternatively, a compound of the formula (75) is reacted with a compound of the formula (76) wherein LG⁴ is a leaving group such as chlorine, bromine, iodine, tosylate, mesylate, and the like and Z⁶ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (77). A compound of the formula (77) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (78).

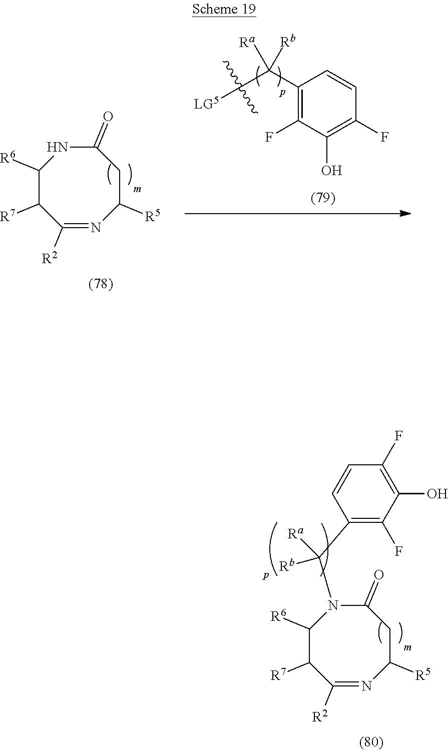

A compound of the formula (78) is reacted with a compound of the formula (79), a known compound or a compound prepared by known methods in which LG⁵ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (80). Alternatively, a compound of the formula (78) is reacted with a compound of the formula (79) wherein LG⁵ is a leaving group such as chlorine, bromine, iodine, tosylate, mesylate, and the like in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (80).

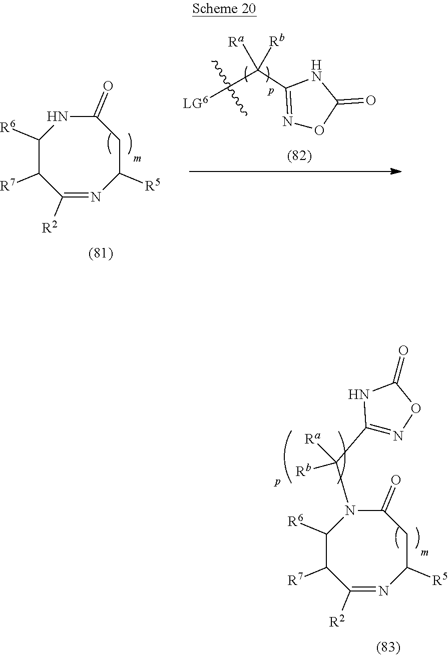

A compound of the formula (81) is reacted with a compound of the formula (82), a known compound or a compound prepared by known methods in which LG⁶ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (83). Alternatively, a compound of the formula (81) is reacted with a compound of the formula (82) wherein LG⁶ is a leaving group such as chlorine, bromine, iodine, tosylate, mesylate, and the like in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (83).

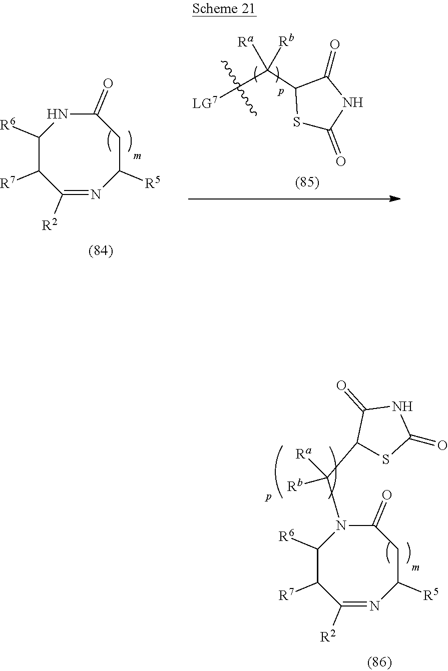

A compound of the formula (84) is reacted with a compound of the formula (85), a known compound or a compound prepared by known methods in which LG⁷ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (86). Alternatively, a compound of the formula (84) is reacted with a compound of the formula (85) wherein LG⁷ is a leaving group such as chlorine, bromine, iodine, tosylate, mesylate, and the like in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (86).

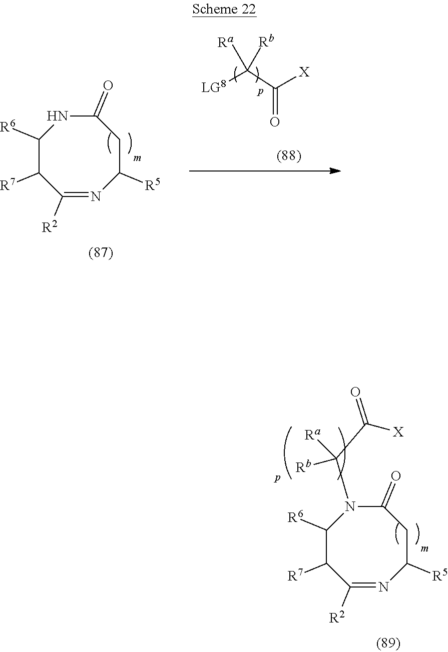

A compound of the formula (87) is reacted with a compound of the formula (88), a known compound or a compound prepared by known methods in which LG⁸ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (89). Alternatively, a compound of the formula (87) is reacted with a compound of the formula (88) wherein LG⁸ is a leaving group such as chlorine, bromine, iodine, tosylate, mesylate, and the like in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (89).

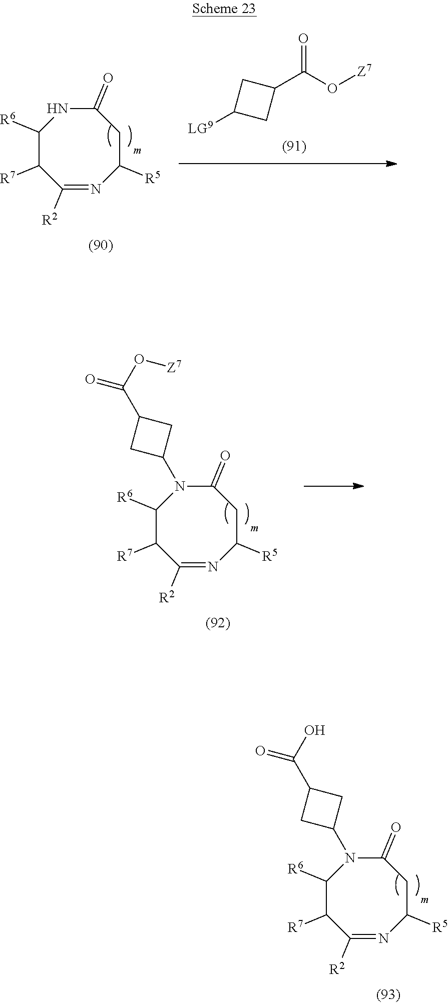

A compound of the formula (90) is reacted with a compound of the formula (91), a known compound or a compound prepared by known methods in which Z⁷ is C1-C6 alkyl and LG⁹ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (92). Alternatively, a compound of the formula (90) is reacted with a compound of the formula (91) wherein LG⁹ is a leaving group such as chlorine, bromine, iodine, tosylate, mesylate, and the like and Z⁷ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (92). A compound of the formula (92) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (93).

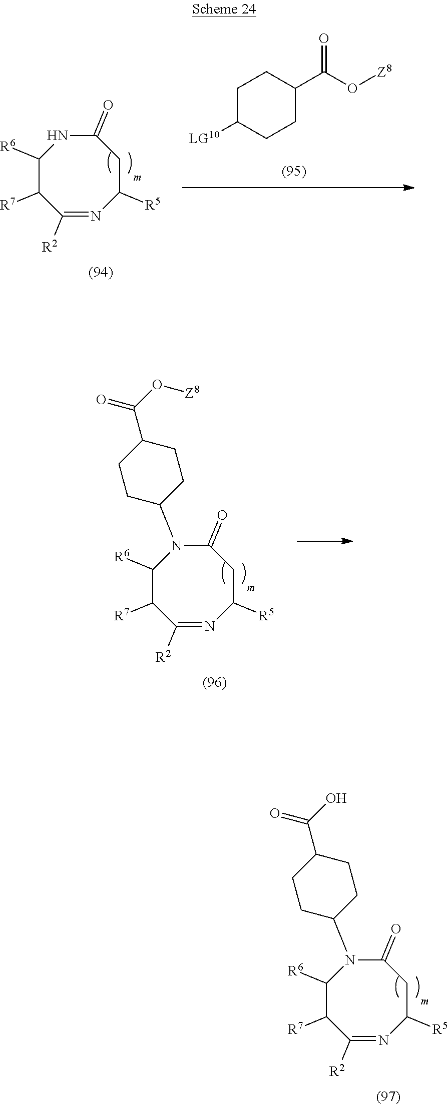

A compound of the formula (94) is reacted with a compound of the formula (95), a known compound or a compound prepared by known methods in which Z⁸ is C1-C6 alkyl and LG¹⁰ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (96). Alternatively, a compound of the formula (94) is reacted with a compound of the formula (95) wherein LG¹⁰ is a leaving group such as chlorine, bromine, iodine, tosylate, mesylate, and the like and Z⁸ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (96). A compound of the formula (96) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (97).

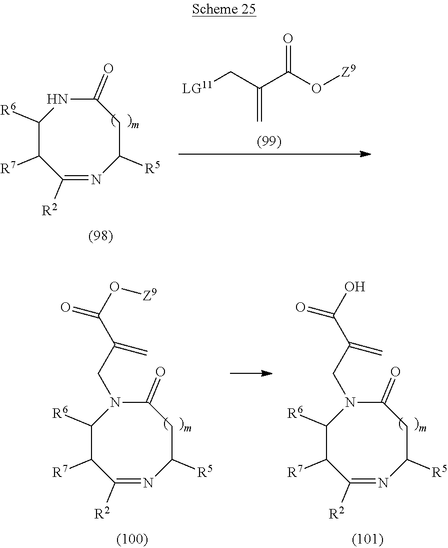

A compound of the formula (98) is reacted with a compound of the formula (99), a known compound or a compound prepared by known methods in which Z⁹ is C1-C6 alkyl and LG¹¹ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (100). Alternatively, a compound of the formula (98) is reacted with a compound of the formula (99) wherein LG¹¹ is a leaving group such as chlorine, bromine, iodine, tosylate, mesylate, and the like and Z⁹ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (100). A compound of the formula (100) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (101).

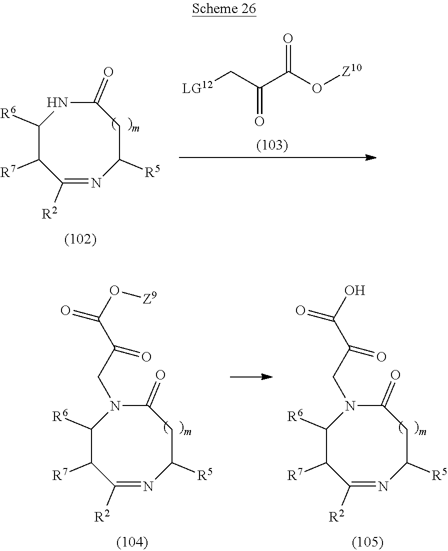

A compound of the formula (102) is reacted with a compound of the formula (103), a known compound or a compound prepared by known methods in which Z¹⁰ is C1-C6 alkyl and LG¹¹ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (104). Alternatively, a compound of the formula (102) is reacted with a compound of the formula (103) wherein LG¹² is a leaving group such as chlorine, bromine, iodine, tosylate, mesylate, and the like and Z¹⁰ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (104). A compound of the formula (104) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (105).

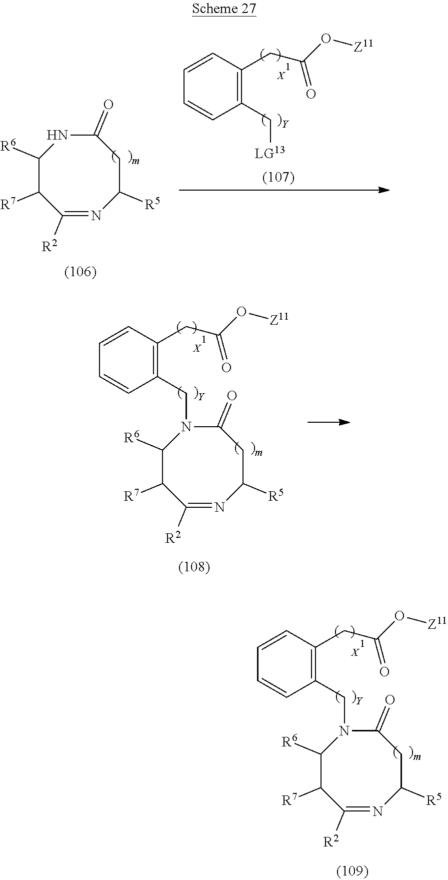

A compound of the formula (106) is reacted with a compound of the formula (107), a known compound or a compound prepared by known methods in which Z¹¹ is C1-C6 alkyl and LG¹³ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (108). Alternatively, a compound of the formula (106) is reacted with a compound of the formula (107) wherein LG¹³ is a leaving group such as chlorine, bromine, iodine, tosylate, mesylate, and the like and Z¹ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (108). A compound of the formula (108) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (109).

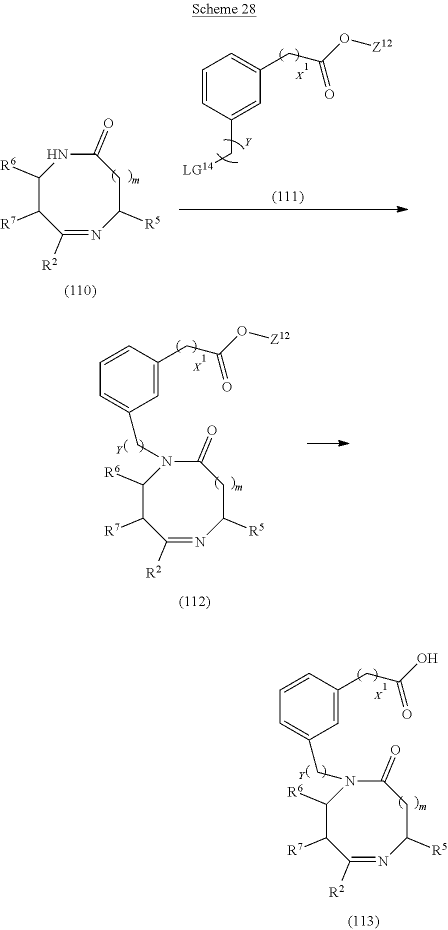

A compound of the formula (110) is reacted with a compound of the formula (111), a known compound or a compound prepared by known methods in which Z¹² is C1-C6 alkyl and LG¹⁴ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (112). Alternatively, a compound of the formula (110) is reacted with a compound of the formula (111) wherein LG¹⁴ is a leaving group such as chlorine, bromine, iodine, tosylate, mesylate, and the like and Z¹² is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (112). A compound of the formula (112) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (113).

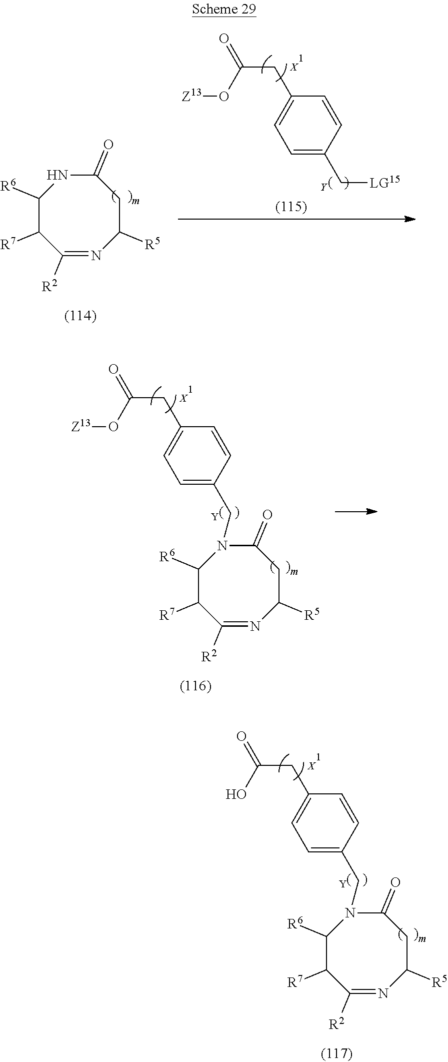

A compound of the formula (114) is reacted with a compound of the formula (115), a known compound or a compound prepared by known methods in which Z¹³ is C1-C6 alkyl and LG¹⁵ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (116). Alternatively, a compound of the formula (114) is reacted with a compound of the formula (115) wherein LG¹⁵ is a leaving group such as chlorine, bromine, iodine, tosylate, mesylate, and the like and Z¹³ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (116). A compound of the formula (116) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (117).

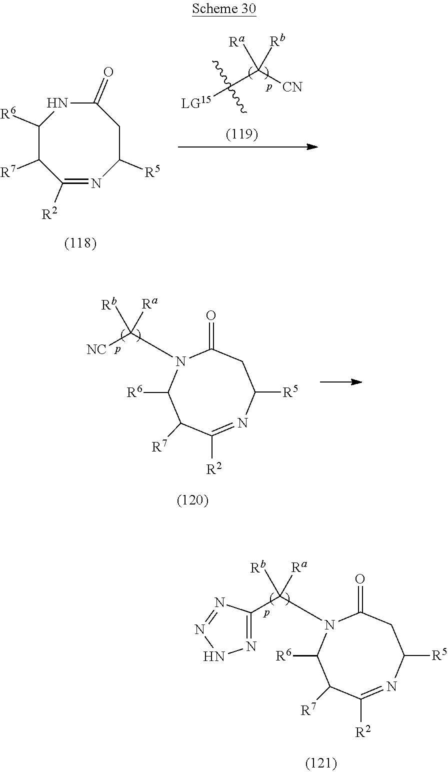

A compound of the formula (118) is reacted with a compound of the formula (119), a known compound or a compound prepared by known methods in which LG¹⁵ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (120). Alternatively, a compound of the formula (118) is reacted with a compound of the formula (119) wherein LG¹⁵ is a leaving group such as chlorine, bromine, iodine, tosylate, mesylate, and the like in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (120). A compound of the formula (120) is reacted with sodium azide in the presence of a solvent such as methanol, ethanol, tetrahydrofuran, 1,4-dioxane, acetonitrile, 1,2-dimethoxyethane, N,N-dimethylformamide, dimethylsulfoxide, N,N-dimethylacetamide and the like, optionally with heating, optionally with microwave irradiation to provide a compound of the formula (121).

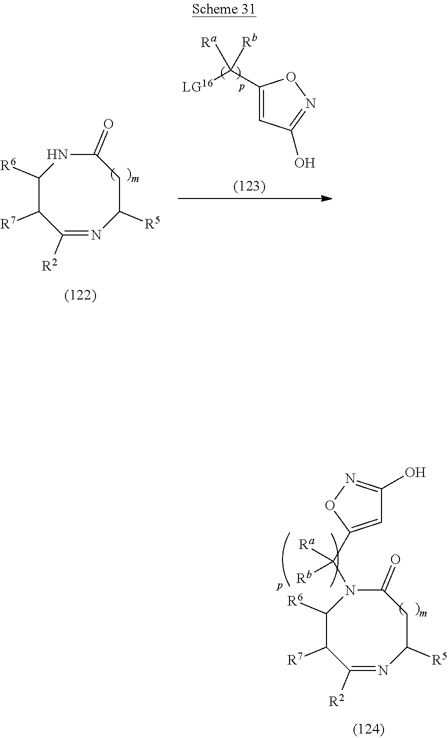

A compound of the formula (122) is reacted with a compound of the formula (123), a known compound or a compound prepared by known methods in which LG¹⁶ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (124). Alternatively, a compound of the formula (122) is reacted with a compound of the formula (123) wherein LG¹⁶ is a leaving group such as chlorine, bromine, iodine, tosylate, mesylate, and the like in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (124).

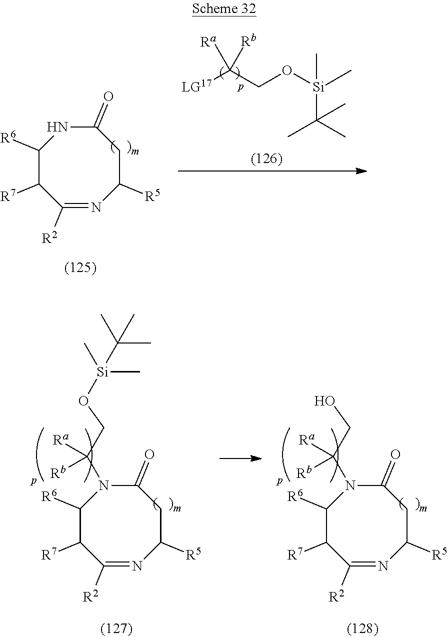

A compound of the formula (125) is reacted with a compound of the formula (126), a known compound or a compound prepared by known methods in which LG¹⁷ is a leaving group such as chlorine, bromine, iodine, tosylate, mesylate, and the like, in the presence of a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (127). A compound of the formula (127) is reacted with tetrabutylammonium fluoride in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (128).

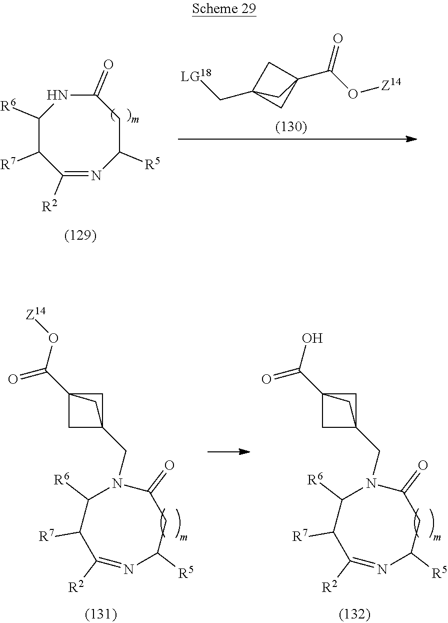

A compound of the formula (129) is reacted with a compound of the formula (130), a known compound or a compound prepared by known methods in which Z¹⁴ is C1-C6 alkyl and LG¹⁸ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (131). Alternatively, a compound of the formula (129) is reacted with a compound of the formula (130) wherein LG¹⁸ is a leaving group such as chlorine, bromine, iodine, tosylate, mesylate, and the like and Z¹⁴ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (131). A compound of the formula (131) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (132).

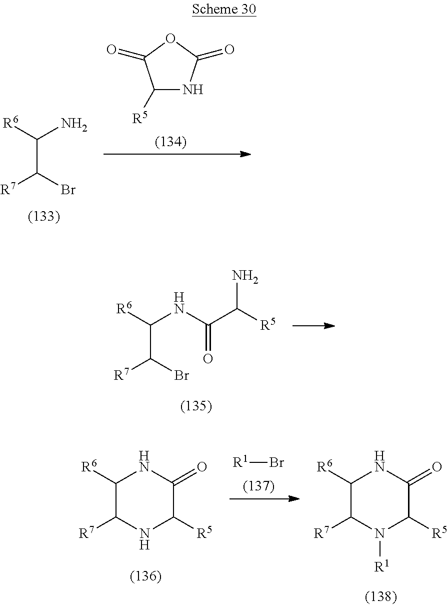

A compound of the formula (133) is reacted with a compound of the formula (134) in the presence of an acid such as trifluoroacetic acid, acetic acid, formic acid, hydrochloric acid, sulfuric acid, and the like, in a solvent such as toluene, benzene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (135). Alternatively, a compound of the formula (133) is reacted with a compound of the formula (134) in the presence of a base such as sodium carbonate, lithium carbonate, potassium carbonate, cesium carbonate, sodium hydroxide, lithium hydroxide, potassium hydroxide, cesium hydroxide and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (135). A compound of the formula (135) is reacted with tri-tert-butylphosphine tetrafluoroborate in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a base such as sodium carbonate, lithium carbonate, potassium carbonate, cesium carbonate, sodium hydroxide, lithium hydroxide, potassium hydroxide, cesium hydroxide and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (136). A compound of the formula (136) is reacted with a compound of the formula (137), a known compound or a compound prepared by known methods, in the presence of a palladium catalyst palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (138).

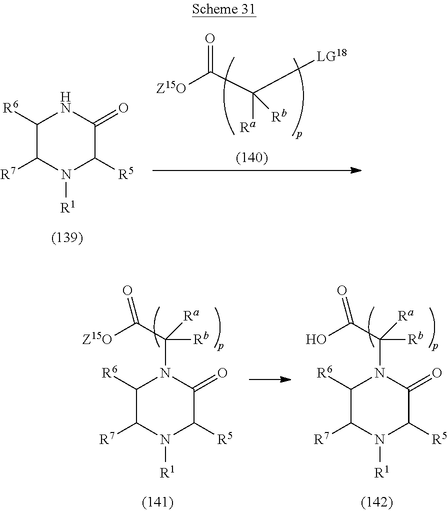

A compound of the formula (139) is reacted with a compound of the formula (140), a known compound or a compound prepared by known methods in which Z¹⁵ is C1-C6 alkyl and LG¹⁹ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (141). Alternatively, a compound of the formula (139) is reacted with a compound of the formula (140) wherein LG¹⁹ is a leaving group such as chlorine, bromine, iodine, tosylate, mesylate, and the like and Z¹⁵ is a C₁-C₆ alkyl, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (141). A compound of the formula (141) is reacted with a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (142).

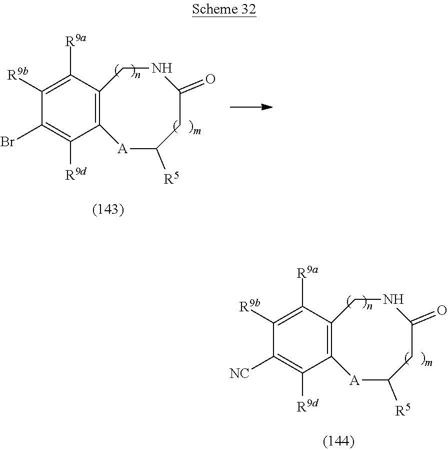

A compound of the formula (143) is reacted a cyanide salt such as sodium cyanide, potassium cyanide, lithium cyanide, copper cyanide, and the like in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (144).

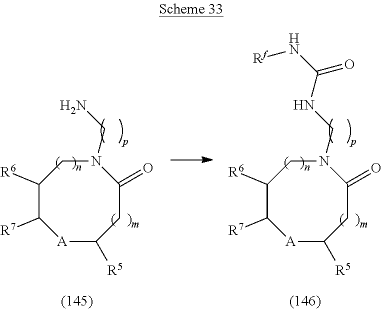

A compound of the formula (145) is reacted a cyanate salt such as potassium cyanate, sodium cyanate, lithium cyanate, and the like, optionally in the presence of an acid such as trifluoroacetic acid, acetic acid, formic acid, hydrochloric acid, sulfuric acid, and the like, optionally in the presence of water, optionally in the presence of a solvent such as toluene, benzene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (146).

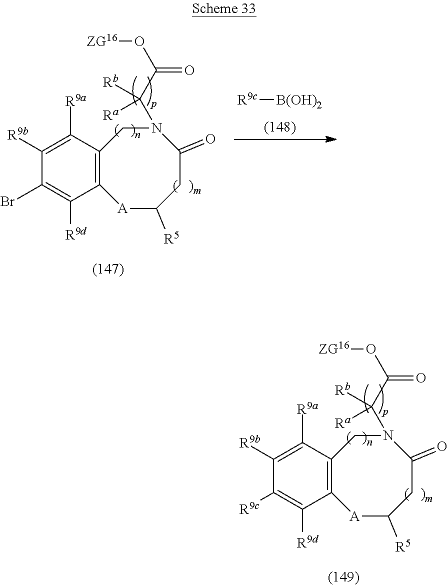

A compound of the formula (147) wherein ZG¹⁶ is a C₁-C₆ alkyl is reacted with a compound of the formula (148), a known compound or a compound prepared by known methods, in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of a base such as sodium carbonate, lithium carbonate, potassium carbonate, cesium carbonate, sodium hydroxide, lithium hydroxide, potassium hydroxide, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (149).

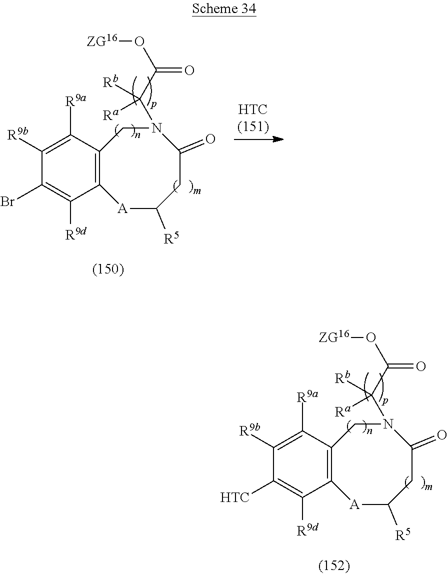

A compound of the formula (150) wherein ZG¹⁷ is a C₁-C₆ alkyl is reacted with a compound of the formula (151), wherein HTC is a C3-C8 heterocycle, in the presence of a palladium catalyst such as palladium (II) acetate, tetrakis(triphenylphosphine)palladium(0), dichlorobis(triphenylphosphine)palladium(II), palladium on carbon, bis(acetonitrile)dichloro palladium(II), 1,1′-bis(diphenylphosphino)ferrocene]dichloropalladium(II), bispalladium-tri(1,3-dibenzylidene)acetone, and the like, in the presence of (2,2′-bis(diphenylphosphino)-1,1′-binaphthyl), in the presence of a base such as potassium tert-butoxide, sodium tert-butoxide, lithium tert-butoxide, and the like, in the presence of a solvent such as toluene, benzene, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (152).

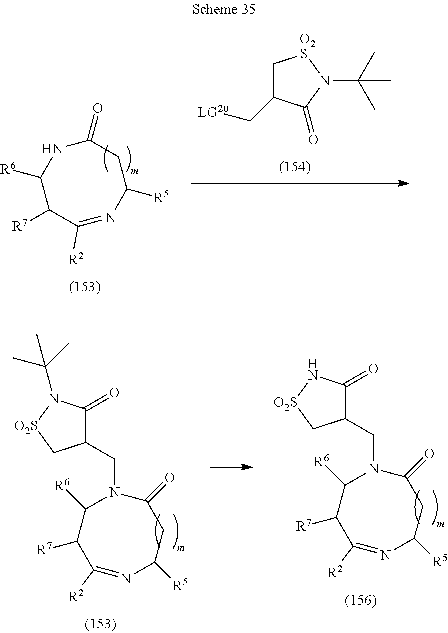

A compound of the formula (153) is reacted with a compound of the formula (154), a known compound or a compound prepared by known methods LG²⁰ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (155). Alternatively, a compound of the formula (153) is reacted with a compound of the formula (154), a known compound or a compound prepared by known methods wherein LG²⁰ is a leaving group such as chlorine, bromine, iodine, tosylate, mesylate, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (155). A compound of the formula (155) is reacted with an acid such as trifluoroacetic acid, acetic acid, formic acid, hydrochloric acid, sulfuric acid, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (156).

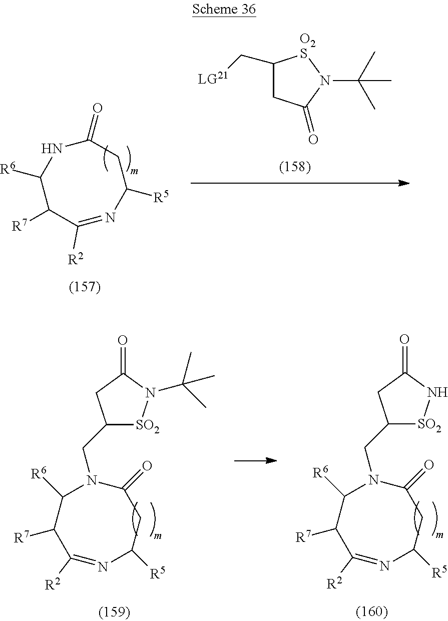

A compound of the formula (157) is reacted with a compound of the formula (158), a known compound or a compound prepared by known methods LG¹ is a leaving group such as chlorine, bromine, iodine, tosylate, mesylate, and the like, optionally in the presence of a base such as triethylamine, N,N-diisopropylethylamine, pyridine, 2,6-dimethylpyridine, and the like, optionally in the presence of benzyltributylammonium bromide, optionally with heating, optionally with microwave irradiation to provide a compound of the formula (159). Alternatively, a compound of the formula (157) is reacted with a compound of the formula (158), a known compound or a compound prepared by known methods wherein LG²¹ is a leaving group such as chlorine, bromine, iodine, tosylate, mesylate, in the presence of a base such as sodium hydride, potassium hydride, lithium diisopropylamide, sodium diisopropylamide, potassium diisopropylamide, and the like, in the presence of a solvent such as tetrahydrofuran, 1,4-dioxane, acetonitrile, methylene chloride, chloroform, 1,2-dichloroethane, 1,2-dimethoxyethane, N,N-dimethylformamide, dimethylsulfoxide, N,N-dimethylacetamide and the like, optionally with heating, optionally with microwave irradiation to provide a compound of the formula (159). A compound of the formula (159) is reacted with an acid such as trifluoroacetic acid, acetic acid, formic acid, hydrochloric acid, sulfuric acid, and the like, in the presence of a solvent such as methanol, ethanol, tetrahydrofuran, 1,4-dioxane, acetonitrile, 1,2-dimethoxyethane, and the like, optionally with heating, optionally with microwave irradiation to provide a compound of the formula (160).

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

The following LC/MS procedure was used for the analysis of the examples described herein. LC/MS data were determined with a Waters Alliance 2695 HPLC/MS (Waters Symmetry C18, 4.6×75 mm, 3.5 μm) with a 2996 diode array detector from 210-400 nm; the solvent system is 5-95% acetonitrile in water (with 0.1% trifluoroacetic acid) over nine minutes using a linear gradient, and retention times are in minutes. Mass spectrometry was performed on a Waters ZQ using electrospray in positive mode. Alternatively, LC/MS data were determined with an Agilent 6120 mass spectrometer with electrospray ionization source (1200 Agilent LC-MS spectrometer, Positive) using a mobile phase flow was 1.0 mL/min with a three minute gradient from 20% aqueous media (0.1% formic acid) to 95% CH₃CN (0.1% formic acid) and a 9.0 min total acquisition time.

Preparative reverse phase HPLC was performed on a Phenomenex LUNA column (19×100 mm, C18, 5 μm) with a 10 minute mobile phase gradient of 10% acetonitrile/water to 90% acetonitrile/water with 0.1% trifluoroacetic acid as buffer using 214 and 254 nm as detection wavelengths. Injection and fraction collection were performed with a Gilson 215 liquid handling apparatus using Trilution LC software.

¹H-NMR's were taken on a Varian 300 MHz NMR using tetramethylsilane (TMS) as internal standard (□=0.00) with peaks reported downfield from TMS. Alternatively, ¹H-NMR's were taken on a Varian 400 MHz NMR using tetramethylsilane (TMS) as internal standard (□=0.00) with peaks reported downfield from TMS.

The examples provided methods for preparing representative compounds of formulas (I) through (XXIX). The skilled practitioner will know how to substitute the appropriate reagents, starting materials and purification methods known to those skilled in the art, in order to prepare additional compounds of the present invention.

### EXAMPLES

Examples 1-101 provides methods for preparing representative compounds of formulas (I) through (XXIX). The skilled practitioner will know how to substitute the appropriate reagents, starting materials and purification methods known to those skilled in the art, in order to prepare additional compounds of the present invention.

### Example 1: Synthesis of 2-((S)-3-((S)-sec-butyl)-8-chloro-2-oxo-5-phenyl-2,3-dihydro-1H-benzo[e][1,4]diazepin-1-yl)acetic acid

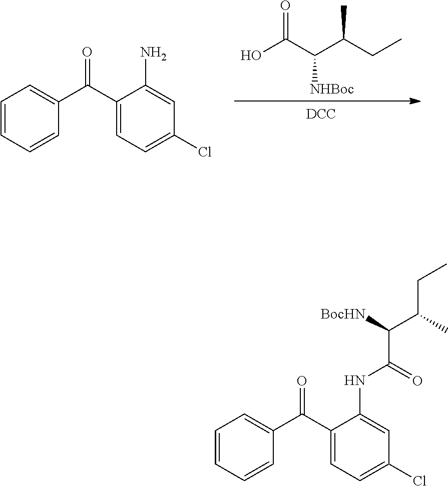

Step 1: Dicyclohexylcarbodiimide 1M in dichloromethane (1.5 mL, 1.5 mmol) was added at 0° C. to a solution of L-N-Boc-isoleucine (350 mg, 1.52 mmol) in 0.77 mL of dichloromethane. The resulting mixture was stirred at 23° C. for one hour before being cooled back to 0° C. A solution of (2-amino-4-chlorophenyl)(phenyl)methanone (351 mg, 1.52 mmol) in 1.4 mL of dichloromethane was then added dropwise and the resulting mixture was stirred at 23° C. for three days. The insoluble material was filtered off and the filtrate evaporated. The residue was purified by flash chromatography on silica gel (dichloromethane/hexanes 5/95 to 100/0 followed by ethyl acetate/dichloromethane 20/80 to 50/50) to give 596 mg of impure tert-butyl ((2S,3S)-1-((2-benzoyl-5-chlorophenyl)amino)-3-methyl-1-oxopentan-2-yl)carbamate, which was used in the next step without further purification. MS (ESI) m/z 467.22 (M+Na)⁺

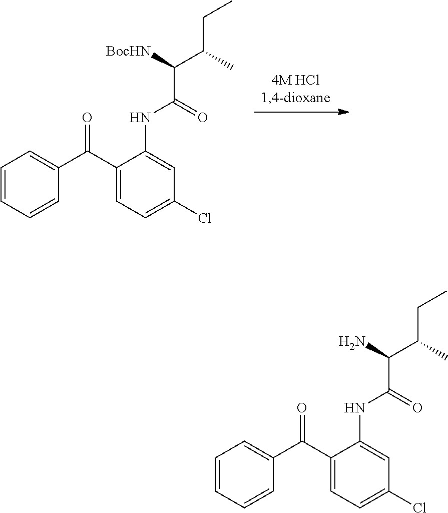 [SMILES: CC[C@H](C)[C@H](NC(=O)OC(C)(C)C)C(=O)Nc1cc(Cl)ccc1C(=O)c1ccccc1 ; "4M HCl1,4-dioxane" ; CC[C@H](C)[C@H](N)C(=O)Nc1cc(Cl)ccc1C(=O)c1ccccc1]

Step 2: Hydrochloric acid 4M in 1,4-dioxane (3.35 mL, 13.4 mmol) was added at 23° C. to a solution of impure tert-butyl ((2S,3S)-1-((2-benzoyl-5-chlorophenyl)amino)-3-methyl-1-oxopentan-2-yl)carbamate (296 mg, 1.34 mmol) in 3.6 mL of 1,4-dioxane. The resulting mixture was stirred at 23° C. for 16 hours before being evaporated. The residue was dissolved in dichloromethane and the solution was washed first with saturated aqueous sodium bicarbonate and then with water before being dried over magnesium sulfate, filtered, and evaporated to give 319 mg of crude (2S,3S)-2-amino-N-(2-benzoyl-5-chlorophenyl)-3-methylpentanamide, which was used in the next step without purification. MS (ESI) m/z 345.2 (M+H)⁺

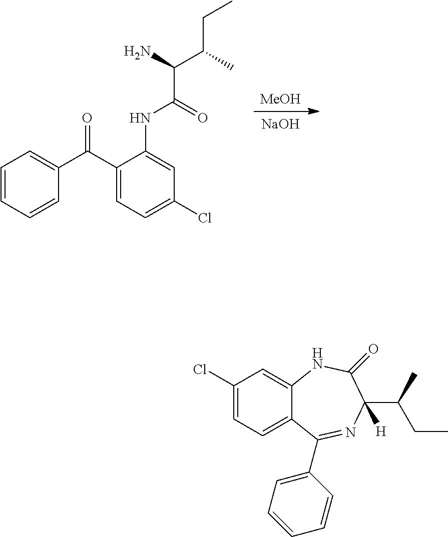 [SMILES: "MeOH" ; CC[C@H](C)[C@H](N)C(=O)Nc1cc(Cl)ccc1C(=O)c1ccccc1 ; "NaOH" ; CC[C@H](C)[C@@H]1N=C(c2ccccc2)c2ccc(Cl)cc2NC1=O]

Step 3: To a solution of crude (2S,3S)-2-amino-N-(2-benzoyl-5-chlorophenyl)-3-methylpentanamide (319 mg, theoretically 0.924 mmol) in 2.64 mL of methanol and 0.26 mL of water were added at 0° C. several drops of 1M aqueous sodium hydroxide to adjust the pH to 8-9. The resulting mixture was stirred at 23° C. for 18 hours before being evaporated. The residue was taken into water and extracted three times with dichloromethane. The combined organic layers were dried over sodium sulfate, filtered, and evaporated. The residue was purified by flash chromatography on silica gel (hexanes/ethyl acetate 95/5 to 40/60) to give 125 mg of (S)-3-((S)-sec-butyl)-8-chloro-5-phenyl-1,3-dihydro-2H-benzo[e][1,4]diazepin-2-one. MS (ESI) m/z 327.22 (M+H)⁺

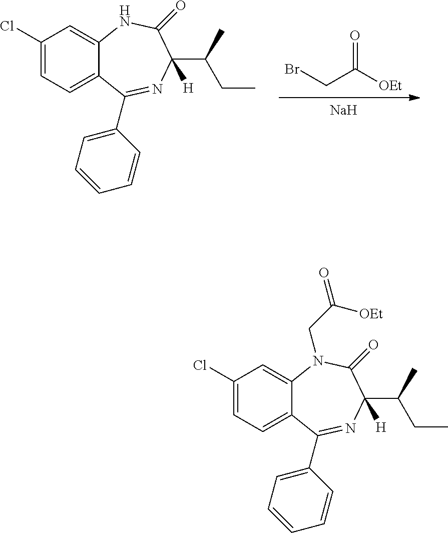

Step 4: Sodium hydride 60% dispersion in oil (31 mg, 0.8 mmol) was added at 0° C. to a solution of (S)-3-((S)-sec-butyl)-8-chloro-5-phenyl-1,3-dihydro-2H-benzo[e][1,4]diazepin-2-one (125 mg, 0.4 mmol) in 1.7 mL of tetrahydrofuran. The resulting mixture was stirred at 0° C. for two and half hours before adding ethyl bromoacetate (0.063 mL, 0.6 mmol). The resulting mixture was stirred at 23° C. for 18 hours before being cooled to 0° C., quenched by addition of water, and extracted three times with ethyl acetate. The combined organic layers were dried over sodium sulfate, filtered, and evaporated. The residues was purified by flash chromatography on silica gel (hexanes/ethyl acetate 95/5 to 50/50) to give 69 mg of ethyl 2-((S)-3-((S)-sec-butyl)-8-chloro-2-oxo-5-phenyl-2,3-dihydro-1H-benzo[e][1,4]diazepin-1-yl)acetate (44% yield). MS (ESI) m/z 413.36 (M+H)⁺

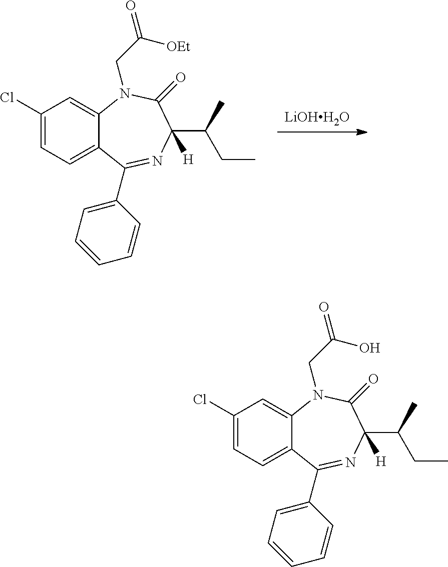 [SMILES: CCOC(=O)CN1C(=O)[C@H]([C@@H](C)CC)N=C(c2ccccc2)c2ccc(Cl)cc21 ; "LiOH·H2O" ; CC[C@H](C)[C@@H]1N=C(c2ccccc2)c2ccc(Cl)cc2N(CC(=O)O)C1=O]

Step 5: Lithium hydroxide monohydrate (38 mg, 0.9 mmol) was added at 23° C. to a solution of ethyl 2-((S)-3-((S)-sec-butyl)-8-chloro-2-oxo-5-phenyl-2,3-dihydro-1H-benzo[e][1,4]diazepin-1-yl)acetate (69 mg, 0.2 mmol) in 4 mL of a 1/1 mixture of 1,4-dioxane and water. The resulting mixture was stirred at 23° C. for 18 hours before being concentrated to half volume. The solution was cooled to 0° C. and the pH was adjusted to 5-6 by addition of 10% aqueous citric acid before being extracted 3 times with ethyl acetate. The combined organic layers were dried over sodium sulfate, filtered, and evaporated. The residue was crystallized and recrystallized three time in dichloromethane/hexanes to give 58 mg 2-((S)-3-((S)-sec-butyl)-8-chloro-2-oxo-5-phenyl-2,3-dihydro-1H-benzo[e][1,4]diazepin-1-yl)acetic acid. ¹H NMR (400 MHz, DMSO-d6) δ 12.83 (s, 1H), 7.63-7.58 (m, 1H), 7.52-7.47 (m, 5H), 7.27-7.21 (m, 2H), 4.62 (d, J=17.6 Hz, 1H), 4.48 (d, J=17.6 Hz, 1H), 3.14 (d, J=10.0 Hz, 1H), 2.37-2.32 (m, 1H), 1.89-1.84 (m, 1H), 1.14-1.06 (m, 1H), 0.87-0.82 (m, 6H). MS (ESI): m/z 385.30 (M+H)⁺.

### Example 2: Synthesis of 2-((S)-3-((S)-sec-butyl)-9-chloro-2-oxo-5-phenyl-2,3-dihydro-1H-benzo[e][1,4]diazepin-1-yl)acetic acid

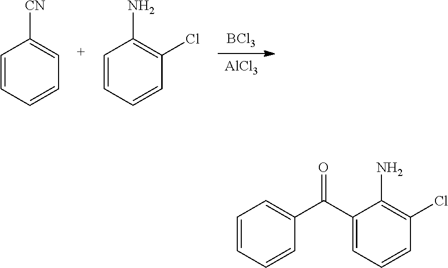

Step 1: 2-Chloroaniline (908 μL, 8.65 mmol) in 9.6 mL of dichloroethane was added dropwise at 0° C. to a solution of boron trichloride 1M in dichloromethane (5.6 mL, 5.6 mmol). The resulting mixture was stirred at 0° C. for 30 minutes before adding benzonitrile (445 μL, 4.32 mmol) followed by aluminum trichloride (748 mg, 5.61 mmol). The resulting mixture was stirred at 23° C. for 30 minutes before being stirred at 90° C. for 18 hours. The reaction mixture was then cooled to 0° C. before being quenched by addition of 10 mL of 2M aqueous hydrochloric acid. The resulting mixture was stirred at 90° C. for 30 minutes, cooled back to 23° C., and extracted three times with dichloromethane. The combined organic layers were dried over sodium sulfate, filtered, and evaporated. The residue was purified by flash chromatography on silica gel (hexanes/ethyl acetate 93/7 to 73/27) to give 534 mg of (2-amino-3-chlorophenyl)(phenyl)methanone MS (ESI) m/z 232.13 (M+H)⁺

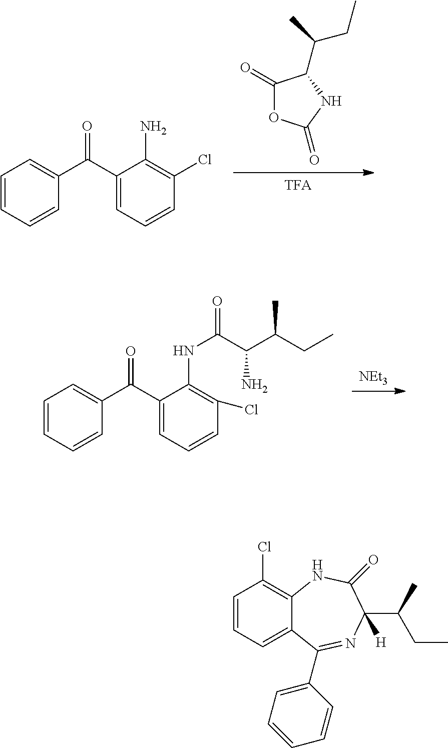 [SMILES: Nc1c(Cl)cccc1C(=O)c1ccccc1 ; CC[C@H](C)[C@@H]1NC(=O)OC1=O ; CC[C@H](C)[C@H](N)C(=O)Nc1c(Cl)cccc1C(=O)c1ccccc1 ; "TFA" ; CC[C@H](C)[C@@H]1N=C(c2ccccc2)c2cccc(Cl)c2NC1=O ; "NEt3"]

Step 2: A mixture of (2-amino-3-chlorophenyl)(phenyl)methanone (367 mg, 1.58 mmol), (S)-4-((S)-sec-butyl)oxazolidine-2,5-dione (598 mg, 3.8 mmol), and trifluoroacetic acid (364 μL, 4.75 mmol) in 7.9 mL of toluene was stirred at 60° C. for 48 hours before adding triethylamine (359 μL, 2.56 mmol). The resulting mixture was then stirred at 80° C. for one hour before being cooled to 23° C. and evaporated. The residue was taken into dichloromethane and the insoluble material was filtered off. The filtrate was evaporated and the residue was purified by flash chromatography on silica gel (hexanes/ethyl acetate 93/7 to 60/40) to give 120 mg (S)-3-((S)-sec-butyl)-9-chloro-5-phenyl-1,3-dihydro-2H-benzo[e][1,4]diazepin-2-one (23% yield). MS (ESI) m/z 327.21 (M+H)⁺

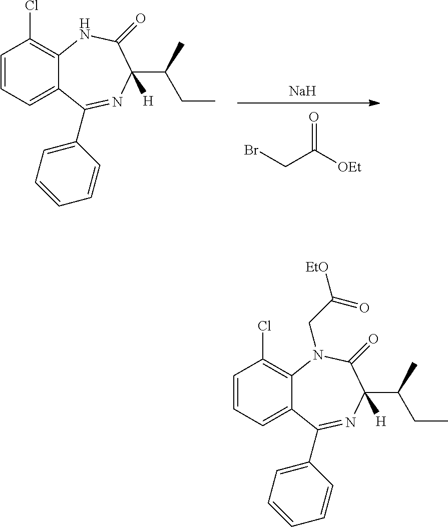

Step 3: Sodium hydride 60% dispersion in oil (58 mg, 1.46 mmol) was added at 0° C. to a solution of (S)-3-((S)-sec-butyl)-9-chloro-5-phenyl-1,3-dihydro-2H-benzo[e][1,4]diazepin-2-one (235 mg, 0.719 mmol) in 3.3 mL of tetrahydrofuran. The resulting mixture was stirred at 0° C. for 30 minutes before adding ethyl bromoacetate (120 μL, 1.08 mmol). The reaction mixture was stirred at 23° C. for two hours before being cooled back to 0° C., quenched by the addition of iced water, and extracted three times with ethyl acetate. The combined organic layers were washed with water, dried over sodium sulfate, filtered, and evaporated. The residue was purified by flash chromatography on silica gel (hexanes/ethyl acetate 93/7 to 50/50) to give 247 mg of ethyl 2-((S)-3-((S)-sec-butyl)-9-chloro-2-oxo-5-phenyl-2,3-dihydro-1H-benzo[e][1,4]diazepin-1-yl)acetate. MS (ESI) m/z 413.08 (M+H)⁺

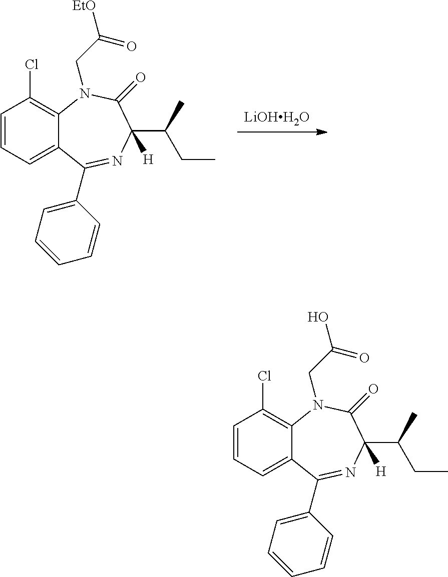 [SMILES: CCOC(=O)CN1C(=O)[C@H]([C@@H](C)CC)N=C(c2ccccc2)c2cccc(Cl)c21 ; "LiOH·H2O" ; CC[C@H](C)[C@@H]1N=C(c2ccccc2)c2cccc(Cl)c2N(CC(=O)O)C1=O]

Step 4: Lithium hydroxide monohydrate (138 mg, 3.29 mmol) was added at 23° C. to a solution of ethyl 2-((S)-3-((S)-sec-butyl)-9-chloro-2-oxo-5-phenyl-2,3-dihydro-1H-benzo[e][1,4]diazepin-1-yl)acetate (247 mg, 0.598 mmol) in 14 mL of a 1/1 mixture of 1,4-dioxane and water. The resulting mixture was stirred at 23° C. for 18 hours before being cooled to 0° C., acidified to pH 5-6 by addition of 10% aqueous citric acid, and extracted three times with ethyl acetate. The combined organic layers were washed with brine, dried over sodium sulfate, filtered, and evaporated. The residue crystallized from dichloromethane/hexanes to give 210 mg of 2-((S)-3-((S)-sec-butyl)-9-chloro-2-oxo-5-phenyl-2,3-dihydro-1H-benzo[e][1,4]diazepin-1-yl)acetic acid. ¹H NMR (400 MHz, DMSO-d6) δ 7.74 (dd, J=8.2, 1.4 Hz, 1H), 7.56-7.54 (m, 2H), 7.46-7.30 (m, 4H), 7.14 (dd, J=8.2, 1.4 Hz, 1H), 4.67 (d, J=17.6 Hz, 1H), 4.14 (d, J=17.6 Hz, 1H), 3.12 (d, J=10.0 Hz, 1H), 2.35-2.28 (m, 1H), 1.86-1.80 (m, 1H), 1.11-0.99 (m, 1H), 0.85-0.80 (m, 6H). MS (ESI): m/z 385.15 (M+H)⁺[0601]Example 4: 2-((S)-3-((S)-sec-butyl)-7-(dimethylamino)-2-oxo-5-phenyl-2,3-dihydro-1H-benzo[e][1,4]diazepin-1-yl)acetic acid.

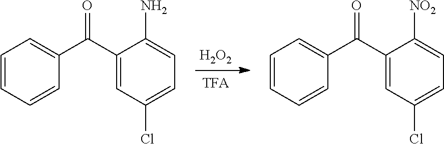

Step 1: Aqueous 30% hydrogen peroxide (14 mL, 125 mmol) was added dropwise to a refluxing solution of (2-amino-5-chlorophenyl)(phenyl)methanone (5.79 g, 25 mmol) in 25 mL of trifluoroacetic acid. The resulting mixture was stirred at reflux for two hours before being cooled to 23° C. and poured into iced water. The mixture was extracted with ethyl acetate and the combined organic layers were washed with saturated aqueous sodium bicarbonate followed by brine, dried over magnesium sulfate, filtered, and evaporated to give 5.53 g of (5-chloro-2-nitrophenyl)(phenyl)methanone. ¹H NMR (400 MHz, DMSO-d6) δ 8.27 (d, J=8.8 Hz, 1H), 7.86 (dd, J=18.5, 2.3 Hz, 2H), 7.73-7.63 (m, 3H), 7.53-7.48 (m, 2H).

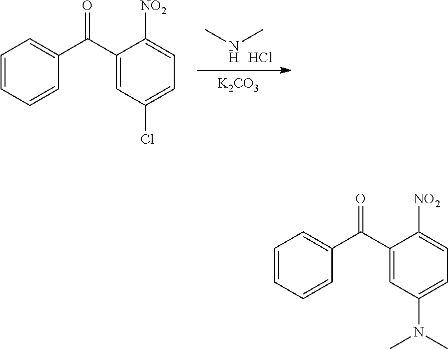

Step 2: A mixture of (5-chloro-2-nitrophenyl)(phenyl)methanone (460 mg, 1.76 mmol), potassium carbonate (972 mg, 7.03 mmol), and dimethylamine hydrochloride (359 mg, 4.4 mmol) in 3 mL of N,N-dimethylformamide was stirred at reflux for 3 hours before being cooled to 23° C. and poured into ice. The precipitate that formed was filtered, rinsed with water, and dried. The residue was purified by flash chromatography on silica gel (hexanes/ethyl acetate 100/0 to 55/45) to give 286 mg of (5-(dimethylamino)-2-nitrophenyl)(phenyl)methanone. ¹H NMR (400 MHz, DMSO-d6) δ 8.08 (d, J=9.5 Hz, 1H), 7.66-7.62 (m, 2H), 7.61-7.56 (m, 1H), 7.48-7.42 (m, 2H), 6.85 (dd, J=9.5, 2.8 Hz, 1H), 6.61 (d, J=2.8 Hz, 1H), 3.05 (s, 6H). MS (ESI) m/z 271.17 (M+H)⁺

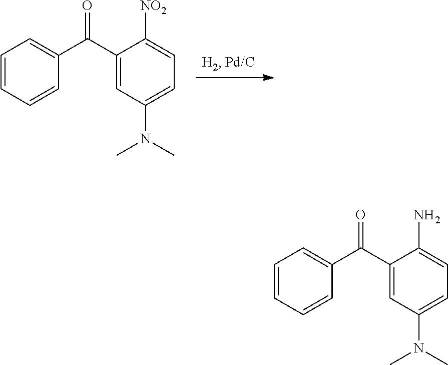 [SMILES: CN(C)c1ccc([N+](=O)[O-])c(C(=O)c2ccccc2)c1 ; "H2, Pd/C" ; CN(C)c1ccc(N)c(C(=O)c2ccccc2)c1]

Step 3: A mixture of (5-(dimethylamino)-2-nitrophenyl)(phenyl)methanone (286 mg, 1.06 mmol) and palladium 10% on carbon in 5 mL of dichloromethane was stirred at 23° C. under 1 atm of hydrogen for 18 hours before being filtered through celite. The celite plug was rinsed with dichloromethane and the filtrate was evaporated. The residue was purified by flash chromatography on silica gel (hexanes/ethyl acetate 100/0 to 55/45) to give (2-amino-5-(dimethylamino)phenyl)(phenyl)methanone. ¹H NMR (400 MHz, DMSO-d6) δ 7.55-7.49 (m, 3H), 7.49-7.41 (m, 2H), 7.00 (dd, J=9.0, 3.0 Hz, 1H), 6.75 (d, J=9.0 Hz, 1H), 6.58 (d, J=2.9 Hz, 1H), 6.48 (br s, 2H), 2.56 (s, 6H); MS (ESI) m/z 241.19 (M+H)⁺

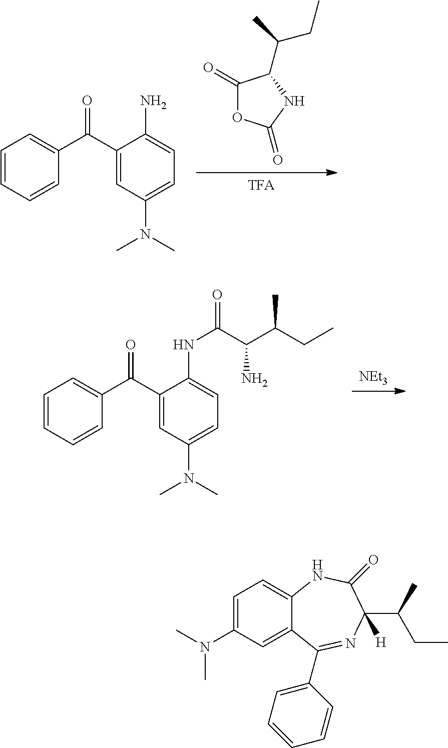

Step 4: A mixture of (2-amino-5-(dimethylamino)phenyl)(phenyl)methanone (124 mg, 0.516 mmol), (S)-4-((S)-sec-butyl)oxazolidine-2,5-dione (97 mg, 0.619 mmol), and trifluoroacetic acid (115 μL, 1.55 mmol) in 2.6 mL of toluene was stirred at 70° C. for 19 hours before adding triethylamine (216 μL, 1.55 mmol). The resulting mixture was then stirred at 80° C. for 30 minutes before being cooled to 23° C., diluted with ethyl acetate, washed with saturated aqueous sodium bicarbonate, brine, dried over magnesium sulfate, filtered, and evaporated. The residues was purified by flash chromatography on silica gel (hexanes/ethyl acetate 100/0 to 0/100) to give 123 mg of (S)-3-((S)-sec-butyl)-7-(dimethylamino)-5-phenyl-1,3-dihydro-2H-benzo[e][1,4]diazepin-2-one. ¹H NMR (400 MHz, DMSO-d6) δ 10.18 (s, 1H), 7.53-7.34 (m, 5H), 7.05 (d, J=3.8 Hz, 2H), 6.44 (d, J=2.4 Hz, 1H), 3.08 (d, J=9.8 Hz, 1H), 2.76 (s, 5H), 2.33-2.19 (m, 1H), 1.91-1.80 (m, 1H), 1.23-1.15 (m, 1H), 0.89 (d, J=6.5 Hz, 3H), 0.84 (t, J=7.4 Hz, 3H); MS (ESI) m/z 336.32 (M+H)⁺

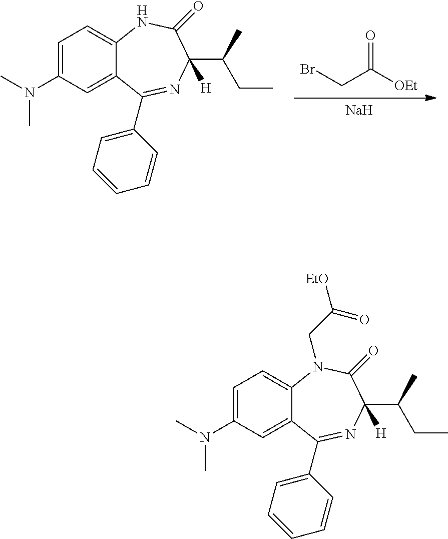

Step 5: Sodium hydride 60% dispersion in oil (29 mg, 0.734 mmol) was added at 0° C. to a solution of (S)-3-((S)-sec-butyl)-7-(dimethylamino)-5-phenyl-1,3-dihydro-2H-benzo[e][1,4]diazepin-2-one (123 mg, 0.367 mmol) in 2 mL of tetrahydrofuran. The resulting mixture was stirred at 0° C. for 2 hours before adding ethyl bromoacetate (61 μL, 0.551 mmol). The resulting mixture was stirred at 23° C. for 16 hours before being quenched by the addition of saturated aqueous ammonium chloride, diluted with ethyl acetate, washed with brine, dried over magnesium sulfate, filtered, and evaporated. The residue was purified by flash chromatography on silica gel (hexanes/ethyl acetate 100/0 to 55/45) to give 108 mg of still impure ethyl 2-((S)-3-((S)-sec-butyl)-7-(dimethylamino)-2-oxo-5-phenyl-2,3-dihydro-1H-benzo[e][1,4]diazepin-1-yl)acetate, which was purified a second time by flash chromatography on silica gel (hexanes/ethyl acetate 100/0 to 50/40) to give 86 mg of pure ethyl 2-((S)-3-((S)-scc-butyl)-7-(dimethylamino)-2-oxo-5-phenyl-2,3-dihydro-1H-benzo[e][1,4]diazepin-1-yl)acetate. ¹H NMR (400 MHz, DMSO-d6) δ 7.58-7.53 (m, 2H), 7.45-7.33 (m, 3H), 7.29 (dd, J=9.1, 5.0 Hz, 1H), 6.96 (dd, J=9.1, 3.0 Hz, 1H), 6.35 (d, J=3.0 Hz, 1H), 4.57 (d, J=17.4 Hz, 1H), 4.43 (d, J=17.4 Hz, 1H), 4.01-3.85 (m, 2H), 3.21-3.15 (m, 1H), 2.77 (s, 6H), 2.30 (ddt, J=13.1, 6.5, 3.2 Hz, 1H), 1.84 (ddd, J=13.2, 7.7, 3.1 Hz, 1H), 1.08-1.03 (m, 1H), 0.99 (t, J=7.1 Hz, 3H), 0.85-0.80 (m, 6H); MS (ESI) m/z 422.19 (M+H)⁺

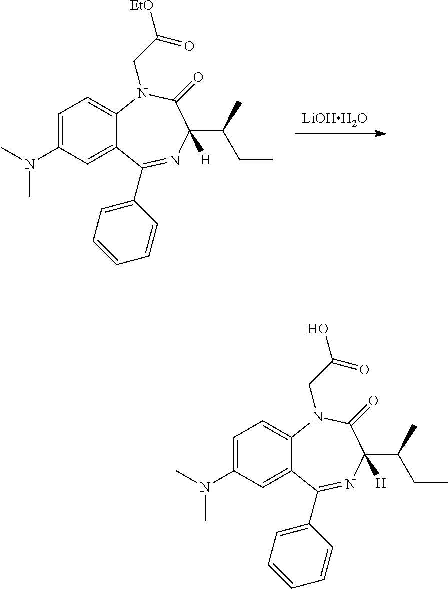

Step 6: Lithium hydroxide monohydrate (43 mg, 1.02 mmol) was added at 23° C. to a solution of ethyl 2-((S)-3-((S)-sec-butyl)-7-(dimethylamino)-2-oxo-5-phenyl-2,3-dihydro-1H-benzo[e][1,4]diazepin-1-yl)acetate (86 mg, 0.204 mmol) in 4 mL of a degassed ⅓ mixture of tetrahydrofuran and water. The resulting mixture was stirred at 23° C. for 20 hours before being neutralized by the addition of Dowex 50W×4 and filtered. The resin was rinsed with ethyl acetate and the filtrated was dried over magnesium sulfate, filtered, and evaporated. The residue was crystallized from ethyl acetate/hexanes to give 21 mg of 2-((S)-3-((S)-sec-butyl)-7-(dimethylamino)-2-oxo-5-phenyl-2,3-dihydro-1H-benzo[e][1,4]diazepin-1-yl)acetic acid.

### Example 5: Synthesis of 2-((S)-3-((S)-sec-butyl)-7-fluoro-2-oxo-5-phenyl-2,3-dihydro-1H-benzo[e][1,4]diazepin-1-yl)acetic acid

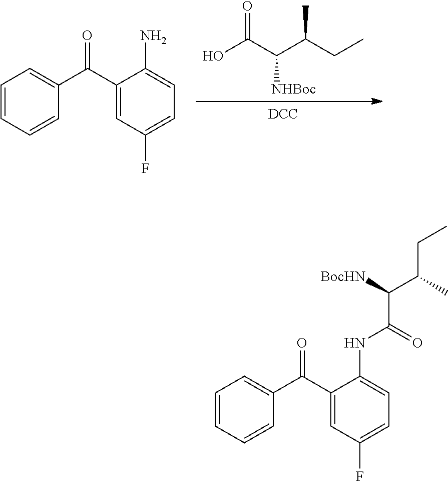

Step 1: A solution of dicyclohexylcarbodiimide 1M in dichloromethane (1.63 mL, 1.63 mmol) was added 0° C. to a solution of L-N-Boc-isoleucine (377 mg, 1.63 mmol) in 0.83 mL of dichloromethane. The resulting mixture was stirred at 23° C. for 2.5 hours before being cooled to 0° C. A solution of (2-amino-5-fluorophenyl)(phenyl)methanone (350 mg, 1.63 mmol) in 1.5 mL of dichloromethane was then added dropwise and the resulting mixture was stirred at 23° C. for 72 hours. The insoluble material was filtered off and the filtrate evaporated. The residue was purified by flash chromatography on silica gel (dichloromethane/hexanes 12/88 followed by dichloromethane/ethyl acetate 90/10) to give 538 mg of impure tert-butyl ((2S,3S)-1-((2-benzoyl-4-fluorophenyl)amino)-3-methyl-1-oxopentan-2-yl)carbamate, which was used in the next step without further purification. MS (ESI) m/z 451.17 (M+Na)⁺

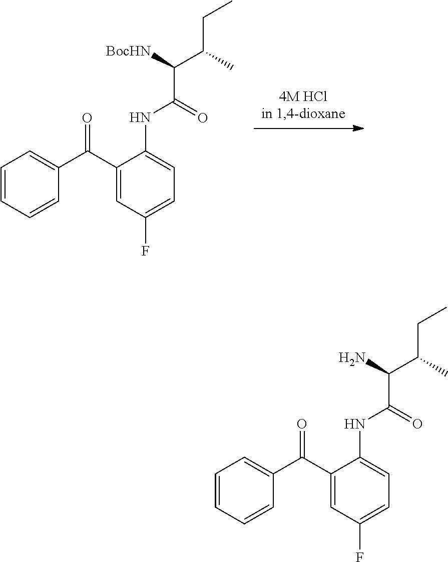 [SMILES: CC[C@H](C)[C@H](NC(=O)OC(C)(C)C)C(=O)Nc1ccc(F)cc1C(=O)c1ccccc1 ; "4M HClin 1,4-dioxane" ; CC[C@H](C)[C@H](N)C(=O)Nc1ccc(F)cc1C(=O)c1ccccc1]

Step 2: Hydrochloric acid 4M in 1,4-dioxane (3.14 mL, 12.56 mmol) was added at 23 ° C. to a solution of impure tert-butyl ((2S,3S)-1-((2-benzoyl-4-fluorophenyl)amino)-3-methyl-1-oxopentan-2-yl)carbamate (538 mg, theoretically 1.256 mmol) in 3.4 mL of 1,4-dioxane. The resulting mixture was stirred at 23° C. for 18 hours before being evaporated. The residue was dissolved in dichloromethane and the solution was washed first with saturated aqueous sodium bicarbonate and then with water before being dried over magnesium sulfate, filtered, and evaporated to give 363 mg of crude (2S,3S)-2-amino-N-(2-benzoyl-5-chlorophenyl)-3-methylpentanaimide, which was used in the next step without purification. MS (ESI) m/z 319.06 (M+H)⁺

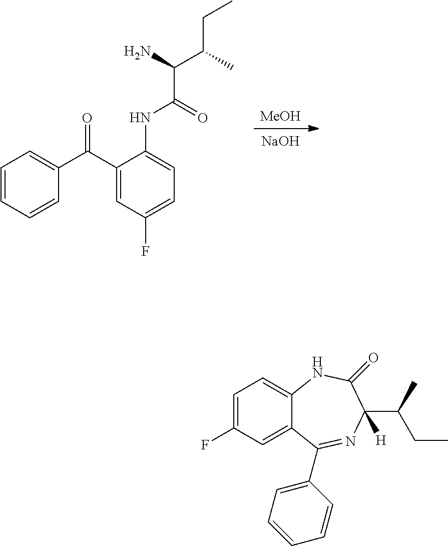 [SMILES: "MeOH" ; CC[C@H](C)[C@H](N)C(=O)Nc1ccc(F)cc1C(=O)c1ccccc1 ; "NaOH" ; CC[C@H](C)[C@@H]1N=C(c2ccccc2)c2cc(F)ccc2NC1=O]

Step 3: To a solution of (2S,3S)-2-amino-N-(2-benzoyl-5-chlorophenyl)-3-methylpentanamide (363 mg, 1.105 mmol) in 3.2 mL of methanol and 0.32 mL of water was added at 0° C. several drops of 1M aqueous sodium hydroxide to adjust the pH to 8-9. The resulting mixture was stirred at 23° C. for 18 hours before being evaporated. The residue was taken into water and extracted three times with dichloromethane. The combined organic layers were dried over sodium sulfate, filtered, and evaporated. The residue was purified by flash chromatography on silica gel (hexanes/ethyl acetate 90/10 to 40/60) to give 72 mg of (S)-3-((S)-sec-butyl)-7-fluoro-5-phenyl-1,3-dihydro-2H-benzo[e][1,4]diazepin-2-one (10% yield over three steps). MS (ESI) m/z 311.21 (M+H)⁺

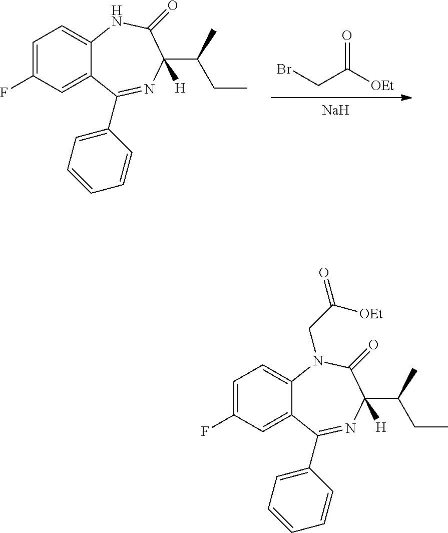

Step 4: Sodium hydride 60% dispersion in oil (18 mg, 0.46 mmol) was added at 0° C. to a solution of (S)-3-((S)-sec-butyl)-7-fluoro-5-phenyl-1,3-dihydro-2H-benzo[e][1,4]diazepin-2-one (72 mg, 0.23 mmol) in 1.1 mL of tetrahydrofuran. The resulting mixture was stirred at 0° C. for two hours before adding ethyl bromoacetate (40 μL, 0.345 mmol). The resulting mixture was stirred at 23° C. for 18 hours before being cooled to 0° C., quenched by addition of water, and extracted three times with ethyl acetate. The combined organic layers were dried over sodium sulfate, filtered, and evaporated. The residues was purified by flash chromatography on silica gel (hexanes/ethyl acetate 100/0 to 60/40) to give 68 mg of ethyl 2-((S)-3-((S)-sec-butyl)-7-fluoro-2-oxo-5-phenyl-2,3-dihydro-1H-benzo[e][1,4]diazepin-1-yl)acetate (76% yield). MS (ESI) m/z 397.27 (M+H)⁺

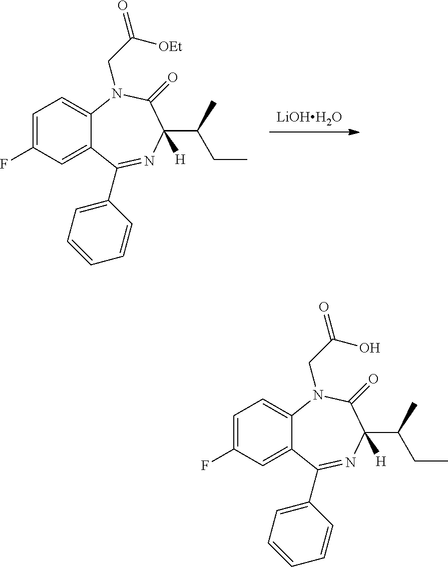 [SMILES: CCOC(=O)CN1C(=O)[C@H]([C@@H](C)CC)N=C(c2ccccc2)c2cc(F)ccc21 ; "LiOH·H2O" ; CC[C@H](C)[C@@H]1N=C(c2ccccc2)c2cc(F)ccc2N(CC(=O)O)C1=O]

Step 5: Lithium hydroxide monohydrate (39 mg, 0.94 mmol) was added at 23° C. to a solution of ethyl 2-((S)-3-((S)-sec-butyl)-7-fluoro-2-oxo-5-phenyl-2,3-dihydro-1H-benzo[c][1,4]diazepin-1-yl)acetate (68 mg, 0.17 mmol) in 4 mL of a 1/1 mixture of 1,4-dioxane and water. The resulting mixture was stirred at 23° C. for 18 hours before being concentrated to half volume. The solution was cooled to 0° C. and the pH was adjusted to 5-6 by addition of 10% aqueous citric acid before being extracted 3 times with ethyl acetate. The combined organic layers were dried over sodium sulfate, filtered, and evaporated. The residue was purified by flash chromatography on silica gel (dichloromethane/methanol 100/0 to 80/20) followed by preparative thin layer chromatography (dichloromethane/methanol 95/5+1 drop acetic acid) to give 37 mg of 2-((S)-3-((S)-sec-butyl)-7-fluoro-2-oxo-5-phenyl-2,3-dihydro-1H-benzo[e][1,4]diazepin-1-yl)acetic acid. 1H NMR (400 MHz, CDCl3) δ 7.60-7.58 (m, 2H), 7.50-7.42 (m, 1H), 7.40-7.36 (m, 2H), 7.36-7.25 (m, 1H), 7.02 (dd, J=12, 2.4 Hz, 111), 4.61-4.50 (m, 2H), 3.26 (d, J=10 Hz, 111), 2.63-2.56 (m, 111), 2.03-1.96 (m, 111), 1.26-1.13 (m, 2H), 0.99-0.92 (m, 6H). MS (ESI): m/z 369.29 (M+H)⁺.

### Example 6: Synthesis of 2-((S)-3-((S)-sec-butyl)-7-methyl-2-oxo-5-phenyl-2,3-dihydro-1H-benzo[e][1,4]diazepin-1-yl)acetic acid

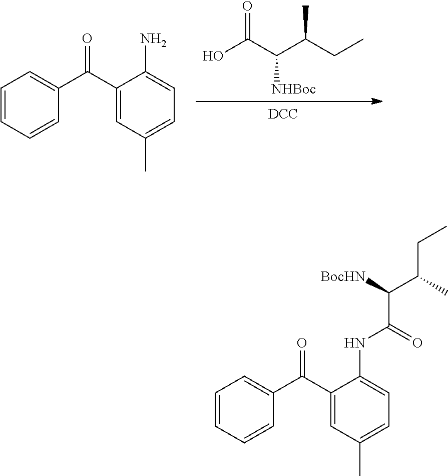

Step 1: A solution of dicyclohexylcarbodiimide 1M in dichloromethane (1.5 mL, 1.5 mmol) was added 0° C. to a solution of L-N-Boc-isoleucine (350 mg, 1.51 mmol) in 0.8 mL of dichloromethane. The resulting mixture was stirred at 23° C. for 20 minutes before being cooled back to 0° C. A solution of (2-amino-5-methylphenyl)(phenyl)methanone (350 mg, 1.51 mmol) in 1.4 mL of dichloromethane was then added dropwise and the resulting mixture was stirred at 23° C. for 24 hours. The insoluble material was filtered off and the filtrate evaporated. The residue was purified by flash chromatography on silica gel (hexanes/ethyl acetate 80/20) to give 331 mg of still impure tert-butyl ((2S,3S)-1-((2-benzoyl-4-methylphenyl)amino)-3-methyl-1-oxopentan-2-yl)carbamate which was used in the next step without further purification. MS (ESI) m/z 447.15 (M+Na)⁺

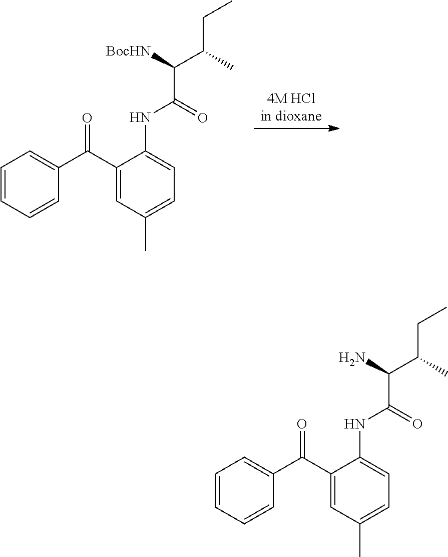 [SMILES: CC[C@H](C)[C@H](NC(=O)OC(C)(C)C)C(=O)Nc1ccc(C)cc1C(=O)c1ccccc1 ; "4M HClin dioxane" ; CC[C@H](C)[C@H](N)C(=O)Nc1ccc(C)cc1C(=O)c1ccccc1]

Step 2: Hydrochloric acid 4M in 1,4-dioxane (2 mL, 8 mmol) was added at 23° C. to a solution of impure tert-butyl ((2S,3S)-1-((2-benzoyl-4-methylphenyl)amino)-3-methyl-1-oxopentan-2-yl)carbamate (330 mg, theoretically 0.78 mmol) in 2.1 mL of 1,4-dioxane. The resulting mixture was stirred at 23° C. for 18 hours before being evaporated. The residue was dissolved in dichloromethane and the solution was washed first with saturated aqueous sodium bicarbonate and then with water before being dried over sodium sulfate, filtered, and evaporated to give 201 mg of crude (2S,3S)-2-amino-N-(2-benzoyl-4-methylphenyl)-3-methylpentanamide, which was used in the next step without purification. MS (ESI) m/z 325.16 (M+H)⁴

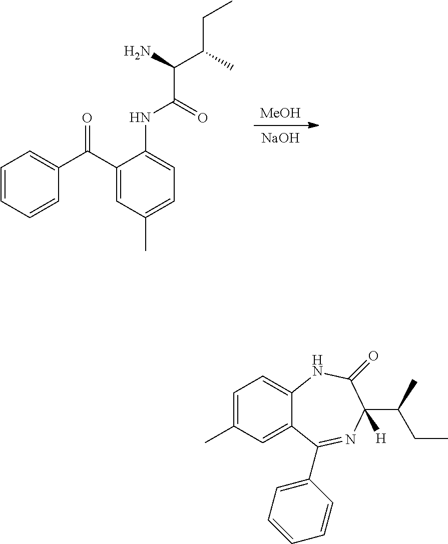 [SMILES: "MeOH" ; CC[C@H](C)[C@H](N)C(=O)Nc1ccc(C)cc1C(=O)c1ccccc1 ; "NaOH" ; CC[C@H](C)[C@@H]1N=C(c2ccccc2)c2cc(C)ccc2NC1=O]

Step 3: To a solution of (2S,3S)-2-amino-N-(2-benzoyl-4-methylphenyl)-3-methylpentanamide (200 mg, theoretically 0.62 mmol)) in 2 mL of methanol and 0.2 mL of water was added at 0° C. several drops of 1M aqueous sodium hydroxide to adjust the pH to 8-9. The resulting mixture was stirred at 23° C. for 18 hours before being evaporated. The residue was taken into water and extracted three times with dichloromethane. The combined organic layers were dried over sodium sulfate, filtered, and evaporated. The residue was purified by flash chromatography on silica gel (hexanes/ethyl acetate 95/5 to 60/40) to give 117 mg (S)-3-((S)-sec-butyl)-7-methyl-5-phenyl-1,3-dihydro-2H-benzo[e][1,4]diazepin-2-one MS (ESI) m/z 307.3 (M+H)⁺

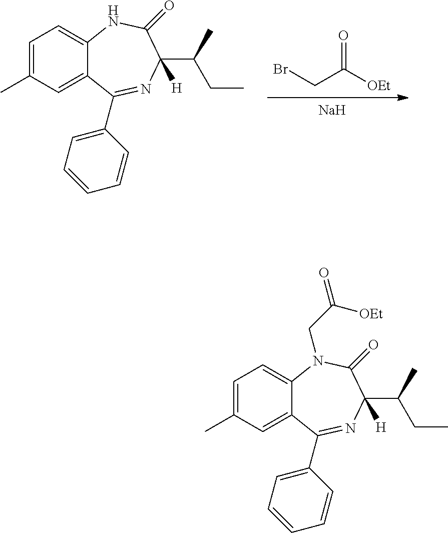

Step 4: Sodium hydride 60% dispersion in oil (28 mg, 0.66 mmol) was added at 0° C. to a solution of (S)-3-((S)-sec-butyl)-7-methyl-5-phenyl-1,3-dihydro-2H-benzo[e][1,4]diazepin-2-one (102 mg, 0.33 mmol) in 1.5 mL of tetrahydrofuran. The resulting mixture was stirred at 0° C. for 2.5 before adding ethyl bromoacetate (0.06 mL, 0.5 mmol). The resulting mixture was stirred at 23° C. for 18 hours before being cooled to 0° C., quenched by addition of water, and extracted three times with ethyl acetate. The combined organic layers were dried over sodium sulfate, filtered, and evaporated. The residues was purified by flash chromatography on silica gel (hexanes/ethyl acetate 95/5 to 60/40) to give 94 mg of ethyl 2-((S)-3-((S)-sec-butyl)-7-methyl-2-oxo-5-phenyl-2,3-dihydro-1H-benzo[e][1,4]diazepin-1-yl)acetate (73% yield). MS (ESI) m/z 393.29 (M+H)⁺

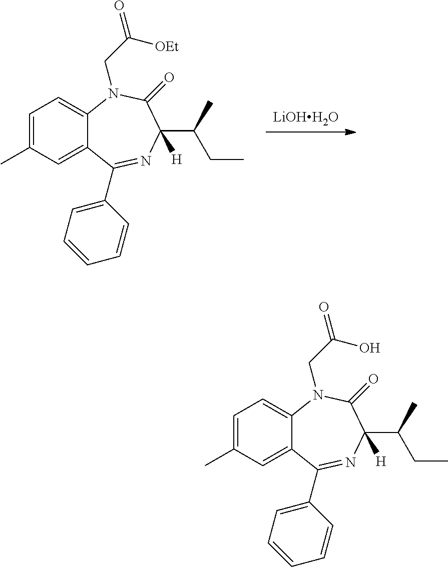 [SMILES: CCOC(=O)CN1C(=O)[C@H]([C@@H](C)CC)N=C(c2ccccc2)c2cc(C)ccc21 ; "LiOH·H2O" ; CC[C@H](C)[C@@H]1N=C(c2ccccc2)c2cc(C)ccc2N(CC(=O)O)C1=O]

Step 5: Lithium hydroxide monohydrate (55 mg, 1.32 mmol) was added at 23° C. to a solution of ethyl 2-((S)-3-((S)-sec-butyl)-7-methyl-2-oxo-5-phenyl-2,3-dihydro-1H-benzo[e][1,4]diazepin-1-yl)acetate (94 mg, 0.24 mmol) in 6 mL of a 1/1 mixture of 1,4-dioxane and water. The resulting mixture was stirred at 23° C. for 18 hours before being concentrated to half volume. The solution was cooled to 0° C. and the pH was adjusted to 5-6 by addition of 10% aqueous citric acid before being extracted 3 times with ethyl acetate. The combined organic layers were dried over sodium sulfate, filtered, and evaporated. The residue was crystallized and recrystallized twice in dichloromethane/hexanes to give 61 mg of 2-((S)-3-((S)-sec-butyl)-7-methyl-2-oxo-5-phenyl-2,3-dihydro-1H-benzo[e][1,4]diazepin-1-yl)acetic acid. 1H NMR (400 MHz, DMSO-d6) δ 12.78 (s, 1H), 7.51-7.38 (m, 7H), 7.00 (s, 1H), 4.59 (d, J=17.2 Hz, 1H), 4.45 (d, J=17.6 Hz, 1H), 3.06 (d, J=10.0 Hz, 1H), 2.37-2.30 (m, 1H), 2.25 (s, 3H), 2.05-1.83 (m, 1H), 1.14-1.05 (m, 1H), 0.85 (t, J=13.6 Hz, 6H). MS (ESI): m/z 365.32 (M+H)⁺.

### Example 7: Synthesis of 2-((S)-3-((S)-sec-butyl)-7-methoxy-2-oxo-5-phenyl-2,3-dihydro-1H-benzo[e][1,4]diazepin-1-yl)acetic acid

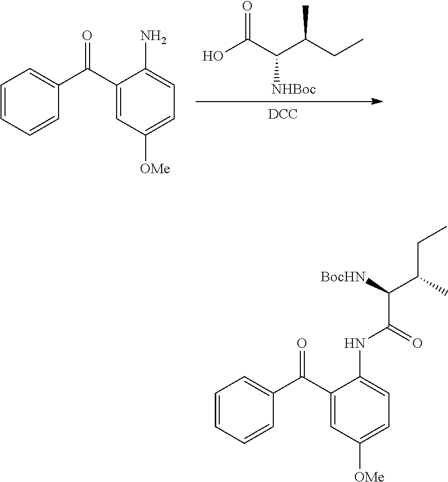

Step 1: A solution of dicyclohexylcarbodiimide 1M in dichloromethane (1 mL, 1 mmol) was added at 23° C. to a solution of L-N-Boc-isoleucine (231 mg, 1 mmol) in 0.5 mL of dichloromethane. The resulting mixture was stirred at 23° ^{c} for 2 hours before adding a solution of (2-amino-5-methoxyphenyl)(phenyl)methanone (229 mg, 1 mmol) in 2 mL of dichloromethane. The resulting mixture was stirred at 23° C. for 18 hours before being filtered. The solid was rinsed with dichloromethane and the filtrate was filtered once more through a short plug of silica. After rinsing the silica plug with ethyl acetate, the filtrate was evaporated to give crude tert-butyl ((2S,3S)-1-((2-benzoyl-4-methoxyphenyl)amino)-3-methyl-1-oxopentan-2-yl)carbamate which was used in the next step without purification.

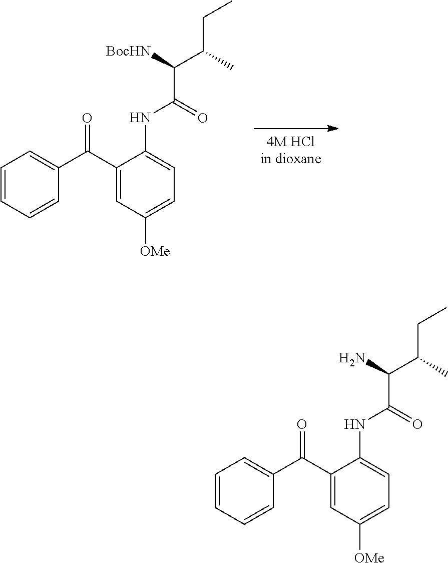 [SMILES: CC[C@H](C)[C@H](NC(=O)OC(C)(C)C)C(=O)Nc1ccc(OC)cc1C(=O)c1ccccc1 ; "4M HClin dioxane" ; CC[C@H](C)[C@H](N)C(=O)Nc1ccc(OC)cc1C(=O)c1ccccc1]

Step 2: Hydrochloric acid 4M in 1,4-dioxane (2.5 mL, 10 mmol) was added at 23° C. to a solution of crude tert-butyl ((2S,3S)-1-((2-benzoyl-4-methoxyphenyl)amino)-3-methyl-1-oxopentan-2-yl)carbamate (theoretically 1 mmol) in 2 mL of 1,4-dioxane. The resulting mixture was stirred at 23° C. for 4 hours before being evaporated. The residue was dissolved in dichloromethane and the solution was washed with saturated aqueous sodium bicarbonate before being dried over magnesium sulfate and evaporated to give (2S,3S)-2-amino-N-(2-benzoyl-4-methoxyphenyl)-3-methylpentanamide which was used crude in the next reaction without purification.

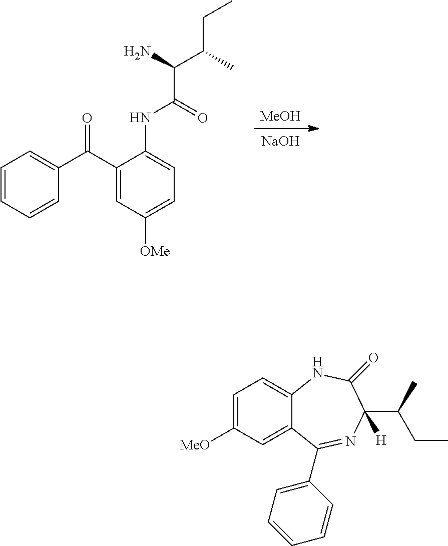 [SMILES: "MeOH" ; CC[C@H](C)[C@H](N)C(=O)Nc1ccc(OC)cc1C(=O)c1ccccc1 ; "NaOH" ; CC[C@H](C)[C@@H]1N=C(c2ccccc2)c2cc(OC)ccc2NC1=O]

Step 3: To a solution of crude 2-((S)-3-((S)-sec-butyl)-7-methoxy-2-oxo-5-phenyl-2,3-dihydro-1H-benzo[e][1,4]diazepin-1-yl)acetic acid (theoretically 1 mmol) in 5 mL of methanol and 0.5 mL of water was added at 0° C. several drops of 1M aqueous sodium hydroxide to adjust the pH to 8-9. The resulting mixture was slowly warmed to 23° C. was stirred for 48 hours before being evaporated. The residue was taken into water and extracted three times with dichloromethane. The combined organic layers were dried over magnesium sulfate, filtered, and evaporated. The residue was purified by flash chromatography on silica gel (hexanes/ethyl acetate 100/0 to 52/48) to give 230 mg of (S)-3-((S)-sec-butyl)-7-methoxy-5-phenyl-1,3-dihydro-2H-benzo[e][1,4]diazepin-2-one (73% yield over three steps). ¹H NMR (400 MHz, DMSO-d6) δ 10.31 (s, 1H), 7.52-7.36 (m, 5H), 7.22-7.12 (m, 2H), 6.69 (d, J=2.5 Hz, 1H), 3.65 (s, 3H), 3.05 (d, J=9.6 Hz, 1H), 2.30 (s, 1H), 1.88 (d, J=9.2 Hz, 1H), 1.24-1.16 (m, 1H), 0.91 (d, J=6.5 Hz, 3H), 0.87 (t, J=7.4 Hz, 3H); MS (ESI) m/z 323.31 (M+H)⁺

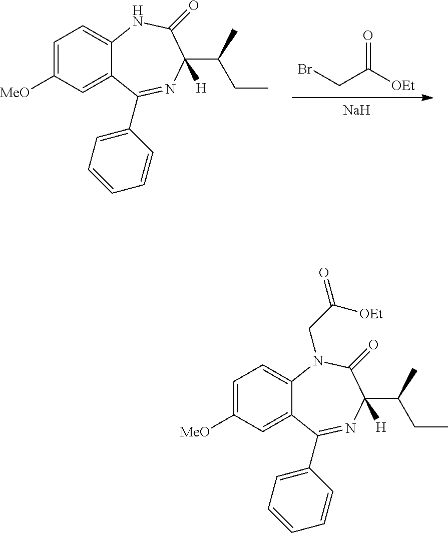

Step 4: Sodium hydride 60% dispersion in oil (57 mg, 1.43 mmol) was added a 0° C. to a solution of (S)-3-((S)-sec-butyl)-7-methoxy-5-phenyl-1,3-dihydro-2H-benzo[e][1,4]diazepin-2-one (230 mg, 0.713 mmol) in 3.5 mL of tetrahydrofuran. The resulting mixture was stirred at 0° C. for 3 hours before adding ethyl bromoacetate (0.12 mL, 1.08 mmol). The resulting mixture was stirred at 23° C. for 18 hours before being cooled to 0° C., quenched by addition of water, and extracted three times with ethyl acetate. The combined organic layers were dried with magnesium sulfate, filtered, and evaporated. The residues was purified by flash chromatography on silica gel (hexanes/ethyl acetate 100/0 to 55/45) to give 236 mg of ethyl 2-((S)-3-((S)-sec-butyl)-7-methoxy-2-oxo-5-phenyl-2,3-dihydro-1H-benzo[e][1,4]diazepin-1-yl)acetate. ¹H NMR (400 MHz, DMSO-d6) δ 7.58-7.53 (m, 2H), 7.50-7.39 (m, 4H), 7.20 (dd, J=9.1, 3.0 Hz, 1H), 6.67 (d, J=2.9 Hz, 1H), 4.63 (d, J=17.5 Hz, 1H), 4.51 (d, J=17.5 Hz, 1H), 3.99-3.92 (m, 2H), 3.68 (s, 3H), 3.18 (d, J=10.1 Hz, 1H), 2.39-2.28 (m, 1H), 1.87 (ddd, J=13.2, 7.5, 3.1 Hz, 1H), 1.18-1.07 (m, 1H), 1.02 (t, J=7.1 Hz, 3H), 0.86 (d, J=7.0 Hz, 3H), 0.85 (t, J=7.4 Hz, 3H); MS (ESI) m/z 409.30 (M+H)⁺

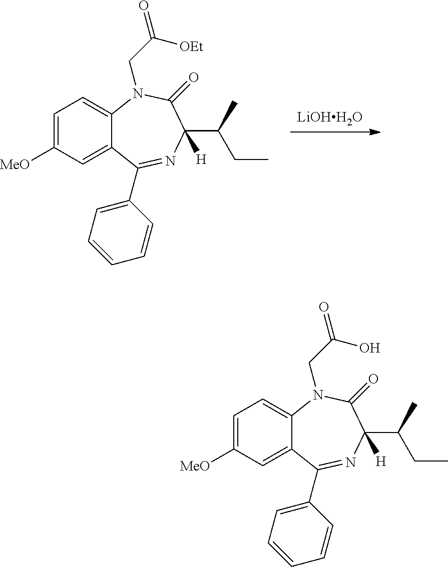 [SMILES: CCOC(=O)CN1C(=O)[C@H]([C@@H](C)CC)N=C(c2ccccc2)c2cc(OC)ccc21 ; "LiOH·H2O" ; CC[C@H](C)[C@@H]1N=C(c2ccccc2)c2cc(OC)ccc2N(CC(=O)O)C1=O]

Step 5: Lithium hydroxide monohydrate (121 mg, 2.89 mmol) was added at 23° C. to a solution of ethyl 2-((S)-3-((S)-sec-butyl)-7-methoxy-2-oxo-5-phenyl-2,3-dihydro-1H-benzo[e][1,4]diazepin-1-yl)acetate (236 mg, 0.578 mmol) in 12 mL of a 1/1 mixture of 1,4-dioxane and water. The resulting mixture was stirred at 23° C. for 18 hours before being concentrate to half volume. The solution was cooled to 0° C. and the pH was adjusted to 5-6 by addition of 10% aqueous citric acid before being extracted 3 times with ethyl acetate. The combined organic layers were dried over magnesium sulfate, filtered, and evaporated. The residue was taken into ethyl acetate and the insoluble material was filtered. The filtrate was evaporated and the residue was crystallized in ethyl acetate/hexanes to give 211 mg of 2-((S)-3-((S)-sec-butyl)-7-methoxy-2-oxo-5-phenyl-2,3-dihydro-1H-benzo[e][1,4]diazepin-1-yl)acetic acid.

### Example 8: Synthesis 2-((S)-3-((S)-sec-butyl)-7-cyano-2-oxo-5-phenyl-2,3-dihydro-1H-benzo[e][1,4]diazepin-1-yl)acetic acid

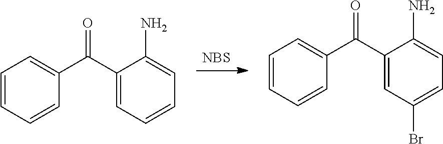

Step 1: N-Bromosuccinimide (541 mg, 3.04 mmol) dissolved in 25 mL of dichloromethane was added at −10° C. to a solution of (2-aminophenyl)(phenyl)methanone (600 mg, 3.04 mmol) in 10 mL of dichloromethane. The resulting mixture was stirred at −10° C. for an hour before being diluted with dichloromethane, washed with saturated aqueous sodium carbonate, dried over sodium sulfate, filtered, and evaporated. The residue was combined with a second batch of product obtained the same way from 600 mg of (2-aminophenyl)(phenyl)methanone and purified by flash chromatography on silica gel (95/5 to 60/40) to give 1.35 g of (2-amino-5-bromophenyl)(phenyl)methanone. MS (ESI) m/z 276.20, 278.11 (M+H)⁺

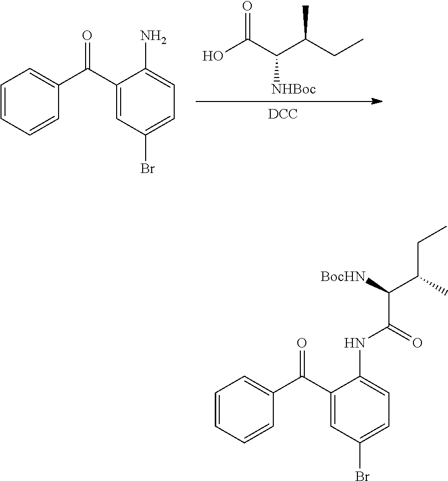

Step 2: A solution of dicyclohexylcarbodiimide 1M in dichloromethane (5.38 mL, 5.38 mmol) was added at 0° C. to a solution of L-N-Boc-isoleucine (1.24 g, 5.38 mmol) in 2.7 mL of dichloromethane. The resulting mixture was warmed to 23° C. before being cooled to 0° C. A solution of (2-amino-5-bromophenyl)(phenyl)methanone (1.35 g, 4.89 mmol) in 4.6 mL of dichloromethane was then added dropwise and the resulting mixture was stirred at 23° C. for 18 hours. The insoluble material was filtered off and the filtrate evaporated. The residue was purified by flash chromatography on silica gel (dichloromethane/hexanes 12/88 to 100/0 followed by ethyl acetate/dichloromethane 80/20) to give 1.475 g of tert-butyl ((2S,3S)-1-((2-benzoyl-4-bromophenyl)amino)-3-methyl-1-oxopentan-2-yl)carbamate, which was used in the next step without further purification. MS (ESI) m/z 489.24 (M+H)⁺

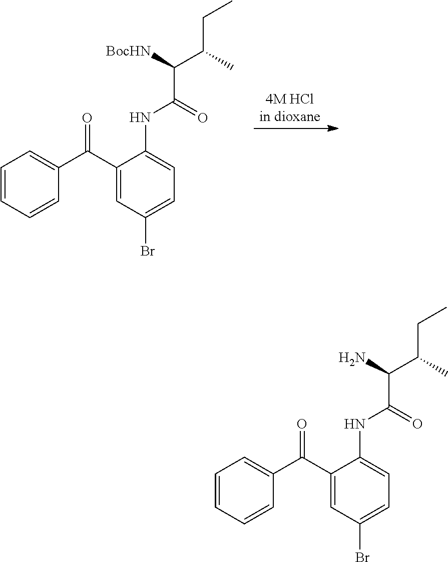 [SMILES: CC[C@H](C)[C@H](NC(=O)OC(C)(C)C)C(=O)Nc1ccc(Br)cc1C(=O)c1ccccc1 ; "4M HClin dioxane" ; CC[C@H](C)[C@H](N)C(=O)Nc1ccc(Br)cc1C(=O)c1ccccc1]

Step 3: Hydrochloric acid 4M in 1,4-dioxane (7.5 mL, 30.1 mmol) was added at 23° C. to a solution of impure tert-butyl ((2S,3S)-1-((2-benzoyl-4-bromophenyl)amino)-3-methyl-1-oxopentan-2-yl)carbamate (1.475 mg, theoretically 3.01 mmol) in 8 mL of 1,4-dioxane. The resulting mixture was stirred at 23° C. for 18 hours before being evaporated. The residue was dissolved in dichloromethane and the solution was washed first with saturated aqueous sodium bicarbonate and then with water before being dried over sodium sulfate, filtered, and evaporated to give 912 mg of crude (2S,3S)-2-amino-N-(2-benzoyl-4-bromophenyl)-3-ethylpentanamide, which was used in the next step without purification. MS (ESI) m/z 389.3 (M+H)⁺

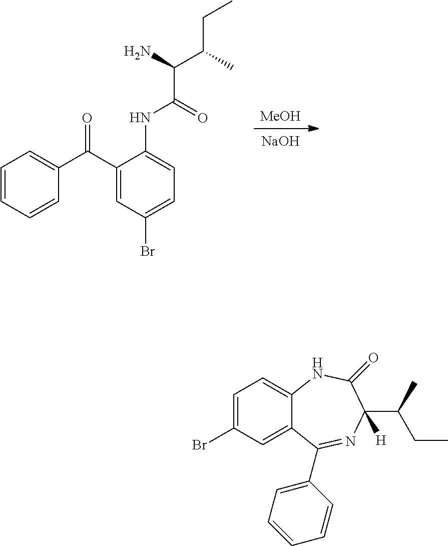 [SMILES: "MeOH" ; CC[C@H](C)[C@H](N)C(=O)Nc1ccc(Br)cc1C(=O)c1ccccc1 ; "NaOH" ; CC[C@H](C)[C@@H]1N=C(c2ccccc2)c2cc(Br)ccc2NC1=O]

Step 4: To a solution of crude (2S,3S)-2-amino-N-(2-benzoyl-4-bromophenyl)-3-methylpentanamide (912 mg, theoretically 2.35 mmol) in 10 mL of methanol and 1 mL of water was added at 0° C. several drops of 1M aqueous sodium hydroxide to adjust the pH to 8-9. The resulting mixture was stirred at 23° C. for 18 hours before being evaporated. The residue was taken into water and extracted three times with dichloromethane. The combined organic layers were dried over sodium sulfate, filtered, and evaporated. The residue was purified by flash chromatography on silica gel (hexanes/ethyl acetate 95/5 to 40/60) to give 542 mg (S)-7-bromo-3-((S)-sec-butyl)-5-phenyl-1,3-dihydro-2H-benzo[e][1,4]diazepin-2-one (30% yield over three steps). MS (ESI) m/z 371.35 (M+H)⁺

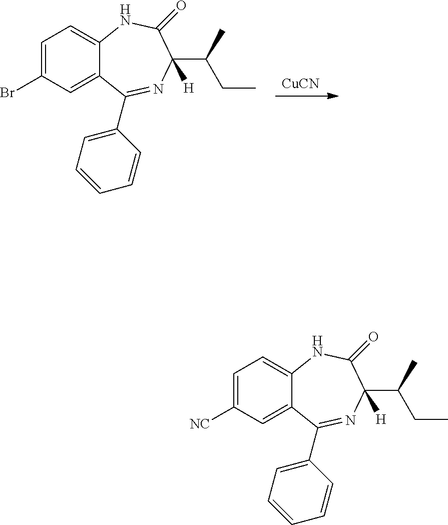

Step 5: A mixture of (S)-7-bromo-3-((S)-sec-butyl)-5-phenyl-1,3-dihydro-2H-benzo[e][1,4]diazepin-2-one (543 mg, 1.46 mmol) and copper cyanide (222 mg, 2.48 mmol) in 9 mL of N,N-dimethylacetamide was stirred under microwave irradiation at 180° C. for 11 hours before being cooled to 23° C. and partitioned between water and dichloromethane. The precipitate that formed was filtered and rinsed with water. The filtrate was washed with water, dried over sodium sulfate, filtered, and evaporated. The residue was purified by flash chromatography on silica gel (hexanes/ethyl acetate 93/7 to 20/80) to give 184 mg of (S)-3-((S)-sec-butyl)-2-oxo-5-phenyl-2,3-dihydro-1H-benzo[e][1,4]diazepine-7-carbonitrile (40% yield). MS (ESI) m/z 318.31 (M+H)⁺

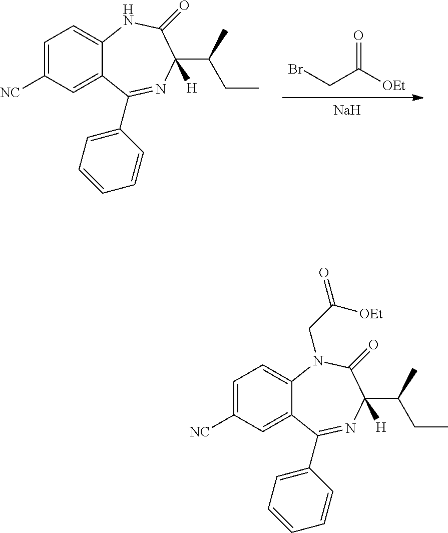

Step 6: Sodium hydride 60% dispersion in oil (47 mg, 1.16 mmol) was added at 0° C. to a solution (S)-3-((S)-sec-butyl)-2-oxo-5-phenyl-2,3-dihydro-1H-benzo[e][1,4]diazepine-7-carbonitrile (184 mg, 0.58 mmol) in 2.7 mL of tetrahydrofuran. The resulting mixture was stirred at 0° C. for two and half hours before adding ethyl bromoacetate (100 μL, 0.87 mmol). The resulting mixture was stirred at 23° C. for 2.5 hours before being quenched by addition of water and extracted three times with ethyl acetate. The combined organic layers were washed with water and brine, dried over sodium sulfate, filtered, and evaporated. The residues was purified by flash chromatography on silica gel (hexanes/ethyl acetate 95/5 to 60/40) to give 221 mg of ethyl 2-((S)-3-((S)-sec-butyl)-7-cyano-2-oxo-5-phenyl-2,3-dihydro-1H-benzo[c][1,4]diazepin-1-yl)acetate (94% yield). MS (ESI) m/z 404.3 (M+H)⁺

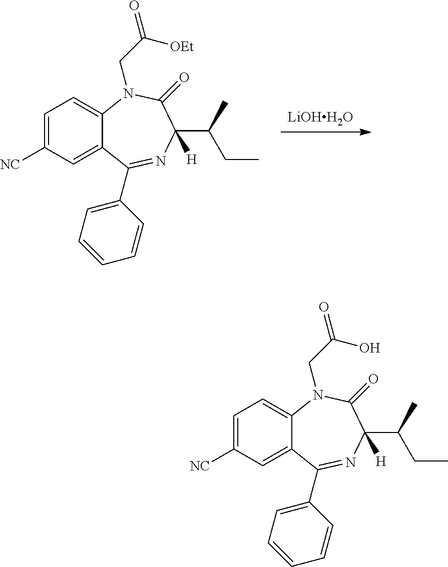 [SMILES: CCOC(=O)CN1C(=O)[C@H]([C@@H](C)CC)N=C(c2ccccc2)c2cc(C#N)ccc21 ; "LiOH·H2O" ; CC[C@H](C)[C@@H]1N=C(c2ccccc2)c2cc(C#N)ccc2N(CC(=O)O)C1=O]

Step 7: Lithium hydroxide monohydrate (127 mg, 3.03 mmol) was added at 23° C. to a solution of ethyl 2-((S)-3-((S)-sec-butyl)-7-cyano-2-oxo-5-phenyl-2,3-dihydro-1H-benzo[e][1,4]diazepin-1-yl)acetate (221 mg, 0.55 mmol) in 18 mL of a 1/1 mixture of 1,4-dioxane and water. The resulting mixture was stirred at 23° C. for five hours before being concentrated to half volume. The solution was cooled to 0° C. and the pH was adjusted to 5-6 by addition of 10% aqueous citric acid before being extracted 3 times with ethyl acetate. The combined organic layers were dried over sodium sulfate, filtered, and evaporated. The residue was purified twice by flash chromatography on silica gel (dichloromethane/methanol 100/0 to 90/10) to give 67 mg of 2-((S)-3-((S)-sec-butyl)-7-cyano-2-oxo-5-phenyl-2,3-dihydro-1H-benzo[e][1,4]diazepin-1-yl)acetic acid. ¹H NMR (400 MHz, CDCl3) δ 7.81-7.78 (m, 1H), 7.65-7.64 (m, 1H), 7.54-7.51 (m, 2H), 7.49-7.45 (m, 1H), 7.41-7.24 (m, 3H), 4.67 (d, J=18 Hz, 1H), 4.57-4.47 (m, 1H), 3.24-3.19 (m, 1H), 2.62-2.50 (m, 1H), 2.04-1.95 (m, 1H), 1.16-1.12 (m, 1H), 0.98-0.91 (m, 6H). MS (ESI): m/z 376.33 (M+H)⁺.

### Example 9: Synthesis of: 2-((S)-3-((S)-sec-butyl)-7-fluoro-5-(4-methoxyphenyl)-2-oxo-2,3-dihydro-1H-benzo[e][1,4]diazepin-1-yl)acetic acid

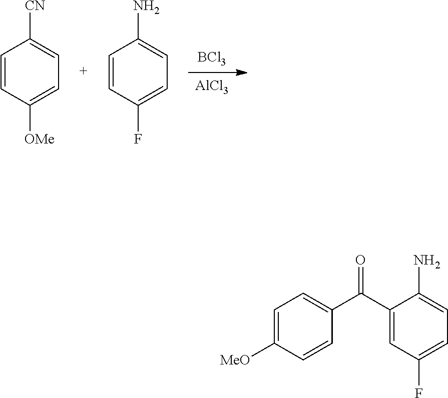

Step 1: 4-Fluoroaniline (758 μL, 8 mmol) in 8.9 mL of dichloroethane was added dropwise at 0° C. to a solution of boron trichloride 1M in dichloromethane (5.2 ml, 5.2 mmol). The resulting mixture was stirred at 0° C. for 30 minutes before adding 4-methoxybenzonitrile (533 mg, 4 mmol) followed by aluminum trichloride (693 mg, 5.2 mmol). The resulting mixture was stirred at 23° C. for 30 minutes before being stirred at 90° C. for 18 hours. The reaction mixture was then cooled to 0° C. before being quenched by addition of 9 mL. of 2M aqueous hydrochloric acid. The resulting mixture was stirred at 90° C. for 30 minutes, cooled back to 23° C., and extracted three times with dichloromethane. The combined organic layers were dried over sodium sulfate, filtered, and evaporated. The residue was purified by flash chromatography on silica gel (hexanes/ethyl acetate 93/7 to 60/40) to give 188 mg of (2-amino-5-fluorophenyl)(4-methoxyphenyl)methanone (10% yield). MS (ESI) m/z 246.16 (M+H)⁺

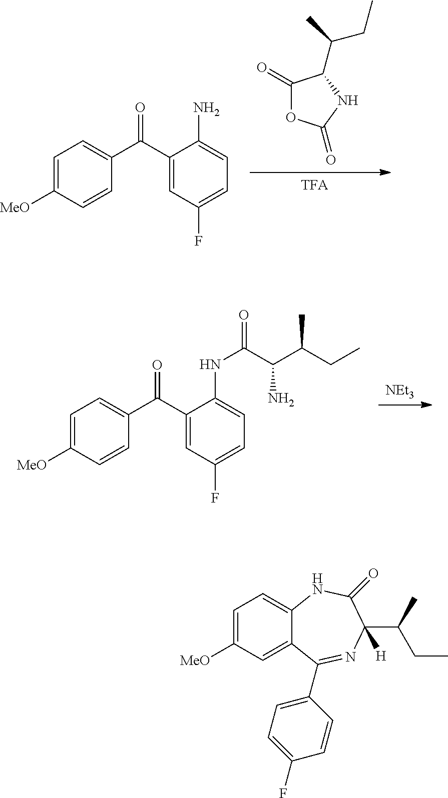 [SMILES: COc1ccc(C(=O)c2cc(F)ccc2N)cc1 ; CC[C@H](C)[C@@H]1NC(=O)OC1=O ; CC[C@H](C)[C@H](N)C(=O)Nc1ccc(F)cc1C(=O)c1ccc(OC)cc1 ; "TFA" ; CC[C@H](C)[C@@H]1N=C(c2ccc(F)cc2)c2cc(OC)ccc2NC1=O ; "NEt3"]

Step 2: A mixture of (2-amino-5-fluorophenyl)(4-methoxyphenyl)methanone (188 mg, 0.77 mmol), (S)-4-((S)-sec-butyl)oxazolidine-2,5-dione (145 mg, 0.92 mmol), and trifluoroacetic acid (176 μL, 2.3 mmol) in 3.8 mL of toluene was stirred at 60° C. for 18 hours before adding triethylamine (321 μL, 2.3 mmol). The resulting mixture was then stirred at 80° C. for three hour before being cooled to 23° C. and evaporated. The residue was purified by flash chromatography on silica gel (hexanes/ethyl acetate 93/7 to 20/80) to give 211 mg (S)-3-((S)-sec-butyl)-5-(4-fluorophenyl)-7-methoxy-1,3-dihydro-2H-benzo[e][1,4]diazepin-2-one. MS (ESI) m/z 341.25 (M+H)⁺

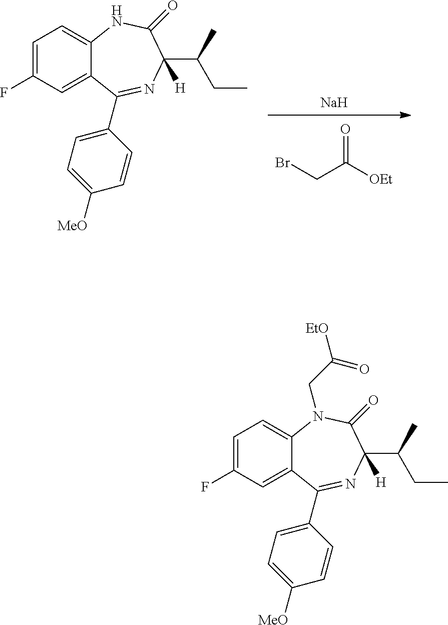

Step 3: Sodium hydride 60% dispersion in oil (50 mg, 1.25 mmol) was added at 0° C. to a solution of (S)-3-((S)-sec-butyl)-5-(4-fluorophenyl)-7-methoxy-1,3-dihydro-2H-benzo[e][1,4]diazepin-2-one (210 mg, 0.627 mmol) in 2.8 mL of tetrahydrofuran. The resulting mixture was stirred at 0° C. for an hour before adding ethyl bromoacetate (103 μL, 0.925 mmol). The reaction mixture was then stirred at 23° C. for 1.5 hours before being cooled back to 0° C., quenched by the addition of iced water, and extracted three times with ethyl acetate. The combined organic layers were washed with water, dried over sodium sulfate, filtered, and evaporated. The residue was purified by flash chromatography on silica gel (hexanes/ethyl acetate 93/7 to 40/60) to give 263 mg of ethyl 2-((S)-3-((S)-scc-butyl)-7-fluoro-5-(4-methoxyphenyl)-2-oxo-2,3-dihydro-1H-benzo[c][1,4]diazepin-1-yl)acetate. MS (ESI) m/z 427.09 (M+H)⁴

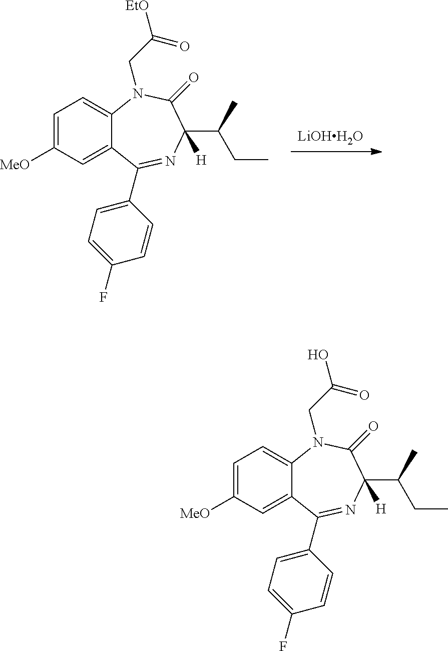 [SMILES: CCOC(=O)CN1C(=O)[C@H]([C@@H](C)CC)N=C(c2ccc(F)cc2)c2cc(OC)ccc21 ; "LiOH·H2O" ; CC[C@H](C)[C@@H]1N=C(c2ccc(F)cc2)c2cc(OC)ccc2N(CC(=O)O)C1=O]

Step 4: Lithium hydroxide monohydrate (142 mg, 3.39 mmol) was added at 23° C. to a solution of ethyl 2-((S)-3-((S)-sec-butyl)-7-fluoro-5-(4-methoxyphenyl)-2-oxo-2,3-dihydro-1H-benzo[e][1,4]diazepin-1-yl)acetate (263 mg, 0.617 mmol) in 14 mL of a 1/1 mixture of 1,4-dioxane and water. The resulting mixture was stirred at 23° C. for 18 hours before being cooled to 0° C., acidified to pH 5-6 by addition of 10% aqueous citric acid, and extracted three times with ethyl acetate. The combined organic layers were washed with brine, dried over sodium sulfate, filtered, and evaporated. The residue was crystallized twice from dichloromethane/hexanes to give 202 mg 2-((S)-3-((S)-sec-butyl)-5-(4-fluorophenyl)-7-methoxy-2-oxo-2,3-dihydro-1H-benzo[e][1,4]diazepin-1-yl)acetic acid. ¹H NMR (400 MHz, DMSO-d6) δ 12.77 (s, 1H), 7.55-7.51 (m, 1H), 7.48-7.43 (m, 3H), 7.01 (dd, J=9.0, 2.6 Hz, 1H), 6.96-6.94 (m, 2H), 4.56 (d, J=17.8 Hz, 1H), 4.42 (d, J=17.8 Hz, 1H), 3.75 (s, 3H), 3.11 (d, J=10.0 Hz, 1H), 2.33-2.28 (m, 1H), 1.86-1.79 (m, 1H), 1.12-1.04 (m, 1H), 0.90-0.80 (m, 6H). MS (EST): m/z 399.13 (M+H)⁺.

### Example 10: Synthesis of 2-((S)-3-((S)-sec-butyl)-5-(4-fluorophenyl)-7-methoxy-2-oxo-2,3-dihydro-1H-benzo[e][1,4]diazepin-1-yl)acetic acid

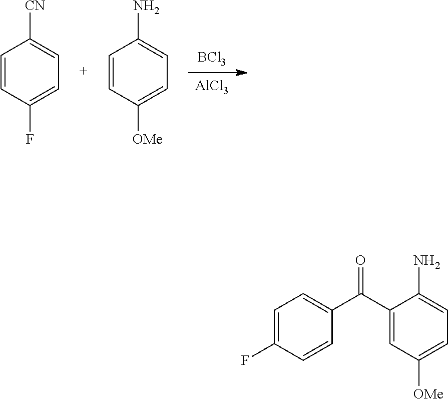

Step 1: 4-Methoxyaniline (2 g, 16.2 mmol) in 8.5 mL of dichloroethane was added dropwise at 0° C. to a solution of boron trichloride 1M in dichloromethane (10.6 mL, 10.6 mmol). The resulting mixture was stirred at 0° C. for 30 minutes before adding 4-fluorobenzonitrile (987 mg, 8.15 mmol) followed by aluminum trichloride (1.41 g, 10.6 mmol). The resulting mixture was stirred at 0° C. for 30 minutes before being stirred at 90° C. for 19 hours. The reaction mixture was then cooled to 23° C. and then to 0° C. before being quenched by addition of 17 mL of 2M aqueous hydrochloric acid. The resulting mixture was stirred at 90° C. for 30 minutes before being cooled to 23° C., extracted three times with dichloromethane. The combined organic layers were dried over sodium sulfate, filtered, and evaporated. The residue was purified by flash chromatography on silica gel (hexanes/ethyl acetate 95/5 to 60/40) to give 1.36 g of (2-amino-5-methoxyphenyl)(4-fluorophenyl)methanone. MS (ESI) m/z 246.17 (M+H)⁺

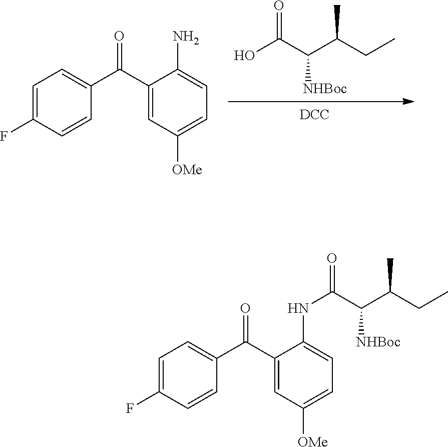

Step 2: A solution of dicyclohexylcarbodiimide 1M in dichloromethane (1.43 mL, 1.43 mmol) was added at 0° C. to a solution of L-N-boc-isoleucine (331 mg, 1.43 mmol) in 0.8 mL of dichloromethane. The resulting mixture was stirred at 23° C. for 30 minutes before being cooled back to 0° C. (2-amino-5-methoxyphenyl)(4-fluorophenyl)methanone (350 mg, 1.43 mmol) in 1.3 mL of dichloromethane was then added and the resulting mixture was stirred at 23° C. for 2 days. The insoluble material that had formed during the reaction was filtered and the filtrate was evaporated. The residue was purified by flash chromatography on silica gel (hexanes/ethyl acetate 80/20) to give impure tert-butyl ((2S,3S)-1-((2-(4-fluorobenzoyl)-4-methoxyphenyl)amino)-3-methyl-1-oxopentan-2-yl) carbamate which was used in the next reaction without further purification. MS (ESI) m/z 459.04 (M+H)⁺

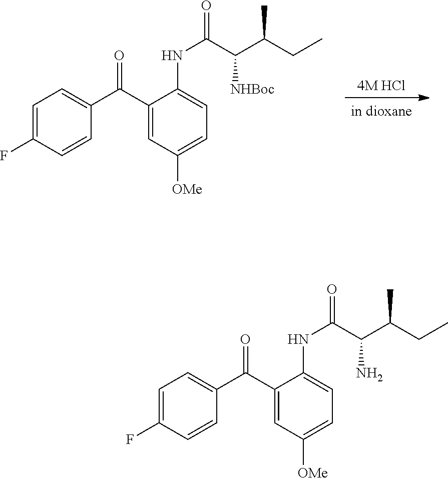 [SMILES: CC[C@H](C)[C@H](NC(=O)OC(C)(C)C)C(=O)Nc1ccc(OC)cc1C(=O)c1ccc(F)cc1 ; "4M HCl" ; "in dioxane" ; CC[C@H](C)[C@H](N)C(=O)Nc1ccc(OC)cc1C(=O)c1ccc(F)cc1]

Step 3: Hydrochloric acid 4M in 1,4-dioxane (2.25 mL, 9 mmol) was added at 23° C. to a solution of impure tert-butyl ((2S,3S)-1-((2-(4-fluorobenzoyl)-4-methoxyphenyl)amino)-3-methyl-1-oxopentan-2-yl)carbamate (theoretically 1.43 mmol) in 2.4 mL of dichloromethane. The resulting mixture was stirred at 23° C. for 18 hours before being evaporated. The residue was dissolved in dichloromethane and the resulting solution was washed with aqueous saturated sodium bicarbonate followed by water, dried over sodium sulfate, filtered, and evaporated to give crude (2S,3S)-2-amino-N-(2-(4-fluorobenzoyl)-4-methoxyphenyl)-3-methylpentanamide which was used in the next step without purification. MS (ESI) m/z 359.19 (M+H)⁺

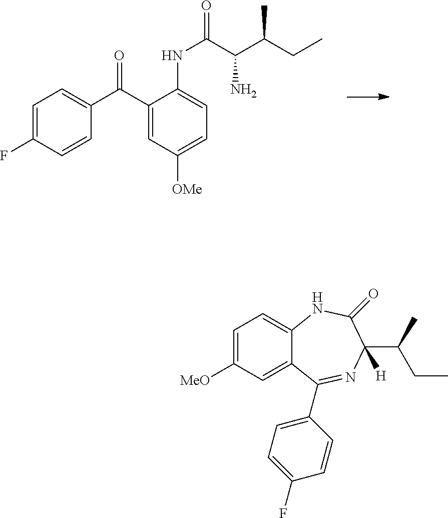 [SMILES: CC[C@H](C)[C@H](N)C(=O)Nc1ccc(OC)cc1C(=O)c1ccc(F)cc1 ; CC[C@H](C)[C@@H]1N=C(c2ccc(F)cc2)c2cc(OC)ccc2NC1=O]

Step 4: (2S,3S)-2-amino-N-(2-(4-fluorobenzoyl)-4-methoxyphenyl)-3-methylpentanamide prepared in step 3 (theoretically 1.43 mmol) was dissolved in 4.2 mL of methanol and 0.42 mL of water. The resulting solution was cooled to 0° C. and the pH was adjusted to 8-9 by addition of aqueous 1M sodium hydroxide. The resulting mixture was allowed to warm to 23° C. and stirred for 18 hours before being evaporated. The residue was partitioned between water and dichloromethane. The aqueous layer was then extracted three times with dichloromethane. The combined organic layers were dried over sodium sulfate, filtered, and evaporated. The residue was purified by flash chromatography on silica gel (hexanes/ethyl acetate 100/0 to 0/100) to give 347 mg of (S)-3-((S)-sec-butyl)-5-(4-fluorophenyl)-7-methoxy-1,3-dihydro-2H-benzo[e][1,4]diazepin-2-one. MS (ESI) m/z 341.31 (M+H)⁺

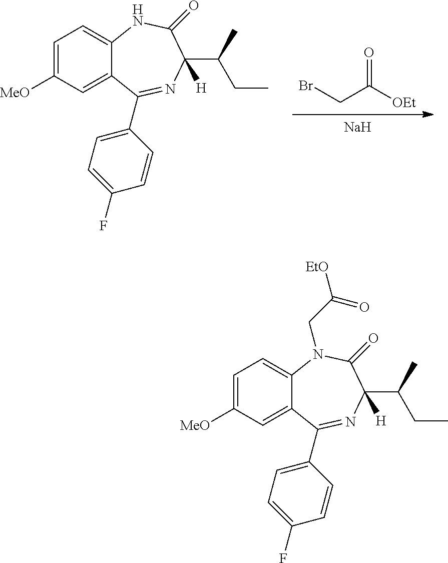

Step 5: Sodium hydride 60% dispersion in oil (82 mg, 1.02 mmol) was added at 0° C. to a solution (S)-3-((S)-sec-butyl)-5-(4-fluorophenyl)-7-methoxy-1,3-dihydro-2H-benzo[e][1,4]diazepin-2-one (347 mg, 1.02 mmol) in 4.7 mL of tetrahydrofuran. The resulting mixture was stirred at 0° C. for two hours before adding ethyl bromoacetate (170 μL, 1.53 mmol). The reaction mixture was then stirred at 23° C. for 2.5 hours before being quenched by the addition of iced water, and extracted three times with ethyl acetate. The combined organic layers were washed with water, dried over sodium sulfate, filtered, and evaporated. The residue was purified by flash chromatography on silica gel (hexanes/ethyl acetate 95/5 to 20/80) to give 256 mg of ethyl 2-((S)-3-((S)-sec-butyl)-5-(4-fluorophenyl)-7-methoxy-2-oxo-2,3-dihydro-1H-benzo[e][1,4]diazepin-1-yl)acetate (59% yield). MS (ESI) m/z 427.37 (M+H)⁺

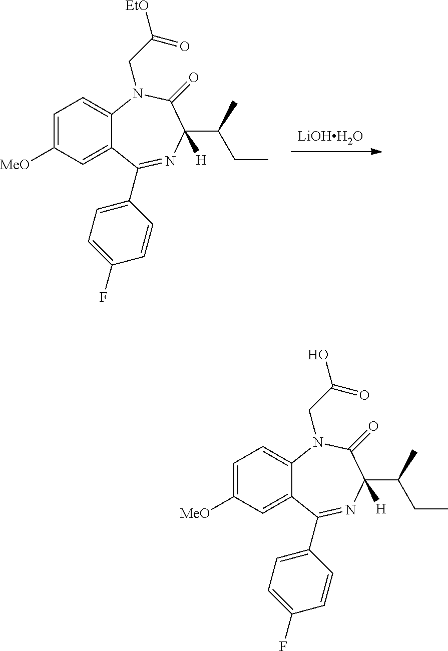 [SMILES: CCOC(=O)CN1C(=O)[C@H]([C@@H](C)CC)N=C(c2ccc(F)cc2)c2cc(OC)ccc21 ; "LiOH·H2O" ; CC[C@H](C)[C@@H]1N=C(c2ccc(F)cc2)c2cc(OC)ccc2N(CC(=O)O)C1=O]

Step 6: Lithium hydroxide monohydrate (139 mg, 3.3 mmol) was added at 23° C. to a solution of ethyl 2-((S)-3-((S)-sec-butyl)-5-(4-fluorophenyl)-7-methoxy-2-oxo-2,3-dihydro-1H-benzo[e][1,4]diazepin-1-yl)acetate (256 mg, 0.6 mmol) in 20 mL of a 1/1 mixture of 1,4-dioxane and water. The resulting mixture was stirred at 23° C. for five hours before being cooled to 0° C., acidified to pH 5-6 by addition of 10% aqueous citric acid, and extracted three times with ethyl acetate. The combined organic layers were dried over magnesium sulfate, filtered, and evaporated to give 167 mg of 2-((S)-3-((S)-sec-butyl)-5-(4-fluorophenyl)-7-methoxy-2-oxo-2,3-dihydro-1H-benzo[e][1,4]diazepin-1-yl)acetic acid. ¹H NMR (400 MHz, DMSO-d6) δ 12.75 (s, 1H), 7.56-7.51 (m, 2H), 7.43 (d, J=8.8 Hz, 1H), 7.23-7.15 (m, 3H), 6.62 (d, J=3.2 Hz, 1H), 4.55 (d, J=17.6 Hz, 1H), 4.36 (d, J=18 Hz, 1H), 3.64 (s, 3H), 3.13 (d, J=10 Hz, 1H), 2.32-2.25 (m, 1H), 1.85-1.79 (m, 1H), 1.09-1.02 (m, 1H), 0.83-0.79 (m, 6H). MS (ESI): m/z 399.36 (M+H)⁺.

### Example 11: 2-((S)-3-((S)-sec-butyl)-7-chloro-5-cyclohexyl-2-oxo-2,3-dihydro-1H-benzo[e][1,4]diazepin-1-yl)acetic acid

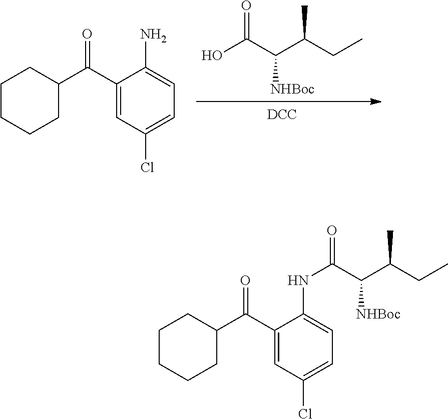

Step 1: A solution of dicyclohexylcarbodiimide 1M in dichloromethane (2.1 mL, 2.1 mmol) was added at 0° C. to a solution of L-N-Boc-isoleucine (486 mg, 2.1 mmol) in 1.2 mL of dichloromethane. The resulting mixture was warmed to 23° C. before being cooled back to 0° C. (2-Amino-5-chlorophenyl)(cyclohexyl)methanone (500 mg, 2.1 mmol) in 1.9 mL of dichloromethane was then added and the reaction mixture was stirred at 23° C. for two days before being filtered. The filtrate was evaporated and the residue was purified by flash chromatography on silica gel (hexanes/dichloromethane 80/20 to 60/40) to give 478 mg of tert-butyl ((2S,3S)-1-((4-chloro-2-(cyclohexanecarbonyl)phenyl)amino)-3-methyl-1-oxopentan-2-yl)carbamate. MS (ESI) m/z 451.03 (M+H)⁺

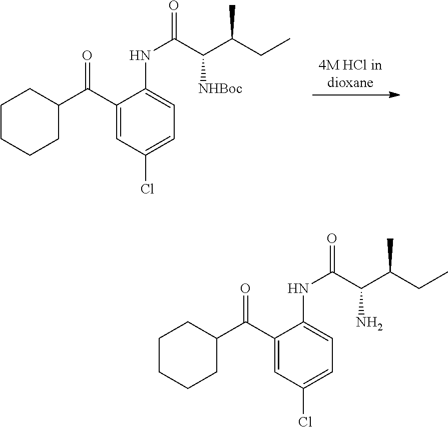 [SMILES: CC[C@H](C)[C@H](NC(=O)OC(C)(C)C)C(=O)Nc1ccc(Cl)cc1C(=O)C1CCCCC1 ; "4M HCl indioxane" ; CC[C@H](C)[C@H](N)C(=O)Nc1ccc(Cl)cc1C(=O)C1CCCCC1]

Step 2: Hydrochloric acid 4M in 1,4-dioxane (2.7 mL, 1.08 mmol) was added at 23° C. to a solution of impure tert-butyl ((2S,3S)-1-((4-chloro-2-(cyclohexanecarbonyl)phenyl)amino)-3-methyl-1-oxopentan-2-yl)carbamate (478 mg, theoretically 1.062 mmol) in 2.8 mL of 1,4-dioxane. The resulting mixture was stirred at 23° C. for 6 hour before being evaporated. The residue was dissolved in dichloromethane and the solution was washed first with saturated aqueous sodium bicarbonate and then with water before being dried over sodium sulfate, filtered, and evaporated to give 310 mg of crude (2S,3S)-2-amino-N-(4-chloro-2-(cyclohexanecarbonyl)phenyl)-3-methylpentanamide, which was used in the next reaction without purification. MS (ESI) m/z 351.22 (M+H)⁺

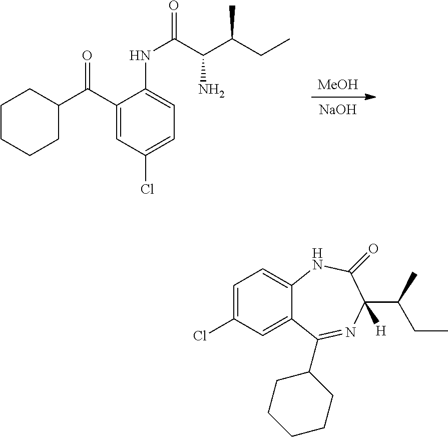 [SMILES: "MeOH" ; CC[C@H](C)[C@H](N)C(=O)Nc1ccc(Cl)cc1C(=O)C1CCCCC1 ; "NaOH" ; CC[C@H](C)[C@@H]1N=C(C2CCCCC2)c2cc(Cl)ccc2NC1=O]

Step 3: To a solution of crude (2S,3S)-2-amino-N-(4-chloro-2-(cyclohexane carbonyl)phenyl)-3-methylpentanamide (theoretically 1.062 mmol) in 3.8 mL of methanol and 0.38 mL of water was added at 0° C. several drops of 1M aqueous sodium hydroxide to adjust the pH to 8-9. The resulting mixture was stirred at 23° C. for 18 hours before being evaporated. The residue was taken into water and extracted three times with dichloromethane. The combined organic layers were dried over sodium sulfate, filtered, and evaporated. The residue was purified by flash chromatography on silica gel (hexanes/ethyl acetate 95/5 to 20/80) to give 86 mg of (S)-3-((S)-sec-butyl)-7-chloro-5-cyclohexyl-1,3-dihydro-2H-benzol 11,4diazepin-2-one. MS (ESI) m/z 333.37 (M+H)⁺

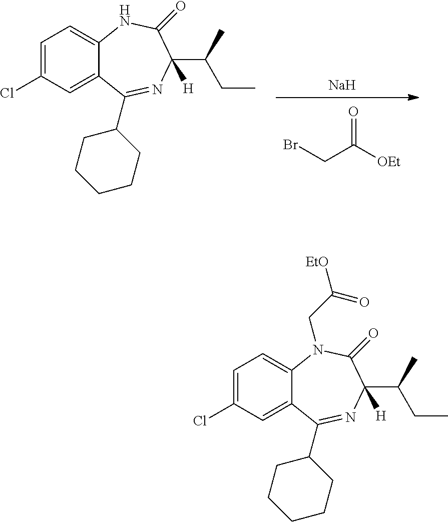

Step 4: Sodium hydride 60% dispersion in oil (20 mg, 0.38 mmol) was added at 0° C. to a solution of (S)-3-((S)-sec-butyl)-7-chloro-5-cyclohexyl-1,3-dihydro-2H-benzo[e][1,4]diazepin-2-one (86 mg, 0.25 mmol) in 1.2 mL of tetrahydrofuran. The resulting mixture was stirred at 0° C. for two hours before adding ethyl bromoacetate (40 μL, 0.38 mmol). The resulting mixture was stirred at 23° C. for 18 hours before being cooled to 0° C., quenched by addition of water, and extracted three times with ethyl acetate. The combined organic layers were dried over sodium sulfate, filtered, and evaporated. The residues was purified by flash chromatography on silica gel (hexanes/ethyl acetate 95/5 to 60/40) to give 79 mg of ethyl 2-((S)-3-((S)-sec-butyl)-7-chloro-5-cyclohexyl-2-oxo-2,3-dihydro-1H-benzo[e][1,4]diazepin-1-yl)acetate (76% yield). MS (ESI) m/z 419.44 (M+H)⁺

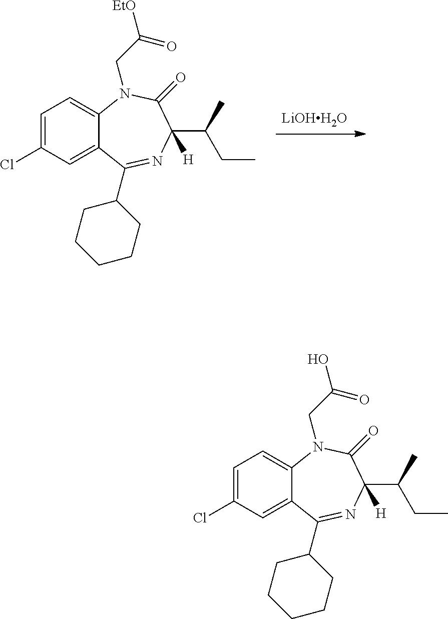 [SMILES: CCOC(=O)CN1C(=O)[C@H]([C@@H](C)CC)N=C(C2CCCCC2)c2cc(Cl)ccc21 ; "LiOH·H2O" ; CC[C@H](C)[C@@H]1N=C(C2CCCCC2)c2cc(Cl)ccc2N(CC(=O)O)C1=O]

Step 6: Lithium hydroxide monohydrate (43 mg, 1.034 mmol) was added at 23° C. to a solution of ethyl 2-((S)-3-((S)-sec-butyl)-7-chloro-5-cyclohexyl-2-oxo-2,3-dihydro-1H-benzo[e][1,4]diazepin-1-yl)acetate (79 mg, 0.188 mmol) in 6 mL of a 1/1 mixture of 1,4-dioxane and water. The resulting mixture was stirred at 23° C. for five hours before being concentrated to half volume. The solution was cooled to 0° C. and the pH was adjusted to 5-6 by addition of 10% aqueous citric acid before being extracted 3 times with ethyl acetate. The combined organic layers were dried over sodium sulfate, filtered, and evaporated. The residue was purified by flash chromatography on silica gel (dichloromethane/methanol 100/0 to 90/10) followed by a second column (hexanes/ethyl acetate 50/50 to 0/100, followed by dichloromethane/methanol 100/0 to 80/20) to give 24 mg of 2-((S)-3-((S)-sec-butyl)-7-chloro-5-cyclohexyl-2-oxo-2,3-dihydro-1H-benzo[e][1,4]diazepin-1-yl)acetic acid. ¹H NMR (400 MHz, CDCl3) δ 7.49 (s, 1H), 7.42 (d, J=8.8 Hz, 1H), 7.17-7.15 (m, 1H), 4.57 (d, J=17.2 Hz, 1H), 4.38 (d, J=18 Hz, 1H), 3.04 (d, J=10 Hz, 1H), 2.72 (m, 1H), 2.43 (m, 1H), 1.95-1.53 (m, 5H), 1.40-1.00 (m, 7H), 0.90-0.81 (m, 6H). MS (ESI): m/z 391.37 (M+H)⁺.

### Example 12: Synthesis of 2-((S)-3-((S)-sec-butyl)-7-chloro-2-oxo-5-(pyridin-4-yl)-2,3-dihydro-1H-benzo[e][1,4]diazepin-1-yl)acetic acid

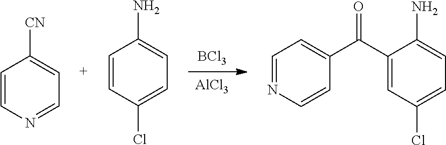

Step 1: 4-Chloroaniline (1.02 g, 8 mmol) in 15 mL of tetrachloroethane was added dropwise at 0° C. to a solution of boron trichloride 1M in dichloromethane (12 mL, 12 mmol). The resulting mixture was stirred at 0° C. for 30 minutes before adding isonicotinonitrile (1 g, 9.6 mmol) followed by aluminum trichloride (1.5 g, 11.2 mmol). The resulting mixture was stirred at 23° C. for 30 minutes before being stirred at 120° C. for four hours and then at 23° C. for 18 hours. The reaction mixture was then cooled to 0° C. before being quenched by addition of 15 mL of 3M aqueous hydrochloric acid, and extracted three times with dichloromethane. The combined organic layers were dried over sodium sulfate, filtered, and evaporated to give 749 mg of (2-amino-5-chlorophenyl)(pyridin-4-yl)methanone. MS (ESI) m/z 233.14 (M+H)⁺

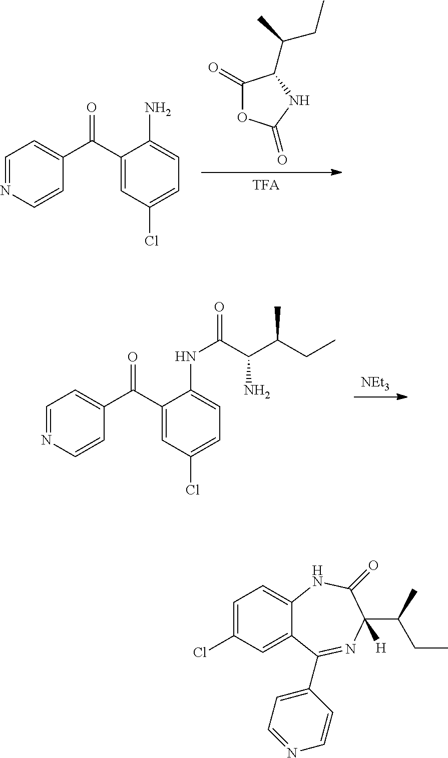 [SMILES: Nc1ccc(Cl)cc1C(=O)c1ccncc1 ; CC[C@H](C)[C@@H]1NC(=O)OC1=O ; CC[C@H](C)[C@H](N)C(=O)Nc1ccc(Cl)cc1C(=O)c1ccncc1 ; "TFA" ; CC[C@H](C)[C@@H]1N=C(c2ccncc2)c2cc(Cl)ccc2NC1=O ; "NEt3"]

Step 2: A mixture of (2-amino-5-chlorophenyl)(pyridin-4-yl)methanone (200 mg, 0.86 mmol), (S)-4-((S)-sec-butyl)oxazolidine-2,5-dione (162 mg, 0.103 mmol), and trifluoroacetic acid (197 μL, 2.58 mmol) in 4.3 mL of toluene was stirred at 60° C. for 19 hours before adding triethylamine (359 μL, 2.56 mmol). The resulting mixture was then stirred at 80° C. for 3 hours before being cooled to 23° C. and evaporated. The residue was taken into dichloromethane and the insoluble material was filtered off. The filtrate was evaporated and the residue was purified by flash chromatography on silica gel (hexanes/ethyl acetate 80/20 to 0/100) to give 119 mg of (S)-3-((S)-sec-butyl)-7-chloro-5-(pyridin-4-yl)-1,3-dihydro-2H-benzo[e][1,4]diazepin-2-one (42% yield). MS (ESI) m/z 328.3 (M+H)⁺

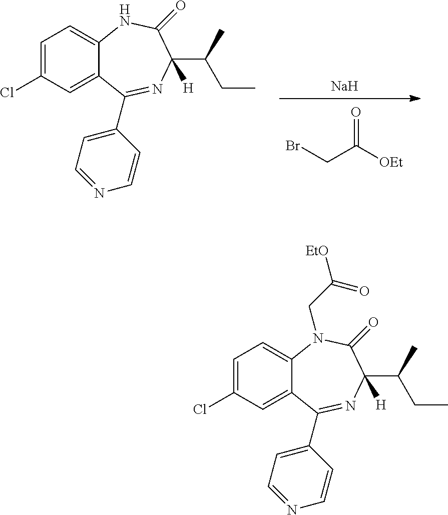

Step 3: Sodium hydride 60% dispersion in oil (30 mg, 0.74 mmol) was added at 0° C. to a solution of (S)-3-((S)-sec-butyl)-7-chloro-5-(pyridin-4-yl)-1,3-dihydro-2H-benzo[e][1,4]diazepin-2-one (119 mg, 0.36 mmol) in 1.7 mL of tetrahydrofuran. The resulting mixture was stirred at 0° C. for 30 minutes before adding ethyl bromoacetate (60 μL, 0.54 mmol). The reaction mixture was stirred at 23° C. for three hours before being cooled back to 0° C., quenched by the addition of iced water, and extracted three times with ethyl acetate. The combined organic layers were washed with water, dried over sodium sulfate, filtered, and evaporated. The residue was purified by flash chromatography on silica gel (hexanes/ethyl acetate 95/5 to 20/80) to give 104 mg of ethyl 2-((S)-3-((S)-sec-butyl)-7-chloro-2-oxo-5-(pyridin-4-yl)-2,3-dihydro-1H-benzo[e][1,4]diazepin-1-yl)acetate. MS (ESI) m/z 414.37 (M+H)⁺

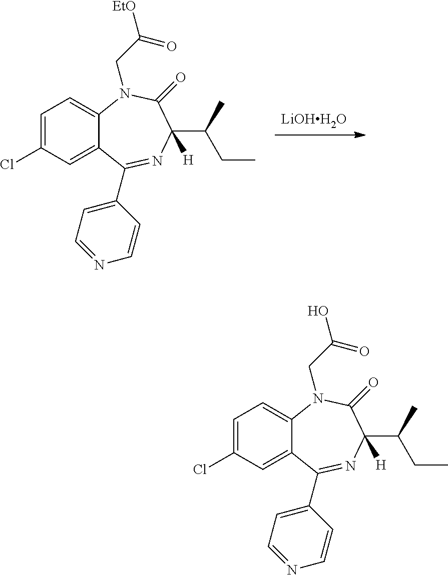 [SMILES: CCOC(=O)CN1C(=O)[C@H]([C@@H](C)CC)N=C(c2ccncc2)c2cc(Cl)ccc21 ; "LiOH·H2O" ; CC[C@H](C)[C@@H]1N=C(c2ccncc2)c2cc(Cl)ccc2N(CC(=O)O)C1=O]

Step 4: Lithium hydroxide monohydrate (58 mg, 1.38 mmol) was added at 23° C. to a solution of ethyl 2-((S)-3-((S)-sec-butyl)-7-chloro-2-oxo-5-(pyridin-4-yl)-2,3-dihydro-1H-benzo[e][1,4]diazepin-1-yl)acetate (104 mg, 0.251 mmol) in 5.7 mL of a 1/1 mixture of 1,4-dioxane and water. The resulting mixture was stirred at 23° C. for 18 hours before being cooled to 0° C., acidified to pH 5-6 by addition of 10% aqueous citric acid, and extracted three times with ethyl acetate. The combined organic layers were washed with brine, dried over sodium sulfate, filtered, and evaporated. The residue crystallized from dichloromethane/hexanes to give 95 mg of 2-((S)-3-((S)-sec-butyl)-7-chloro-2-oxo-5-(pyridin-4-yl)-2,3-dihydro-1H-benzo[e][1,4]diazepin-1-yl)acetic acid. 1H NMR (400 MHz, DMSO-d6) δ 12.88 (s, 1H), 8.64 (dd, J=4.6, 1.8 Hz, 2H), 7.68 (dd, J=8.8, 2.4 Hz, 1H), 7.57 (d, J=9.2 Hz, 1H), 7.42 (dd, J=4.8, 1.6 Hz, 2H), 7.28 (d, J=2.4 Hz, 1H), 4.62 (d, J=17.6 Hz, 1H), 4.45 (d, J=17.6 Hz, 1H), 3.23 (s, 1H), 2.36-2.30 (m, 1H), 1.88-1.82 (m, 1H), 1.14-1.06 (m, 1H), 0.86-0.83 (m, 6H). MS (ESI): m/z 386.36 (M+H)⁺.

### Example 13: Synthesis of 2-((S)-3-((S)-sec-butyl)-7-chloro-2-oxo-5-(pyridin-2-yl)-2,3-dihydro-1H-benzo[e][1,4]diazepin-1-yl)acetic acid

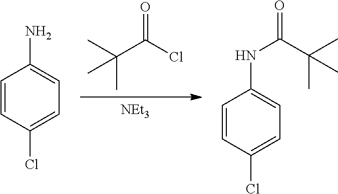

Step 1: To a solution of 4-chloroaniline (2.55 g, 20 mmol) in 57 mL of dichloromethane was added at 23° C. triethylene (4.2 mL, 30 mmol) followed by pivaloyl chloride (3 mL, 24 mmol). The resulting mixture was stirred at 23° C. for 17 hours before being quenched by addition of 20 mL of 1M aqueous hydrochloric acid and extracted with dichloromethane. The organic layer was washed with saturated aqueous sodium bicarbonate followed by brine, then dried over magnesium sulfate, filtered, and evaporated to give 3.9 g of N-(4-chlorophenyl)pivalamide (92% yield). ¹H NMR (400 MHz, DMSO-d6) δ 9.25 (s, 1H), 7.64-7.58 (m, 2H), 7.30-7.24 (m, 2H), 1.15 (s, 9H); MS (ESI) m/z 212.20 (M+H)⁺

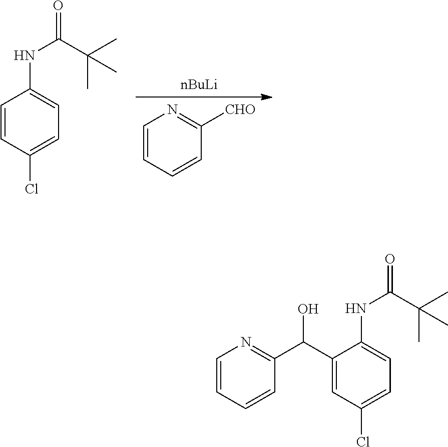

Step 2: n-Butyllithium 1.6M in hexanes (9.1 mL, 14.5 mmol) was added at 0° C. to a solution of N-(4-chlorophenyl) pivalamide (1.02 g, 4.83 mmol) in 5 mL of tetrahydrofuran. The resulting mixture was stirred at 0° C. for an hour before adding picolinaldehyde (920 μL, 9.66 mmol) in 5 mL of tetrahydrofuran. The mixture was warmed to 23° C. over an hour before being quenched by addition of water and extracted with ethyl acetate. The organic layer was washed with saturated aqueous sodium bicarbonate, brine, dried over magnesium sulfate, filtered, and evaporated. The residue was purified by flash chromatography on silica gel (hexanes/ethyl acetate 100/0 to 65/35) to give 0.97 g of N-(4-chloro-2-(hydroxy(pyridin-2-yl)methyl)phenyl)pivalamide. ¹H NMR (400 MHz, DMSO-d6) δ 10.42 (s, 1H), 8.42 (ddd, J=4.9, 1.8, 0.9 Hz, 1H), 7.82 (td, J=7.7, 1.8 Hz, 1H), 7.74 (d, J=8.7 Hz, 1H), 7.60 (dd, J=7.9, 1.3 Hz, 1H), 7.50 (d, J=2.6 Hz, 1H), 7.30-7.25 (m, 1H), 7.21 (dd, J=8.7, 2.6 Hz, 1H), 6.58 (d, J=3.9 Hz, 1H), 5.84 (d, J=3.9 Hz, 1H), 1.22 (s, 9H); MS (ESI) m/z 319.22 (M+

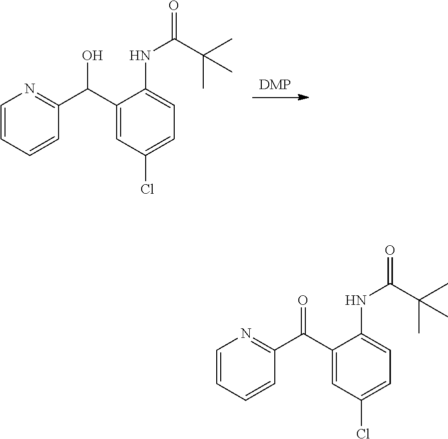

Step 3: Dess Martin periodinane (DMP) (1.68 g, 3.97 mmol) was added at 23° C. to a solution of N-(4-chloro-2-(hydroxy(pyridin-2-yl)methyl)phenyl)pivalamide (970 mg, 3.05 mmol) in 100 mL of dichloromethane. The resulting mixture was stirred at 23° C. for 18 hours before being quenched by addition of saturated aqueous sodium bicarbonate. The organic layer was dried over magnesium sulfate, filtered, and evaporated. The residue was purified by flash chromatography on silica gel (hexanes/ethyl acetate 100/0 to 65/35) to give 850 mg of N-(4-chloro-2-picolinoyl phenyl)pivalamide (88% yield). ¹H NMR (400 MHz, DMSO-d6) δ 9.78 (s, 1H), 8.51 (dt, J=4.7, 1.2 Hz, 1H), 7.95 (td, J=7.7, 1.7 Hz, 1H), 7.87 (dt, J=7.9, 1.1 Hz, 1H), 7.62-7.48 (m, 4H), 0.82 (s, 9H); MS (ESI) m/z 317.18 (M+H)⁺

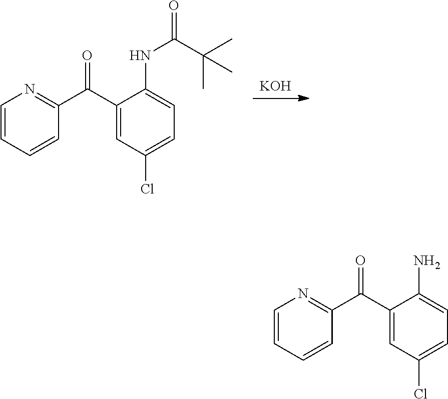

Step 4: To a solution of N-(4-chloro-2-picolinoylphenyl)pivalamide (850 mg, 2.68 mmol) in 27 mL of methanol was added at 23° C. aqueous 40% potassium hydroxide (5.2 mL). The resulting mixture was stirred at 65° C. for 24 hours before being cooled to 23° C. and evaporated. The residue was taken into ethyl acetate, washed with brine, dried over magnesium sulfate, filtered, and evaporated. The residue was purified by flash chromatography on silica gel (hexanes/ethyl acetate 100/0 to 65/35) to give 660 mg of (2-amino-5-chlorophenyl)(pyridin-2-yl)methanone (quantitative yield). ¹H NMR (400 MHz, DMSO-d6) δ 8.63 (ddd, J=4.8, 1.7, 0.9 Hz, 1H), 7.97 (td, J=7.7, 1.7 Hz, 1H), 7.73 (dt, J=7.8, 1.1 Hz, 1H), 7.54 (ddd, J=7.7, 4.8, 1.2 Hz, 1H), 7.47 (d, J=2.6 Hz, 1H), 7.38 (br s, 2H), 7.26 (dd, J=9.0, 2.6 Hz, 1H), 6.84 (d, J=9.0 Hz, 1H); MS (ESI) m/z 233.12 (M+H)⁺

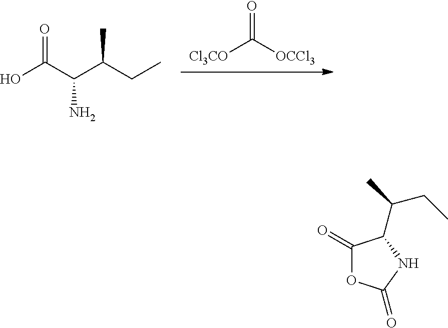

Step 5: A mixture of L-isoleucine (2.67 g, 10.4 mmol) and triphosgene (4.83 g, 16.3 mmol) in 136 mL of tetrahydrofuran was stirred at reflux for three hours before being cooled to 23° C. and concentrated. The residue was taken into hexanes and kept at −20° C. The liquid was pipetted out and the solid was taken into hexanes and tetrahydrofuran. The mixture was passed through silica gel which was then rinsed with dichloromethane. The filtrate was evaporated to give 1.95 g of (S)-4-((S)-sec-butyl)oxazolidine-2,5-dione. ¹H NMR (400 MHz, DMSO-d6) δ 9.03 (s, 1H), 4.34 (dd, J=4.1, 1.2 Hz, 1H), 1.73 (ddtd, J=10.9, 8.4, 6.7, 4.3 Hz, 1H), 1.29 (dddd, J=14.9, 12.3, 7.5, 4.9 Hz, 1H), 1.21-1.09 (m, 1H), 0.86 (d, J=6.9 Hz, 3H), 0.80 (t, J=7.4 Hz, 3H).

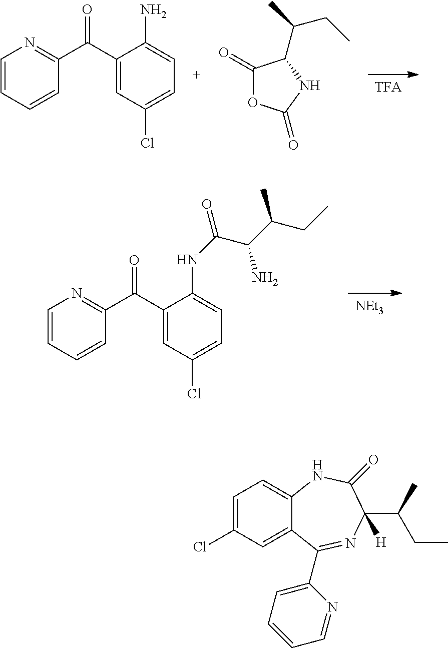 [SMILES: Nc1ccc(Cl)cc1C(=O)c1ccccn1 ; CC[C@H](C)[C@@H]1NC(=O)OC1=O ; CC[C@H](C)[C@H](N)C(=O)Nc1ccc(Cl)cc1C(=O)c1ccccn1 ; "TFA" ; CC[C@H](C)[C@@H]1N=C(c2ccccn2)c2cc(Cl)ccc2NC1=O ; "NEt3"]

Step 6: A mixture of (2-amino-5-chlorophenyl)(pyridin-2-yl)methanone (237 mg, 1.02 mmol) prepared in step 4 and (S)-4-((S)-sec-butyl)oxazolidine-2,5-dione (192 mg, 1.22 mmol) prepared in step 5 and trifluoroacetic acid (227 μL, 3.06 mmol) in 5.1 mL of toluene was stirred at 60° C. for 4.5 hours before adding triethylamine (430 μL, 3.06 mmol). The resulting mixture was then stirred at 80° C. for 19 hours before being cooled to 23° C. The residue was purified by flash chromatography on silica gel (hexanes/ethyl acetate 90/10 to 40/60) to give 194 mg of (S)-3-((S)-sec-butyl)-7-chloro-5-(pyridin-2-yl)-1,3-dihydro-2H-benzo[e][1,4]diazepin-2-one. ¹H NMR (400 MHz, DMSO-d6) δ 10.60 (s, 1H), 8.50 (ddd, J=4.9, 1.8, 0.9 Hz, 1H), 7.99 (dt, J=7.9, 1.2 Hz, 1H), 7.90 (td, J=7.7, 1.8 Hz, 1H), 7.54 (dt, J=8.8, 2.2 Hz, 1H), 7.49-7.42 (m, 1H), 7.30 (d, J=2.5 Hz, 1H), 7.17 (d, J=8.8 Hz, 1H), 3.15 (d, J=9.3 Hz, 1H), 2.27 (dt, J=9.3, 6.4 Hz, 1H), 1.85 (ddt, J=15.2, 7.6, 4.2 Hz, 1H), 1.18 (ddd, J=13.5, 9.3, 7.2 Hz, 1H), 0.90 (d, J=6.5 Hz, 3H), 0.84 (t, J=7.5 Hz, 3H); MS (ESI) m/z 328.26 (M+H)⁺

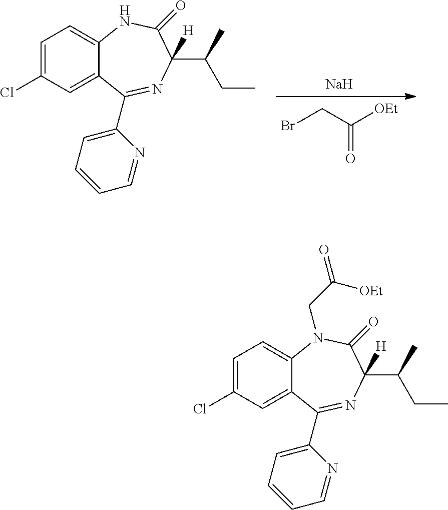

Step 7: Sodium hydride 60% dispersion in oil (47 mg, 1.17 mmol) was added at 0° C. to a solution of (S)-3-((S)-sec-butyl)-7-chloro-5-(pyridin-2-yl)-1,3-dihydro-2H-benzo[e][1,4]diazepin-2-one (197 mg, 0.592 mmol) in 3 mL of tetrahydrofuran. The resulting mixture was stirred at 0° C. for 3 hours before adding ethyl bromoacetate (98 μL, 0.888 mmol). The resulting mixture was stirred at 23° C. for 16 hours before being quenched with saturated aqueous ammonium chloride and extracted with ethyl acetate. The combined organic layers were washed with brine, dried over magnesium sulfate, filtered, and evaporated. The residue was purified by flash chromatography on silica gel (hexanes/ethyl acetate 100/0 to 55/45) to give 203 mg of impure ethyl 2-((S)-3-((S)-sec-butyl)-7-chloro-2-oxo-5-(pyridin-2-yl)-2,3-dihydro-TH-benzo[e][1,4]diazepin-1-yl)acetate which was purified again by flash chromatography on silica gel (hexanes/ethyl acetate 100/0 to 70/30) to give 66 mg of pure material. ¹H NMR (400 MHz, DMSO-d6) δ 8.52 (ddd, J=4.8, 1.8, 0.9 Hz, 1H), 8.08-8.05 (m, 1H), 7.90 (td, J=7.7, 1.8 Hz, 1H), 7.61 (dd, J=8.9, 2.6 Hz, 1H), 7.48-7.40 (m, 2H), 7.38 (t, J=2.3 Hz, 1H), 4.50 (d, J=4.8 Hz, 2H), 3.25 (d, J=9.3 Hz, 1H), 2.36-2.22 (m, 1H), 1.83 (ddd, J=13.3, 7.6, 3.1 Hz, 1H), 1.18-1.06 (m, OH), 0.87-0.80 (m, 5H); MS (ESI) m/z 414.35 (M+H)⁺

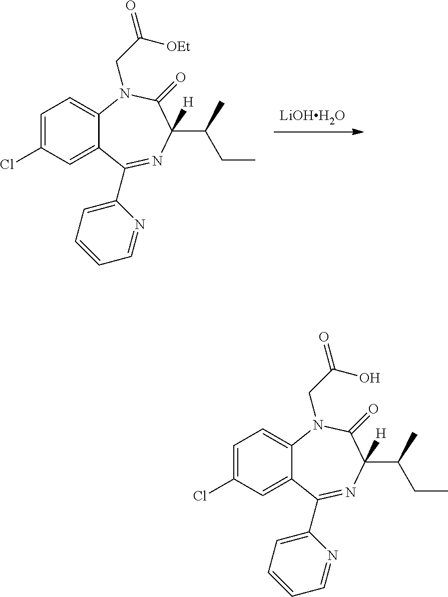 [SMILES: CCOC(=O)CN1C(=O)[C@H]([C@@H](C)CC)N=C(c2ccccn2)c2cc(Cl)ccc21 ; "LiOH·H2O" ; CC[C@H](C)[C@@H]1N=C(c2ccccn2)c2cc(Cl)ccc2N(CC(=O)O)C1=O]

Step 8: A mixture of pure ethyl 2-((S)-3-((S)-sec-butyl)-7-chloro-2-oxo-5-(pyridin-2-yl)-2,3-dihydro-1H-benzo[e][1,4]diazepin-1-yl)acetate (66 mg, 0.16 mmol) and lithium hydroxide monohydrate 34 mg, 0.8 mmol) in 3.2 mL of tetrahydrofuran and 10 mL of water was stirred at 23° C. for 48 hours before being quenched by addition of Dowex 50W×8 chloride form. The mixture was filtered and the resin was washed with tetrahydrofuran. The filtrate was evaporated and then extracted with ethyl acetate. The combined organic layers were dried over magnesium sulfate, filtered, and evaporated. The residue was taken into dichloromethane and hexanes. After evaporation, the sticky residue was taken into 1 mL of ethyl acetate and 3 mL of hexanes and allowed to stand at 23° C. for two days. The supernatant was pipetted out and the solid material was dried under vacuum to give 25 mg of 2-((S)-3-((S)-sec-butyl)-7-chloro-2-oxo-5-(pyridin-2-yl)-2,3-dihydro-1H-benzo[e][1,4]diazepin-1-yl)acetic acid. 1H NMR (400 MHz, DMSO-d6) δ 12.83 (s, 1H), 8.56-8.49 (m, 1H), 8.06 (dd, J=8.3, 7.0 Hz, 1H), 7.91 (td, J=7.7, 1.7 Hz, 1H), 7.62 (dd, J=8.9, 2.6 Hz, 1H), 7.46 (ddd, J=7.5, 4.8, 1.2 Hz, 1H), 7.43-7.36 (m, 2H), 4.48-4.32 (m, 2H), 3.26 (dd, J=18.53, 9.04 Hz, 1H), 2.32 (dt, J=9.7, 6.5 Hz, 1H), 1.84 (ddd, J=13.3, 7.6, 3.0 Hz, 1H), 1.11 (dtd, J=11.0, 9.0, 8.2, 5.6 Hz, 1H), 0.94-0.77 (m, 6H). MS (ESI): m/z 386.37 (M+H)⁺.

### Example 14: Synthesis 2-((S)-3-((S)-sec-butyl)-7-chloro-5-isopropyl-2-oxo-2,3-dihydro-1H-benzo[e][1,4]diazepin-1-yl)acetic acid

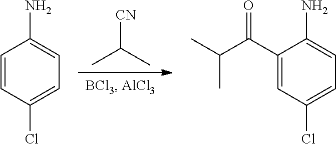

Step 1: 4-Chloroaniline (7.38 g, 57.9 mmol) in 30 mL of dichloroethane was added dropwise at 0° C. to a solution of boron trichloride 1M in dichloromethane (37.62 mL, 37.62 mmol). The resulting mixture was stirred at 0° C. for 30 minutes before adding isobutyronitrile (2.63 mL, 28.94 mmol) followed by aluminum trichloride (6.02 g, 37.62 mL). The resulting mixture was stirred at 0° C. before being stirred at 90° C. for 18 hours. The reaction mixture was then cooled to 23° C. and then to 0° C. before being quenched by addition of 60 mL of 2M aqueous hydrochloric acid. The resulting mixture was stirred at 90° C. for 30 minutes before being cooled to 23° C., extracted three times with dichloromethane. The combined organic layers were dried over sodium sulfate, filtered, and evaporated. The residue was purified by flash chromatography on silica gel (hexanes/ethyl acetate 95-5 to 60/40) to give 1.64 g of 1-(2-amino-5-chlorophenyl)-2-methylpropan-1-one. MS (ESI) m/z 198.07 (M+H)⁺

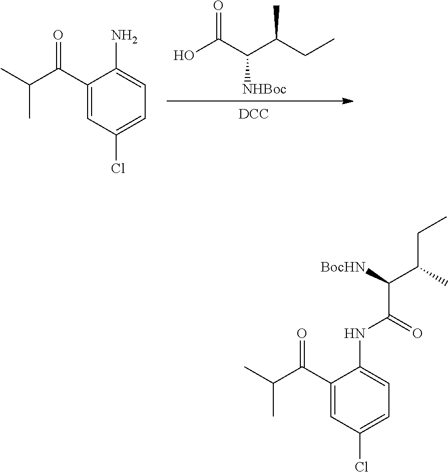

Step 2: A solution of dicyclohexylcarbodiimide 1M in dichloromethane (3.04 mL, 3.04 mmol) was added at 0° C. to a solution of L-N-boc-isoleucine (703 mg, 3.04 mmol) in 1.5 mL of dichloromethane. The resulting mixture was stirred at 23° C. for 30 minutes before being cooled back to 0° C. 1-(2-Amino-5-chlorophenyl)-2-methylpropan-1-one (600 mg, 3.04 mmol) in 2.8 mL of dichloromethane was then added and the resulting mixture was stirred at 23° C. for 2 days. The insoluble material that had formed during the reaction was filtered and the filtrate was evaporated. The residue was purified by flash chromatography on silica gel (hexanes/ethyl acetate 80/20 to 40/60) to give 862 mg of impure tert-butyl ((2S,3S)-1-((4-chloro-2-isobutyrylphenyl)amino)-3-methyl-1-oxopentan-2-yl)carbamate which was used in the next reaction without further purification. MS (ESI) m/z 433.14 (M+Na)⁺

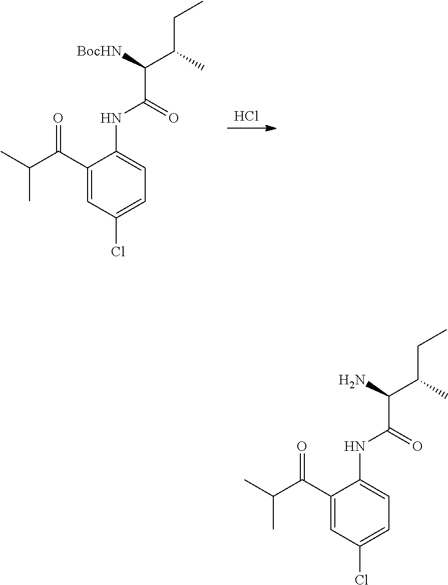 [SMILES: CC[C@H](C)[C@H](NC(=O)OC(C)(C)C)C(=O)Nc1ccc(Cl)cc1C(=O)C(C)C ; Cl ; CC[C@H](C)[C@H](N)C(=O)Nc1ccc(Cl)cc1C(=O)C(C)C]

Step 3: Hydrochloric acid 4M in 1,4-dioxane (5.25 mL, 21 mmol) was added at 23° C. to a solution of impure tert-butyl ((2S,3S)-1-((4-chloro-2-isobutyrylphenyl)amino)-3-methyl-1-oxopentan-2-yl)carbamate (862 mg, theoretically 2.1 mmol) in 5.6 mL of dichloromethane. The resulting mixture was stirred at 23° C. for 18 hours before being evaporated. The residue was dissolved in dichloromethane and the resulting solution was washed with aqueous saturated sodium bicarbonate followed by water, dried over sodium sulfate, filtered, and evaporated to give 429 mg of crude (2S,3S)-2-amino-N-(4-chloro-2-isobutyrylphenyl)-3-methylpentanamide which was used in the next step without further purification. MS (ESI) m/z 311.18 (M+H)⁺

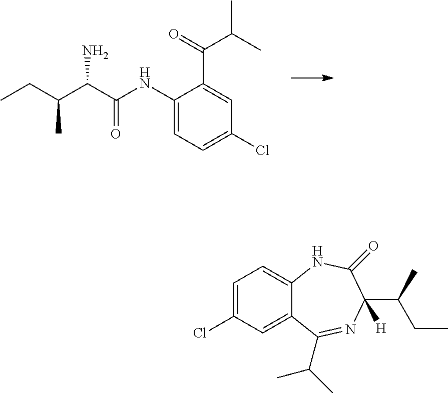 [SMILES: CC[C@H](C)[C@H](N)C(=O)Nc1ccc(Cl)cc1C(=O)C(C)C ; CC[C@H](C)[C@@H]1N=C(C(C)C)c2cc(Cl)ccc2NC1=O]

Step 2: (2S,3S)-2-amino-N-(4-chloro-2-isobutyrylphenyl)-3-methylpentanamide (429 mg, 1.38 mmol) was dissolved in 4 mL of methanol and 0.4 mL of water. The resulting solution was cooled to 0° C. and the pH was adjusted to 8-9 by addition of aqueous 1M sodium hydroxide. The resulting mixture was allowed to warm to 23° C. and stirred for 18 hours before being evaporated. The residue was partitioned between water and dichloromethane. The aqueous layer was then washed three times with dichloromethane. The combined organic layers were dried over sodium sulfate, filtered, and evaporated. The residue was purified by flash chromatography on silica gel (hexanes/ethyl acetate 95/5 to 60/40) to give 210 mg of (S)-3-((S)-sec-butyl)-7-chloro-5-isopropyl-1,3-dihydro-2H-benzo[e][1,4]diazepin-2-one. MS (ESI) m/z 293.28 (M+H)⁺

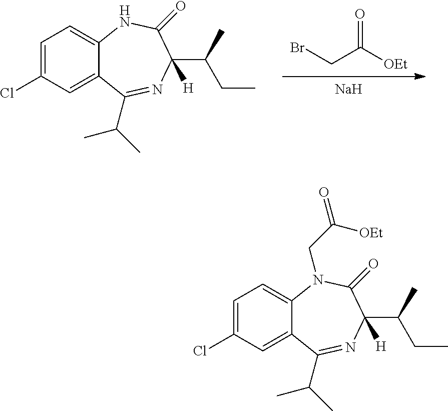

Step 5: Sodium hydride 60% dispersion in oil (58 mg, 1.44 mmol) was added at 0° C. to a solution of (S)-3-((S)-sec-butyl)-7-chloro-5-isopropyl-1,3-dihydro-2H-benzo[e][1,4]diazepin-2-one (210 mg, 0.71 mmol) in 3.4 mL of tetrahydrofuran. The resulting mixture was stirred at 0° C. for two and a half hours before adding ethyl bromoacetate (0.12 mL, 1.08 mmol). The resulting mixture was slowly warmed to 23° C. and stirred for 18 hours, before being cooled back to 0° C., quenched by the addition of iced water, and extracted three times with ethyl acetate. The combined organic layers were dried over sodium sulfate, filtered, and evaporated. The residue was purified by flash chromatography on silica gel (hexanes/ethyl acetate 95/5 to 60/40) to give 255 mg of ethyl 2-((S)-3-((S)-sec-butyl)-7-chloro-5-isopropyl-2-oxo-2,3-dihydro-1H-benzo[e][1,4]diazepin-1-yl)acetate. MS (ESI) m/z 379.33 (M+H)⁺

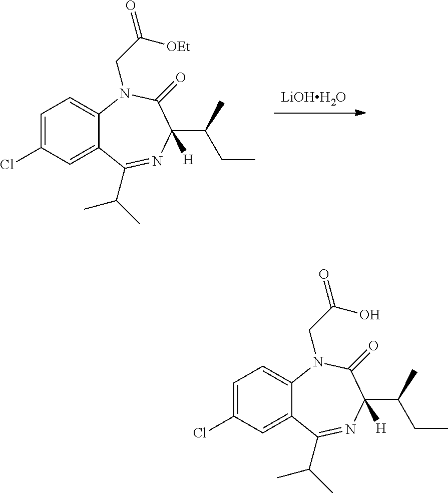 [SMILES: CCOC(=O)CN1C(=O)[C@H]([C@@H](C)CC)N=C(C(C)C)c2cc(Cl)ccc21 ; "LiOH·H2O" ; CC[C@H](C)[C@@H]1N=C(C(C)C)c2cc(Cl)ccc2N(CC(=O)O)C1=O]

Step 6: Lithium hydroxide monohydrate (153 mg, 3.7 mmol) was added at 23° C. to a solution of ethyl 2-((S)-3-((S)-sec-butyl)-7-chloro-5-isopropyl-2-oxo-2,3-dihydro-1H-benzo[e][1,4]diazepin-1-yl)acetate (255 mg, 0.673 mmol) in 24.2 mL of a 1/1 mixture of 1,4-dioxane and water. The resulting mixture was stirred at 23° C. for 18 hours before being cooled to 0° C., acidified to pH 5-6 by addition of 10% aqueous citric acid, and extracted three times with ethyl acetate. The combined organic layers were dried over sodium sulfate, filtered, and evaporated. The residue was purified by flash chromatography on silica gel (hexanes/ethyl acetate 95/5 to 20/80 followed by dichloromethane/methanol 100/0 to 80/20) to give 85 mg of 2-((S)-3-((S)-sec-butyl)-7-chloro-5-isopropyl-2-oxo-2,3-dihydro-1H-benzo[e][1,4]diazepin-1-yl)acetic acid. 1H NMR (400 MHz, CDCl3) δ 7.50 (d, J=2.4 Hz, 11H), 7.43 (dd, J=2.8, 2.4 Hz, 114), 7.15 (d, J=8.8 Hz, 11H), 4.451 (dd, J=17.6, 50 Hz, 214), 3.13-3.07 (m, 114), 3.04 (d, J=9.6 Hz, 1H), 2.47-2.39 (m, 114), 1.26 (d, J=6.4 Hz, 311), 1.14-0.99 (m, 214), 0.97-0.93 (m, 3H), 0.91-0.83 (m, 614). MS (ESI): m/z 351.37 (M+H)⁺.

### Example 15: Synthesis of: 2-((3S)-3-((S)-sec-butyl)-7-chloro-2-oxo-5-phenyl-2,3,4,5-tetrahydro-1H-benzo[e][1,4]diazepin-1-yl)acetic acid

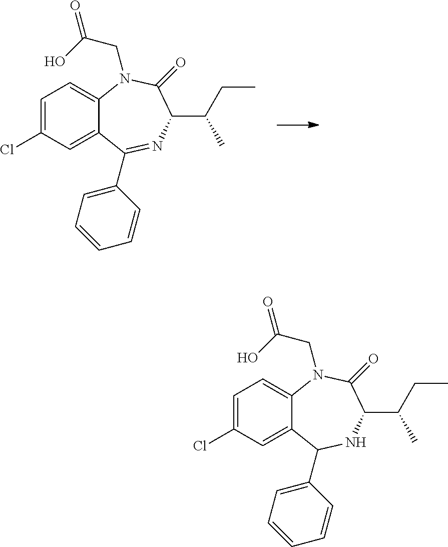

Step 1: Sodium cyanoborohydride (9 mg, 0.143 mmol) and acetic acid (9 μL, 0.143 mmol) were added sequentially at 0° C. to a solution of 2-((S)-3-((S)-sec-butyl)-7-chloro-2-oxo-5-phenyl-2,3-dihydro-1H-benzo[e][1,4]diazepin-1-yl)acetic acid (50 mg, 0.13 mmol) in 1.5 mL of methanol. The resulting mixture was stirred for 18 hours before adding a second portion of sodium cyanoborohydride (9 mg, 0.143 mmol) and acetic acid (9 mL, 0.143 mmol). The reaction mixture was stirred at 23° C. for 18 hours before being quenched by addition of a 1/1 mixture of saturated aqueous sodium bicarbonate and water, extracted three times with dichloromethane, dried over sodium sulfate, filtered, and evaporated. The residue was purified by flash chromatography on silica gel (hexanes/ethyl acetate 100/0 to 20/80 followed by dichloromethane/methanol 100/0 to 80/20) to give 28 mg of 2-((3S)-3-((S)-sec-butyl)-7-chloro-2-oxo-5-phenyl-2,3,4,5-tetrahydro-1H-benzo[e][1,4]diazepin-1-yl)acetic acid. ¹H NMR (400 MHz, DMSO-d6) δ 7.41-7.26 (m, 7H), 6.24 (s, 1H), 5.59 (s, 1H), 4.48 (d, J=17.0 Hz, 1H), 4.35 (d, J=17.0 Hz, 1H), 2.77 (m, 1H), 1.82 (m, 1H), 1.64 (m, 1H), 1.13-1.06 (m, 1H), 0.75-0.69 (m, 6H). MS (ESI): m/z 387.2 (M+H)⁺.

### Example 16: Synthesis of: 2-((S)-3-((S)-sec-butyl)-7-chloro-2-oxo-5-phenyl-2,3-dihydro-1H-benzo[e][1,4]diazepin-1-yl)-N-(methylsulfonyl)acetamide

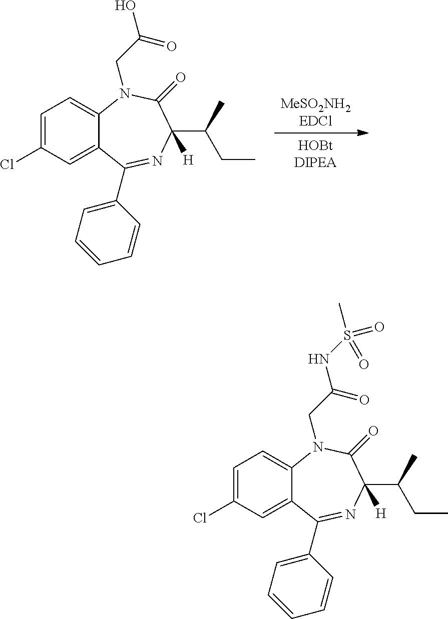 [SMILES: CC[C@H](C)[C@@H]1N=C(c2ccccc2)c2cc(Cl)ccc2N(CC(=O)O)C1=O ; "HOBtDIPEA" ; "MeSO2NH2EDCl" ; CC[C@H](C)[C@@H]1N=C(c2ccccc2)c2cc(Cl)ccc2N(CC(=O)NS(C)(=O)=O)C1=O]

Step 1: A mixture of 2-((S)-3-((S)-sec-butyl)-7-chloro-2-oxo-5-phenyl-2,3-dihydro-1H-benzo[e][1,4]diazepin-1-yl)acetic acid (50 mg, 1.3 mmol), methanesulfonamide (25 mg, 0.26 mmol), 1-hydroxybenzotriazole hydrate (41 mg, 0.3 mmol), N-(3-dimethylaminopropyl)-N′-ethylcarbodiimide hydrochloride (58 mg, 0.3 mmol), and diisopropylethylamine (52 μL, 0.3 mmol) in 1.5 mL of dichloromethane and 1.5 mL of dimethylformamide was stirred at 23° C. for 48 hours before being partitioned between water and dichloromethane. The organic layer was washed with water, dried over sodium sulfate, filtered, and evaporated. A second batch of product was prepared the same way. The combined crude residue was purified by flash chromatography on silica gel (hexanes/ethyl acetate 95/5 to 0/100 followed by dichloromethane/methanol 100/0 to 80/20) and then by preparative thin layer chromatography (dichloromethane/methanol 95/5) to give 24 mg of 2-((S)-3-((S)-sec-butyl)-7-chloro-2-oxo-5-phenyl-2,3-dihydro-1H-benzo[c][1,4]diazepin-1-yl)-N-(methylsulfonyl)acetamide. ¹H NMR (400 MHz, DMSO-d6) δ 7.59 (dd, J=8.8, 2.4 Hz, 1H), 7.52-7.49 (m, 2H), 7.47-7.37 (m, 4H), 7.10 (d, J=2.8 Hz, 1H), 4.29 (d, J=17.0 Hz, 1H), 4.14 (d, J=17.0 Hz, 1H), 3.08 (d, J=10.0 Hz, 1H), 2.95 (s, 3H), 2.34-2.31 (m, 1H), 1.91-1.84 (m, 1H), 1.13-1.06 (m, 1H), 0.86-0.82 (m, 6H). MS (ESI): m/z 462.19 (M+H)⁺.

### Example 17: Synthesis of target 29: (S)-1-((2H-tetrazol-5-yl)methyl)-3-((S)-sec-butyl)-7-chloro-5-phenyl-1,3-dihydro-2H-benzo[e][1,4]diazepin-2-one

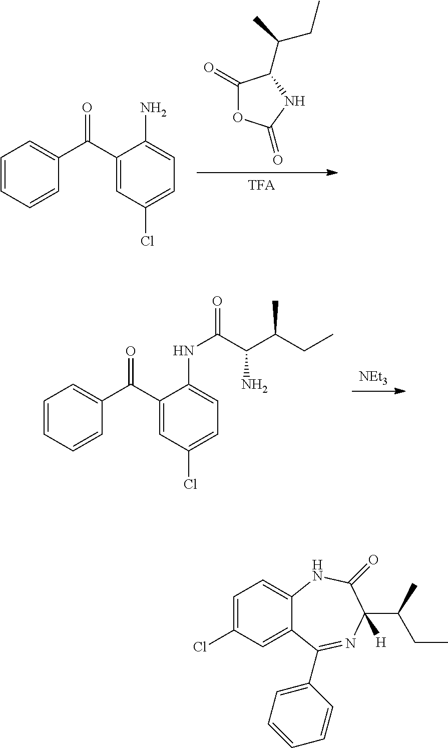 [SMILES: Nc1ccc(Cl)cc1C(=O)c1ccccc1 ; CC[C@H](C)[C@@H]1NC(=O)OC1=O ; CC[C@H](C)[C@H](N)C(=O)Nc1ccc(Cl)cc1C(=O)c1ccccc1 ; "TFA" ; CC[C@H](C)[C@@H]1N=C(c2ccccc2)c2cc(Cl)ccc2NC1=O ; "NEt3"]

Step 1: A mixture of (2-amino-5-chlorophenyl)(phenyl)methanone (923 mg, 3.98 mmol), (S)-4-((S)-sec-butyl)oxazolidine-2,5-dione (751 mg, 4.78 mmol), and trifluoroacetic acid (590 mL, 4.75 mmol) in 13 mL of toluene was stirred at 65° C. for 17 hours before adding triethylamine (1.1 mL, 7.96 mmol). The resulting mixture was then stirred at 80° C. for 30 minutes before being cooled to 23° C. and evaporated. The residue was purified by flash chromatography on silica gel (hexanes/ethyl acetate 100/0 to 60/40) to give 1.06 mg of (S)-3-((S)-sec-butyl)-7-chloro-5-phenyl-1,3-dihydro-2H-benzo[e][1,4]diazepin-2-one. ¹H NMR (400 MHz, DMSO-d6) δ 10.61 (s, 1H), 7.59 (dd, J=8.7, 2.5 Hz, 1H), 7.49-7.32 (m, 5H), 7.26-7.16 (m, 2H), 3.06 (d, J=9.4 Hz, 1H), 2.32-2.18 (m, 1H), 1.90-1.77 (m, 1H), 1.22-1.14 (m, 1H), 0.89 (d, J=6.5 Hz, 3H), 0.84 (t, J=7.4 Hz, 3H); MS (ESI) m/z 326.84 (M+H)⁺

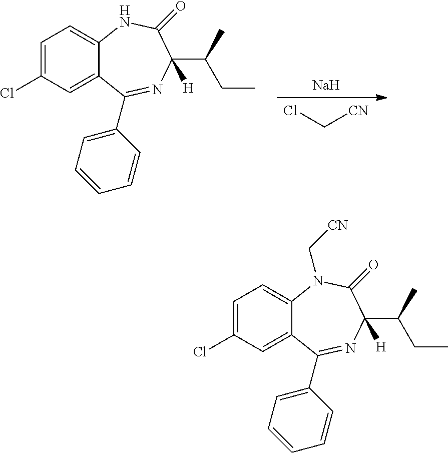

Step 2: Sodium hydride 60% dispersion in oil (40 mg, 0.997 mmol) was added at 0° C. to a solution of (S)-3-((S)-sec-butyl)-7-chloro-5-phenyl-1,3-dihydro-2H-benzo[e][1,4]diazepin-2-one (163 mg, 0.499 mmol) in 2.5 mL of tetrahydrofuran. The resulting mixture was stirred at 0° C. for two hours before adding 2-chloroacetonitrile (47 μL, 0.749 mmol). The reaction mixture was stirred at 23° C. for 20 hours before being quenched by the addition of saturated aqueous ammonium chloride, and extracted with ethyl acetate. The combined organic layers were dried over magnesium sulfate, filtered, and evaporated. The residue was purified by flash chromatography on silica gel (hexanes/ethyl acetate 100/0 to 65/35) to give 99 mg of 2-((S)-3-((S)-sec-butyl)-7-chloro-2-oxo-5-phenyl-2,3-dihydro-1H-benzo[el[1,4]diazepin-1-yl)acetonitrile. ¹H NMR (400 MHz, DMSO-d6) δ 7.77 (dd, J=8.8, 2.5 Hz, 1H), 7.64 (d, J=8.9 Hz, 1H), 7.49-7.40 (m, 5H), 7.27 (d, J=2.5 Hz, 1H), 5.03 (d, J=17.9 Hz, 1H), 4.89 (d, J=17.8 Hz, 1H), 3.27-3.25 (m, 1H), 2.36-2.25 (m, 1H), 1.90-1.78 (m, 1H), 1.11 (t, J=7.1 Hz, 1H), 0.90-0.80 (m, 6H); MS (ESI) m/z 366.26 (M+H)⁺

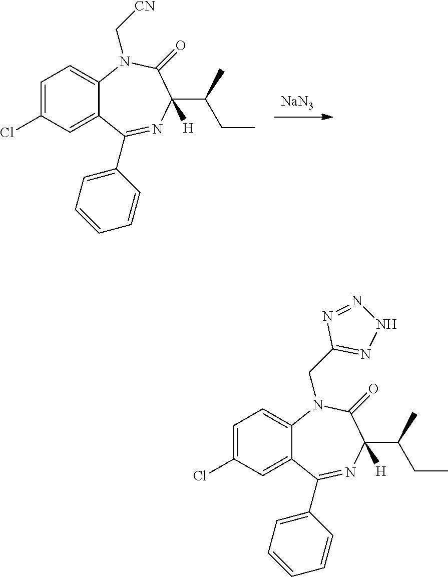 [SMILES: CC[C@H](C)[C@@H]1N=C(c2ccccc2)c2cc(Cl)ccc2N(CC#N)C1=O ; CC[C@H](C)[C@@H]1N=C(c2ccccc2)c2cc(Cl)ccc2N(Cc2nn[nH]n2)C1=O ; "NaN3"]

Step 3: A mixture of 2-((S)-3-((S)-sec-butyl)-7-chloro-2-oxo-5-phenyl-2,3-dihydro-1H-benzo[e][1,4]diazepin-1-yl)acetonitrile (99 mg, 0.27 mmol), ammonium chloride (19 mg, 0.35 mmol) and sodium azide (23 mg, 0.35 mmol) in 2.7 mL of dimethylformanilide was stirred at 125° C. for 21 hours before being cooled to 23° C. and partitioned between water and ethyl acetate. The organic layer was washed with brine, dried over magnesium sulfate, filtered, and evaporated. The residue was purified by flash chromatography on silica gel (dichloromethane(methanol+2% of ammonium hydroxide) to give 30 mg of impure (S)-1-((2H-tetrazol-5-yl)methyl)-3-((S)-sec-butyl)-7-chloro-5-phenyl-1,3-dihydro-2H-benzo[c][1,4]diazepin-2-one, which was purified a second time (hexanes/ethyl acetate 100/0 to 30/70). The aqueous layer from the extraction of the reaction mixture was acidified to pH2 by addition of 1M aqueous hydrochloric acid and extracted with ethyl acetate. The combined organic layers were dried over magnesium sulfate, filtered, and evaporated. The residue was purified by flash chromatography on silica gel (hexanes/ethyl acetate 100/0 to 30/70). The two batches of product were combined and crystallized from ethyl acetate/hexanes to give 21 mg of (S)-1-((2H-tetrazol-5-yl)methyl)-3-((S)-sec-butyl)-7-chloro-5-phenyl-1,3-dihydro-2H-benzo[c][1,4]diazepin-2-one. ¹H NMR (400 MHz, DMSO-d6) δ 7.78 (d, J=8.9 Hz, 1H), 7.69 (dd, J=8.9, 2.6 Hz, 1H), 7.50-7.37 (m, 5H), 7.17 (d, J=2.4 Hz, 1H), 5.38 (d, J=16.5 Hz, 1H), 5.24 (d, J=16.4 Hz, 1H), 3.21 (dd, J=9.8, 7.1 Hz, 1H), 2.36-2.20 (m, 1H), 1.92-1.72 (m, 111), 1.24-0.99 (m, 2H), 0.88-0.75 (m, 6H). HPLC: Waters XBridge C18, 4.6×150 mm, (10% MeCN in 0.1% aqeuous TFA) (100% MeCN in 0.1% aquous TFA) over 10 min at 1 mL/min, and 100% MeCN in 0.1% aquous TFA for 5 min at 1 mL/min; Detection PDA at 254 nm; RT=11.58 minutes, 99.4% purity. MS (ESI): m/z 408.95 (M+H)⁺.

### Example 18: Synthesis of 1-(2-((S)-3-((S)-sec-Butyl)-7-chloro-2-oxo-5-phenyl-2,3-dihydro-1 H-benzo[e][1,4]diazepin-1-yl)ethyl)sulfamide

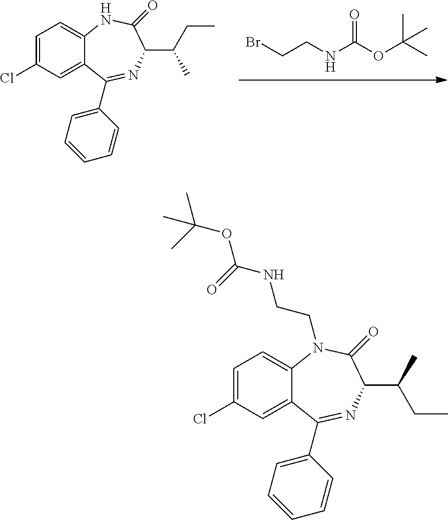 [SMILES: CC[C@H](C)[C@@H]1N=C(c2ccccc2)c2cc(Cl)ccc2NC1=O ; CC(C)(C)OC(=O)NCCBr ; CC[C@H](C)[C@@H]1N=C(c2ccccc2)c2cc(Cl)ccc2N(CCNC(=O)OC(C)(C)C)C1=O]

Step 1: Potassium tert-butoxide (0.412 g, 3.67 mmol) was added at 0° C. to a solution of (S)-3-((S)-sec-butyl)-7-chloro-5-phenyl-1,3-dihydro-2H-benzo[e][1,4]diazepin-2-one (1 g, 3.06 mmol) in 6 mL of N,N-dimethylformamide. The resulting mixture was stirred at 0° C. for 15 minutes before adding tert-butyl (2-bromoethyl)carbamate (994 μL, 4.44 mmol) in 4 mL of N,N-dimethylformamide. The reaction mixture was then stirred at 23° C. for 18 hours (h) before being quenched by the addition of 1.5 mL of water, followed by extraction with 50 mL of ethyl acetate. The organic layer was washed with saturated aqueous sodium bicarbonate, brine, and then dried over sodium sulfate, filtered, and evaporated. The crude residue of tert-butyl (2-((S)-3-((S)-sec-butyl)-7-chloro-2-oxo-5-phenyl-2,3-dihydro-1H-benzo[e][1,4]diazepin-1-yl)ethyl)carbamate was used in the next step without further purification or characterization.

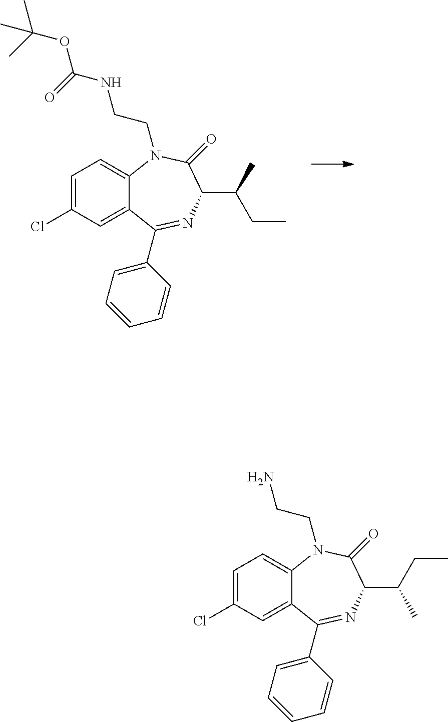 [SMILES: CC[C@H](C)[C@@H]1N=C(c2ccccc2)c2cc(Cl)ccc2N(CCNC(=O)OC(C)(C)C)C1=O ; CC[C@H](C)[C@@H]1N=C(c2ccccc2)c2cc(Cl)ccc2N(CCN)C1=O]

Step 2: To a solution of crude tert-butyl (2-((S)-3-((S)-sec-butyl)-7-chloro-2-oxo-5-phenyl-2,3-dihydro-1H-benzo[e][1,4]diazepin-1-yl)ethyl)carbamate prepared in step 1 (theoretically 3.06 mmol) in 6 mL of dichloromethane, was added 2 mL of trifluoroacetic acid at 23° C. The reaction mixture was then then stirred at 23° C. for 18 hours before being evaporated. The residue was diluted with ethyl acetate and the resulting solution was washed with saturated aqueous sodium bicarbonate, brine, dried over sodium sulfate, filtered, and evaporated. The residue was purified by flash chromatography on silica gel (dichloromethane/methanol 99/1 to 80/20) to give 0.893 g of (S)-1-(2-aminoethyl)-3-((S)-sec-butyl)-7-chloro-5-phenyl-1,3-dihydro-2H-benzo[e][1,4]diazepin-2-one (79% yield over two steps).

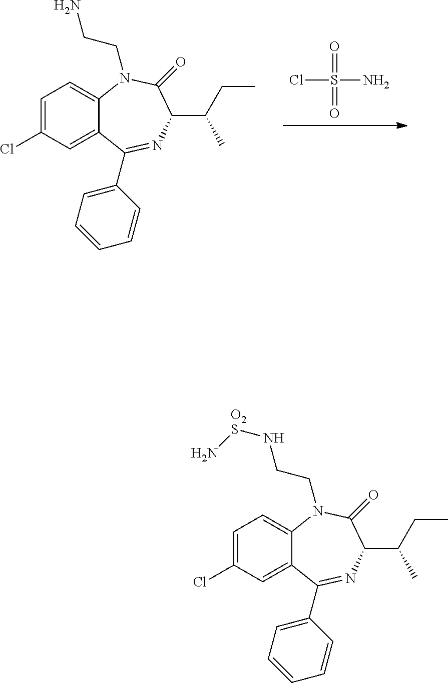

Step 3: To a solution of (S)-1-(2-aminoethyl)-3-((S)-sec-butyl)-7-chloro-5-phenyl-1,3-dihydro-2H-benzo[e][1,4]diazepin-2-one (0.15 g, 0.405 mmol) in 10 mL of dichloromethane, was added at 23° C. diisopropylethylamine (706 μL, 4.055 mmol), followed by sulfamoyl chloride (0.353 g, 3.041 mmol). The resulting mixture was stirred at 23° C. for 18 hours before being diluted with ethyl acetate, washed with saturated aqueous sodium bicarbonate, brine, dried over sodium sulfate, filtered, and evaporated. The residue was purified by flash chromatography on silica gel (dichloro-methane/methanol 99/1 to 90/10) to give 34 mg of 1-(2-((S)-3-((S)-sec-butyl)-7-chloro-2-oxo-5-phenyl-2,3-dihydro-1H-benzo[e][1,4]diazepin-1-yl)ethyl)sulfamide. 1H NMR (400 MHz, DMSO-d6): Q 7.73 (d, J=1.6 Hz, 2H), 7.56-7.44 (m, 5H), 7.23 (s, 1H), 6.64 (t, J=6.2 HZ, 1H), 6.53 (s, 2H), 4.19-4.12 (m, 1H), 3.88-3.81 (m, 1H), 3.14 (d, J=10.4 Hz, 1H), 2.92-2.87 (m, 2H), 2.41-2.35 (m, 1H), 1.92-1.87 (m, 1H), 1.90-1.10 (m, 1H), 0.91-0.87 (m, 6H). MS (ESI): m/z 449.09 (M+H)⁺.

### Example 19: Synthesis of (S)-3-((S)-sec-Butyl)-7-chloro-1-(2-hydroxyethyl)-5-phenyl-1,3-dihydro-2H-benzo[e][1,4]diazepin-2-one

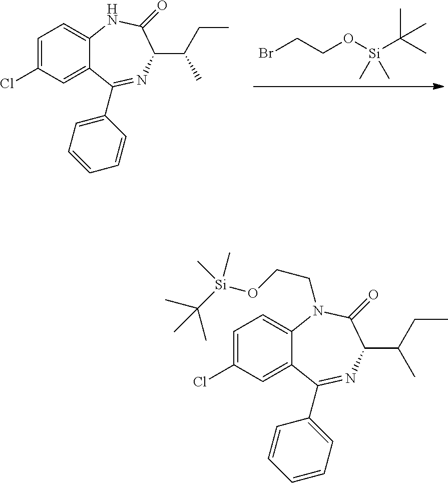 [SMILES: CC[C@H](C)[C@@H]1N=C(c2ccccc2)c2cc(Cl)ccc2NC1=O ; CC(C)(C)[Si](C)(C)OCCBr ; CCC(C)[C@@H]1N=C(c2ccccc2)c2cc(Cl)ccc2N(CCO[Si](C)(C)C(C)(C)C)C1=O]

Step 1: (2-Bromoethoxy)(tert-butyl)dimethylsilane (410 μL, 1.9 mmol), potassium carbonate (0.317 g, 2.3 mmol), and sodium iodide (0.023 g, 0.15 mmol) were added at 23° C. to a solution of (S)-3-((S)-sec-butyl)-7-chloro-5-phenyl-1,3-dihydro-2H-benzo[e][1,4]diazepin-2-one (0.5 g, 1.5 mmol) in 25 mL of acetonitrile. The resulting mixture was stirred at reflux for 48 h before being evaporated. The residue was purified by flash chromatography on silica gel (hexanes/ethyl acetate 95/5 to 60/40) to give 0.34 g of (S)-3-((S)-sec-butyl)-1-(2-((tert-butyldimethylsilyl)oxy)ethyl)-7-chloro-5-phenyl-1,3-dihydro-2H-benzo[e][1,4]diazepin-2-one. ¹H NMR (400 MHz, DMSO-d6): □ 7.86 (dd, J=9.2, 2.8 Hz, 1H), 7.69 (dd, J=8.8, 2.8 Hz, 1H), 7.55-7.43 (m, 5H), 7.23 (d, J=2.4 Hz, 1H), 4.14-4.08 (m, 1H), 3.87-3.73 (m, 2H), 3.59-3.54 (m, 1H), 3.13 (d, J=10.4 Hz, 1H), 2.40-2.36 (m, 1H), 1.90-1.86 (m, 1H), 0.91-0.86 (m, 6H), 0.73 (s, 9H), −0.08 (s, 3H), −0.09 (s, 3H);MS (ESI): m/z 485.18 (M+H)⁺.

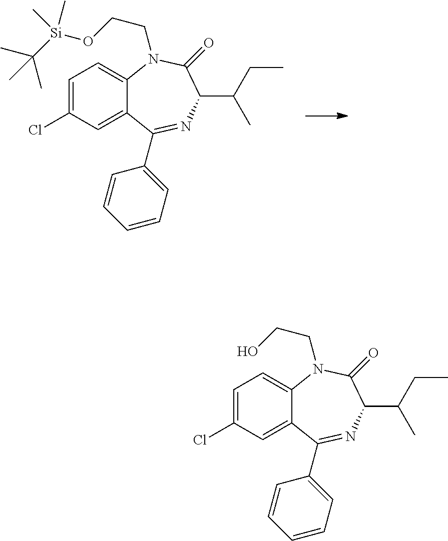 [SMILES: CCC(C)[C@@H]1N=C(c2ccccc2)c2cc(Cl)ccc2N(CCO[Si](C)(C)C(C)(C)C)C1=O ; CCC(C)[C@@H]1N=C(c2ccccc2)c2cc(Cl)ccc2N(CCO)C1=O]

Step 2: Tert-butylammonium fluoride 1M in tetrahydrofuran (488 μL, 0.69 mmol) was added at 0° C. to a solution of (S)-3-((S)-sec-butyl)-1-(2-((tert-butyldimethylsilyl)oxy)ethyl)-7-chloro-5-phenyl-1,3-dihydro-2H-benzo[e][1,4]diazepin-2-one prepared in Step 1 (90.167 mg, 0.34 mmol) in 5 mL of tetrahydrofuran. The resulting mixture was allowed to warm to 23° C. over 90 min before being evaporated. The residue was diluted with ethyl acetate, and the solution was washed with brined, dried over sodium sulfate, filtered, and evaporated. The residue was purified by flash chromatography on silica gel (hexanes/ethyl acetate 95/5 to 0/100) to give 0.11 g of (S)-3-((S)-sec-butyl)-7-chloro-1-(2-hydroxyethyl)-5-phenyl-1,3-dihydro-2H-benzo[e][1,4]diazepin-2-one. ¹H NMR (400 MHz, DMSO-d6): Q 7.83 (d, J=8.8 Hz, 1H), 7.70 (dd, J=9.2, 2.8 Hz, 1H), 7.53-7.44 (m, 5H), 7.19 (d, J=2.4 Hz, 1H), 4.74-4.71 (m, 1H), 4.12-4.02 (m, 1H), 3.81-3.75 (m, 1H), 3.50-3.38 (m, 2H), 3.13 (d, J=10.0 Hz, 1H), 2.41-2.35 (m, 1H), 1.92-1.86 (m, 1H), 1.12-1.06 (m, 1H), 0.91-0.86 (m, 6H). MS (ESI): m/z 371.04 (M+H)⁺.

### Example 20: Synthesis of (S)-3-(7-chloro-3-isopropyl-2-oxo-5-phenyl-2,3-dihydro-1H-benzo[e][1,4]-diazepin-1-yl)-N-(methylsulfonyl)propanamide

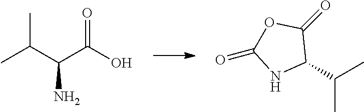

Step 1: Triphosgene (9 g, 30.3 mmol) was added at 23° C. to a solution of L-valine (4.44 g, 37.9 mmol) in 250 mL of tetrahydrofuran. The resulting mixture was stirred at reflux for 3 h before being cooled to 23° C. and evaporated. The residue was taken in 20 mL of tetrahydrofuran and then 450 mL of hexanes were added. The solution was cooled to −20° C. before being decanted in order to remove the dark oil that had separated. The process was repeated twice and the solution was cooled again to −20° C. The precipitate that formed was filtered, rinsed with hexanes, and dried under vacuum to give 2.61 g of (S)-4-isopropyloxazolidine-2,5-dione (60% yield). ¹H NMR (400 MHz, DMSO-d6): □ 9.07 (s, 1H), 4.32 (dt, J=4.2, 1.2 Hz, 1H), 2.02 (tdd, J=6.8, 4.2, 0.9 Hz, 1H), 0.92 (dd, J=6.8, 0.9 Hz, 3H), 0.82 (dd, J=6.8, 0.9 Hz, 3H).

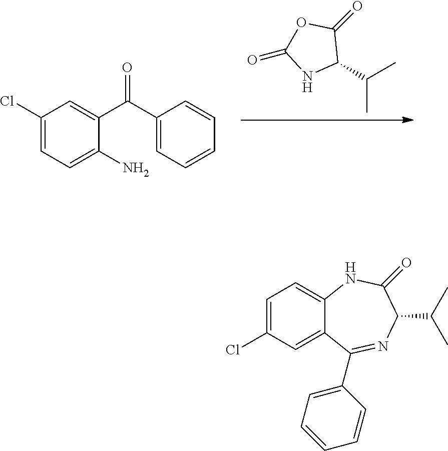

Step 2: Trifluoroacetic acid (1.2 mL, 16.3 mmol) was added at 23° C. to a solution of (2-amino-5-chloro-phenyl)(phenyl)methanone (1.89 g, 8.17 mmol) and (S)-4-isopropyloxazolidine-2,5-dione prepared in Step 1 (1.4 g, 9.8 mmol) in 27 ml of toluene. The mixture was stirred at 65° C. for 23 hours before adding triethylamine (2.3 mL, 16.3 mmol). The resulting solution was then stirred at 80° C. for 3 hours being cooled to 23° C. and evaporated. The residue was purified by flash chromatography on silica gel (hexanes, ethyl acetate 100/0 to 60/40) to give 2.3 g of (S)-7-chloro-3-isopropyl-5-phenyl-1,3-dihydro-2H-benzo[e][1,4]diazepin-2-one. 1H NMR (400 MHz, DMSO-d6): □ 10.64 (s, 1H), 7.61 (dd, J=8.7, 2.5 Hz, 1H), 7.51-7.39 (m, 5H), 7.24 (m, 2H), 3.03 (d, J=8.8 Hz, 1H), 2.52-2.41 (m, 1H), 1.08 (d, J=6.5 Hz, 3H), 0.94 (d, J=6.5 Hz, 3H). MS (ESI) m/z=313.12 (M+H)⁺.

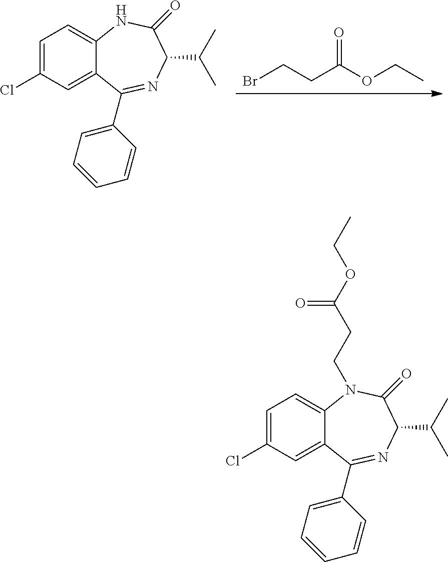

Step 3: Sodium hydride 60% in oil (0.138 g, 3.46 mmol) was added at 0° C. to a solution of (S)-7-chloro-3-isopropyl-5-phenyl-1,3-dihydro-2H-benzo[e][1,4]diazepin-2-one (0.542 g, 1.73 mmol) in 9 mL of tetrahydrofuran. The resulting mixture was stirred at 0° C. for 3 h before adding ethyl 3-bromo-propanoate (330 μL, 2.6 mmol). The solution was then stirred at 23° C. for 18 hours before being cooled to 0° C., quenched by the addition of water, and extracted with ethyl acetate. The organic layer was washed with brine, dried over magnesium sulfate, filtered, and evaporated. The residue was purified by purified by flash chromatography on silica gel (hexanes, ethyl acetate 100/0 to 55/45) to give 0.456 g of ethyl(S)-3-(7-chloro-3-isopropyl-2-oxo-5-phenyl-2,3-dihydro-1H-benzo[e][1,4]diazepin-1-yl)propanoate. ¹H NMR (400 MHz, DMSO-d6): δ 7.74-7.64 (m, 2H), 7.55-7.39 (m, 5H), 7.19 (t, J=1.5 Hz, 1H), 4.39 (dt, J=14.2, 6.3 Hz, 1H), 3.90 (dt, J=14.2, 6.3 Hz, 1H), 3.70 (q, J=6.7 Hz, 2H), 3.03 (dd, J=9.6, 0.8 Hz, 1H), 2.54-2.44 (m, 1H), 2.39 (t, J=6.3 Hz, 2H), 1.02 (d, J=6.7 Hz, 3H), 0.90 (t, J=6.7, 3H), 0.89 (d, J=6.7 Hz, 3H).MS (ESI) m/z=413.11 (M+H)⁺.

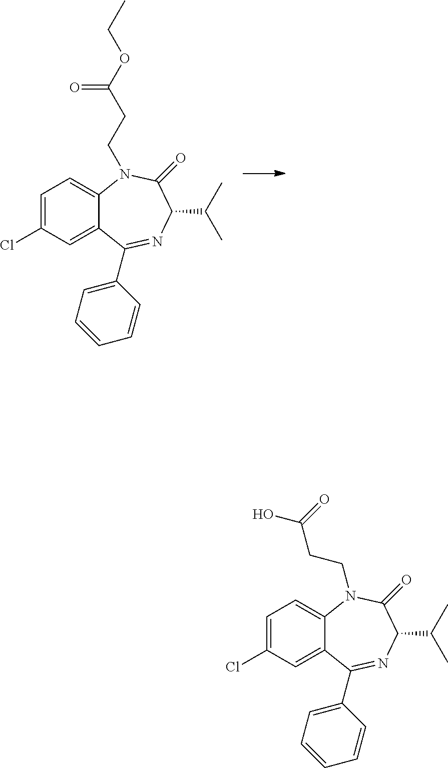 [SMILES: CCOC(=O)CCN1C(=O)[C@H](C(C)C)N=C(c2ccccc2)c2cc(Cl)ccc21 ; CC(C)[C@@H]1N=C(c2ccccc2)c2cc(Cl)ccc2N(CCC(=O)O)C1=O]

Step 4: Lithium hydroxide monohydrate (0.231 g, 5.5 mmol) was added at 23° C. to a solution of ethyl(S)-3-(7-chloro-3-isopropyl-2-oxo-5-phenyl-2,3-dihydro-1H-benzo[e][1,4]diazepin-1-yl)propanoate prepared in Step 3 (0.456 g, 1.1 mmol) in 11 mL of 1,4-dioxane and 11 mL of water. The resulting mixture was stirred at 23° C. for 19 h before being evaporated to half volume. The pH was adjusted to 5-6 by the addition of aqueous 10% citric acid and the resulting solution was extracted with ethyl acetate. The organic layer was evaporated and the residue was triturated with hexanes to give 0.319 g of (S)-3-(7-chloro-3-isopropyl-2-oxo-5-phenyl-2,3-dihydro-1H-benzo[e][1,4]-diazepin-1-yl)propanoic acid. 1H NMR (400 MHz, DMSO-d6): □ 12.12 (s, 1H), 7.73-7.65 (m, 2H), 7.53-7.46 (m, 3H), 7.46-7.40 (m, 2H), 7.16 (d, J=2.4 Hz, 1H), 4.34 (dt, J=13.9, 6.9 Hz, 1H), 3.88 (dt, J=13.9, 6.9 Hz, 1H), 3.02 (d, J=9.5 Hz, 1H), 2.58-2.49 (m, 1H), 2.31 (t, J=6.9 Hz, 2H), 1.02 (d, J=6.7 Hz, 4H), 0.89 (d, J=6.5 Hz, 3H). MS (ESI) m/z=385.10 (M+H)⁺.

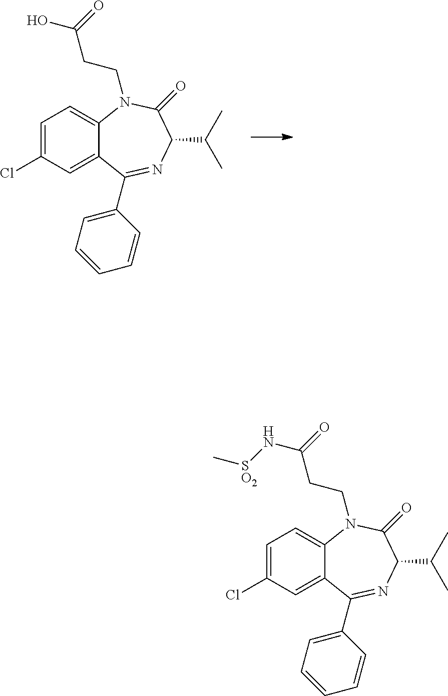 [SMILES: CC(C)[C@@H]1N=C(c2ccccc2)c2cc(Cl)ccc2N(CCC(=O)O)C1=O ; CC(C)[C@@H]1N=C(c2ccccc2)c2cc(Cl)ccc2N(CCC(=O)NS(C)(=O)=O)C1=O]

Step 5: A solution of (S)-3-(7-chloro-3-isopropyl-2-oxo-5-phenyl-2,3-dihydro-1H-benzo[e][1,4]diazepin-1-yl)propanoic acid prepared in Step 4 (0.142 g, 0.369 mmol), methanesulfonamide (0.053 g, 0.553 mmol), 1-ethyl-3-(3-dimethylaminopropyl)carbodiimide hydrochloride (0.085 g, 0.443 mmol), and 4-dimethylaminopyridine (0.009 g, 74 □mol) in 4 ml of dichloromethane was stirred at 23° C. for 22 hours before being purified by flash chromatography on silica gel (dichloromethane/methanol 94/6 to 90/10). The slightly impure product recovered by washed with ethanol and then methanol to give 0.093 g of (S)-3-(7-chloro-3-isopropyl-2-oxo-5-phenyl-2,3-dihydro-1H-benzo[e][1,4]diazepin-1-yl)-N-(methylsulfonyl)propanamide, Compound 30r (55% yield). ¹H NMR (400 MHz, DMSO-d6): □ 11.56 (s, 1H), 7.75-7.64 (m, 2H), 7.57-7.37 (m, 5H), 7.25-7.13 (m, 1H), 4.37 (dt, J=13.9, 6.8 Hz, 1H), 3.89 (dt, J=13.8, 6.8 Hz, 1H), 3.03 (d, J=9.5 Hz, 1H), 2.95 (s, 3H), 2.55-2.48 (m, 1H), 2.41-2.33 (m, 2H), 1.02 (d, J=6.6 Hz, 3H), 0.89 (d, J=6.6 Hz, 3H). MS (ESI): m/z 462.12 (M+H)⁺.m.p. 115-118° C.

### Example 21: Synthesis of (S)-1-(2-(2H-Tetrazol-5-yl)ethyl)-7-chloro-3-isopropyl-5-phenyl-1,3-dihydro-2H-benzo[e][1,4]diazepin-2-one

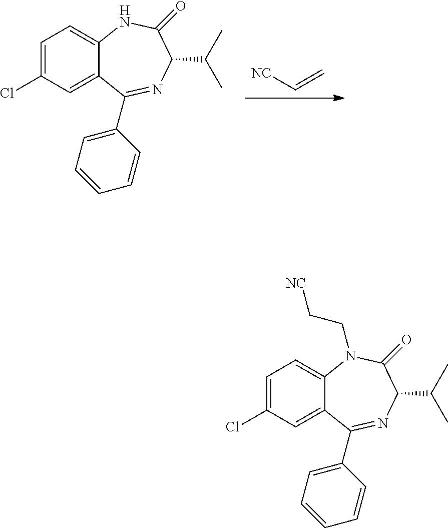

Step 1: Potassium carbonate (0.276 g, 2 mmol), followed by acrylonitrile (167 μL, 2 mmol) were added at 23° C. to a solution of (S)-7-chloro-3-isopropyl-5-phenyl-1,3-dihydro-2H-benzo[e][1,4]diazepin-2-one (0.313 g, 1 mmol) in 6 mL of N,N-dimethylformamide. The resulting mixture was stirred at 23° C. for 16 h before being filtered through Celite®. The filtrate was partitioned between ethyl acetate and water. The organic layer was washed with brine, dried over magnesium sulfate, filtered, and evaporated. The residue was purified by flash chromatography on silica gel (ethyl acetate/[hexanes/dichloromethane 1/1]1/99 to 10/90) to give 0.194 g of (S)-3-(7-chloro-3-isopropyl-2-oxo-5-phenyl-2,3-dihydro-1H-benzo[e][1,4]-diazepin-1-yl)propanenitrile. ¹H NMR (400 MHz, CDCl3) □ 0.98 (d, J=6.8 Hz, 3H), 1.12 (d, J=6.4 Hz, 3H), 2.54-2.80 (m, 3H), 3.05 (d, J=9.6 Hz, 1H), 3.87-3.94 (m, 1H), 4.43-4.49 (m, 1H), 7.32-7.67 (m, 8H).MS (ESI): m/z 366.12 (M+H)⁺.

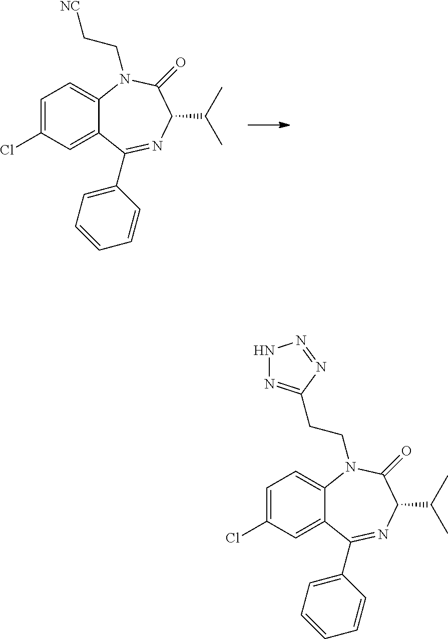 [SMILES: CC(C)[C@@H]1N=C(c2ccccc2)c2cc(Cl)ccc2N(CCC#N)C1=O ; CC(C)[C@@H]1N=C(c2ccccc2)c2cc(Cl)ccc2N(CCc2nn[nH]n2)C1=O]

Step 2: Triethylammonium chloride (0.256 g, 1.85 mmol) and sodium azide (0.121 g, 1.85 mmol) were added at 23° C. to a solution of (S)-3-(7-chloro-3-isopropyl-2-oxo-5-phenyl-2,3-dihydro-1H-benzo[e][1,4]diazepin-1-yl)propanenitrile prepared in Step 1 (0.194 g, 0.53 mmol) in 4 mL of N,N-dimethylformamide. The resulting mixture was microwaved at 130° C. for 8 hours before being cooled to 23° C. and partitioned between ethyl acetate and water. The organic layer was washed with brine, dried over sodium sulfate, filtered, and evaporated. The residue showed residual N,N-dimethylformamide by ¹H NMR, soit was dissolved into dichloromethane and the resulting solution was washed twice with cold water. The organic layer was dried over sodium sulfate, filtered, and evaporated to give 0.087 g of (S)-1-(2-(2H-tetrazol-5-yl)ethyl)-7-chloro-3-isopropyl-5-phenyl-1,3-dihydro-2H-benzo[e][1,4]-diazepin-2-one. ¹H NMR (400 MHz, CDCl3): D 0.97 (d, J=6 Hz, 3H), 1.11 (d, J=6.4 Hz, 3H), 2.71-2.79 (m, 1H), 2.96-3.16 (m, 2H), 3.06 (d, J=10 Hz, 1H), 4.08-4.15 (m, 1H), 4.51-4.58 (m, 1H), 7.23 (m, 1H), 7.34-7.45 (m, 6H), 7.56-7.59 (dd, J=9.2, 2.8 Hz, 1H). MS (ESI): m/z 409.01 (M+H)⁺.

### Example 22: Synthesis of 1-(2-((S)-3-((S)-sec-Butyl)-7-chloro-2-oxo-5-phenyl-2,3-dihydro-1H-benzo[e]-[1,4]diazepin-1-yl)ethyl)urea

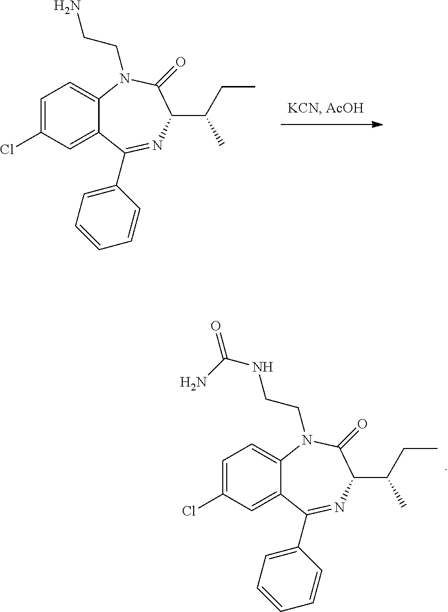 [SMILES: CC[C@H](C)[C@@H]1N=C(c2ccccc2)c2cc(Cl)ccc2N(CCN)C1=O ; CC[C@H](C)[C@@H]1N=C(c2ccccc2)c2cc(Cl)ccc2N(CCNC(N)=O)C1=O]

Acetic acid (49 μL, 0.811 mmol) was added at 23° C. to a solution of (S)-1-(2-aminoethyl)-3-((S)-sec-butyl)-7-chloro-5-phenyl-1,3-dihydro-2H-benzo[e][1,4]diazepin-2-one (0.15 g, 0.405 mmol) in 4.5 mL of water. The resulting mixture was sonicated to facilitate the dissolution of the starting material. Potassium cyanate (0.033 g, 0.405 mmol) was then added and the reaction mixture was stirred at 23° C. for 18 hours before being filtered. The residue was rinsed with water, dried under vacuum and purified by flash chromatography on silica gel (dichloromethane/methanol 99/1 to 90/10) to give 0.054 g of 1-(2-((S)-3-((S)-sec-butyl)-7-chloro-2-oxo-5-phenyl-2,3-dihydro-1H-benzo[e][1,4]diazepin-1-yl)-ethyl)urea. 1H NMR (400 MHz, DMSO-d6): □ 7.59-7.53 (m, 3H), 7.49-7.39 (m, 4H), 7.27 (d, J=2.0 Hz, 1H), 5.15 (s, 1H), 4.28-4.21 (m, 1H), 3.89-3.82 (m, 1H), 3.31-3.19 (m, 2H), 3.16 (d, J=10.0 Hz, 1H), 2.61-2.54 (m, 1H), 2.01-1.94 (m, 2H), 1.19-1.12 (m, 1H), 0.98-0.92 (m, 6H). MS (ESI): m/z 413.09 (M+H)⁺.

### Example 29: Synthesis of 3-((S)-3-((S)-sec-Butyl)-7-chloro-2-oxo-5-phenyl-2,3-dihydro-1 H-benzo-[e][1,4]diazepin-1-yl)-N-(methylsulfonyl)propanamide

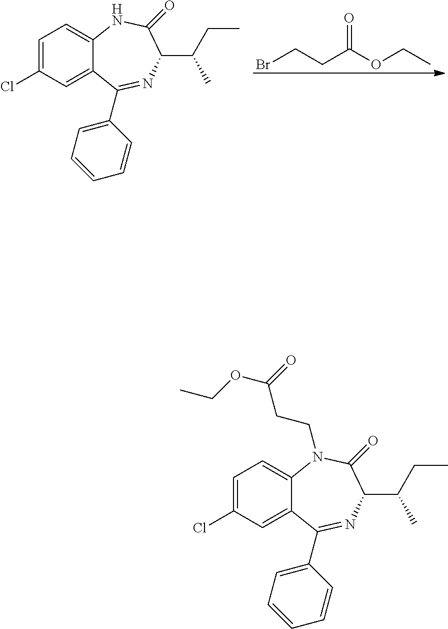

Step 1: Sodium hydride 60% in oil (0.083 g, 2.08 mmol) was added at 0° C. to a solution of (S)-3-((S)-sec-butyl)-7-chloro-5-phenyl-1,3-dihydro-2H-benzo[e][1,4]diazepin-2-one (0.34 g, 1.04 mmol) in 5 mL of tetrahydrofuran. The resulting mixture was stirred at 0° C. for 3 hours before adding ethyl 3-bromopropanoate (200 μL, 1.56 mmol). The solution was then stirred at 23° C. for 3 days before being cooled to 0° C., quenched by the addition of water, and extracted with ethyl acetate. The organic layer was washed with brine, dried over magnesium sulfate, filtered, and evaporated. The residue was purified by purified by flash chromatography on silica gel (hexanes, ethyl acetate 100/0 to 55/45) to give 0.41 g of ethyl 3-((S)-3-((S)-sec-butyl)-7-chloro-2-oxo-5-phenyl-2,3-dihydro-1H-benzo[e][1,4]diazepin-1-yl)propanoate. ¹H NMR (400 MHz, DMSO-d6): □ 7.74-7.64 (m, 2H), 7.57-7.38 (m, 5H), 7.20 (d, J=2.6 Hz, 1H), 4.38 (dt, J=13.3, 6.3 Hz, 1H), 3.90 (dt, J=13.6, 6.5 Hz, 1H), 3.71 (q, J=6.2 Hz, 2H), 3.09 (d, J=10.0 Hz, 1H), 2.39 (t, J=6.5 Hz, 2H), 2.37-2.28 (m, 1H), 1.90-1.78 (m, 1H), 1.13-1.02 (m, 1H), 0.90 (t, J=7.1 Hz, 3H), 0.86 (d, J=6.6 Hz, 3H), 0.84 (t, J=7.5 Hz, 3H).MS (ESI): m/z 427.08 (M+H)⁺.

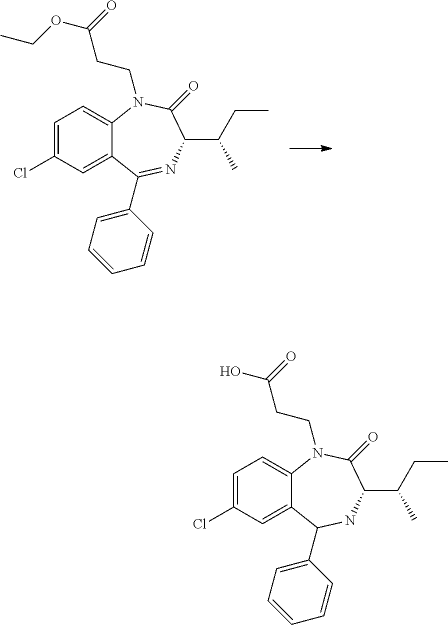

Step 2: Lithium hydroxide monohydrate (0.197 g, 4.69 mmol) was added at 23° C. to a solution of ethyl 3-((S)-3-((S)-sec-butyl)-7-chloro-2-oxo-5-phenyl-2,3-dihydro-1H-benzo[e][1,4]diazepin-1-yl)-propanoate (0.41 g, 0.937 mmol) in 9.4 mL of 1,4-dioxane and 9.4 mL of water. The resulting mixture was stirred at 23° C. for 19 hours before being evaporated to half volume. The pH was adjusted to 5-6 by the addition of aqueous 10% citric acid and the resulting solution was extracted with ethyl acetate. The organic layer was evaporated and the residue was triturated with hexanes to give 0.319 g of 3-((S)-3-((S)-sec-butyl)-7-chloro-2-oxo-5-phenyl-2,3-dihydro-1H-benzo[e][1,4]diazepin-1-yl)propanoic acid (55%). ¹H NMR (400 MHz, DMSO-d6) □ 12.13 (s, 1H), 7.73-7.64 (m, 2H), 7.52-7.46 (m, 3H), 7.46-7.40 (m, 2H), 7.16 (d, J=2.4 Hz, 1H), 4.33 (dt, J=13.7, 6.6 Hz, 1H), 3.88 (dt, J=13.9, 6.9 Hz, 1H), 3.08 (d, J=9.9 Hz, 1H), 2.39-2.33 (m, 1H), 2.30 (t, J=6.9 Hz, 2H), 1.91-1.78 (m, 1H), 1.16-1.03 (m, 1H), 0.86 (d, J=6.5 Hz, 3H), 0.83 (t, J=7.6 Hz, 3H).MS (ESI): m/z 323.31 (M+H)⁺.

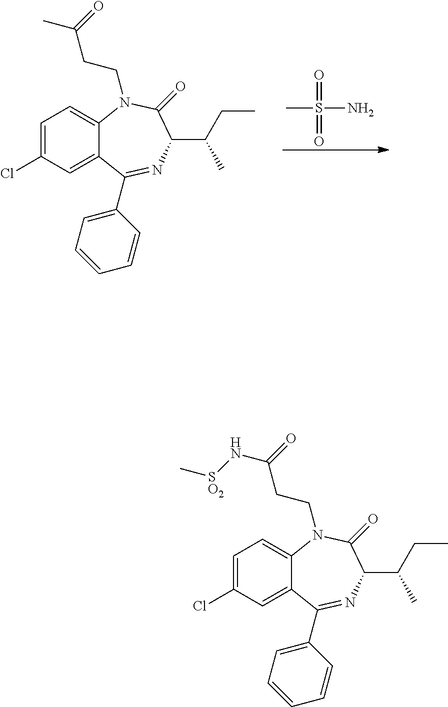

Step 3: A solution of 3-((S)-3-((S)-sec-butyl)-7-chloro-2-oxo-5-phenyl-2,3-dihydro-1H-benzo[e][1,4]-diazepin-1-yl)propanoic acid prepared in Step 2 (0.100 g, 0.25 mmol), methanesulfonamide (0.036 g, 0.376 mmol), 1-ethyl-3-(3-dimethylaminopropyl)carbodiimide hydrochloride (0.058 g, 0.301 mmol), and 4-dimethylaminopyridine (0.006 g, 50 mmol) in 1.2 mL of dichloromethane was stirred at 23° C. for 22 hours before being purified by flash chromatography on silica gel (dichloromethane/methanol 100/0 to 85/15). The slightly impure product recovered by washed with ethanol and then methanol to give 0.045 g of 3-((S)-3-((S)-sec-butyl)-7-chloro-2-oxo-5-phenyl-2,3-dihydro-1H-benzo[e][1,4]diazepin-1-yl)-N-(methylsulfonyl)propanamide. ¹H NMR (400 MHz, DMSO-d6): □ 11.56 (s, 1H), 7.74-7.65 (m, 2H), 7.57-7.38 (m, 5H), 7.18 (d, J=2.5 Hz, 1H), 4.36 (dt, J=13.8, 6.8 Hz, 1H), 3.89 (dt, J=13.8, 6.8 Hz, 1H), 3.09 (d, J=10.0 Hz, 1H), 2.95 (s, 3H), 2.38 (t, J=6.8 Hz, 2H), 2.35-2.26 (m, 1H), 1.90-1.78 (m, 1H), 1.16-1.01 (m, 1H), 0.87 (d, J=6.4 Hz, 3H), 0.84 (t, J=7.4 Hz, 3H). MS (ESI): m/z 476.12 (M+H)⁺.m.p. 113-115° C.

### Example 23: Synthesis of (S)-1-(2-(2H-Tetrazol-5-yl)ethyl)-3-((S)-sec-butyl)-7-chloro-5-phenyl-1,3-dihydro-2H-benzo[e][1,4]diazepin-2-one

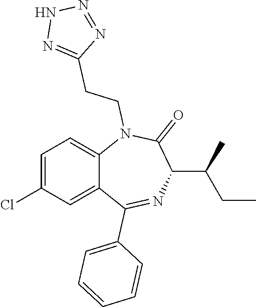 [SMILES: CC[C@H](C)[C@@H]1N=C(c2ccccc2)c2cc(Cl)ccc2N(CCc2nn[nH]n2)C1=O]

Step 1: Potassium carbonate (0.276 g, 2 mmol), followed by acrylonitrile (167 μL, 2 mmol) were added at 23° C. to a solution of (S)-3-((S)-sec-butyl)-7-chloro-5-phenyl-1,3-dihydro-2H-benzo[e][1,4]-diazepin-2-one (0.327 g, 1 mmol) in 6 mL of N,N-dimethylformamide. The resulting mixture was stirred at 23° C. for 16 hours before being filtered through Celite®. The filtrate was partitioned between ethyl acetate and water. The organic layer was washed with brine, dried over magnesium sulfate, filtered, and evaporated. The residue was purified by flash chromatography on silica gel (ethyl acetate/[hexanes/dichloromethane 1/1]1/99 to 10/90) to give 0.191 g of 3-((S)-3-((S)-sec-butyl)-7-chloro-2-oxo-5-phenyl-2,3-dihydro-1H-benzo[e][1,4]diazepin-1-yl)propanenitrile (50% yield). ¹H NMR (400 MHz, CDCl3): Q 0.92-0.98 (m, 6H), 1.13-1.19 (m, 1H), 1.96-2.03 (m, 1H), 2.54-2.68 (m, 3H), 3.14 (d, J=10.4 Hz, 1H), 3.87 (m, 1H), 4.43 (m, 1H), 7.31-7.66 (m, 8H).MS (ESI): m/z 380.12(M+H)⁺.

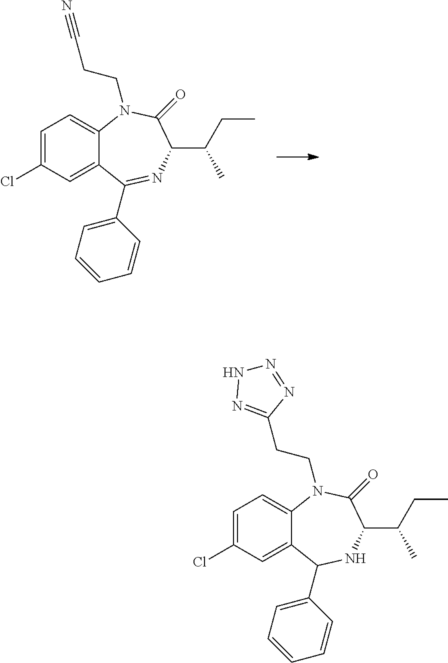

Step 2: Triethylammonium chloride (0.203 g, 1.47 mmol) and sodium azide (0.096 g, 1.47 mmol) were added at 23° C. to a solution of 3-((S)-3-((S)-sec-butyl)-7-chloro-2-oxo-5-phenyl-2,3-dihydro-1H-benzo[c][1,4]diazepin-1-yl)propanenitrile prepared in Step 1 (0.161 g, 0.42 mmol) in 4 mL of N,N-dimethylformamide. The resulting mixture was microwaved at 130° C. for 8 hours before being cooled to 23° C. and partitioned between ethyl acetate and water. The organic layer was washed with brine, dried over sodium sulfate, filtered, and evaporated. The residue showed residual N,N-dimethylformamide by ¹H NMR, soit was dissolved into dichloromethane and the resulting solution was washed twice with cold water. The organic layer was dried over sodium sulfate, filtered, and evaporated to give 0.057 g of (S)-1-(2-(2H-tetrazol-5-yl)ethyl)-3-((S)-sec-butyl)-7-chloro-5-phenyl-1,3-dihydro-2H-benzolell 1,4]diazepin-2-one. ¹H NMR (400 MHz, CDCl3): Q 0.90-0.97 (m, 6H), 1.16-1.24 (m, 1H), 1.94-1.99 (m, 1H), 2.57 (m, 1H), 3.00-3.20 (m, 2H), 3.15 (d, J=10.4 Hz, 1H), 4.08-4.15 (m, 1H), 4.52-4.59 (m, 1H), 7.36-7.45 (m, 6H), 7.57-7.50 (dd, J=9.2, 2.4 Hz, 1H). MS (ESI): m/z 423.07 (M+H)⁺.

### Example 24: Synthesis of 2-((S)-3-((S)-sec-butyl)-7-chloro-2-oxo-5-phenyl-2,3-dihydro-1H-benzo[e][1,4]diazepin-1-yl)-N-sulfamoylacetamide

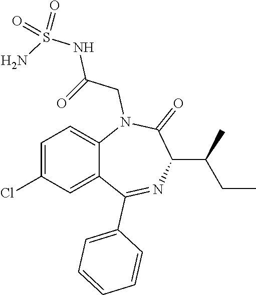 [SMILES: CC[C@H](C)[C@@H]1N=C(c2ccccc2)c2cc(Cl)ccc2N(CC(=O)NS(N)(=O)=O)C1=O]

1.1 eq. sulfamide, 1.5 eq. N-(3-dimethylaminopropyl)-N′-ethylcarbodiimide hydrochloride, 1.1 eq. hydroxybenzotriazole, and excess N,N-diisopropylethylamine was added to 2-((S)-3-((S)-sec-butyl)-7-chloro-2-oxo-5-phenyl-2,3-dihydro-1H-benzo[e][1,4]diazepin-1-yl)acetic acid (20 mg, 0.052 mmol, 1 eq) in 2 mL CH₂Cl₂. After stirring for 18 hours, EtOAc was added to the reaction and it was washed with saturated NH₄Cl and brine. Then, it was purified by Combi-Flash with a gradient of ethyl acetate: hexanes from 2:8 to 5:5 to afford 10 mg of the title compound. ¹H NMR (300 MHz, CDCl₃): δ (ppm) 7.60-7.50 (m, 4H), 7.50-7.36 (m, 3H), 7.31-7.28 (m, 1H), 4.52-4.28 (m, 2H), 3.28-3.22 (m, 1H), 2.56-2.40 (m, 1H), 2.06-1.90 (m, 1H), 1.30-1.13 (m, 1H), 0.98-0.92 (m, 6H); Calculated for C₂₁H₂₃ClN₄O₄S, 462.11; observed (M+H)⁺463.6.

### Example 25: Synthesis of 2-((S)-3-((S)-sec-butyl)-7-chloro-2-oxo-5-phenyl-2,3-dihydro-1H-benzo[e][1,4]diazepin-1-yl)-N-((trifluoromethyl)sulfonyl)acetamide

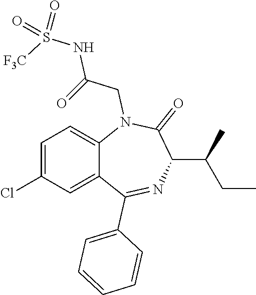 [SMILES: CC[C@H](C)[C@@H]1N=C(c2ccccc2)c2cc(Cl)ccc2N(CC(=O)NS(=O)(=O)C(F)(F)F)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide, except trifluoromethanesulfonamide was substituted for sulfamide and then purified by Combi-Flash to afford 23.5 mg of the title compound. ¹H NMR (300 MHz, CDCl₃): δ (ppm) 7.56-7.49 (m, 4H), 7.45-7.32 (m, 3H), 7.25-7.23 (m, 1H), 4.44-4.32 (m, 1H), 4.26-4.15 (m, 1H), 3.21-3.14 (m, 1H), 2.00-1.80 (m, 1H), 1.20-1.02 (m, 1H), 0.92-0.80 (m, 6H); Calculated for C₂₂H₂₁ClF₃N₃O₄S, 515.09; observed (M+H)⁺516.7.

### Example 26: Synthesis of 2-((S)-3-((S)-sec-butyl)-7-chloro-2-oxo-5-phenyl-2,3-dihydro-1H-benzo[c][1,4]diazepin-1-yl)-N-(N,N-dimethylsulfamoyl)acetamide

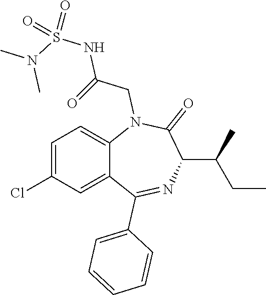 [SMILES: CC[C@H](C)[C@@H]1N=C(c2ccccc2)c2cc(Cl)ccc2N(CC(=O)NS(=O)(=O)N(C)C)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except N,N-dimethylsulfamide was substituted for sulfamide and then purified by Combi-Flash to afford 14.5 mg of the title compound. ¹H NMR (300 MHz, CDCl₃): δ (ppm) 7.63-7.58 (m, 2H), 7.57-7.51 (m, 1H), 7.50-7.38 (m, 4H), 7.33-7.30 (m, 1H), 4.63-4.55 (m, 1H), 4.28-4.20 (m, 1H), 3.26 (d, J=9.9 Hz, 1H), 2.89 (s, 6H), 1.10-0.95 (m, 6H); Calculated for C₂₃H₂₇ClN₄O₄S, 490.14; observed (M+H)⁺491.7.

### Example 27: Synthesis of 2-((S)-3-((S)-sec-butyl)-7-chloro-2-oxo-5-phenyl-2,3-dihydro-1H-benzo[e][1,4]diazepin-1-yl)-N-(tert-butylsulfonyl)acetamide

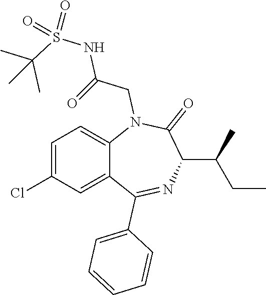 [SMILES: CC[C@H](C)[C@@H]1N=C(c2ccccc2)c2cc(Cl)ccc2N(CC(=O)NS(=O)(=O)C(C)(C)C)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except was substituted for sulfamide tert-butylsulfonamide, and then purified by Combi-Flash to afford 10.5 mg of the title compound. Calculated for C₂₅H₃₀ClN₃O₄S, 503.16; observed (M+H)⁺504.7.

### Example 28: Synthesis of 2-((S)-3-((S)-sec-butyl)-7-chloro-2-oxo-5-phenyl-2,3-dihydro-1H-benzo[c][1,4]diazepin-1-yl)-N-(cyclopropylsulfonyl)acctamide

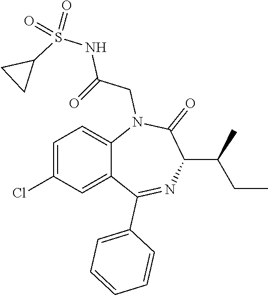 [SMILES: CC[C@H](C)[C@@H]1N=C(c2ccccc2)c2cc(Cl)ccc2N(CC(=O)NS(=O)(=O)C2CC2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except cyclopropanesulfonamide was substituted for sulfamide and then purified by Combi-Flash to afford 12.9 mg of the title compound. ¹H NMR (300 MHz, CDCl₃): δ (ppm) 7.62-7.31 (m, 8H), 4.70-4.60 (m, 1H), 4.35-4.25 (m, 1H), 3.30-3.20 (m, 1H), 1.34-1.18 (m, 4H), 1.09-0.92 (m, 10H); Calculated for C₂₄H₂₆ClN₃O₄S, 487.13; observed (M+H)⁺488.7.

### Example 28: Synthesis of 2-((S)-3-((S)-sec-butyl)-7-chloro-2-oxo-5-phenyl-2,3-dihydro-1H-benzo[e][1,4]diazepin-1-yl)-N-((difluoromethyl)sulfonyl)acetamide

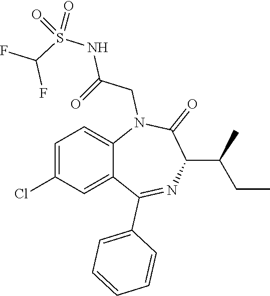 [SMILES: CC[C@H](C)[C@@H]1N=C(c2ccccc2)c2cc(Cl)ccc2N(CC(=O)NS(=O)(=O)C(F)F)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except difluoromethanesulfonanide was substituted for sulfainide, and then purified by Combi-Flash to afford 23.4 mg of the title compound. Calculated for C22H₂₂ClF₂N₃O₄S, 497.10; observed (M+H)⁺498.7.

### Example 29: Synthesis of 2-((S)-3-((S)-sec-butyl)-7-chloro-2-oxo-5-phenyl-2,3-dihydro-1H-benzo[e][1,4]diazepin-1-yl)-N-(phenylsulfonyl)acetamide

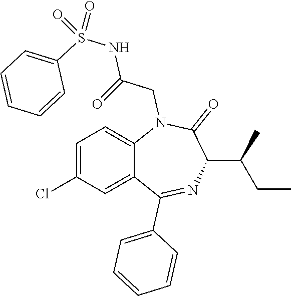 [SMILES: CC[C@H](C)[C@@H]1N=C(c2ccccc2)c2cc(Cl)ccc2N(CC(=O)NS(=O)(=O)c2ccccc2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except benzenesulfonamide was substituted for sulfamide, and then purified by Combi-Flash to afford 19.0 of the title compound. ¹H NMR (300 MHz, CDCl₃): δ (ppm) 7.90-7.82 (m, 2H), 7.52-7.43 (m, 4H), 7.43-7.32 (m, 6H), 7.23-7.21 (m, 1H), 4.50-4.20 (m, 211), 3.20-3.10 (m, 1H), 2.60-2.40 (m, 1H), 2.05-1.85 (m, 1H), 1.25-1.05 (m, 1H), 0.97-0.84 (m, 6H); Calculated for C₂₇H₂₆ClN₃O₄S, 523.13; observed (M+H)⁺524.7.

### Example 30: Synthesis of N-(benzylsulfonyl)-2-((S)-3-((S)-sec-butyl)-7-chloro-2-oxo-5-phenyl-2,3-dihydro-1H-benzo[e][1,4]diazepin-1-yl)acetamide

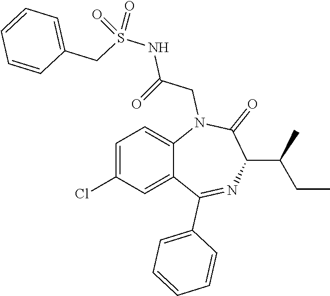 [SMILES: CC[C@H](C)[C@@H]1N=C(c2ccccc2)c2cc(Cl)ccc2N(CC(=O)NS(=O)(=O)Cc2ccccc2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except alpha-toluenesulfonamide was substituted for sulfamide, and then purified by Combi-Flash to afford 23.7 mg of the title compound. Calculated for C₂₈H₂₈ClN₃O₄S, 537.15; observed (M+H)⁺538.8.

### Example 31: Synthesis of 2-((S)-3-((S)-sec-butyl)-7-chloro-2-oxo-5-phenyl-2,3-dihydro-1H-benzo[e][1,4]diazepin-1-yl)-N-(thiophen-2-ylsulfonyl)acetamide

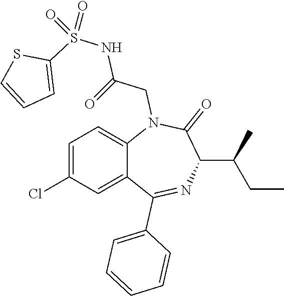 [SMILES: CC[C@H](C)[C@@H]1N=C(c2ccccc2)c2cc(Cl)ccc2N(CC(=O)NS(=O)(=O)c2cccs2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except thiophene-2-sulfonamide was substituted for sulfamide, and then purified by Combi-Flash to afford 25.4 mg of the title compound. ¹H NMR (300 MHz, CDCl₃): δ (ppm) 7.68-7.58 (m, 1H), 7.55-7.44 (m, 5H), 7.44-7.34 (m, 311), 7.25-7.22 (m, 1H), 6.98-6.90 (m, 1H), 4.50-4.32 (m, 2H), 3.20 (d, J=9.9 Hz, 1H), 2.62-2.48 (m, 1H), 2.10-1.90 (m, 1H), 1.25-1.05 (m, 1H), 0.99-0.90 (m, 6H); Calculated for C₂₅H₂₄ClN₃O₄S₂, 529.09; observed (M+H)⁺530.7.

### Example 32: Synthesis of 2-((S)-3-((S)-sec-butyl)-7-chloro-2-oxo-5-phenyl-2,3-dihydro-1H-benzo[e][1,4]diazepin-1-yl)-N-((5-chlorothiophen-2-yl)sulfonyl)acetamide

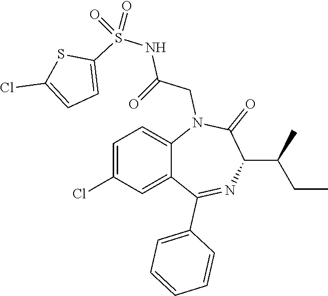 [SMILES: CC[C@H](C)[C@@H]1N=C(c2ccccc2)c2cc(Cl)ccc2N(CC(=O)NS(=O)(=O)c2ccc(Cl)s2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except 5-chlorothiophene-2-sulfonamide was substituted for sulfamide, and then purified by Combi-Flash to afford 24.9 mg of the title compound. ¹H NMR (300 MHz, CDCl₃): δ (ppm) 7.52-7.44 (m, 4H), 7.44-7.32 (m, 4H), 7.25-7.20 (m, 1H), 6.72-6.64 (m, 1H), 4.54-4.20 (m, 2H), (m, 1H), 3.20 (d, J=10.5 Hz, 1H), 2.55-2.45 (m, 1H), 2.00-1.85 (m, 1H), 1.25-1.05 (m, 1H), 0.97-0.86 (m, 6H); Calculated for C₂₅H₂₃Cl₂N₃O₄S₂, 563.05; observed (M+H)⁺ 564.7.

### Example 33: Synthesis of 2-((S)-3-((S)-sec-butyl)-7-chloro-2-oxo-5-phenyl-2,3-dihydro-1H-benzo[e][1,4]diazepin-1-yl)-N-((1-methyl-1H-imidazol-4-yl)sulfonyl)acetamide

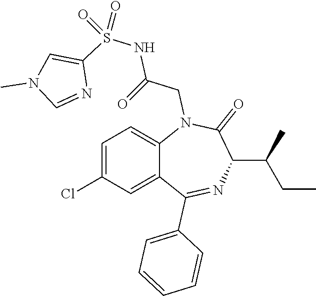 [SMILES: CC[C@H](C)[C@@H]1N=C(c2ccccc2)c2cc(Cl)ccc2N(CC(=O)NS(=O)(=O)c2cn(C)cn2)C1=O]

The title compound was prepared according to the procedure of 2-((S)-3-((S)-sec-butyl)-7-chloro-2-oxo-5-phenyl-2,3-dihydro-1H-benzoe|111,41diazepin-1-yl)-N-sulfamoylacetamide except 1-methylimidazole-2-sulfonamide was substituted for sulfamide, and then purified by Combi-Flash to afford 4.3 mg of the title compound. ¹H NMR (300 MHz, CDCl₃): δ (ppm) 7.61 (s, 1H), 7.57-7.51 (m, 2H), 7.50-7.34 (m, 5H), 7.26-7.23 (m, 1H), 7.23-7.20 (m, 1H), 4.62-6.52 (m, 1H), 4.43-4.32 (m, 1H), 3.66 (s, 3H), 3.18-3.12 (m, 1H), 2.60-2.40 (m, 1H), 2.00-1.85 (m, 1H), 1.20-1.00 (m, 1H), 0.93-0.82 (m, 6H); Calculated for C₂₅H₂₆ClN₅O₄S, 527.14; observed (M+H)⁺528.7.

### Example 34: Synthesis of 2-((S)-3-((S)-sec-butyl)-7-chloro-2-oxo-5-phenyl-2,3-dihydro-1H-benzo[e][1,4]diazepin-1-yl)-N-((tetrahydro-2H-pyran-4-yl)sulfonyl)acetamide

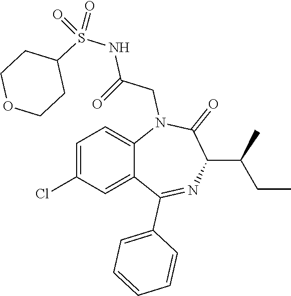 [SMILES: CC[C@H](C)[C@@H]1N=C(c2ccccc2)c2cc(Cl)ccc2N(CC(=O)NS(=O)(=O)C2CCOCC2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except oxane-4-sulfonamide was substituted for sulfamide, and then purified by HPLC to afford 23.0 mg of the title compound. ¹H NMR (300 MHz, CDCl₃): δ (ppm) 7.59-7.53 (m, 2H), 7.53-7.34 (m, 5H), 7.28-7.24 (m, 1H), 4.55-4.46 (m, 1H), 4.29-4.19 (m, 1H), 4.10-4.00 (m, 2H), 3.76-3.62 (m, 1H0, 3.39-3.24 (m, 2H), 3.20 (d, J=9.9 Hz, 1H), 2.10-1.80 (m, 6H), 1.24-1.09 (m, 1H), 0.96-0.88 (m, 6H); Calculated for C₂₆H₃₀ClN₃O₅S, 531.16; observed (M+H)⁺532.8.

### Example 35: Synthesis of 2-((S)-3-((S)-sec-butyl)-7-chloro-2-oxo-5-phenyl-2,3-dihydro-1H-benzo[e][1,4]diazepin-1-yl)-N-(pyridin-2-ylsulfonyl)acetamide

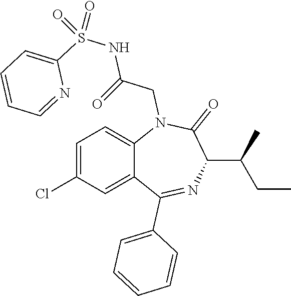 [SMILES: CC[C@H](C)[C@@H]1N=C(c2ccccc2)c2cc(Cl)ccc2N(CC(=O)NS(=O)(=O)c2ccccn2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetanide except pyridine-2-sulfonamide was substituted for sulfamide, and then purified by HPLC to afford 27.0 mg of the title compound. ¹H NMR (300 MHz, CDCl₃): δ (ppm) 8.65-8.58 (m, 1H), 8.13-8.06 (m, 1H), 7.92-7.83 (m, 1H), 7.54-7.32 (m, 8H), 7.20-7.16 (m, 1H), 4.66-4.55 (m, 1H), 4.44-4.34 (m, 1H), 3.20-3.00 (m, 1H), 2.50-2.32 (m, 1H), 1.98-1.80 (m, 1H), 1.22-1.02 (m, 1H), 0.85 (t, J=7.7 Hz, 3H), 0.77 (d, J=6.3 Hz, 3H); Calculated for C₂₆H₂₅ClN₄O₄S, 524.13; observed (M+H)⁺525.8.

### Example 36: Synthesis of 2-((S)-3-((S)-sec-butyl)-7-chloro-2-oxo-5-phenyl-2,3-dihydro-1H-benzo[e][1,4]diazepin-1-yl)-N-(pyridin-3-ylsulfonyl)acetamide

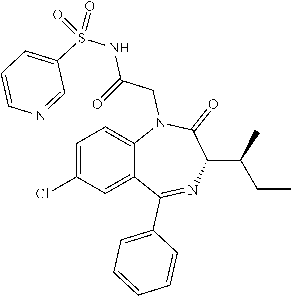 [SMILES: CC[C@H](C)[C@@H]1N=C(c2ccccc2)c2cc(Cl)ccc2N(CC(=O)NS(=O)(=O)c2cccnc2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except pyridine-3-sulfonamide was substituted for sulfamide, and then purified by HPLC to afford 25.3 mg of the title compound. ¹H NMR (300 MHz, CDCl₃): δ (ppm) 8.34-8.28 (m, 1H), 7.54-7.23 (m, 10H), 7.21-7.19 (m, 1H), 4.48-4.38 (m, 1H), 4.28-4.18 (m, 1H), 3.09 (d, J=10.5 Hz, 1H), 2.44-2.26 (m, 1H), 1.98-1.80 (m, 1H), 1.22-1.00 (m, 1H), 0.85 (t, J=7.4 Hz, 3H), 0.72 (d, J=6.3 Hz, 3H); Calculated for C₂₆H₂₅ClN₄O₄S, 524.13; observed (M+H)⁺525.8.

### Example 37: Synthesis of 2-((S)-3-((S)-sec-butyl)-7-chloro-2-oxo-5-phenyl-2,3-dihydro-1H-benzo[e][1,4]diazepin-1-yl)-N-(pyridin-4-ylsulfonyl)acetamide

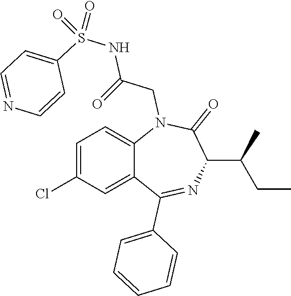 [SMILES: CC[C@H](C)[C@@H]1N=C(c2ccccc2)c2cc(Cl)ccc2N(CC(=O)NS(=O)(=O)c2ccncc2)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetanide except pyridine-4-sulfonamide was substituted for sulfamide, and then purified by HPLC to afford 26.3 of the title compound. ¹H NMR (300 MHz, CDCl₃): δ (ppm) 8.78-8.70 (m, 1H), 7.87-7.80 (m, 2H), 7.54-7.28 (m, 8H), 7.22-7.18 (m, 1H), 4.48-4.39 (m, 11H), 4.27-4.18 (m, 11H), 3.10 (d, J=9.9 Hz, 1H), 2.44-2.28 (m, 1H), 1.98-1.80 (m, 1H), 1.16-0.98 (m, 1H), 0.86 (t, J=7.4 Hz, 3H), 0.74 (d, J=6.3 Hz, 3H); Calculated for C₂₆H₂₅ClN₄O₄S, 524.13; observed (M+H)⁺525.8.

### Example 38: Synthesis of 2-((S)-3-((S)-sec-butyl)-7-chloro-2-oxo-5-phenyl-2,3-dihydro-1H-benzo[e][1,4]diazepin-1-yl)-N-((2-fluorophenyl)sulfonyl)acetamide

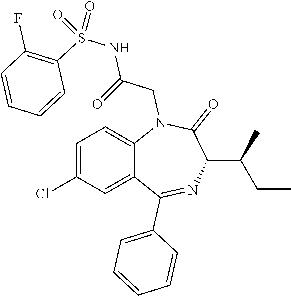 [SMILES: CC[C@H](C)[C@@H]1N=C(c2ccccc2)c2cc(Cl)ccc2N(CC(=O)NS(=O)(=O)c2ccccc2F)C1=O]

The title compound was prepared according to the procedure of 2-((S)-3-((S)-sec-butyl)-7-chloro-2-oxo-5-phenyl-2,3-dihydro-1H-benzo[e][1,4]diazepin-1-yl)-N-sulfamoylacetamide except 2-fluorobenzenesulfonamide was substituted for sulfamide, and then purified by HPLC and then prep TLC to afford 14.0 mg of the title compound. Calculated for C27H₂₅ClFN₃O₄S, 541.12; observed (M+H)⁺542.8.

### Example 39: Synthesis of 3-((S)-3-((S)-sec-butyl)-7-chloro-2-oxo-5-phenyl-2,3-dihydro-1H-benzo[e][1,4]diazepin-1-yl)-N-sulfamoylpropanamide

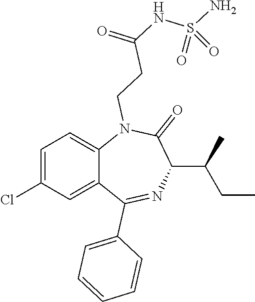 [SMILES: CC[C@H](C)[C@@H]1N=C(c2ccccc2)c2cc(Cl)ccc2N(CCC(=O)NS(N)(=O)=O)C1=O]

3-((S)-3-((S)-sec-butyl)-7-chloro-2-oxo-5-phenyl-2,3-dihydro-1H-benzo[e][1,4]diazepin-1-yl)propanoic acid (20 mg, 0.05 mmol, 1 eq),sulfamide (5 mg, 1.1 eq), 1.5 eq. N-(3-dimethylaminopropyl)-N′-ethylcarbodiimide hydrochloride (14 mg, 0.08 mmol), hydroxybenzotriazole (12 mg, 0.08 eq), and excess N,N-diisopropylethylamine (35 ml, 0.2 mmol) was added to the intermediate in CH₂Cl₂, and stirred for 18 hours. Then, the reaction was purified by HPLC to afford the title compound. ¹H NMR (300 MHz, CDCl₃): δ (ppm) 7.60-7.34 (m, 7H), 7.25-7.22 (m, 1H), 4.50-4.38 (m, 1H), 3.94-3.82 (m, 1H), 3.14-3.07 (m, 1H), 2.68-2.54 (m, 1H), 2.54-2.42 (m, 1H), 2.42-2.30 (m, 1H), 1.98-1.85 (m, 1H), 1.64-1.50 (m, 1H), 1.12-0.86 (m, 6H); Calculated for C₂₂H₂₅ClN₄O₄S, 476.13; observed (M+H)⁺477.7.

### Example 40: Synthesis of 3-((S)-3-((S)-sec-butyl)-7-chloro-2-oxo-5-phenyl-2,3-dihydro-1H-benzo[e][1,4]diazepin-1-yl)-N-(N,N-dimethylsulfamoyl)propanamide

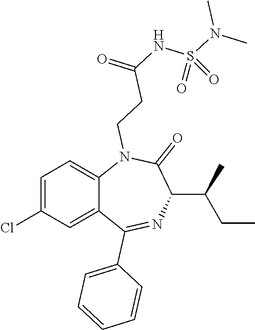 [SMILES: CC[C@H](C)[C@@H]1N=C(c2ccccc2)c2cc(Cl)ccc2N(CCC(=O)NS(=O)(=O)N(C)C)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except N,N-dimethylsulfamide was substituted for sulfamide, and then purified by HPLC to afford the title compound. ¹H NMR (300 MHz, CDCl₃): δ (ppm) 7.56-7.48 (m, 3H), 7.45-7.34 (m, 4H), 7.24-7.22 (m, 1H), 4.40-4.30 (m, 1H), 4.00-3.85 (m, 1H), 3.12-3.05 (m, 1H), 2.75 (s, 6H), 2.55-2.30 (m, 3H), 2.00-1.80 (m, 1H), 1.65-1.50 (m, 1H), 1.10-0.86 (m, 6H); Calculated for C-₂₄H₂₆ClN₄O₄S, 504.16; observed (M+H)⁺505.7.

### Example 41: Synthesis of 3-((S)-3-((S)-sec-butyl)-7-chloro-2-oxo-5-phenyl-2,3-dihydro-1H-benzo|el1,4]diazepin-1-yl)-N-(tert-butylsulfonyl)propanamide

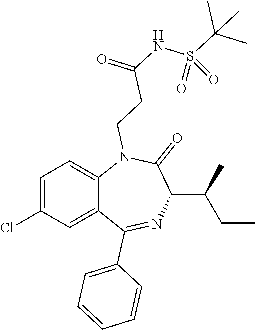 [SMILES: CC[C@H](C)[C@@H]1N=C(c2ccccc2)c2cc(Cl)ccc2N(CCC(=O)NS(=O)(=O)C(C)(C)C)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except tert-butylsulfonamide was substituted for sulfamide, and then purified by HPLC to afford the title compound. ¹H NMR (300 MHz, CDCl₃): δ (ppm) 7.60-7.34 (m, 7H), 7.24-7.18 (m, 1H), 4.40-4.30 (m, 1H), 4.00-3.90 (m, 1H), 3.18-3.08 (m, 1H), 2.60-2.40 (m, 3H), 2.00-1.80 (m, 1H), 1.65-1.50 (m, 1H), 1.30 (s, 9H), 1.12-0.86 (m, 6H); Calculated for C₂₆H₃₂ClN₃O₄S, 517.18; observed (M+H)⁺518.7.

### Example 42: Synthesis of 3-((S)-3-((S)-sec-butyl)-7-chloro-2-oxo-5-phenyl-2,3-dihydro-1H-benzo[e][1,4]diazepin-1-yl)-N-(cyclopropylsulfonyl)propanamide

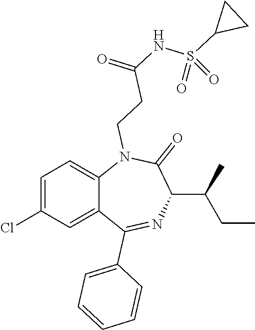 [SMILES: CC[C@H](C)[C@@H]1N=C(c2ccccc2)c2cc(Cl)ccc2N(CCC(=O)NS(=O)(=O)C2CC2)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except cyclopropanesulfonamide was substituted for sulfamide, and then purified by HPLC with a gradient of acetonitrile in water from 10% to 90% to afford the title compound. ¹H NMR (300 MHz, CDCl₃): δ (ppm) 7.62-7.34 (m, 7H), 7.25-7.10 (m, 1H), 4.44-4.30 (m, 1H), 4.00-3.88 (m, 1H), 3.15-3.08 (m, 1H), 2.66-2.40 (m, 4H), 2.00-1.80 (m, 1H), 1.65-1.50 (m, 1H), 1.24-0.74 (m, 10H); Calculated for C₂₅H₂₈ClN₃O₄S, 501.15; observed (M+H)⁺502.7.

### Example 43: Synthesis of 3-((S)-3-((S)-sec-butyl)-7-chloro-2-oxo-5-phenyl-2,3-dihydro-1H-benzo|ele1,4]diazepin-1-yl)-N-(phenylsulfonyl)propanamide

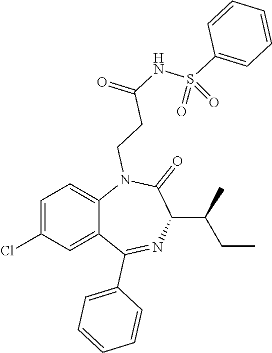 [SMILES: CC[C@H](C)[C@@H]1N=C(c2ccccc2)c2cc(Cl)ccc2N(CCC(=O)NS(=O)(=O)c2ccccc2)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except benzenesulfonamide was substituted for sulfamide, and then purified by HPLC to afford the title compound. ¹H NMR (300 MHz, CDCl₃): δ (ppm) 7.94-7.88 (m, 2H), 7.58-7.28 (m, 10H), 7.21-7.17 (m, 1H), 4.30-4.18 (m, 1H), 3.90-3.76 (m, 1H), 3.10-3.02 (m, 1H), 2.54-2.36 (m, 2H), 2.31-2.18 (m, 1H), 2.00-1.80 (m, 1H), 1.60-1.45 (m, 1H), 1.08-0.86 (m, 6H); Calculated for C-₂₈H₂₈ClN₃O₄S, 537.15; observed (M+H)⁺538.7.

### Example 44: Synthesis of N-(benzylsulfonyl)-3-((S)-3-((S)-sec-butyl)-7-chloro-2-oxo-5-phenyl-2,3-dihydro-1H-benzo[e][1,4]diazepin-1-yl)propanamide

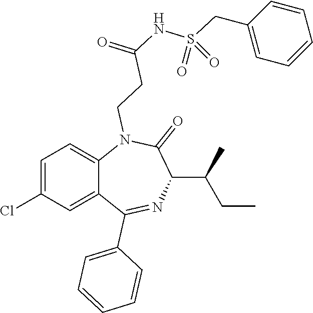 [SMILES: CC[C@H](C)[C@@H]1N=C(c2ccccc2)c2cc(Cl)ccc2N(CCC(=O)NS(=O)(=O)Cc2ccccc2)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except alpha-toluenesulfonamide was substituted for sulfamide, and then purified by HPLC to afford the title compound. ¹H NMR (300 MHz, CDCl₃): δ (ppm) 7.61-7.49 (m, 3H), 7.42-7.30 (m, 5H), 7.30-7.20 (m, 3H), 7.17-7.15 (m, 2H), 4.40-4.18 (m, 3H), 3.96-3.84 (m, 1H), 3.14-3.07 (m, 1H), 2.54-2.36 (m, 3H), 2.00-1.80 (m, 1H), 1.64-1.45 (m, 1H), 1.12-0.88 (m, 6H); Calculated for C₂₉H₃₀ClN₃O₄S, 551.16; observed (M+H)⁺552.8.

### Example 45: Synthesis of 3-((S)-3-((S)-sec-butyl)-7-chloro-2-oxo-5-phenyl-2,3-dihydro-1H-benzole∥1,4]diazepin-1-yl)-N-( thiophen-2-ylsulfonyl)propanamide (62059I)

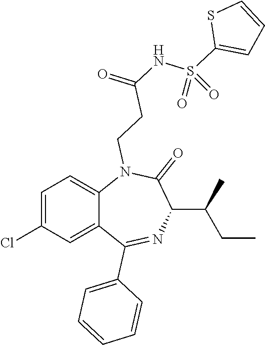 [SMILES: CC[C@H](C)[C@@H]1N=C(c2ccccc2)c2cc(Cl)ccc2N(CCC(=O)NS(=O)(=O)c2cccs2)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except thiophene-2-sulfonamide was substituted for sulfamide, and then purified by HPLC with a gradient of acetonitrile in water from 65% to 75% to afford the title compound. ¹H NMR (300 MHz, CDCl₃): δ (ppm) 7.78-7.73 (m, 1H), 7.62-7.57 (m, 1H), 7.55-7.32 (m, 7H), 7.24-7.20 (m, 1H), 7.05-7.00 (m, 1H), 4.36-4.20 (m, 1H), 3.95-3.82 (m, 1H), 3.12-3.04 (m, 1H), 2.60-2.00 (m, 3H), 2.00-1.80 (m, 1H), 1.64-1.45 (m, 1H), 1.12-0.87 (m, 6H); Calculated for C₂₆H₂₆ClN₃O₄S₂, 543.11; observed (M+H)⁺544.7.

### Example 46: Synthesis of 3-((S)-3-((S)-sec-butyl)-7-chloro-2-oxo-5-phenyl-2,3-dihydro-1H-benzo[e][1,4]diazepin-1-yl)-N-((5-chlorothiophen-2-yl)sulfonyl)propanamide

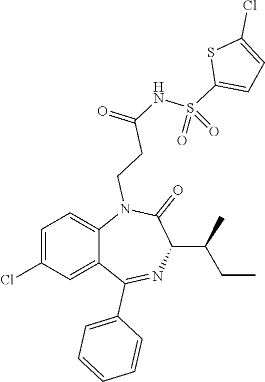 [SMILES: CC[C@H](C)[C@@H]1N=C(c2ccccc2)c2cc(Cl)ccc2N(CCC(=O)NS(=O)(=O)c2ccc(Cl)s2)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropanamide except 5-chlorothiophene-2-sulfonamide was substituted for sulfamide, and then purified by HPLC with a gradient from 70% to 82% in 11 minutes to afford the title compound. ¹H NMR (300 MHz, CDCl₃): δ (ppm) 7.56-7.34 (m, 8H), 7.24-7.21 (m, 1H), 6.86-6.83 (m, 1H), 4.40-4.26 (m, 1H), 3.95-3.83 (m, 1H), 3.13-3.06 (m, 1H), 2.60-2.20 (m, 3H), 2.00-1.80 (m, 1H), 1.64-1.45 (m, 1H), 1.12-0.88 (m, 6H); Calculated for C₂₆H₂₅Cl₂N₃O₄S₂, 577.07; observed (M+H)⁺578.7.

### Example 47: Synthesis of 3-((S)-3-((S)-sec-butyl)-7-chloro-2-oxo-5-phenyl-2,3-dihydro-1H-benzo[e][1,4]diazepin-1-yl)-N-((1-methyl-1H-imidazol-4-yl)sulfonyl)propanamide

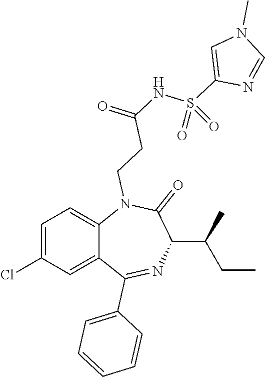 [SMILES: CC[C@H](C)[C@@H]1N=C(c2ccccc2)c2cc(Cl)ccc2N(CCC(=O)NS(=O)(=O)c2cn(C)cn2)C1=O]

The title compound was prepared according to the procedure for preparation 3-((S)-3-((S)-sec-butyl)-7-chloro-2-oxo-5-phenyl-2,3-dihydro-1H-benzo[e][1,4]diazepin-1-yl)-N-sulfamoylpropananide except 1-methylimnidazole-2-sulfonanide was substituted for sulfanide, and then purified by HPLC with a gradient from 60% to 67% in 8 minutes to afford the title compound. ¹H NMR (300 MHz, CDCl₃): δ (ppm) 7.54-7.29 (m, 9H), 7.21-7.18 (m, 1H), 4.40-4.26 (m, 1H). 3.90-3.79 (m, 1H), 3.66 (s, 3H), 3.10-3.03 (m, 1H), 2.60-2.30 (m, 3H), 2.00-1.80 (m, 1H), 1.60-1.50 (m, 1H), 1.10-0.86 (m, 6H); Calculated for C₂₆H₂₈ClN₅O₄S, 541.16; observed (M+H)⁺542.8.

### Example 48: Synthesis of 2-((3S)-3-((S)-sec-butyl)-7-chloro-2-oxo-5-phenyl-2,3,4,5-tetrahydro-1H-benzo[e][1,4]diazepin-1-yl)-N-(phenylsulfonyl)acetamide 0

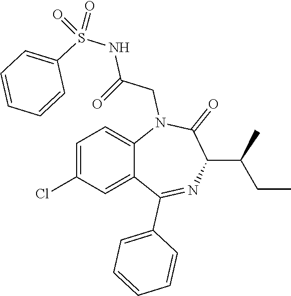 [SMILES: CC[C@H](C)[C@@H]1N=C(c2ccccc2)c2cc(Cl)ccc2N(CC(=O)NS(=O)(=O)c2ccccc2)C1=O]

3 eq. sodium triacetoxyborohydride (24 mg, 0.12 mmol) was added to 2-((S)-3-((S)-sec-butyl)-7-chloro-2-oxo-5-phenyl-2,3-dihydro-1H-benzo[e][1,4]diazepin-1-yl)-N-(phenylsulfonyl)acetamide (20 mg, 0.04 mmol) in 1,2-dichloroethane at 0° C. The reaction was allowed to go to 23° C. and was stirred for 4 days. Sodium cyanoborohydride (2 eq) was added to further the slow reaction. Upon completion, the reaction was quenched with saturated NaHCO₃, and then purified by HPLC with a gradient from 55% to 61% in 6 minutes to afford the title compound. ¹H NMR (300 MHz, CDCl₃): δ (ppm) 8.03-7.97 (m, 2H), 7.61-7.53 (m, 1H), 7.51-7.31 (m, 7H), 7.24-7.18 (m, 1H), 7.11-7.06 (m, 1H), 6.54 (d, J=2.4 Hz, 1H), 5.47 (s, 1H), 4.45 (s, 2H), 3.12 (d, J=9.3 Hz, 1H), 1.90-1.75 (m, 1H), 1.72-1.58 (m, 1H), 1.24-1.06 (m, 1H), 0.86-0.72 (m, 6H); Calculated for C₂₇H₂₈ClN₃O₄S, 525.15; observed (M+H)⁺526.8.

### Example 49: Synthesis of 2-((3S)-3-((S)-sec-butyl)-7-chloro-2-oxo-5-phenyl-2,3,4,5-tetrahydro-1H-benzo[e][1,4]diazepin-1-yl)-N-(thiophen-2-ylsulfonyl)acetamide Q

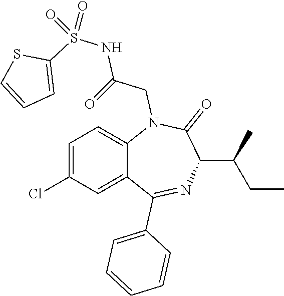 [SMILES: CC[C@H](C)[C@@H]1N=C(c2ccccc2)c2cc(Cl)ccc2N(CC(=O)NS(=O)(=O)c2cccs2)C1=O]

The title compound was prepared according to the procedure for 2-((3S)-3-((S)-sec-butyl)-7-chloro-2-oxo-5-phenyl-2,3,4,5-tetrahydro-1 H-benzo[e][1,4]diazepin-1-yl)-N-(phenyl sulfonyl)acetamide using 2-((S)-3-((S)-sec-butyl)-7-chloro-2-oxo-5-phenyl-2,3-dihydro-1H-benzo[e][1,4]diazepin-1-yl)-N-(thiophen-2-ylsulfonyl)acetamide to provide the title compound. 1H NMR (300 MHz, CDCl₃): δ (ppm) 7.82-7.79 (m, 1H), 7.62-7.58 (m, 1H), 7.50-7.44 (m, 2H), 7.42-7.32 (m, 3H), 7.25-7.18 (m, 1H), 7.12-7.06 (m, 1H), 7.05-7.00 (m, 1H), 6.57-6.3 (m, 1H), 5.55 (s, 1H), 4.56-4.40 (m, 2H), 3.17-3.10 (m, 1H), 1.92-1.78 (m, 1H), 1.74-1.58 (m, 1H), 1.22-1.06 (m, 1H), 0.86-0.74 (m, 6H); Calculated for C₂₅H₂₆ClN₃O₄S₂, 531.11; observed (M+H)⁺532.7.

### Example 50: Synthesis of 3-((3S)-3-((S)-sec-butyl)-7-chloro-2-oxo-5-phenyl-2,3,4,5-tetrahydro-1H-benzo[c][1,4]diazepin-1-yl)-N-(phenylsulfonyl)propanamide (isomer 1) and 3-((3R)-3 -((S)-scc-butyl)-7-chloro-2-oxo-5-phenyl-2,3,4,5-tetrahydro-1H-benzo[c][1,4]diazepin-1-yl)-N-(phenylsulfonyl)propanamide (isomer 2)

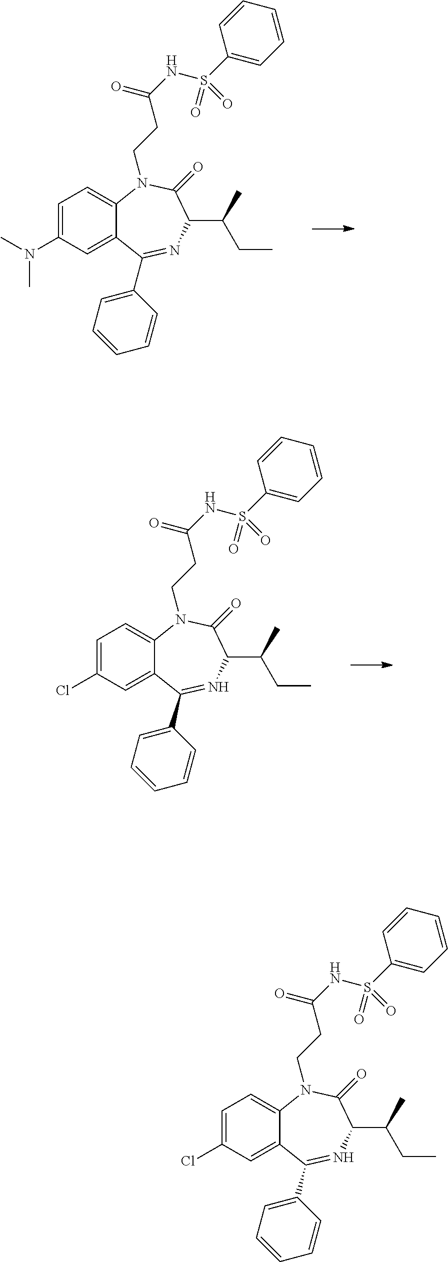

3-((3S)-3-((S)-sec-butyl)-7-chloro-2-oxo-5-phenyl-2,3,4,5-tetrahydro-1H-benzo[e][1,4]diazepin-1-yl)-N-(phenylsulfonyl)propanamide (isomer 1) and 3-((3R)-3-((S)-sec-butyl)-7-chloro-2-oxo-5-phenyl-2,3,4,5-tetrahydro-1H-benzo[e][1,4]diazepin-1-yl)-N-(phenylsulfonyl)propanamide (isomer 2): The title compounds were prepared according to the procedure of 2-((3S)-3-((S)-sec-butyl)-7-chloro-2-oxo-5-phenyl-2,3,4,5-tetrahydro-1H-benzo[e][1,4]diazepin-1-yl)-N-(phenylsulfonyl)acetamide using 3-((S)-3-((S)-sec-butyl)-7-chloro-2-oxo-5-phenyl-2,3-dihydro-1H-benzo[e][1,4]diazepin-1-yl)-N-(phenylsulfonyl)propanamide to provide the two isomers. Isomers one: ¹H NMR (300 MHz, CDCl₃): δ (ppm) 8.00-7.94 (m, 2H), 7.56-7.49 (m, 1H), 7.46-7.34 (m, 7H), 7.25-7.18 (m, 1H), 7.16-7.10 (m, 1H), 6.54-6.50 (m, 1H), 5.10 (s, 1H), 4.36-4.24 (m, 1H), 3.90-3.76 (m, 1H), 2.98-2.93 (m, 1H), 2.80-2.40 (m, 3H)1.98-1.84 (m, 1H), 1.40-1.30 (m, 1H), 1.00-0.94 (m, 3H), 0.87-0.76 (m, 3H); Calculated for C₂₈H₃₀ClN₃O₄S, 539.16; observed (M+H)⁺540.8. Isomers-2. 1H NMR (300 MHz, CDCl₃): δ (ppm) 8.00-7.95 (m, 2H), 7.56-7.49 (m, 1H), 7.45-7.32 (m, 7H), 7.25-7.18 (m, 1H), 7.16-7.10 (m, 1H), 6.52 (d, J=2.1 Hz, 1H), 5.10 (s, 1H), 4.36-4.24 (m, 1H), 3.88-3.76 (m, 1H), 2.96 (d, J=9.3 Hz, 1H), 2.80-2.76 (m, 1H), 2.74-2.40 (m, 1H), 1.96-1.84 (m, 1H), 1.74-1.60 (m, 1H), 1.20-1.06 (m, 1H), 0.88-0.80 (m, 6H); Calculated for C₂₈H₃₀ClN₃O₄S, 539.16; observed (M+H)⁺540.8.

### Example 51: Synthesis of 3-((3S)-3-((S)-sec-butyl)-7-chloro-2-oxo-5-phenyl-2,3,4,5-tetrahydro-1H-benzo[e][1,4]diazepin-1-yl)-N-(thiophen-2-ylsulfonyl)propanamide (isomer 1) and 3-((3R)-3-((S)-sec-butyl)-7-chloro-2-oxo-5-phenyl-2,3,4,5-tetrahydro-1H-benzo[e][1,4]diazepin-1-yl)-N-(thiophen-2-ylsulfonyl)propanamide (isomer 2)

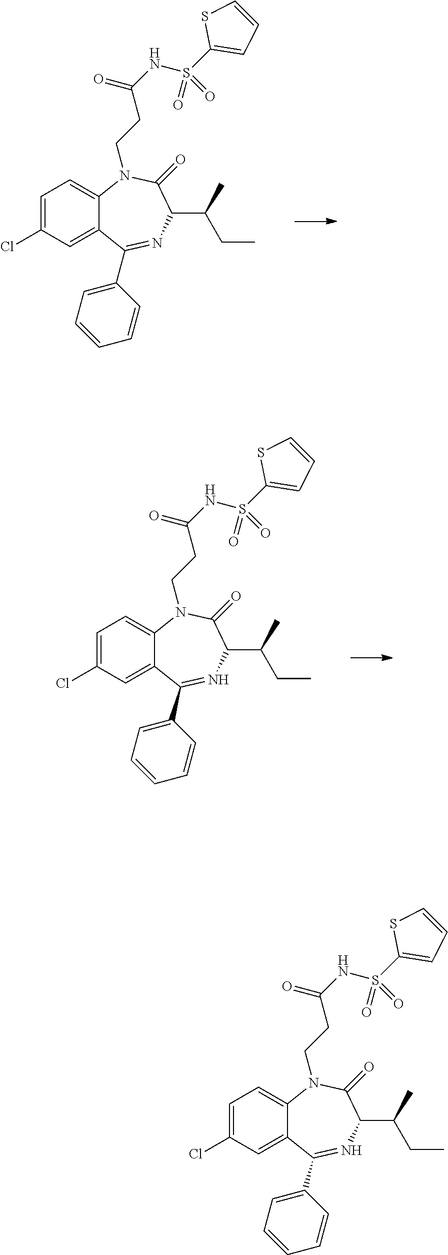

The title compounds were prepared according to the procedure of 2-((3S)-3-((S)-sec-butyl)-7-chloro-2-oxo-5-phenyl-2,3,4,5-tetrahydro-1H-benzo[e][1,4]diazepin-1-yl)-N-(phenylsulfonyl)acetaride using 3-((S)-3-((S)-sec-butyl)-7-chloro-2-oxo-5-phenyl-2,3-dihydro-1H-benzo[e][1,4]diazepin-1-yl)-N-(thiophen-2-ylsulfonyl)propanamide to provide the two isomers. 3-((3S)-3-((S)-sec-butyl)-7-chloro-2-oxo-5-phenyl-2,3,4,5-tetrahydro-1H-benzo[e][1,4]diazepin-1-yl)-N-(thiophen-2-ylsulfonyl)propanamide (isomer 1) ¹H NMR (300 MHz, CDCl₃): δ (ppm) 7.80-7.76 (m, 1H), 7.58-7.54 (m, 1H), 7.44-7.30 (m, 6H), 7.21-7.13 (m, 1H), 7.01-6.96 (m, 1H), 6.54-6.50 (m, 1H), 5.04 (s, 1H), 4.37-4.24 (m, 1H), 3.94-3.80 (m, 1H), 2.95 (d, J=9.3 Hz, 1H), 2.86-2.72 (m, 1H), 2.70-2.40 (m, 1H), 2.00-1.82 (m, 2H), 1.42-1.28 (m, 1H), 0.96 (d, J=6.3 Hz, 3H), 0.80 (t, J=7.2 Hz, 3H); Calculated for C₂₆H₂₈ClN₃O₄S₂, 545.12; observed (M+H)⁺546.8. 3-((3R)-3-((S)-sec-butyl)-7-chloro-2-oxo-5-phenyl-2,3,4,5-tetrahydro-1H-benzo[e]I[1,4]diazepin-1-yl)-N-(thiophen-2-ylsulfonyl)propanamide (isomer 2): ¹H NMR (300 MHz, CDCl₃): δ (ppm) 7.82-7.76 (m, 1H), 7.59-7.54 (m, 1H), 7.44-7.28 (m, 6H), 7.22-7.15 (m, 1H), 7.03-6.96 (m, 1H), 6.58-6.52 (m, 1H), 5.09 (s, 1H), 4.40-4.26 (m, 1H), 3.94-3.82 (m, 1H), 3.00 (d, J=9.3 Hz, 1H), 2.84-2.72 (m, 1H), 2.70-2.40 (m, 1H), 1.98-1.85 (m, 1H), 1.76-1.60 (m, 1H), 1.22-1.06 (m, 1H), 0.90-0.80 (m, 6H); Calculated for C-₂₆H₂₈ClN₃O₄S₂, 545.12; observed (M+H)⁺546.8.

### Example 52: Synthesis of 3-(7-chloro-2-oxo-5-phenyl-3-(tetrahydro-2H-pyran-4-yl)-2,3-dihydro-1H-benzo[e][1,4]diazepin-1-yl)propanoic acid

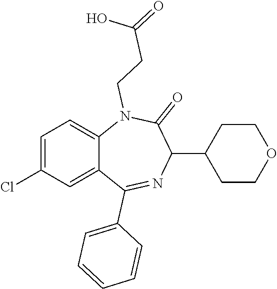 [SMILES: O=C(O)CCN1C(=O)C(C2CCOCC2)N=C(c2ccccc2)c2cc(Cl)ccc21]

1 eq 2-((tert-butoxycarbonyl)amino)-2-(tetrahydro-2H-pyran-4-yl)acctic acid (112 mg, 0.43 mmol, 1 eq),and 1.2 eq N,N′-dicyclohexylcarbodiimide (107 mg) was added to (2-amino-5-chlorophenyl)(phenyl)methanone (100 mg, 1.43 mmol) in toluene. Upon completion, the reaction was filtered and the filtrated was redissolved in formic acid to remove the Boc group. The formic acid was evaporated off and the reaction was redissolved in methanol and stirred for 18 hours. Upon completion, the cyclization reaction was purified by HPLC to afford the intermediate, 7-chloro-5-phenyl-3-(tetrahydro-2H-pyran-4-yl)-11H-benzo[c][1,4]diazepin-2(3H)-one (42.5 mg) as a yellow sticky solid. To the intermediate (20 mg, 0.06 mmol) in acetonitrile, 2 eq potassium carbonate (16 mg), 1.6 eq ethyl 3-bromopropionate (10 ml), and 0.5 eq benzyltributylammonium bromide (9 mg) was added. The reaction was heated to 90° C. for 3 days. Upon completion, it was purified by Combi-Flash (30% ethyl acetate in hexanes) to afford ethyl 3-(7-chloro-2-oxo-5-phenyl-3-(tetrahydro-2H-pyran-4-yl)-2,3-dihydro-1H-benzo[e][1,4]diazepin-1-yl)propanoate (16.1 mg) as a clear oil. This intermediate was treated with 3 eq 1M LiOH in tetrahydrofuran and stirred for 18 hours. The reaction was purified by HPLC with a gradient from 50% to 54% in 10 minutes to afford 3-(7-chloro-2-oxo-5-phenyl-3-(tetrahydro-2H-pyran-4-yl)-2,3-dihydro-1H-benzo[e][1,4]diazepin-1-yl)propanoic acid. ¹H NMR (300 MHz, CDCl₃): δ (ppm) 7.68-7.63 (m, 1H), 7.63-7.56 (m, 3H), 7.50-7.42 (m, 1H), 7.32 (d, J=2.4 Hz, 1H), 4.80-4.68 (m, 1H), 4.13-4.05 (m, 1H), 4.03-3.85 (m, 2H), 3.67-3.54 (m, 2H), 2.29 (d, J=9.9 Hz, 1H), 2.94-2.78 (m, 1H), 2.72-2.58 (m, 1H), 2.50-2.38 (m, 1H), 2.27-2.17 (m, 1H), 2.02-1.94 (m, 1H), 1.42-1.10 (m, 2H); Calculated for C23H₂₃ClN₂O₄, 426.13; observed (M+H)⁺427.6.

### Example 53: Synthesis of 2-(7-chloro-2-oxo-5-phenyl-3-(tetrahydro-2H-pyran-4-yl)-2,3-dihydro-1H-benzo[e][1,4]diazepin-1-yl)acetic acid

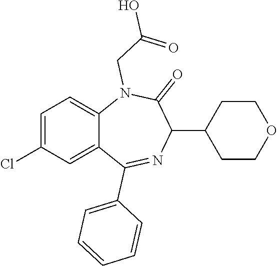 [SMILES: O=C(O)CN1C(=O)C(C2CCOCC2)N=C(c2ccccc2)c2cc(Cl)ccc21]

According to the procedure for preparation of compound 3-(7-chloro-2-oxo-5-phenyl-3-(tetrahydro-2H-pyran-4-yl)-2,3-dihydro-1H-benzo[e][1,4]diazepin-1-yl)propanoic acid, the intermediate 7-chloro-5-phenyl-3-(tetrahydro-2H-pyran-4-yl)-1H-benzo[e][1,4]diazepin-2(3H)-one (16 mg, 0.046 mmol) was treated with 2 eq potassium carbonate (31 mg, 0.24 mmol), 1.6 eq methyl 2-bromoacetate (6 μl, 0.068 mmol), and 0.5 eq benzyltributylammonium bromide (8 mg, 0.023 mmol) in acetonitrile at 23° C. After Combi-Flash purification with a gradient of 30% ethyl acetate in hexanes, the resulting intermediate methyl 2-(7-chloro-2-oxo-5-phenyl-3-(tetrahydro-2H-pyran-4-yl)-2,3-dihydro-1H-benzo[c][1,4]diazepin-1-yl)acetate was dissolved in tetrahydrofuran and treated with 3 eq 1M LiOH. Upon completion, the reaction was purified by HPLC to afford 2-(7-chloro-2-oxo-5-phenyl-3-(tetrahydro-2H-pyran-4-yl)-2,3-dihydro-1H-benzo[e][1,4]diazepin-1-yl)acetic acid. ¹H NMR (300 MHz, CDCl₃): δ (ppm) 7.58-7.53 (m, 2H), 7.53-7.34 (m, 5H), 7.30-7.25 (m, 1H), 4.52-4.47 (m, 2H), 4.08-3.99 (m, 1H), 3.97-3.88 (m, 1H), 3.62-3.48 (m, 2H), 3.27 (d, J=9.6 Hz, 1H), 2.30-2.40 (m, 1H), 1.99-1.72 (m, 2H), 1.36-1.14 (m, 2H); Calculated for C22H₂₁ClN₂O₄, 412.12; observed (M+H)⁺413.6.

### Example 54: Synthesis of (S)-3-(7-chloro-2-oxo-3-(pentan-3-yl)-5-phenyl-2,3-dihydro-1H-benzo[e][1,4]diazepin-1-yl)-N-(methylsulfonyl)propanamide

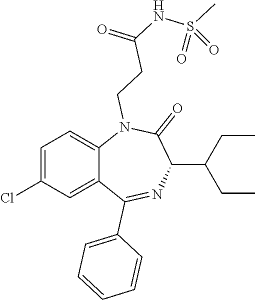 [SMILES: CCC(CC)[C@@H]1N=C(c2ccccc2)c2cc(Cl)ccc2N(CCC(=O)NS(C)(=O)=O)C1=O]

1.1 eq. methanesulfonamide (3 mg), 1.1 eq. N-(3-dimethylaminopropyl)-N′-ethylcarbodiimide hydrochloride (5 mg), 1.1 eq. hydroxybenzotriazole (4 mg), and excess N,N-diisopropylethylamine (2 eq) was added to (S)-3-(7-chloro-2-oxo-3-(pentan-3-yl)-5-phenyl-2,3-dihydro-1H-benzo[e][1,4]diazepin-1-yl)propanoic acid in CH₂C2. Upon completion, the reaction was purified by HPLC with a gradient from 70% to 80% in 10 minutes to afford the product (5.4 mg). ¹H NMR (300 MHz, CDCl₃): δ (ppm) 7.60-7.50 (m, 3H), 7.50-7.35 (m, 4H), 7.25-7.22 (m, 1H), 4.42-4.30 (m, 1H), 3.98-3.86 (m, 1H), 3.25 (d, J=9.9 Hz, 1H), 2.52-2.36 (m, 3H), 1.84-1.70 (m, 1H), 1.66-1.52 (m, 1H), 1.52-1.37 (m, 1H), 1.34-1.16 (m, 1H), 0.88-0.74 (m, 6H); Calculated for C-₂₄H₂₈ClN₃O₄S, 489.15; observed (M+H)⁺490.8.

### Example 55: Synthesis of (S)-N-(benzylsulfonyl)-3-(7-chloro-2-oxo-3-(pentan-3-yl)-5-phenyl-2,3-dihydro-1H-benzo[e][1,4]diazepin-1-yl)propanamide

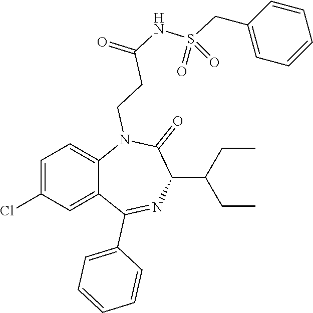 [SMILES: CCC(CC)[C@@H]1N=C(c2ccccc2)c2cc(Cl)ccc2N(CCC(=O)NS(=O)(=O)Cc2ccccc2)C1=O]

According to the procedure for preparation of compound (S)-3-(7-chloro-2-oxo-3-(pentan-3-yl)-5-phenyl-2,3-dihydro-1H-benzo[e][1,4]diazepin-1-yl)-N-(methylsulfonyl)propanamide, the intermediate (S)-3-(7-chloro-2-oxo-3-(pentan-3-yl)-5-phenyl-2,3-dihydro-1H-benzo[e][1,4]diazepin-1-yl)propanoic acid was treated with 1.1 eq. alpha-toluenesulfonamide. 1.1 eq. N-(3-dimethylaminopropyl)-N′-ethylcarbodiimide hydrochloride, and 1.1 eq. hydroxybenzotriazole, and then purified by HPLC to afford (S)-N-(benzylsulfonyl)-3-(7-chloro-2-oxo-3-(pentan-3-yl)-5-phenyl-2,3-dihydro-1H-benzo[c][1,4]diazepin-1-yl)propanamide. ¹H NMR (300 MHz, CDCl₃): δ (ppm) 7.60-7.50 (m, 3H), 7.45-7.32 (m, 4H), 7.32-7.23 (m, 4H), 7.20-7.20 (m, 1H), 4.36-4.16 (m, 3H), 3.97-3.82 (m, 1H), 3.26 (d, J=9.9 Hz, 1H), 2.50-2.30 (m, 3H), 1.85-1.70 (m, 1H), 1.67-1.53 (m, 1H), 1.53-1.36 (m, 1H), 1.36-1.16 (m, 1H), 0.90-0.72 (m, 6H); Calculated for C₃₀H₃₂ClN₃O₄S, 565.18; observed (M+H)⁺566.9.

### Example 56: Synthesis of (S)-3-(7-chloro-2-oxo-3-(pentan-3-yl)-5-phenyl-2,3-dihydro-1H-benzo[e][1,4]diazepin-1-yl)-N-sulfamoylpropanamide

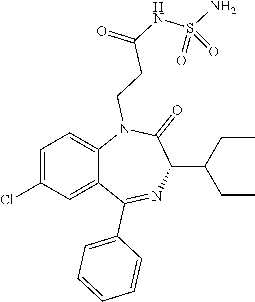 [SMILES: CCC(CC)[C@@H]1N=C(c2ccccc2)c2cc(Cl)ccc2N(CCC(=O)NS(N)(=O)=O)C1=O]

According to the procedure for preparation of compound (S)-3-(7-chloro-2-oxo-3-(pentan-3-yl)-5-phenyl-2,3-dihydro-1H-benzole1|1,4]diazepin-1-yl)-N-(methylsulfonyl)propanamide, the intermediate (S)-3-(7-chloro-2-oxo-3-(pentan-3-yl)-5-phenyl-2,3-dihydro-1H-benzo[e][1,4]diazepin-1-yl)propanoic acid was treated with 1.1 eq. sulfamide, 1.1 eq. N-(3-dimethylaminopropyl)-N′-ethylcarbodiimide hydrochloride, and 1.1 eq. hydroxybenzotriazole, and then purified by HPLC to afford (S)-3-(7-chloro-2-oxo-3-(pentan-3-yl)-5-phenyl-2,3-dihydro-1H-benzo[e][1,4]diazepin-1-yl)-N-sulfamoylpropanamide. ¹H NMR (300 MHz, CDCl₃): δ (ppm) 7.62-7.50 (m, 3H), 7.50-7.35 (m, 4H), 7.25-7.20 (m, 1H), 4.50-4.34 (m, 1H), 3.95-3.80 (m, 1H), 3.25 (d, J=9.9 Hz, 1H), 2.68-2.54 (m, 1H), 2.48-2.30 (m, 211), 1.84-1.69 (m, 1H), 1.65-1.51 (m, 1H), 1.51-1.37 (m, 1H), 1.30-1.16 (m, 1H), 0.90-0.72 (m, 6H); Calculated for C₂₃H₂₇ClN₄O₄S, 490.14; observed (M+H)⁺491.8.

### Example 57: Synthesis of (S)-2-(7-chloro-2-oxo-3-(pentan-3-yl)-5-phenyl-2,3-dihydro-1H-benzo[e][1,4]diazepin-1-yl)-N-(methylsulfonyl)acetamide

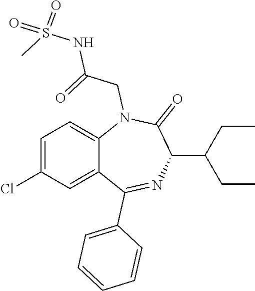 [SMILES: CCC(CC)[C@@H]1N=C(c2ccccc2)c2cc(Cl)ccc2N(CC(=O)NS(C)(=O)=O)C1=O]

1.1 eq. methanesulfonamide, 1.1 eq. N-(3-dimethylaminopropyl)-N′-ethylcarbodiimide hydrochloride, 1.1 eq. hydroxybenzotriazole, and excess N,N-diisopropylethylamine was added to (S)-2-(7-chloro-2-oxo-3-(pentan-3-yl)-5-phenyl-2,3-dihydro-1H-benzo[e][1,4]diazepin-1-yl)acetic acid in CH₂Cl₂. Upon completion, the reaction was purified by HPLC to afford (S)-2-(7-chloro-2-oxo-3-(pentan-3-yl)-5-phenyl-2,3-dihydro-1H-benzo[e][1,4]diazepin-1-yl)-N-(methylsulfonyl)acetamide. ¹H NMR (300 MHz, CDCl₃): δ (ppm) 7.62-7.55 (m, 2H), 7.55-7.30 (m, 6H), 4.56-4.46 (m, 1H), 4.32-4.24 (m, 1H), 3.43-3.37 (m, 1H), 3.24-3.20 (m, 3H), 2.50-2.36 (m, 1H), 1.90-1.70 (m, 1H), 1.70-1.52 (m, 1H), 1.52-1.37 (m, 1H), 1.37-1.20 (m, 1H), 0.89-0.78 (m, 6H); Calculated for C₂₃H₂₆ClN₃O₄S, 475.13; observed (M+H)⁺ 476.7.

### Example 58 Synthesis of (S)-2-(7-chloro-2-oxo-3-(pentan-3-yl)-5-phenyl-2,3-dihydro-1H-benzo[e][1,4]diazepin-1-yl)-N-(phenylsulfonyl)acetamide

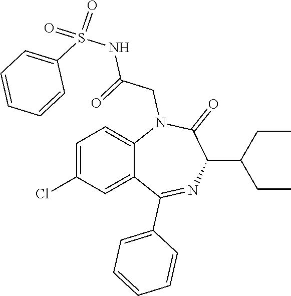 [SMILES: CCC(CC)[C@@H]1N=C(c2ccccc2)c2cc(Cl)ccc2N(CC(=O)NS(=O)(=O)c2ccccc2)C1=O]

According to the procedure for preparation of compound (S)-2-(7-chloro-2-oxo-3-(pentan-3-yl)-5-phenyl-2,3-dihydro-1H-benzo[e][1,4]diazepin-1-yl)-N-(methylsulfonyl)acetamide, the intermediate (S)-3-(7-chloro-2-oxo-3-(pentan-3-yl)-5-phenyl-2,3-dihydro-1 H-benzo[e][1,4]diazepin-1-yl)propanoic acid was treated with 1.1 eq. benzenesulfonamide, 1.1 eq. N-(3-dimethylaminopropyl)-N′-ethylcarbodiimide hydrochloride, and 1.1 eq. hydroxybenzotriazole, and then purified by HPLC to afford (S)-2-(7-chloro-2-oxo-3-(pentan-3-yl)-5-phenyl-2,3-dihydro-1H-benzo[e][1,4]diazepin-1-yl)-N-(phenylsulfonyl)acetamide. ¹H NMR (300 MHz, CDCl₃): δ (ppm) 8.01-7.95 (m, 2H), 7.60-7.29 (m, 1OH), 7.22-7.19 (m, 1H), 4.52-4.44 (m, 1H), 4.24-4.12 (m, 1H), 3.25 (d, J=10.5 Hz, 1H), 2.38-2.25 (m, 1H), 1.80-1.66 (m, 1H), 1.63-1.46 (m, 1H), 1.30-1.06 (m, 2H), 0.82-0.66 (m, 6H); Calculated for C₂₅H₂₈ClN₃O₄S, 537.15; observed (M+H)⁺538.8.

### Example 59: Synthesis of 2-((S)-3-((R)-1-(tert-butoxy)ethyl)-7-chloro-2-oxo-5-phenyl-2,3-dihydro-1Hbenzo[e][1,4]diazepin-1-yl)acetic acid

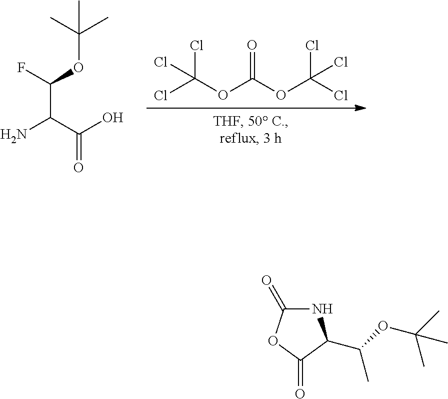

Step-1: (S)-4-((R)-1-(tert-butoxy)ethyl) oxazolidine-2,5-dione: A round bottom flask was equipped with magnetic stir bar, condenser, and N₂ inlet, was added (2S, 3R)-2-amino-3-(tert-butoxy) butanoic acid (265 mg, 1.515 mmol) in dry tetrahydrofuran (3 mL) and heated at 50° C. The solution of Triphosgene (150 mg, 0.505 mmol) in dry tetrahydrofuran (5 mL) was added into the reaction by dropping funnel and the reaction mixture was heated at reflux for 3 hours. Reaction mixture was cooled down to 23° C., evaporated (at bath temperature is off) to dryness to obtain (S)-4-((R)-1-(tert-butoxy)ethyl) oxazolidine-2,5-dione (2) (304 mg, 100% yield) as a white solid and was directly taken for next step without further purification.

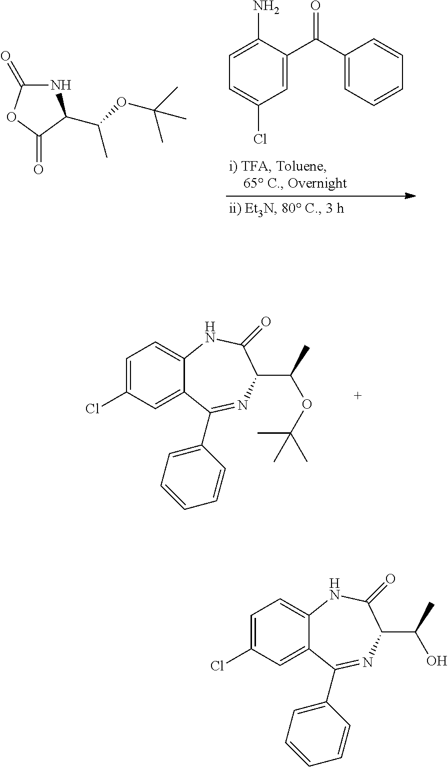 [SMILES: C[C@@H](OC(C)(C)C)[C@@H]1NC(=O)OC1=O ; Nc1ccc(Cl)cc1C(=O)c1ccccc1 ; C[C@@H](OC(C)(C)C)[C@@H]1N=C(c2ccccc2)c2cc(Cl)ccc2NC1=O ; C[C@@H](O)[C@@H]1N=C(c2ccccc2)c2cc(Cl)ccc2NC1=O]

Step-2: (S)-3-((R)-1-(tert-butoxy)ethyl)-7-chloro-5-phenyl-1H-benzo[e][1,4]diazepin-2(3H)-one and (S)-7-chloro-3-((R)-1-hydroxyethyl)-5-phenyl-1H-benzo[e][1,4]diazepin-2(3H)-one: A round bottom flask was equipped with magnetic stir bar, condenser, and N₂ inlet, was added (S)-4-((R)-1-(tert-butoxy)ethyl) oxazolidine-2,5-dione (304 mg, 1.5123 mmol) and 2-amino-5-chlorobenzophenone (292 mg, 1.2603 mmol) in dry toluene (8 mL). Trifluoroacetic acid (TFA) (0.194 mL, 2.5206 mmol) was added to the reaction mixture and stirred at 65° C. for 18 hours. Then triethylamine (Et₃N) (0.35 mL, 2.5206 mmol) was added to the reaction mixture and stirred at 80° C. for 3 hours. The reaction mixture was cooled down to 23° C., evaporated, quenched with 1N HCl and extracted with ethyl acetate. Organic layer was washed with sat. NaHCO₃ solution and brine solution, dried over anhy.Na₂SO₄ and concentrated. Crude was purified by Combi-flash (0-30% ethyl acetate in dichloromethane) and the (S)-3-((R)-1-(tert-butoxy)ethyl)-7-chloro-5-phenyl-1H-benzo[e][1,4]diazepin-2(3H)-one (4, 66-O₄-OtBu, 35 mg, 7.5% yield) was obtained as off-white solid; Calculated for C₂₁H₂₃ClN₂O₂, 370.14; observed (M+H)⁺371.6 and (S)-7-chloro-3-((R)-1 -hydroxyethyl)-5-phenyl-1H-benzo[c][1,4]diazepin-2(3H)-one (5, 66-O₄-OH, 60 mg, 15.2% yield) was obtained as colorless liquid; Calculated for C₁₇H₁₅ClN₂O₂, 314.08; observed (M+H)⁺315.5

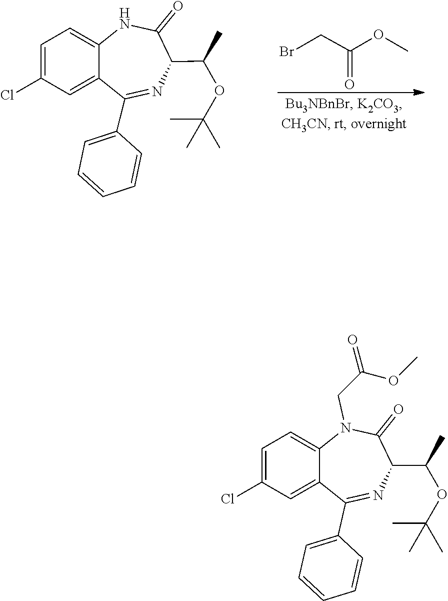

Step 3: A round bottom flask was equipped with magnetic stir bar, and N₂ inlet, was added (S)-3-((R)-1-(tert-butoxy)ethyl)-7-chloro-5-phenyl-1H-benzo[e][1,4]diazepin-2(3H)-one (4, 35 mg, 0.0943 mmol) in dry acetonitrile (2 mL). Benzyltributylammonium bromide (16.8 mg, 0.0471 mmol), potassium carbonate (65.1 mg, 0.4715 mmol) and methyl 2-bromoacetate (0.014 mL, 0.1414 mmol) was added to the reaction mixture and stirred at 23° C. for 18 hours. The reaction mixture was filtered and evaporated on rotavapor. The resulting material was purified by Combi-flash (0-30% ethyl acetate in Hexanes) and the methyl 2-((S)-3-((R)-1-(tert-butoxy)ethyl)-7-chloro-2-oxo-5-phenyl-2,3-dihydro-1H-benzo[e][1,4]diazepin-1-yl)acetate (30 mg) was obtained as colorless liquid. Calculated for C₂₄H₂₇ClN₂O₄, 442.17; observed (M+H)⁺443.7

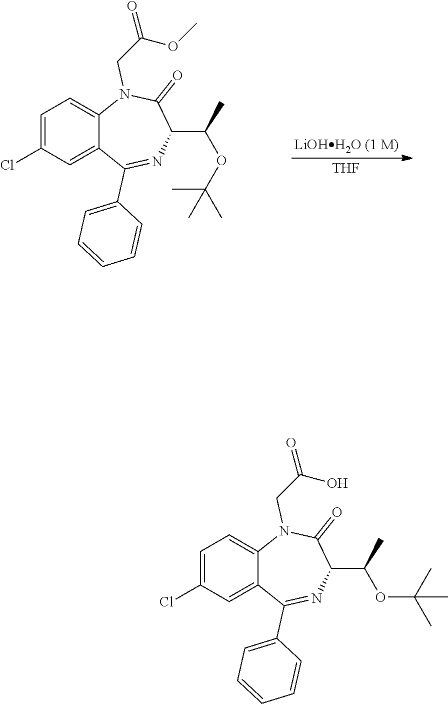 [SMILES: COC(=O)CN1C(=O)[C@H]([C@@H](C)OC(C)(C)C)N=C(c2ccccc2)c2cc(Cl)ccc21 ; C[C@@H](OC(C)(C)C)[C@@H]1N=C(c2ccccc2)c2cc(Cl)ccc2N(CC(=O)O)C1=O ; "THF"]

Step 4: A round bottom flask was equipped with magnetic stir bar, was added methyl 2-((S)-3-((R)-1-(tert-butoxy)ethyl)-7-chloro-2-oxo-5-phenyl-2,3-dihydro-1H-benzo[e][1,4]diazepin-1-yl)acetate (6, 30 mg 0.0677 mmol) in tetrahydrofuran (2 mL). LiOH·H₂O (1 M) aqueous solution (0.20 mL, 0.203 mmol) was added to the reaction mixture and stirred at 23° C. for 18 hours. LC-MS showed desired product. Reaction mixture was concentrated on rotavapor. Crude was purified by preparative HPLC and desired product 2-((S)-3-((R)-1-(tert-butoxy)ethyl)-7-chloro-2-oxo-5-phenyl-2,3-dihydro-1Hbenzo[e][1,4]diazepin-1-yl)acetic acid (20 mg) was obtained as off-white solid. ¹H NMR (300 MHz, CDCl₃): δ (ppm) 7.56-7.61 (m, 3H), 7.45-7.50 (m, 2H), 7.28-7.33 (m, 3H), 4.74-4.81 (m, 1H), 4.59-4.68 (m, 1H), 4.43-4.52 (m, 1H), 3.69 (d, J=9.37 Hz, 1H), 1.39 (s, 9H), 1.33 (d, J=5.86 Hz, 3H); Calculated for C₂₃H₂₅ClN₂O₄, 428.15; observed (M+H)⁺429.7.

### Example 60: Synthesis of 2-((S)-7-chloro-3-((R)-1-hydroxyethyl)-2-oxo-5-phenyl-2,3-dihydro-1H-benzo[e][1,4]diazepin-1-yl)acetic acid

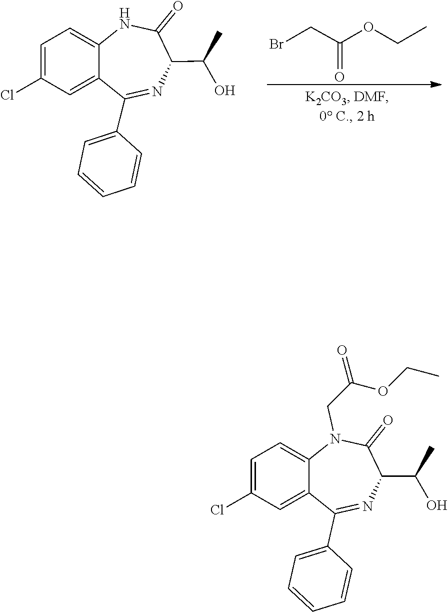

Step 1: 2-((S)-7-chloro-3-((R)-1-hydroxyethyl)-2-oxo-5-phenyl-2,3-dihydro-1H-benzo[e][1,4]diazepin-1-yl)acetate (7): A round bottom flask was equipped with magnetic stir bar, and N₂ inlet, was added (S)-7-chloro-3-((R)-1-hydroxyethyl)-5-phenyl-1H-benzo[e][1,4]diazepin-2(3H)-one (5, 30 mg, 0.0953 mmol) in dry N,N-dimethylformamide (1.5 mL). Resulting solution was cooled to 0 ° C., K₂CO₃ (26.3 mg, 0.1906 mmol) and ethyl 2-bromoacetate (0.012 ml, 0.1143 mmol) was added to the reaction mixture and stirred at 0° C. for 2 hours. The reaction mixture was quenched with ice cold water and extracted with ethyl acetate. The organic layer was dried over Na₂SO₄ and concentrated on rotavapor. The crude was purified by Combi-flash (0-30% ethyl acetate in Hexanes) and ethyl 2-((S)-7-chloro-3-((R)-1-hydroxyethyl)-2-oxo-5-phenyl-2, 3-dihydro-1H-benzo[e][1,4]diazepin-1-yl)acetate (27 mg) was obtained as colorless liquid. Calculated for C₂₁H₂₁ClN₂O₄, 400.12; observed (M+H)⁺401.6

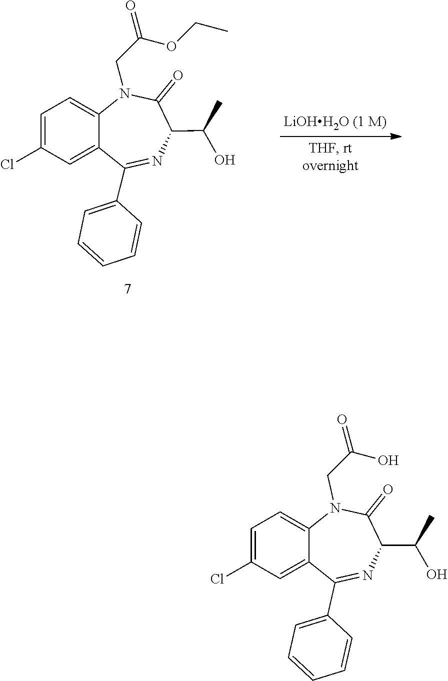 [SMILES: CCOC(=O)CN1C(=O)[C@H]([C@@H](C)O)N=C(c2ccccc2)c2cc(Cl)ccc21 ; C[C@@H](O)[C@@H]1N=C(c2ccccc2)c2cc(Cl)ccc2N(CC(=O)O)C1=O]

Step 2: 2-((S)-7-chloro-3-((R)-1-hydroxyethyl)-2-oxo-5-phenyl-2,3-dihydro-1H-benzo[e][1,4]diazepin-1-yl)acetic acid: The title compound was prepared according to the procedure of 2-((S)-3-((S)-sec-butyl)-5-(4-fluorophenyl)-7-methoxy-2-oxo-2,3-dihydro-1H-benzo[e][1,4]diazepin-1-yl)acetic acid using 2-((S)-7-chloro-3-((R)-1-hydroxyethyl)-2-oxo-5-phenyl-2,3-dihydro-1H-benzo[e][1,4]diazepin-1-yl)acetate to provide the title compound as a off-white solid, 48% yield. ¹H NMR (300 MHz, CDCl₃): δ (ppm) 7.63 (dd, J=13.47, 8.20 Hz, 2H), 7.57 (d, J=7.62 Hz, 2H), 7.44-7.50 (m, 2H), 7.35 (d, J=8.79 Hz, 1H), 7.30 (d, J=1.76 Hz, 1H), 4.61-4.76 (m, 2 H), 4.58 (br. s, 1H), 4.37-4.45 (m, 1H), 3.68 (d, J=6.44 Hz, 1H), 1.30 (d, J=5.86 Hz, 3H); Calculated for C₁₉H₁₇ClN₂O₄, 372.09; observed (M+H)⁺ 373.5.

### Example 61: Synthesis of (S)-2-(7-chloro-3-cyclopropyl-2-oxo-5-phenyl-2,3-dihydro-1H-benzo[e][1,4]diazepin-1-yl)acetic acid

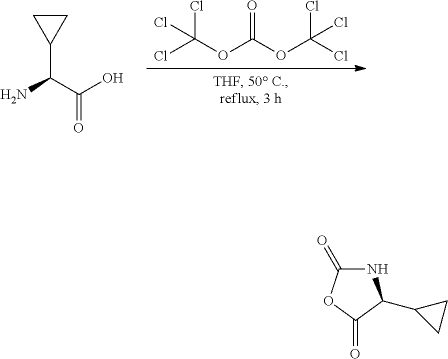

Step 1: A round bottom flask was equipped with magnetic stir bar, condenser, and N₂ inlet, was added (S)-2-amino-2-cyclopropylacetic acid (174 mg, 1.515 mmol) in dry tetrahydrofuran (3 mL) and heated at 50° C. The solution of Triphosgene (150 mg, 0.505 mmol) in dry tetrahydrofuran (5 mL) was added into the reaction by dropping funnel and the reaction mixture was heated at reflux for 3 hours. The reaction mixture was cooled down to 23° C., evaporated to dryness to obtain (S)-4-cyclopropyloxazolidine-2,5-dione (213 mg) as a white solid and was directly taken for next step without further purification.

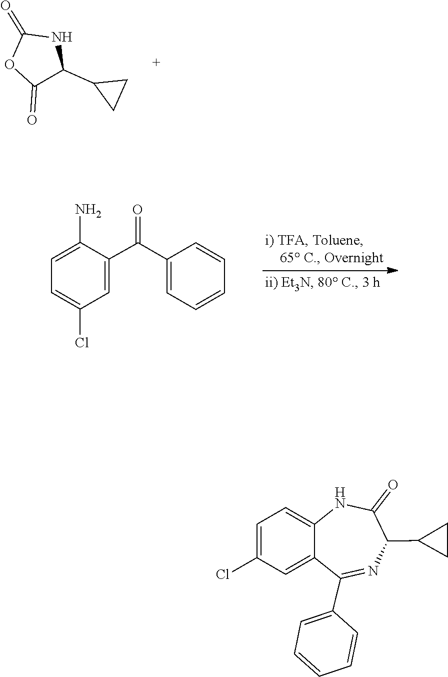 [SMILES: O=C1N[C@@H](C2CC2)C(=O)O1 ; Nc1ccc(Cl)cc1C(=O)c1ccccc1 ; O=C1Nc2ccc(Cl)cc2C(c2ccccc2)=N[C@H]1C1CC1]

Step 2: A round bottom flask was equipped with magnetic stir bar, condenser, and N₂ inlet, was added (S)-4-cyclopropyloxazolidine-2,5-dione (212 mg, 1.502 mmol) and 2-amino-5-chlorobenzophenone (290 mg, 1.2517 mmol) in dry toluene (8 mL). Trifluoroacetic acid (TFA) (0.192 mL, 2.5034 mmol) was added to the reaction mixture and stirred at 65° C. for 18 hours. Then triethylamine (Et₃N) (0.35 mL, 2.5034 mmol) was added to the reaction mixture and stirred at 80° C. for 3 hours. The reaction mixture was cooled down to 23° C., evaporated, quenched with 1 N HCl and extracted with ethyl acetate. Organic layer was washed with sat. NaHCO₃ solution and brine solution, dried over anhy.Na₂SO₄ and concentrated on rotavapor. The crude was purified by Combi-flash (0-30% ethyl acetate in Hexanes) and the (S)-7-chloro-3-cyclopropyl-5-phenyl-1H-benzo[e][1,4]diazepin-2(3H)-one (210 mg) was obtained as off-white solid; Calculated for C₁₈H₁₅ClN₂O, 310.09; observed (M+H)⁺311.5

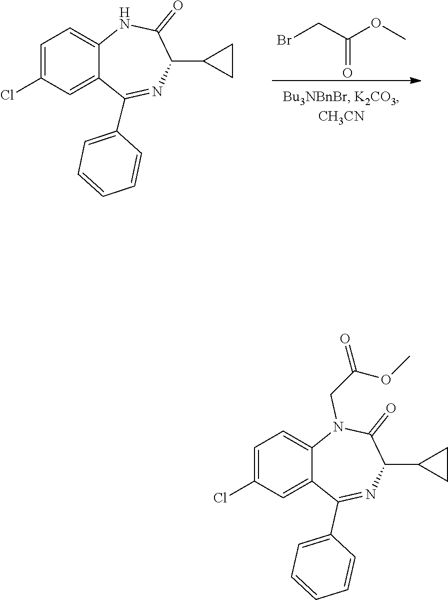

Step 3: A round bottom flask was equipped with magnetic stir bar, and N₂ inlet, was added (S)-7-chloro-3-cyclopropyl-5-phenyl-1H-benzo[e][1,4]diazepin-2(3H)-one (30 mg, 0.0965 mmol) in dry acetonitrile (2 mL). Benzyltributylammonium bromide (17 mg, 0.0482 mmol), potassium carbonate (66 mg, 0.4825 mmol) and methyl 2-bromoacetate (0.014 mL, 0.1447 mmol) was added to the reaction mixture and stirred at 23° C. for 18 hours. The reaction mixture was filtered and evaporated on rotavapor. The crude was purified by Combi-flash (0-30% ethyl acetate in Hexanes) and the (S)-methyl 2-(7-chloro-3-cyclopropyl-2-oxo-5-phenyl-2,3-dihydro-1H-benzo[e][1,4]diazepin-1-yl)acetate (32 mg) was obtained as white solid. Calculated for C₂₁H₁₉ClN₂O₃, 382.11; observed (M+H)⁺383.6

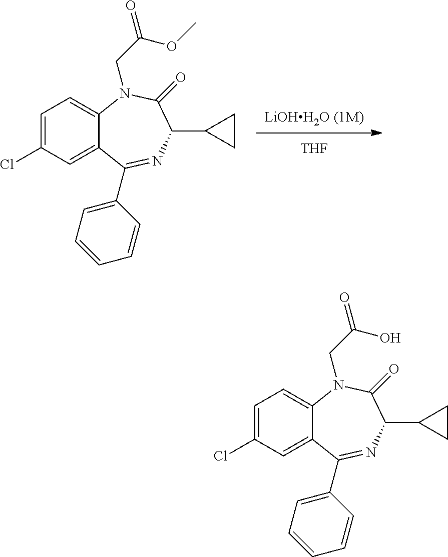 [SMILES: COC(=O)CN1C(=O)[C@H](C2CC2)N=C(c2ccccc2)c2cc(Cl)ccc21 ; "THF" ; O=C(O)CN1C(=O)[C@H](C2CC2)N=C(c2ccccc2)c2cc(Cl)ccc21]

Step 4: A round bottom flask was equipped with magnetic stir bar, was added (S)-methyl 2-(7-chloro-3-cyclopropyl-2-oxo-5-phenyl-2,3-dihydro-1H-benzo[e][1,4]diazepin-1-yl)acetate (32 mg, 0.0835 mmol) in tetrahydrofuran (2 mL). LiOH·H₂O (1 M) aqueous solution (0.25 mL, 0.2507 mmol) was added to the reaction mixture and stirred at 23° C. for 18 hours. The reaction mixture was concentrated on rotavapor. The crude was purified by preparative HPLC and (S)-2-(7-chloro-3-cyclopropyl-2-oxo-5-phenyl-2,3-dihydro-1H-benzo[e][1,4]diazepin-1-yl)acetic acid (13 mg) was obtained as off-white solid. ¹H NMR (300 MHz, CDCl₃): δ (ppm) 7.62 (d, J=7.62 Hz, 4H), 7.45-7.50 (m, 3H), 7.37 (d, J=8.79 Hz, 1H), 4.66-4.74 (m, 1H), 4.44-4.51 (m, 1H), 3.12 (d, J=8.79 Hz, 1H), 1.82-1.89 (m, 1H), 0.76-0.85 (m, 2H), 0.37-0.43 (m, 1H), 0.19-0.27 (m, 1H); Calculated for C₂₀H₁₇CIN₂O₃, 368.09; observed (M+H)⁺369.5

### Example 62: Synthesis of (S)-ethyl 3-(7-chloro-3-cyclopropyl-2-oxo-5-phenyl-2,3-dihydro-1H-benzo[e][1,4]diazepin-1-yl)propanoate

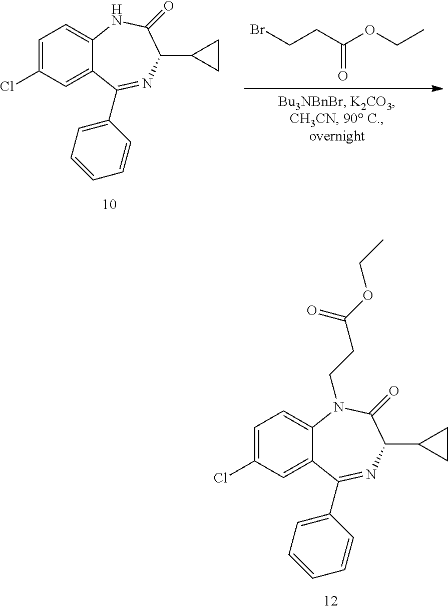

Step 1: A round bottom flask was equipped with magnetic stir bar, condenser, and N₂ inlet, was added (S)-7-chloro-3-cyclopropyl-5-phenyl-1H-benzo[e][1,4]diazepin-2(3H)-one (30 mg, 0.0965 mmol) in dry acetonitrile (2 mL). Benzyltributylammonium bromide (17 mg, 0.0482 mmol), potassium carbonate (66 mg, 0.4825 mmol) and ethyl 3-bromopropanoate (0.018 mL, 0.1447 mmol) was added to the reaction mixture and stirred at 90° C. for 18 hours. The reaction mixture was filtered and evaporated on rotavapor. The crude was purified by Combi-flash (0-30% ethyl acetate in Hexanes) and (S)-ethyl 3-(7-chloro-3-cyclopropyl-2-oxo-5-phenyl-2,3-dihydro-1H-benzo[e][1,4]diazepin-1-yl)propanoate (37 mg) was obtained as colorless gum. Calculated for C₂₃H₂₃ClN₂O₃, 410.14; observed (M+H)⁺411.7

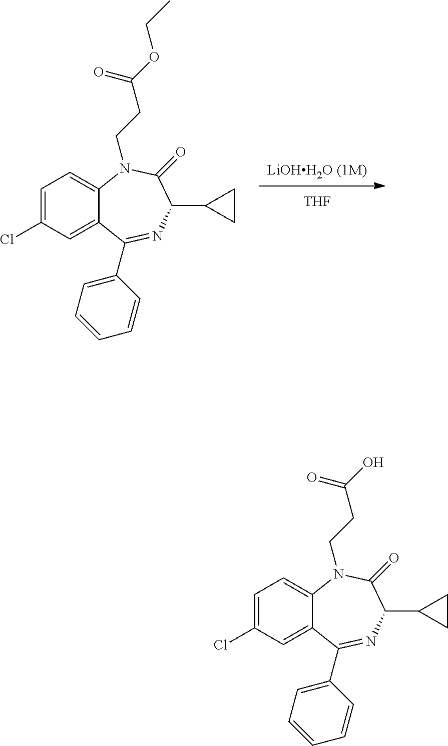 [SMILES: CCOC(=O)CCN1C(=O)[C@H](C2CC2)N=C(c2ccccc2)c2cc(Cl)ccc21 ; "THF" ; O=C(O)CCN1C(=O)[C@H](C2CC2)N=C(c2ccccc2)c2cc(Cl)ccc21]

Step 2: (S)-3-(7-chloro-3-cyclopropyl-2-oxo-5-phenyl-2,3-dihydro-1H-benzo[e][1,4]diazepin-1-yl)propanoic acid: The title compound was prepared according to the method of 2-((S)-3-((S)-sec-butyl)-8-chloro-2-oxo-5-phenyl-2,3-dihydro-1H-benzole111,41diazepin-1-yl)acetic acid using 3-(7-chloro-3-cyclopropyl-2-oxo-5-phenyl-2,3-dihydro-1H-benzo[e][1,4]diazepin-1-yl)propanoate to provide the title compound as a pale yellow solid, 75% yield. ¹H NMR (300 MHz, CDCl₃): δ (ppm) 7.74 (d, J=9.37 Hz, 1H), 7.65 (d, J=7.03 Hz, 2H), 7.44-7.55 (m, 4H), 7.27-7.31 (m, 1H), 4.82-4.93 (m, 1H), 3.83-3.95 (m, 1H), 3.02 (d, J=9.96 Hz, 1H), 2.65-2.74 (m, 1H), 2.33-2.43 (m, 1H), 1.91-2.00 (m, 1H), 0.83-0.96 (m, 2H), 0.34-0.43 (m, 1 H), 0.17-0.27 (m, 1H); Calculated for C₂₁H₁₉ClN₂O₃, 382.11; observed (M+H)⁺383.6

### Example 63: Synthesis of 2-(7-chloro-2-oxo-5-phenyl-3-(trifluoromethyl)-2,3-dihydro-1H-benzo[e][1,4]diazepin-1-yl)acetic acid

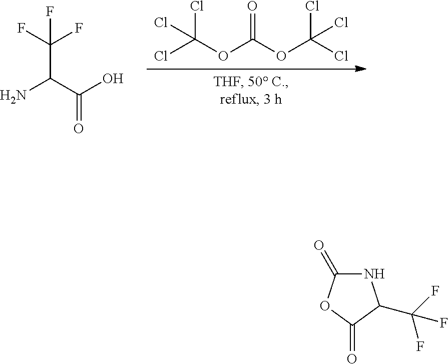

Step 1: 4-(trifluoromethyl)oxazolidine-2,5-dione: The title compound was prepared according to the method of (S)-4-((S)-sec-butyl)oxazolidine-2,5-dione using 2-amino-3,3,3-trifluoropropanoic acid.

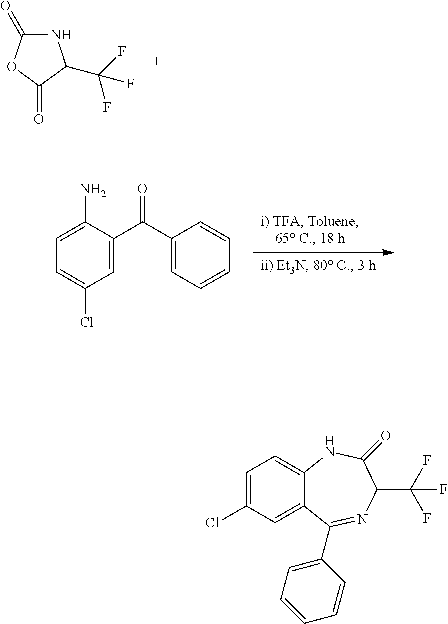

The title compound was prepared according to the procedure of (S)-3-((S)-sec-butyl)-9-chloro-5-phenyl-1,3-dihydro-2H-benzo[e][1,4]diazepin-2-one using 4-(trifluoromethyl)oxazolidine-2,5-dione and (2-amino-5-chlorophenyl)(phenyl)methanone to provide the title compound as an off-white solid, 13% yield. Calculated for C₁₆H₁₀ClF₃N₂O, 338.04; observed (M+H)⁺339.5

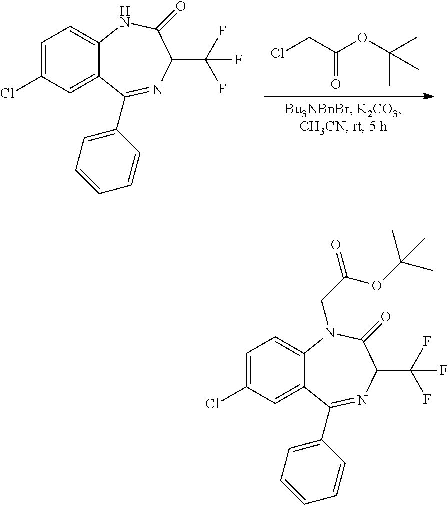

Step 3: A round bottom flask was equipped with magnetic stir bar, and N₂ inlet, was added 7-chloro-5-phenyl-3-(trifluoromethyl)-11H-benzo[e][1,4]diazepin-2(3H)-one (15, 38 mg, 0.112 mmol) in dry acetonitrile (2 mL). Benzyltributylammonium bromide (8 mg, 0.022 mmol), potassium carbonate (23 mg, 0.168 mmol) and tert-butyl 2-chloroacetate (0.017 mL, 0.123 mmol) was added to the reaction mixture and stirred at 23° C. for 5 hours. The reaction mixture was filtered and evaporated on rotavapor. The crude was purified by Combi-flash (0-30% ethyl acetate in Hexanes) and the tert-butyl 2-(7-chloro-2-oxo-5-phenyl-3-(trifluoromethyl)-2,3-dihydro-1H-benzo[e][1,4]diazepin-1 -yl)acetate (23 mg) was obtained as colorless liquid. Calculated for C₂₂H₂₀ClF₃N₂O₃, 452.11; observed (M+H)⁺453.7

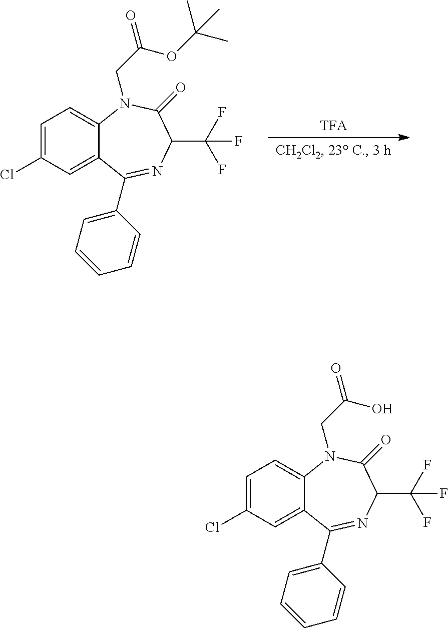 [SMILES: CC(C)(C)OC(=O)CN1C(=O)C(C(F)(F)F)N=C(c2ccccc2)c2cc(Cl)ccc21 ; "CH2Cl2, 23° C., 3 h" ; "TFA" ; O=C(O)CN1C(=O)C(C(F)(F)F)N=C(c2ccccc2)c2cc(Cl)ccc21]

Step 4: A round bottom flask was equipped with magnetic stir bar, was added 2-(7-chloro-2-oxo-5-phenyl-3-(trifluoromethyl)-2,3-dihydro-1H-benzole111,41diazepin-1-yl)acetate (16, 23 mg, 0.050 mmol) in CH₂Cl₂ (4 mL). Trifluoroacetic acid (TFA) (1 mL) was added to the reaction mixture and stirred at 23° C. for 3 hours. The reaction mixture was concentrated on rotavapor. The crude was purified by preparative HPLC and desired product 2-(7-chloro-2-oxo-5-phenyl-3-(trifluoromethyl)-2,3-dihydro-1H-benzo[e][1,4]diazepin-1-yl)acetic acid (12 mg) was obtained as off-white solid. ¹H NMR (300 MHz, MeOD): δ (ppm) 7.60-7.72 (m, 3H), 7.52-7.59 (m, 2H), 7.43-7.50 (m, 2H), 7.29 (d, J=2.34 Hz, 1H), 4.73-4.81 (m, 1H), 4.58-4.61 (m, 1H), 4.53-4.57 (m, 1 H); Calculated for C₁₈H₁₂ClF₃N₂O₃, 396.05; observed (M+H)⁺ 397.6

### Example 64: Synthesis of (S)-3-(2-oxo-3-(pentan-3-yl)-5,7-diphenyl-2,3-dihydro-1H-benzo[e][1,4]diazepin-1-yl)propanoic acid

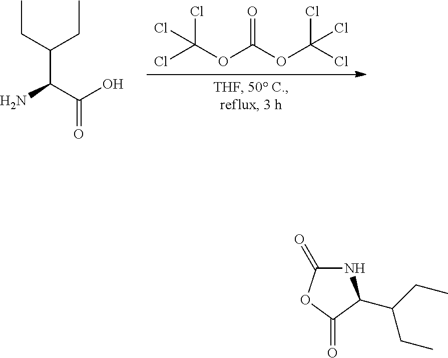

Step 1: (S)-4-(pentan-3-yl)oxazolidine-2,5-dione: The title compound was prepared according to the procedure of (S)-3-((S)-sec-butyl)-9-chloro-5-phenyl-1,3-dihydro-2H-benzo[e][1,4]diazepin-2-one using (S)-2-amino-3-ethylpentanoic acid to provide the title compound as a. colorless liquid. Calculated for C₂₅H₂₉BrN₂O₃, 484.14; observed (M+H)⁺485.8

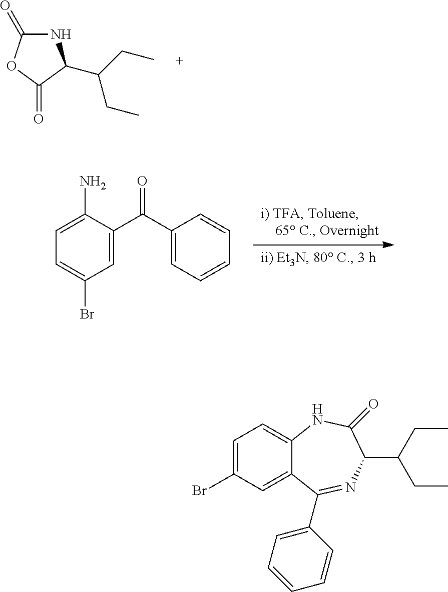

Step 2: (S)-7-bromo-3-(pentan-3-yl)-5-phenyl-1,3-dihydro-2H-benzo[c][1,4]diazepin-2-one: The title compound was prepared according to the procedure of (S)-3-((S)-sec-butyl)-9-chloro-5-phenyl-1,3-dihydro-2H-benzo[e][1,4]diazepin-2-one using (S)-4-(pentan-3-yl)oxazolidine-2,5-dione and (2-amino-5-bromophenyl)(phenyl)methanone to provide the title compound as a colorless liquid. Calculated for C₂₅H₂₉BrN₂O₃, 484.14; observed (M+H)⁺485.8

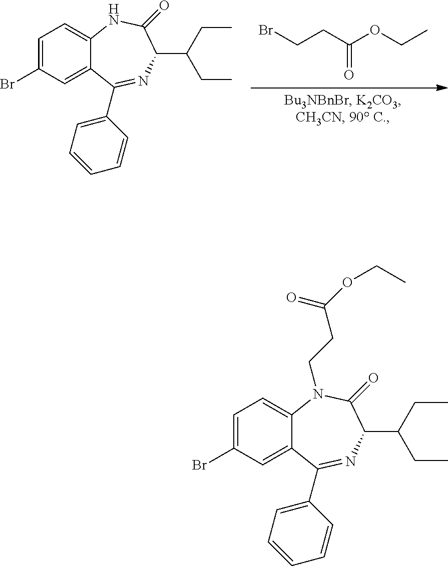

Step 3: (S)-ethyl 3-(7-bromo-2-oxo-3-(pentan-3-yl)-5-phenyl-2,3-dihydro-1H-benzo[e][1,4]diazepin-1-yl)propanoate (21, 67-82): The title compound was prepared according to the procedure of ethyl 2-((S)-3-((S)-sec-butyl)-9-chloro-2-oxo-5-phenyl-2,3-dihydro-1H-benzo[e][1,4]diazepin-1-yl)acetate using (S)-7-bromo-3-(pentan-3-yl)-5-phenyl-1,3-dihydro-2H-benzo[el[1,4]diazepin-2-one and ethyl 3-bromopropanoate to provide the title compound as a colorless liquid. Calculated for C₂₅H₂₉BrN₂O₃, 484.14; observed (M+H)⁺485.8

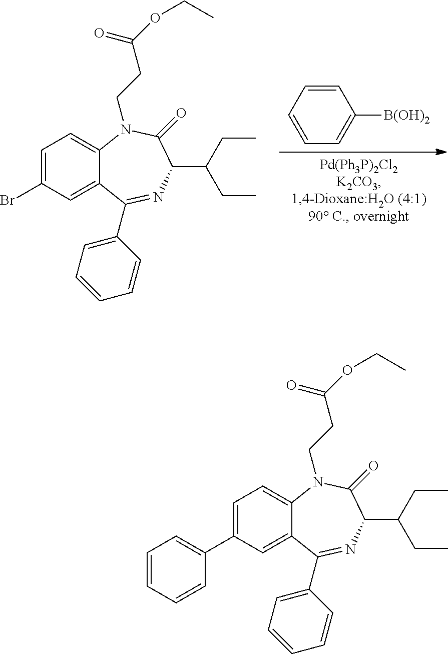

Step 4: A sealable tube was charged with (S)-ethyl 3-(7-bromo-2-oxo-3-(pentan-3-yl)-5-phenyl-2,3-dihydro-1H-benzo[e][1,4]diazepin-1-yl)propanoate (40 mg, 0.0826 mmol), phenylboronic acid (15 mg, 0.1239 mmol) and potassium carbonate (K₂CO₃) (17 mg, 0.1239 mmol) in 1,4-dioxane:H₂O (1.6: 0.4 mL). Then degassed with Argon gas for 5 min, after that Bis(triphenylphosphine)palladium(II) dichloride (PdCl₂(PPh₃)₂) (17 mg, 0.0247 mmol) was added to the reaction mixture and again degassed with Argon gas for 5 min. The tube was sealed with a teflon septum screw-cap and stirred at 90° C. for 18 hours. The reaction mixture was cooled, filtered through celite bed and evaporated on rotavapor. The crude material was purified by Combi-flash (0-30% ethyl acetate in Hexanes) and the (S)-ethyl 3-(2-oxo-3-(pentan-3-yl)-5,7-diphenyl-2,3-dihydro-1H-benzo[c][1,4]diazepin-1-yl)propanoate (34 mg) was obtained as colorless liquid. Calculated for C₃₁H₃₄N₂O₃, 482.26; observed (M+H)⁺484.9

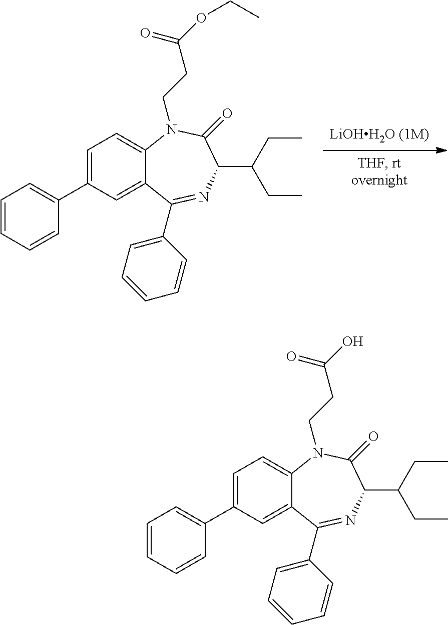 [SMILES: CCOC(=O)CCN1C(=O)[C@H](C(CC)CC)N=C(c2ccccc2)c2cc(-c3ccccc3)ccc21 ; "LiOH·H2O (1M)" ; "THF, rtovernight" ; CCC(CC)[C@@H]1N=C(c2ccccc2)c2cc(-c3ccccc3)ccc2N(CCC(=O)O)C1=O]

Step 4: (S)-3-(2-oxo-3-(pentan-3-yl)-5,7-diphenyl-2,3-dihydro-1H-benzo[e][1,4]diazepin-1-yl)propanoic acid: The title compound was prepared according to the procedure of 2-((S)-3-((S)-sec-butyl)-5-(4-fluorophenyl)-7-methoxy-2-oxo-2,3-dihydro-1H-benzo[e][1,4]diazepin-1-yl)acetic acid using (S)-ethyl 3-(2-oxo-3-(pentan-3-yl)-5,7-diphenyl-2,3-dihydro-1H-benzo[e][1,4]diazepin-1-yl)propanoate to provide the title compound as a pale yellow solid. ¹H-NMR (300 MHz, CDCl₃) δ (ppm) 8.04 (dd, J=8.50, 2.05 Hz, 1H), 7.64-7.72 (m, 4H), 7.51-7.56 (m, 3H), 7.41-7.50 (m, 5H), 4.91-5.05 (m, 1H), 3.91-4.05 (m, 1H), 3.64 (d, J=11.14 Hz, 1H), 2.68-2.79 (m, 1H), 2.56-2.67 (m, 1H), 2.30-2.42 (m, 1H), 1.88-1.99 (m, 1H), 1.73 (dt, J=14.36, 7.47 Hz, 1H), 1.57-1.66 (m, 1H), 1.37 (dt, J=14.07, 7.03 Hz, 1H), 0.82-0.96 (m, 6H); Calculated for C₂₉H₃₀N₂O₃, 454.23; observed (M+H)⁺455.8

### Example 65: Synthesis of (S)-3-(7-cyclopropyl-2-oxo-3-(pentan-3-yl)-5-phenyl-2,3-dihydro-1H-benzo[e][1,4]diazepin-1-^{y}l)propanoic acid

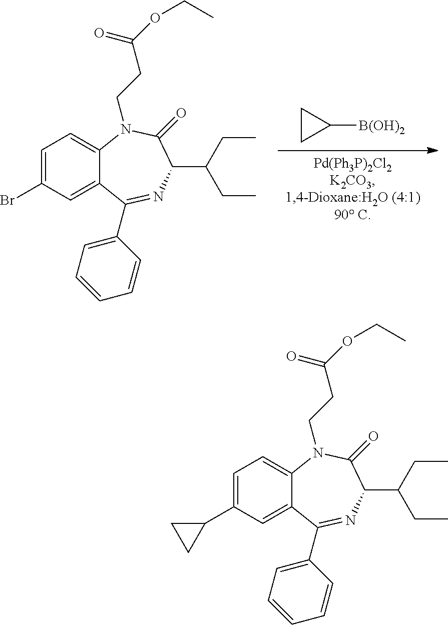

Step 1: (S)-ethyl 3-(7-cyclopropyl-2-oxo-3-(pentan-3-yl)-5-phenyl-2,3-dihydro-1H-benzo[e][1,4]diazepin-1-yl)propanoate (23, 67-92): The ttile compound was prepared according to the procedure of (S)-ethyl 3-(2-oxo-3-(pentan-3-yl)-5,7-diphenyl-2,3-dihydro-1H-benzo[e][1,4]diazepin-1-yl)propanoate using ethyl(S)-3-(7-bromo-2-oxo-3-(pentan-3-yl)-5-phenyl-2,3-dihydro-1H-benzo[e][1,4]diazepin-1-yl)propanoate and cyclopropylboronic acid to provide the title compound as a colorless liquid. Calculated for C₂₅H₃₄N₂O₃, 446.26; observed (M+H)⁺447.9

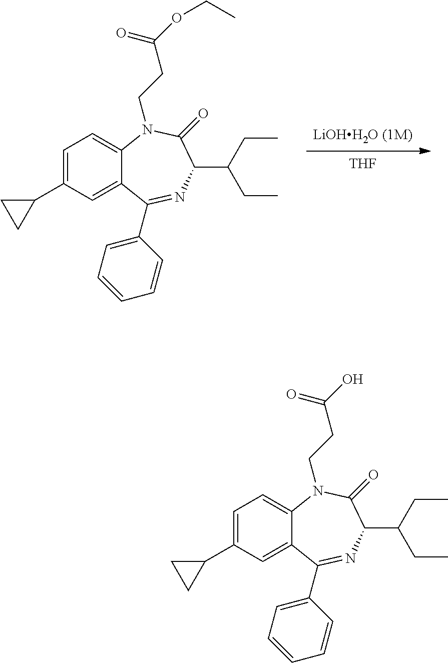 [SMILES: CCOC(=O)CCN1C(=O)[C@H](C(CC)CC)N=C(c2ccccc2)c2cc(C3CC3)ccc21 ; "LiOH·H2O (1M)" ; "THF" ; CCC(CC)[C@@H]1N=C(c2ccccc2)c2cc(C3CC3)ccc2N(CCC(=O)O)C1=O]

Step 2: (S)-3-(7-cyclopropyl-2-oxo-3-(pentan-3-yl)-5-phenyl-2,3-dihydro-1H-benzo[e][1,4]diazepin-1-yl)propanoic acid: The title compound was prepared according to the procedure of 2-((S)-3-((S)-sec-butyl)-5-(4-fluorophenyl)-7-methoxy-2-oxo-2,3-dihydro-1H-benzo[e][1,4]diazepin-1-yl)acetic acid using (S)-ethyl 3-(7-cyclopropyl-2-oxo-3-(pentan-3-yl)-5-phenyl-2,3-dihydro-1H-benzo[e][1,4]diazepin-1-yl)propanoate to provide the title compound as a pale yellow solid. ¹H-NMR (300 MHz, CDCl₃) δ (ppm) 7.69-7.74 (m, 1H), 7.55-7.63 (m, 3H), 7.47-7.54 (m, 3H), 7.06 (s, 1H), 4.93-5.03 (m, 1H), 3.84-3.93 (m, 1H), 3.58 (d, J=11.14 Hz, 1H), 2.67-2.78 (m, 1H), 2.52-2.61 (m, 1H), 2.26 (ddd, J=14.65, 9.38, 4.69 Hz, 1H), 1.85-1.97 (m, 2H), 1.68-1.76 (m, 1H), 1.56-1.66 (m, 1H), 1.29-1.40 (m, 1H), 1.01-1.12 (m, 2H), 0.83-0.95 (m, 6 H), 0.61-0.74 (m, 2H); Calculated for C₂₆H₃₀N₂O₃, 418.23; observed (M+H)⁺419.8

### Example 66: Synthesis of (S)-3-(7-morpholino-2-oxo-3-(pentan-3-yl)-5-phenyl-2,3-dihydro-1H-benzo[e][1,4]diazepin-1-yl)propanoic acid

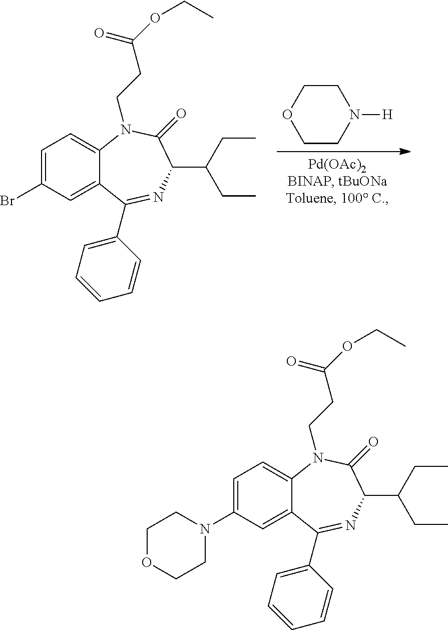

### (S)-ethyl 3-(7-morpholino-2-oxo-3-(pentan-3-yl)-5-phenyl-2,3-dihydro-1H-benzo[e][1,4]diazepin-1-yl)propanoate

Step 1: A Sealable tube was charged with (S)-ethyl 3-(7-bromo-2-oxo-3-(pentan-3-yl)-5-phenyl-2,3-dihydro-1H-benzo[e][1,4]diazepin-1-yl)propanoate (32 mg, 0.066 mmol), morpholine (6.9 mg, 0.079 mmol), (2,2′-bis(diphenylphosphino)-1,1′-binaphthyl) (BINAP, 4 mg, 0.006 mmol) and tBuONa (16 mg, 0.165 mmol) in dry toluene (2 mL). Then degassed with Argon gas for 5 min, after that Palladium (ii) acetate (Pd(OAc)₂ (2 mg, 0.006 mmol) was added to the reaction mixture and again degassed with Argon gas for 5 min. The tube was sealed with teflon septum screw-cap and stirred at 100° C. for 18 hours. The reaction mixture was filtered through celite bed and evaporated on rotavapor. The crude material was purified by Combi-flash (0-50% ethyl acetate in Hexanes) and the (S)-ethyl 3-(7-morpholino-2-oxo-3-(pentan-3-yl)-5-phenyl-2,3-dihydro-1H-benzo[c][1,4]diazepin-1-yl)propanoate (18 mg) was obtained as colorless liquid. Calculated for C₂₉H₃₇N₃O₄, 491.28; observed (M+H)⁺493.0

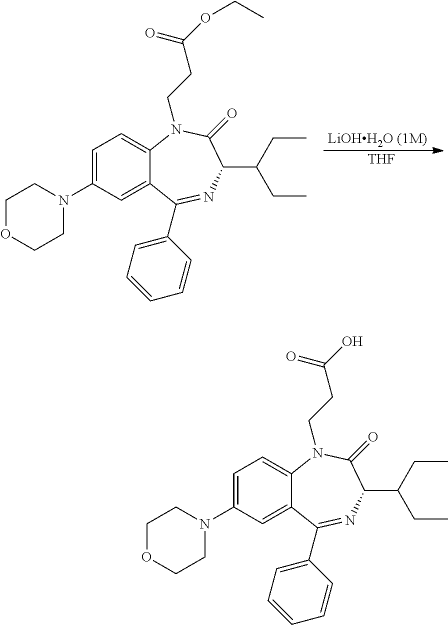 [SMILES: CCOC(=O)CCN1C(=O)[C@H](C(CC)CC)N=C(c2ccccc2)c2cc(N3CCOCC3)ccc21 ; "LiOH·H2O (1M)" ; "THF" ; CCC(CC)[C@@H]1N=C(c2ccccc2)c2cc(N3CCOCC3)ccc2N(CCC(=O)O)C1=O]

Step 2: (S)-3-(7-morpholino-2-oxo-3-(pentan-3-yl)-5-phenyl-2,3-dihydro-1H-benzo[e][1,4]diazepin-1-yl)propanoic acid: The title of compound was prepared according to the procedure of 2-((S)-3-((S)-sec-butyl)-5-(4-fluorophenyl)-7-methoxy-2-oxo-2,3-dihydro-1H-benzo[e][1,4]diazepin-1-yl)acetic acid using (S)-ethyl 3-(7-morpholino-2-oxo-3-(pentan-3-yl)-5-phenyl-2,3-dihydro-1H-benzo[e][1,4]diazepin-1-yl)propanoate to provide the title compound as an orange solid. ¹H-NMR (300 MHz, CDCl₃) δ (ppm) 7.63-7.73 (m, 3H), 7.46-7.56 (m, 3H), 7.34-7.39 (m, 1H), 6.69 (d, J=2.93 Hz, 1H), 4.91-5.01 (m, 1H), 3.82 (t, J=4.69 Hz, 5H), 3.04-3.21 (m, 5H), 2.68-2.76 (m, 1H), 2.52-2.61 (m, 1H), 2.22-2.32 (m, 1H), 1.86-1.95 (m, 1H), 1.67-1.79 (m, 1H), 1.55-1.64 (m, 1H), 1.30-1.40 (m, 1H), 0.84-0.93 (m, 6H); Calculated for C₂₇H₃₃N₃O₄, 463.25; observed (M+H)⁺464.9

### Example 67: Synthesis of (S)-3-(7-chloro-3-cyclopropyl-2-oxo-5-phenyl-2,3-dihydro-1H-benzo[e][1,4]diazepin-1-yl)-N-(methylsulfonyl)propanamide

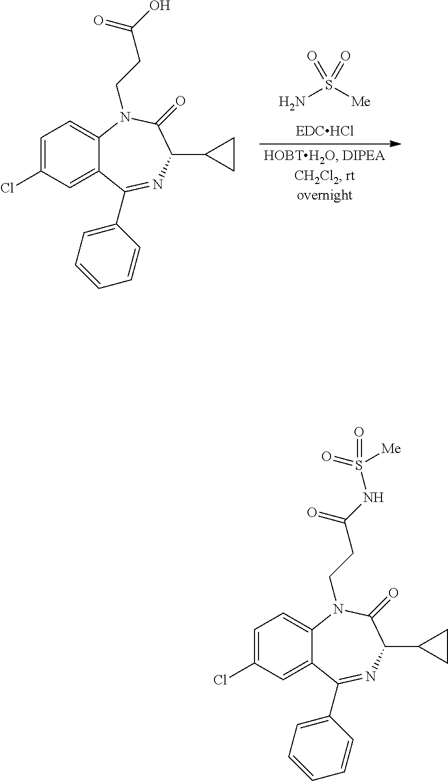 [SMILES: O=C(O)CCN1C(=O)[C@H](C2CC2)N=C(c2ccccc2)c2cc(Cl)ccc21 ; CS(N)(=O)=O ; CS(=O)(=O)NC(=O)CCN1C(=O)[C@H](C2CC2)N=C(c2ccccc2)c2cc(Cl)ccc21]

A 20 ml vial was charged with (S)-3-(7-chloro-3-cyclopropyl-2-oxo-5-phenyl-2,3-dihydro-1H-benzo[e][1,4]diazepin-1-yl)propanoic acid (14 mg, 0.0365 mmol) in CH₂Cl₂, was added 1-ethyl-3-(3-dimethylaninopropyl)carbodiiinide HCl (11 ng, 0.0547 mmol), hydroxybenzotriazole (7 mg, 0.0438 mmol), and N, N-diisopropylethylamine (0.013 ml, 0.073 mmol) and stirred at 23° C. for 10 min. After that methane sulfonamide (5 mg, 0.0438 mmol) was added to the reaction mixture and stirred at 23° C. for 18 hours. The reaction mixture was quenched with sat. NH₄Cl aqueous solution and extracted with ethyl acetate. The organic layer was washed with brine solution, dried over anhy.Na₂SO₄ and concentrated on rotavapor. The crude material was purified by preparative HPLC and the (S)-3-(7-chloro-3-cyclopropyl-2-oxo-5-phenyl-2,3-dihydro-1H-benzo[e][1,4]diazepin-1-yl)-N-(methylsulfonyl)propanamide (2 mg) was obtained as white solid:; ¹H-NMR (300 MHz, CDCl₃) δ (ppm) 7.61-7.73 (m, 4H), 7.47-7.56 (m, 2H), 7.41 (d, J=8.79 Hz, 1H), 7.28-7.31 (m, 1H), 4.46-4.56 (m, 1H), 3.98-4.07 (m, 1H), 2.98 (d, J=9.37 Hz, 1H), 2.84 (s, 3H), 2.50-2.60 (m, 1H), 1.87-1.98 (m, 2H), 0.78-0.89 (m, 2H), 0.35-0.43 (m, 1H), 0.18-0.25 (m, 1H); Calculated for C₂₂H₂₂CIN₃O₄S, 459.1; observed (M+H)⁺ 460.7

### Example 68: Synthesis of (S)-3-(7-chloro-3-cyclopropyl-2-oxo-5-phenyl-2,3-dihydro-1H-benzo[e][1,4]diazepin-1-yl)-N-(pyridin-2-ylsulfonyl)propanamide

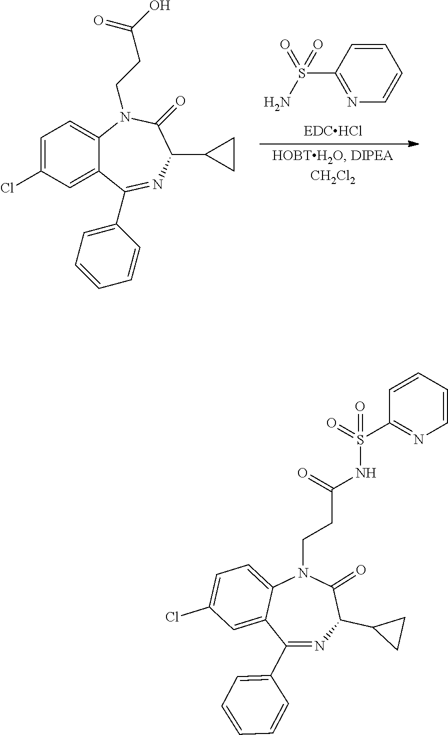 [SMILES: O=C(O)CCN1C(=O)[C@H](C2CC2)N=C(c2ccccc2)c2cc(Cl)ccc21 ; NS(=O)(=O)c1ccccn1 ; O=C(CCN1C(=O)[C@H](C2CC2)N=C(c2ccccc2)c2cc(Cl)ccc21)NS(=O)(=O)c1ccccn1]

(S)-3-(7-chloro-3-cyclopropyl-2-oxo-5-phenyl-2,3-dihydro-1H-benzo[c][1,4]diazepin-1-yl)-N-(pyridin-2-ylsulfonyl)propanamide: The title compound was prepared according to the method of (S)-3-(7-chloro-3-cyclopropyl-2-oxo-5-phenyl-2,3-dihydro-1H-benzo[e][1,4]diazepin-1-yl)-N-(methylsulfonyl)propanamide using (S)-3-(7-chloro-3-cyclopropyl-2-oxo-5-phenyl-2,3-dihydro-1H-benzo[e][1,4]diazepin-1-yl)propanoic acid and pyridine-2-sulfonamide to provide the title compound as a white solid. ¹H-NMR (300 MHz, CDCl₃) δ (ppm) 8.57 (d, J=4.10 Hz, 1H), 8.05 (d, J=8.20 Hz, 1H), 7.87 (t, J=7.91 Hz, 1H), 7.52-7.65 (m, 4H), 7.45-7.51 (m, 3H), 7.31 (d, J=8.79 Hz, 1H), 7.19-7.23 (m, 1H), 4.37-4.48 (m, 1H), 3.86-3.98 (m, 1H), 2.96 (d, J=8.79 Hz, 1H), 2.63-2.69 (m, 2H), 1.84-1.91 (m, 1H), 0.71-0.85 (m, 2H), 0.32-0.45 (m, 1H), 0.14-0.25 (m, 1H); Calculated for C₂₆H₂₃ClN₄O₄S, 522.11; observed (M+H)⁺ 523.8

### Example 68: Synthesis of (S)-3-(3-(tert-butyl)-8-chloro-4-oxo-1-phenyl-3H-benzo[e][1,4]diazepin-5(4H)-yl)-N-(methylsulfonyl)propanamide

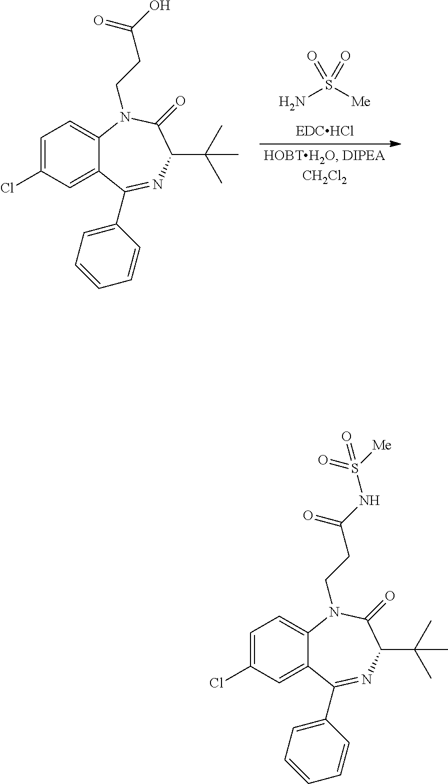 [SMILES: CC(C)(C)[C@@H]1N=C(c2ccccc2)c2cc(Cl)ccc2N(CCC(=O)O)C1=O ; CS(N)(=O)=O ; CC(C)(C)[C@@H]1N=C(c2ccccc2)c2cc(Cl)ccc2N(CCC(=O)NS(C)(=O)=O)C1=O]

(S)-3-(3-(tert-butyl)-8-chloro-4-oxo-1-phenyl-3H-benzo[e][1,4]diazepin-5(4H)-yl)-N-(methylsulfonyl)propanamide: The title compound was prepared according to the method of (S)-3-(7-chloro-3-cyclopropyl-2-oxo-5-phenyl-2,3-dihydro-1H-benzo[e][1,4]diazepin-1-yl)-N-(methylsulfonyl)propanamide using (S)-3-(3-(tert-butyl)-7-chloro-2-oxo-5-phenyl-2,3-dihydro-1H-benzo[e][1,4]diazepin-1-yl)propanoic acid and methanesulfonamide to provide the title compound as a off-white solid. ¹H-NMR (300 MHz, CDCl₃) δ (ppm) 7.63-7.66 (m, 2H), 7.56-7.62 (m, 1H), 7.41-7.51 (m, 3H), 7.33 (d, J=2.34 Hz, 1H), 7.25 (d, J=1.76 Hz, 1H), 4.54-4.64 (m, 1H), 3.93-4.04 (m, 1H), 3.25 (s, 1H), 2.91 (s, 3H), 2.73-2.83 (m, 1H), 2.45-2.56 (m, 1H), 1.24 (s, 9H); Calculated for C₂₃H₂₆ClN₃O₄S, 475.13; observed (M+H)⁺476.7

### Example 69: Synthesis of (S)-3-(3-(tert-butyl)-8-chloro-4-oxo-1-phenyl-3H-benzo[e][1,4]diazepin-5(4H)-yl)-N-(pyridin-2-ylsulfonyl)propanamide

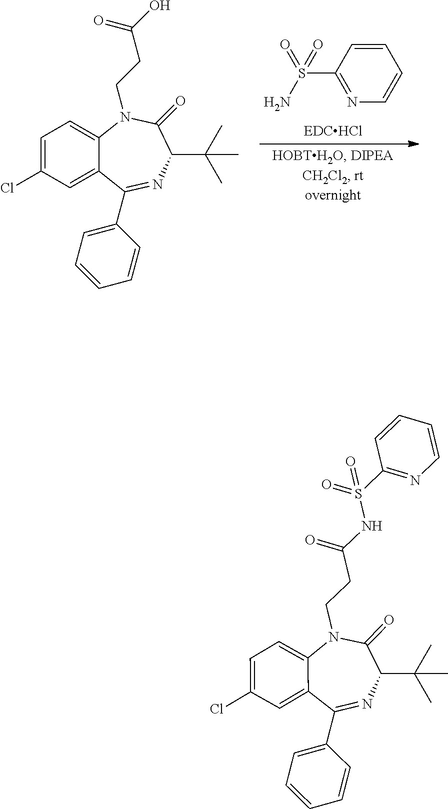 [SMILES: CC(C)(C)[C@@H]1N=C(c2ccccc2)c2cc(Cl)ccc2N(CCC(=O)O)C1=O ; NS(=O)(=O)c1ccccn1 ; CC(C)(C)[C@@H]1N=C(c2ccccc2)c2cc(Cl)ccc2N(CCC(=O)NS(=O)(=O)c2ccccn2)C1=O]

(S)-3-(3-(tert-butyl)-8-chloro-4-oxo-1-phenyl-3H-benzo[e][1,4]diazepin-5(4H)-yl)-N-(pyridin-2-ylsulfonyl)propanamide: The title compound was prepared according to the method of (S)-3-(7-chloro-3-cyclopropyl-2-oxo-5-phenyl-2,3-dihydro-1H-benzo[e][1,4]diazepin-1-yl)-N-(methylsulfonyl)propanamide using (S)-3-(3-(tert-butyl)-7-chloro-2-oxo-5-phenyl-2,3-dihydro-1H-benzo[e][1,4]diazepin-1-yl)propanoic acid and pyridine-2-sulfonamide to provide the title compound as an off-white solid. ¹H-NMR (300 MHz, CDCl₃) δ (ppm) 8.55 (d, J=5.27 Hz, 1H), 8.06 (d, J=8.20 Hz, 1H), 7.85-7.91 (m, 1H), 7.57-7.61 (m, 2H), 7.50-7.54 (m, 3H), 7.41-7.49 (m, 3H), 7.31 (d, J=8.79 Hz, 1H), 4.36-4.45 (m, 1H), 3.88-3.97 (m, 1H), 3.13 (s, 1H), 2.56-2.65 (m, 2H), 1.20 (s, 9H); Calculated for C₂₇H₂₇ClN₄O₄S, 538.14; observed (M+H)⁺539.8

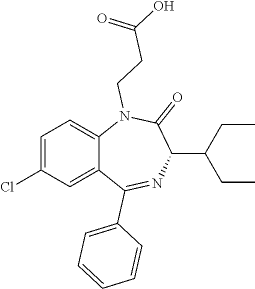 [SMILES: CCC(CC)[C@@H]1N=C(c2ccccc2)c2cc(Cl)ccc2N(CCC(=O)O)C1=O]

### Example 70: Synthesis of (S)-3-(7-chloro-2-oxo-3-(pentan-3-yl)-5-phenyl-2,3-dihydro-1H-benzo[e][1,4]diazepin-1-yl)propanoic acid

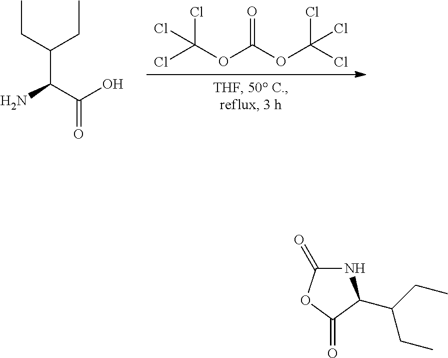

Step 1: A round bottom flask was equipped with magnetic stir bar, condenser, and N₂ inlet, was added (S)-2-Amino-3-ethylpentanoic acid (220 ng, 1.516 mmol) in dry tetrahydrofuran (3 mL) and heated at 50° C. The solution of Triphosgene (150 mg, 0.505 mmol) in dry tetrahydrofuran (5 mL) was added into the reaction by dropping funnel and the reaction mixture was heated at reflux for 3 hours. The reaction mixture was cooled down to 23° C., evaporated to dryness to obtain 4-(pentan-3-yl) oxazolidine-2,5-dione (259 mg, 100% yield) as an oil and was directly taken for next step without further purification.

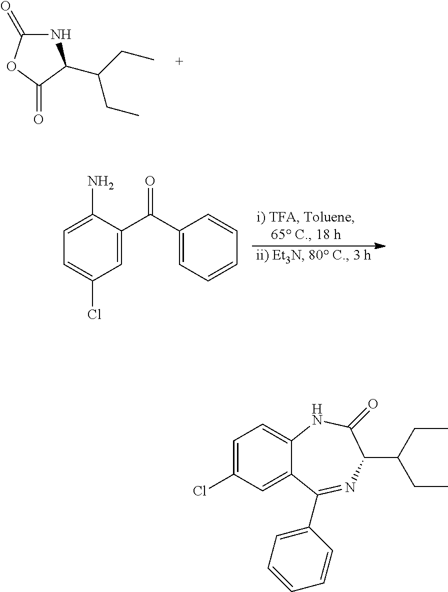

Step 2: A round bottom flask was equipped with magnetic stir bar, condenser, and N₂ inlet, was added 4-(pentan-3-yl) oxazolidine-2,5-dione (259 mg, 1.512 mmol) and 2-amino-5-chlorobenzophenone (3, 292 mg, 1.260 mmol) in dry toluene (8 mL). Trifluoroacetic acid (TFA) (0.194 mL, 2.52 mmol) was added to the reaction mixture and stirred at 65° C. for 18 hours. Then triethylamine (Et₃N) (0.35 ML, 2.52 mmol) was added to the reaction mixture and stirred at 80° C. for 3 hours. The reaction mixture was cooled down to rt, evaporated, quenched with 1N HCl and extracted with ethyl acetate. The organic layer was washed with sat. NaHCO₃ solution and brine solution, dried over anhy.Na₂SO₄ and concentrated. The crude material was purified by Combi-flash (0-30% ethyl acetate in Hexanes) and the (S)-7-chloro-3-(pentan-3-yl)-5-phenyl-1H-benzo[c][1,4]diazepin-2(3H)-one (300 mg) was obtained as colorless liquid. ¹H NMR (300 MHz, CDCl3): δ (ppm) 7.49-7.53 (m, 2H), 7.37-7.48 (m, 4H), 7.32 (d, J=2.34 Hz, 1H), 7.14 (d, J=8.79 Hz, 1H), 3.38 (d, J=9.37 Hz, 1H), 2.40-2.51 (m, 1H), 1.86-1.99 (m, 1H), 1.62-1.76 (m, 2H), 1.36-1.49 (m, 1H), 0.90-0.96 (m, 6H); Calculated for C₂₀H₂₁ClN₂O, 340.13; observed (M+H)⁺341.6.

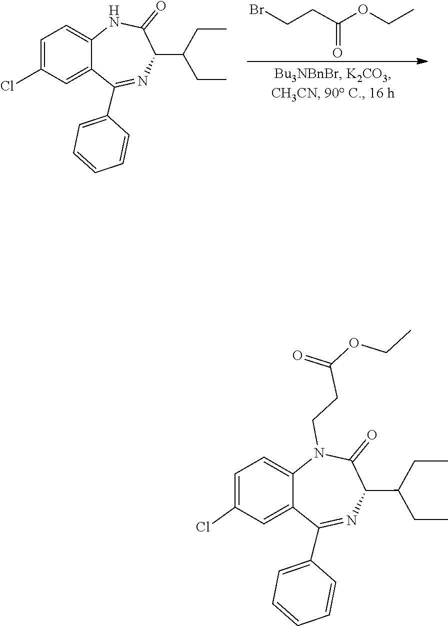

Step 3: A round bottom flask was equipped with magnetic stir bar, condenser, and N₂ inlet, was added (S)-7-chloro-3-(pentan-3-yl)-5-phenyl-1H-benzo[e][1,4]diazepin-2(3H)-one (1 gr, 2.941 mmol) in dry acetonitrile (12 mL). Benzyltributylammonium bromide (0.155 gr, 0.441 mmol), potassium carbonate (0.801 gr, 5.882 mmol) and ethyl 3-bromopropanoate (0.403 mL, 3.235 mmol) was added to the reaction mixture and stirred at 90° C. for 16 hours. The reaction mixture was filtered and evaporated on rotavapor. The crude material was purified by Combi-flash (0-40% ethyl acetate in dichloromethane) and the (S)-ethyl 3-(7-chloro-2-oxo-3-(pentan-3-yl)-5-phenyl-2,3-dihydro-1H-benzo[e][1,4]diazepin-1-yl)propanoate 1.105 gr, 86% yield) was obtained as colorless liquid. ¹H NMR (300 MHz, CDCl₃): δ (ppm) 7.59-7.64 (m, 2H), 7.49-7.55 (m, 1H), 7.36-7.48 (m, 4H), 7.27-7.30 (m, 1H), 4.44-4.57 (m, 1H), 3.83-3.99 (m, 3H), 3.31 (d, J=9.37 Hz, 1H), 2.46-2.64 (m, 3 H), 1.78-1.92 (m, 1H), 1.61-1.71 (m, 1H), 1.46-1.58 (m, 1H), 1.25-1.38 (m, 1H), 1.03-1.13 (m, 3H), 0.82-0.92 (m, 6H); Calculated for C₂₅H₂₉ClN₂O₃, 440.19; observed (M+H)⁺441.8.

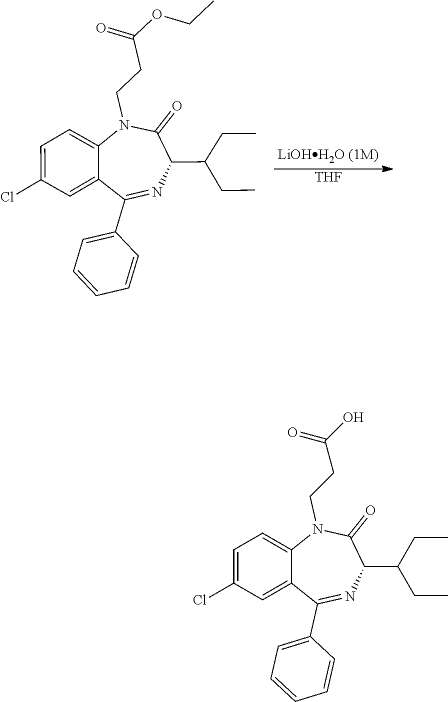 [SMILES: "THF" ; CCOC(=O)CCN1C(=O)[C@H](C(CC)CC)N=C(c2ccccc2)c2cc(Cl)ccc21 ; CCC(CC)[C@@H]1N=C(c2ccccc2)c2cc(Cl)ccc2N(CCC(=O)O)C1=O]

Step 4: A round bottom flask was equipped with magnetic stir bar, was added (S)-ethyl 3-(7-chloro-2-oxo-3-(pentan-3-yl)-5-phenyl-2,3-dihydro-1H-benzo[e][1,4]diazepin-1-yl)propanoate (1.81 g, 4.105 mmol) in tetrahydrofuran (15 mL). LiOH·H₂O (1 M) aqueous solution (9 mL, 9.031 mmol) was added to the reaction mixture and stirred at 23° C. for 18 hours. The reaction mixture was concentrated to half volume on rotavapor. The solution was cooled down to 0° C. and the pH was adjusted to 3-4 by addition of 10% citric acid monohydrate aqueous solution and extracted with ethyl acetate, dried over anhydrous Na₂SO₄ and evaporated on rotavapor. The crude material was purified by Combi-flash (0-30% EtOAc/Hexanes)benzodiazepine intermediate was isolated, followed by run Combi-flash in MeOH/CH₂Cl₂ (0-10%) and (S)-3-(7-chloro-2-oxo-3-(pentan-3-yl)-5-phenyl-2,3-dihydro-1H-benzo[e][1,4]diazepin-1-yl)propanoic acid (1.3 g) was obtained as off white solid. The enantiomer excess (ee) was determined as 94.6% on a 1B N-3, 3 μm, 4.6×150 mm colunm with hexane/EtOH/TFA 90/10/0.1, 1.0 mL/min, 25° C. gradient. ¹H NMR (300 MHz, CDCl₃): δ (ppm) 7.49-7.57 (m, 3H), 7.33-7.46 (m, 4H), 7.25-7.28 (m, 1H), 4.44-4.59 (m, 1H), 3.79-3.96 (m, 1H), 3.29 (d, J=9.96 Hz, 1H), 2.47-2.55 (m, 3H), 1.78-1.90 (m, 1H), 1.59-1.72 (m, 1H), 1.43-1.55 (m, 1H), 1.26-1.33 (m, 1H), 0.79-0.90 (m, 6H); Calculated for C₂₃H₂₅ClN₂O₃, 412.16; observed (M+H)⁺413.8.

### Example 71: Synthesis of (S)-3-(7-chloro-3-isopropyl-2-oxo-5-phenyl-2,3-dihydro-1H-benzo[e][1,4]diazepin-1-yl)propanoic acid

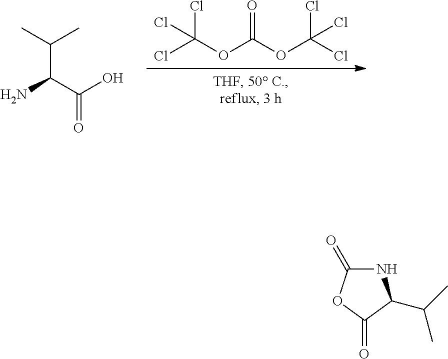

Step 1: (S)-4-isopropyloxazolidine-2,5-dione was prepared according to the procedure for 4-(pentan-3-yl) oxazolidine-2,5-dione except (s)-valine was substituted for (S)-2-Amino-3-ethylpentanoic acid.

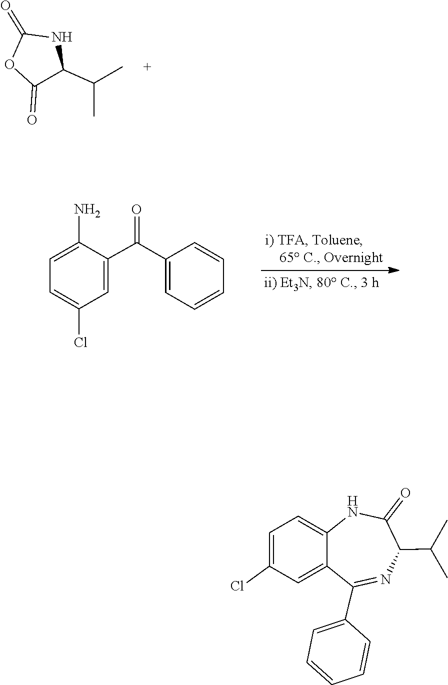

Step 2: (S)-7-chloro-3-isopropyl-5-phenyl-1,3-dihydro-2H-benzo[e][1,4]diazepin-2-one was prepared according to the procedure for (S)-7-chloro-3-(pentan-3-yl)-5-phenyl-1H-benzo[e][1,4]diazepin-2(3H)-one expect (S)-4-isopropyloxazolidine-2,5-dione was substituted for 4-(pentan-3-yl) oxazolidine-2,5-dione,

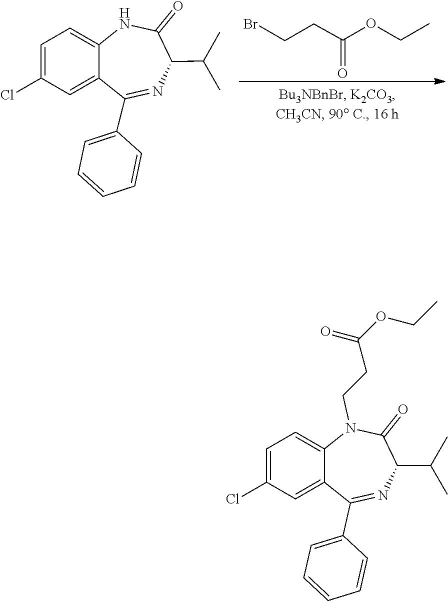

Step 3: ethyl(S)-3-(7-chloro-3-isopropyl-2-oxo-5-phenyl-2,3-dihydro-1H-benzo[e][1,4]diazepin-1-yl)propanoate was prepared according to the procedure for (S)-ethyl 3-(7-chloro-2-oxo-3-(pentan-3-yl)-5-phenyl-2,3-dihydro-1H-benzo[e][1,4]diazepin-1-yl)propanoate except (S)-7-chloro-3-isopropyl-5-phenyl-1,3-dihydro-2H-benzo[e][1,4]diazepin-2-one was substituted for(S)-ethyl 3-(7-chloro-2-oxo-3-(pentan-3-yl)-5-phenyl-2,3-dihydro-1H-benzo[c][1,4]diazepin-1-yl)propanoate.

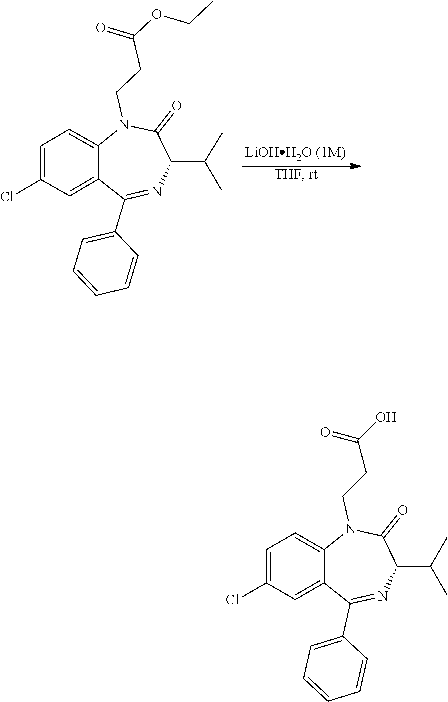 [SMILES: CCOC(=O)CCN1C(=O)[C@H](C(C)C)N=C(c2ccccc2)c2cc(Cl)ccc21 ; "THF, rt" ; CC(C)[C@@H]1N=C(c2ccccc2)c2cc(Cl)ccc2N(CCC(=O)O)C1=O]

Step 4: (S)-3-(7-chloro-3-isopropyl-2-oxo-5-phenyl-2,3-dihydro-1H-benzo[e][1,4]diazepin-1-yl)propanoic acid was prepared according to the procedure for (S)-3-(7-chloro-2-oxo-3-(pentan-3-yl)-5-phenyl-2,3-dihydro-1H-benzo[e][1,4]diazepin-1-yl)propanoic acid except ethyl(S)-3-(7-chloro-3-isopropyl-2-oxo-5-phenyl-2,3-dihydro-1H-benzo[e][1,4]diazepin-1-yl)propanoate was substituted for (S)-ethyl 3-(7-chloro-2-oxo-3-(pentan-3-yl)-5-phenyl-2,3-dihydro-1H-benzo[e][1,4]diazepin-1-yl)propanoate. ¹H NMR (300 MHz, CDCl₃): δ (ppm) 7.49-7.60 (m, 3 H), 7.33-7.46 (m, 4H), 7.26-7.28 (m, 1H), 4.47-4.59 (m, 1H), 3.80-3.94 (m, 1H), 3.03 (d, J=9.96 Hz, 1H), 2.68-2.82 (m, 1H), 2.46-2.57 (m, 2H), 1.09-1.16 (m, 3H), 0.96-1.04 (m, 3H); Calculated for C₂₁H₂₁ClN₂O₃, 384.12; observed (M+H)⁺385.7.

### Example 72: Synthesis of (S)-3-(7-bromo-2-oxo-3-(pentan-3-yl)-5-phenyl-2,3-dihydro-1H-benzo[e][1,4]diazepin-1-yl)propanoic acid

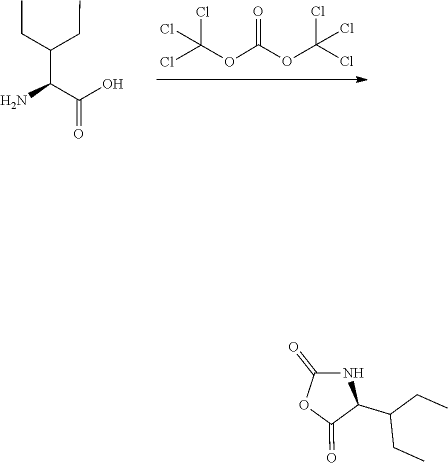

Step 1: Synthesis of (S)-4-(pentan-3-yl)oxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-3-ethylpentanoic acid to provide the title compound as a white solid.

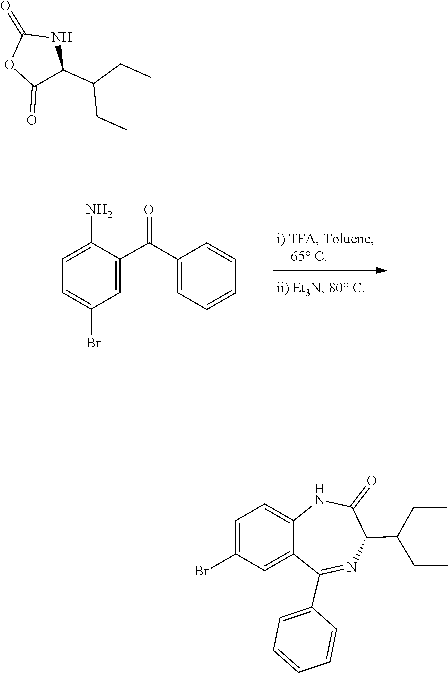 [SMILES: CCC(CC)[C@@H]1NC(=O)OC1=O ; Nc1ccc(Br)cc1C(=O)c1ccccc1 ; CCC(CC)[C@@H]1N=C(c2ccccc2)c2cc(Br)ccc2NC1=O]

Step 2: Synthesis of (S)-7-bromo-3-(pentan-3-yl)-5-phenyl-1H-benzo[e][1,4]diazepin-2(3H)-one (20, 67-54): The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-(pentan-3-yl)oxazolidine-2,5-dione and 2-amino-5-chlorobenzophenone was replaced with 2-amino-5-bromobenzophenone to provide the title compound as colorless liquid. Calculated for C₂₀H₂₁BrN₂O, 384.08; observed (M+H)⁺385.6

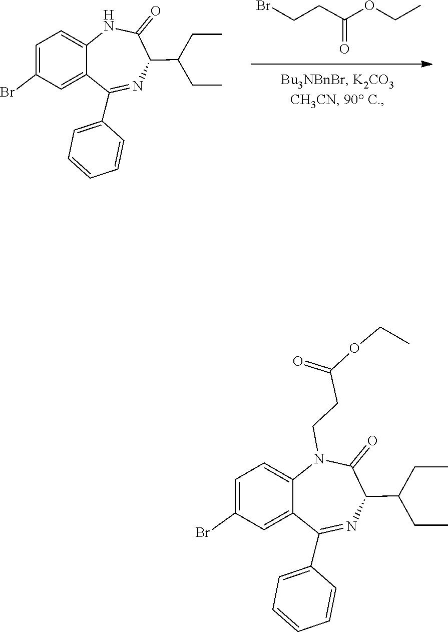

Step 3: Synthesis of (S)-ethyl 3-(7-bromo-2-oxo-3-(pentan-3-yl)-5-phenyl-2,3-dihydro-1H-benzo[e][1,4]diazepin-1-yl)propanoate (21, 67-82): The title compound was prepared according to the procedure for (S)-methyl 2-(7-chloro-3-cyclopropyl-2-oxo-5-phenyl-2,3-dihydro-1H-benzo|e111,41diazepin-1-yl)acetate except that (S)-7-chloro-3-cyclopropyl-5-phenyl-1H-benzo[e][1,4]diazepin-2(3H)-one was replaced with (S)-7-bromo-3-(pentan-3-yl)-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₅H₂₉BrN₂O₃, 484.14; observed (M+H)⁺485.8

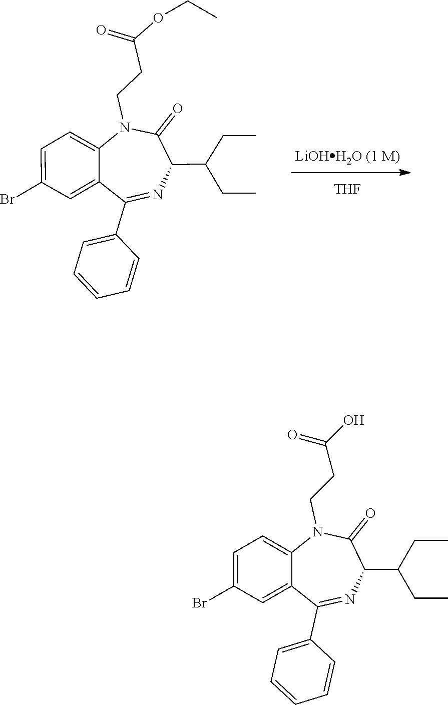 [SMILES: CCOC(=O)CCN1C(=O)[C@H](C(CC)CC)N=C(c2ccccc2)c2cc(Br)ccc21 ; CCC(CC)[C@@H]1N=C(c2ccccc2)c2cc(Br)ccc2N(CCC(=O)O)C1=O ; "THF"]

Step 4 Synthesis of (S)-3-(7-bromo-2-oxo-3-(pentan-3-yl)-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(7-bromo-2-oxo-3-(pentan-3-yl)-5-phenyl-2,3-dihydro-1H-benzo[e][1,4]diazepin-1-yl)propanoate to provide the title compound as an off white solid. ¹H-NMR (300 MHz, MeOD) δ (ppm) 7.84 (dd, J=8.79, 2.34 Hz, I H), 7.57-7.63 (m, 4H), 7.46-7.53 (m, 2H), 7.38 (d, J=2.34 Hz, 1H), 4.54 (dt, J=14.06, 6.15 Hz, 1H), 3.98-4.09 (m, 1H), 3.45 (d, J=10.54 Hz, 1H), 2.44-2.56 (m, 3H), 1.77-1.89 (m, 1H), 1.64 (dt, J=14.50, 7.10 Hz, 1H), 1.45-1.56 (m, 1H), 1.29-1.41 (m, 1H), 0.81-0.92 (m, 6H); Calculated for C₂₃H₂₅BrN₂O₃, 456.10; observed (M+H)⁺457.7

### Example 74: Synthesis of (S)-2-(3-(tert-butyl)-7-chloro-2-oxo-5-phenyl-2,3-dihydro-1H-benzo[e][1,4]diazepin-1-yl)acetic acid

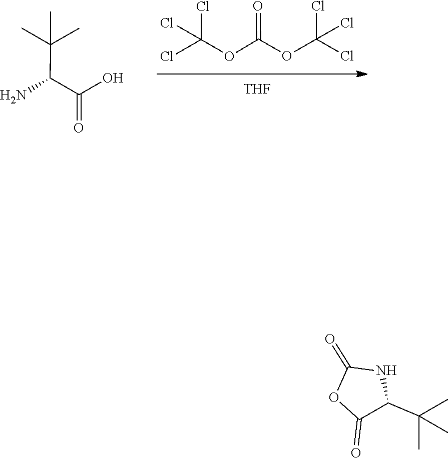

Step 1: (R)-4-(tert-butyl)oxazolidine-2,5-dione: The title compound was prepared according to the procedure of (S)-4-cyclopropyloxazolidine-2,5-dione except that (S)-2-amino-2-cyclopropylacetic acid was replaced with L-tert-Leucine to provide the title compound as a white solid.

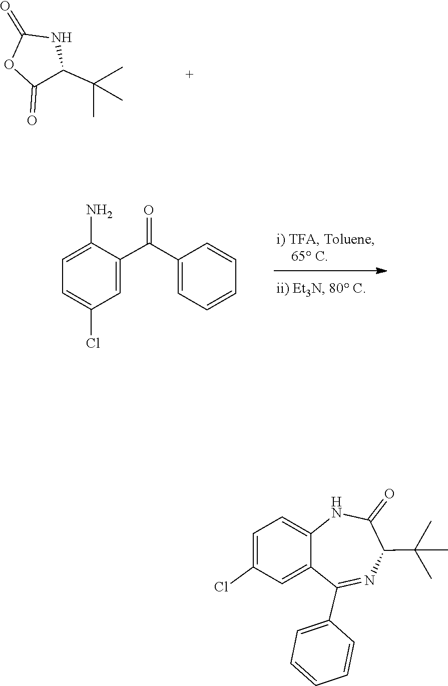 [SMILES: CC(C)(C)[C@H]1NC(=O)OC1=O ; Nc1ccc(Cl)cc1C(=O)c1ccccc1 ; CC(C)(C)[C@@H]1N=C(c2ccccc2)c2cc(Cl)ccc2NC1=O]

Step 2: Synthesis of (S)-3-(tert-butyl)-7-chloro-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (R)-4-(tert-butyl)oxazolidine-2,5-dione to provide the title compound as a white solid. Calculated for C₁₉H₁₉ClN₂O, 326.12; observed (M+H)⁺327.6

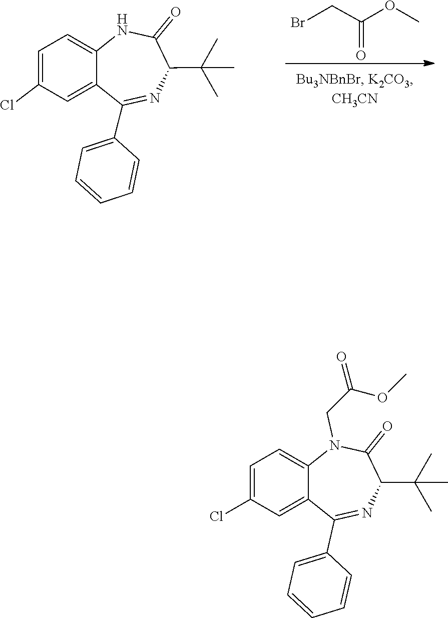

Step 3: Synthesis of (S)-methyl 2-(3-(tert-butyl)-7-chloro-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with(S)-3-(tert-butyl)-7-chloro-5-phenyl-H-benzo[e][1,4]diazepin-2(3H)-one to provide the title compound as a white solid. Calculated for C₂₂H₂₃ClN₂O₃, 398.14; observed (M+H)⁺399.6

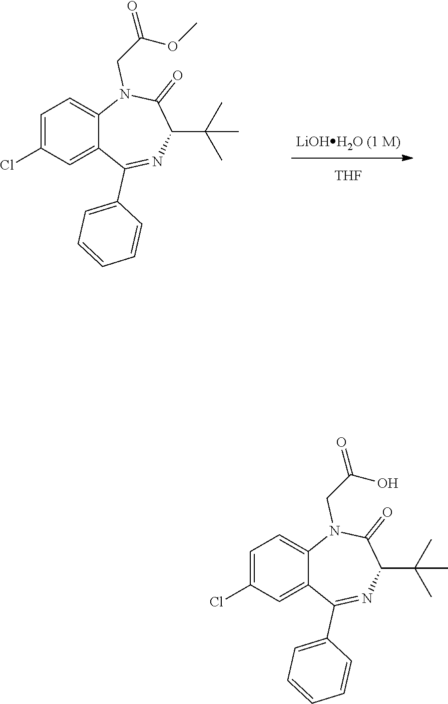 [SMILES: COC(=O)CN1C(=O)[C@H](C(C)(C)C)N=C(c2ccccc2)c2cc(Cl)ccc21 ; CC(C)(C)[C@@H]1N=C(c2ccccc2)c2cc(Cl)ccc2N(CC(=O)O)C1=O ; "THF"]

Step 4: Synthesis of (S)-2-(3-(tert-butyl)-7-chloro-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced (S)-methyl 2-(3-(tert-butyl)-7-chloro-2-oxo-5-phenyl-2,3-dihydro-1H-benzo[e][1,4]diazepin-1-yl)acetate to provide the title compound as a pale yellow solid. ¹H-NMR (300 MHz, CDCl₃) δ (ppm) 7.53-7.63 (m, 2H), 7.49 (d, J=7.03 Hz, 2H), 7.41 (d, J=7.62 Hz, 2H), 7.32 (d, J=8.79 Hz, 1H), 7.17-7.23 (m, 1H), 4.38-4.58 (m, 2H), 3.41 (s, 1H), 1.18 (s, 9H); Calculated for C₂₁H₂₁CIN₂O₃, 384.12; observed (M+H)⁺385.6

### Example 75: Synthesis of (S)-3-(3-(tert-butyl)-7-chloro-2-oxo-5-phenyl-2,3-dihydro-1H-benzo[e][1,4]diazepin-1-yl)propanoic acid

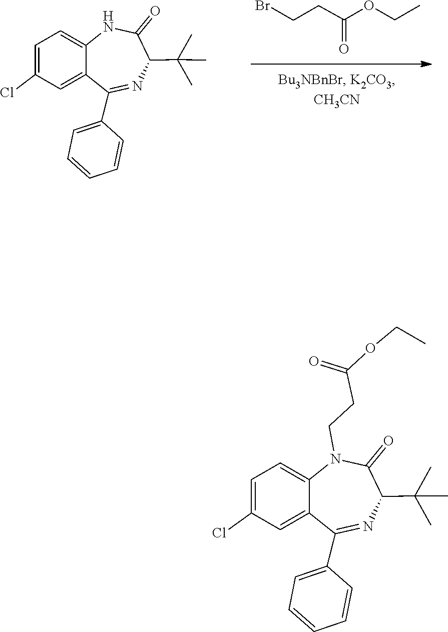

Step 1: Synthesis of (S)-ethyl 3-(3-(tert-butyl)-7-chloro-2-oxo-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-3-(tert-butyl)-7-chloro-5-phenyl-1H-benzo[e][1,4]diazepin-2(3H)-one and methyl 2-bromoacetate was replaced with ethyl 3-bromopropanoate to provide the title compound as a white solid. Calculated for C₂₄H₂₇ClN₂O₃, 426.17; observed (M+H)⁺427.6

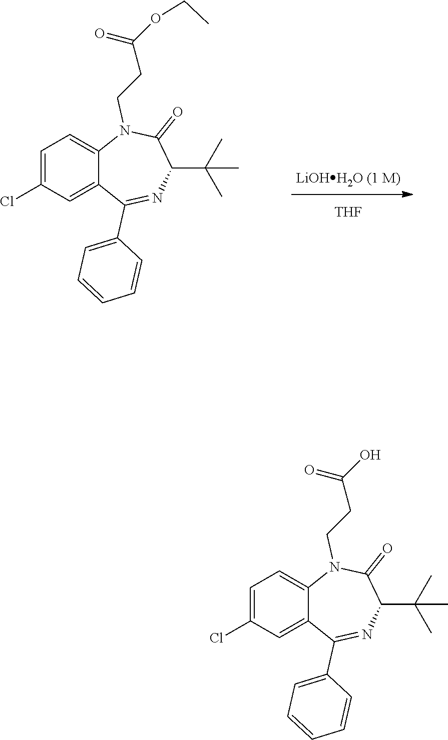 [SMILES: CCOC(=O)CCN1C(=O)[C@H](C(C)(C)C)N=C(c2ccccc2)c2cc(Cl)ccc21 ; CC(C)(C)[C@@H]1N=C(c2ccccc2)c2cc(Cl)ccc2N(CCC(=O)O)C1=O ; "THF"]

Step 2: Synthesis of (S)-3-(3-(tert-butyl)-7-chloro-2-oxo-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(3-(tert-butyl)-7-chloro-2-oxo-5-phenyl-2,3-dihydro-1H-benzo[e][1,4]diazepin-1-yl)propanoate to provide the title compound as pale yellow solid. ¹H-NMR (300 MHz, MeOD) δ (ppm) 7.55-7.60 (m, 4H), 7.45-7.51 (m, 1H), 7.37-7.43 (m, 2H), 7.15 (d, J=1.76 Hz, 1H), 4.40-4.50 (m, 1H), 3.93 (dt, J=13.91, 6.81 Hz, 1H), 3.16 (s, 1H), 2.36-2.47 (m, 2H), 1.13 (s, 9H); Calculated for C₂₂H₂₃ClN₂O₃, 398.14; observed (M+H)⁺399.6.

### Example 76: Synthesis of 2-((S)-7-chloro-3-((R)-1-methoxyethyl)-2-oxo-5-phenyl-2,3-dihydro-1H-benzo[e][1,4]diazepin-1-yl)acetic acid

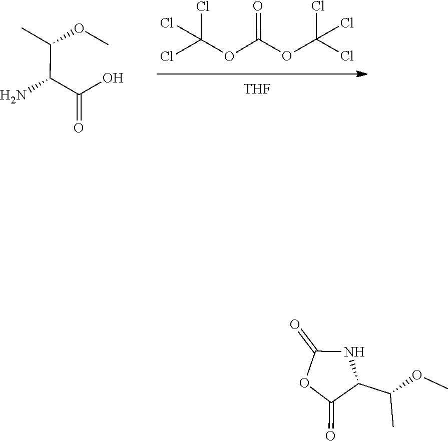

Step 1: Synthesis of (R)-4-((R)-1-methoxyethyl)oxazolidine-2,5-dione: The title compound was prepared according to the procedure of (S)-4-cyclopropyloxazolidine-2,5-dione except that (S)-2-amino-2-cyclopropylacetic acid was replaced with O-methyl-D-threonine to provide the title compound as a white solid.

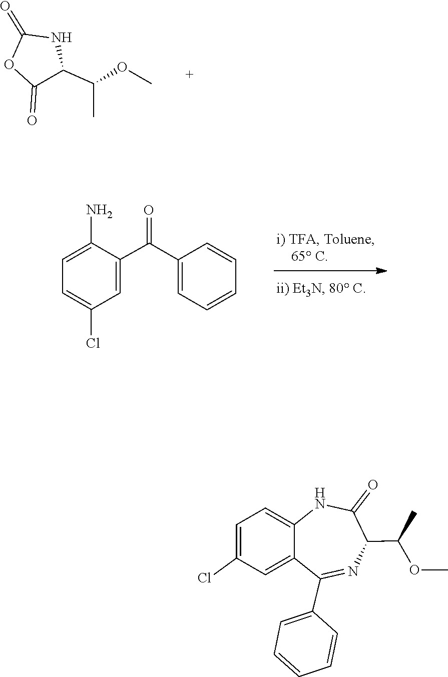 [SMILES: CO[C@H](C)[C@H]1NC(=O)OC1=O ; Nc1ccc(Cl)cc1C(=O)c1ccccc1 ; CO[C@H](C)[C@@H]1N=C(c2ccccc2)c2cc(Cl)ccc2NC1=O]

Step 2: Synthesis of (S)-7-chloro-3-((R)-1-methoxyethyl)-5-phenyl-1H-benzo[e][1,4]diazepin-2(3H)-one: The title compound was prepared according to the procedure of (S)-7-chloro-3-cyclopropyl-5-phenyl-1H-benzo[c][1,4]diazepin-2(3H)-one except that (S)-4-cyclopropyloxazolidine-2,5-dione was replaced with (R)-4-((R)-1-methoxyethyl)oxazolidine-2,5-dione to provide the title compound as a colorless liquid. Calculated for C₁₈H₁₇ClN₂O₂, 328.10; observed (M+H)⁺329.6

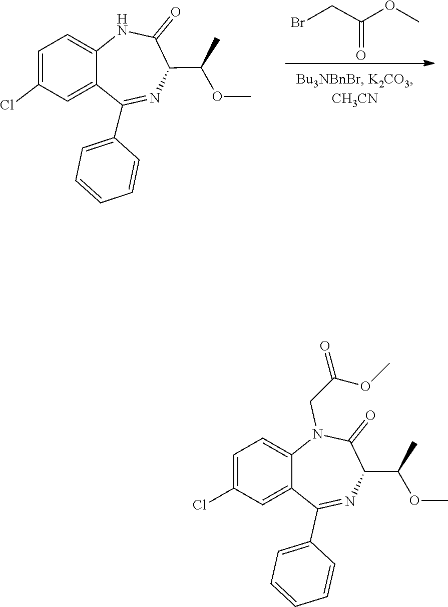

Step 3 Synthesis of methyl 2-((S)-7-chloro-3-((R)-1-methoxyethyl)-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzolel|1,4]diazepin-2(3H)-one was replaced with (S)-7-chloro-3-((R)-1-methoxyethyl)-5-phenyl-1H-benzo[e][1,4]diazepin-2(3H)-one to provide the title compound as a colorless liquid. Calculated for C₂₁H₂₁ClN₂O₄, 400.12; observed (M+H)⁺401.8

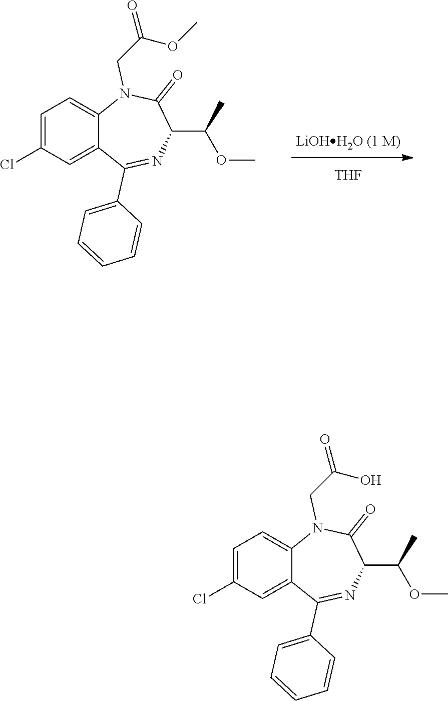 [SMILES: COC(=O)CN1C(=O)[C@H]([C@@H](C)OC)N=C(c2ccccc2)c2cc(Cl)ccc21 ; CO[C@H](C)[C@@H]1N=C(c2ccccc2)c2cc(Cl)ccc2N(CC(=O)O)C1=O ; "THF"]

Step 4: Synthesis of 2-((S)-7-chloro-3-((R)-1-methoxyethyl)-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with methyl 2-((S)-7-chloro-3-((R)-1-methoxyethyl)-2-oxo-5-phenyl-2,3-dihydro-1H-benzo[e][1,4]diazepin-1-yl)acetate to provide the title compound as an Off-white solid. ¹H NMR (300 MHz, CDCl₃): δ (ppm) 7.57-7.65 (m, 4H), 7.48 (d, J=7.62 Hz, 2 H), 7.28-7.36 (m, 2H), 4.58-4.67 (m, 1H), 4.40-4.48 (m, 1H), 4.32-4.39 (m, 1H), 3.69 (d, J=8.79 Hz, 1H), 3.57 (s, 3H), 1.31 (d, J=5.86 Hz, 3H); Calculated for C₂₀H₁₉ClN₂O₄, 386.10; observed (M+H)⁺387.6.

### Example 77: Synthesis of (S)-2-(7-chloro-3-(cyclopropylmethyl)-2-oxo-5-phenyl-2,3-dihydro-1H-benzo[e][1,4]diazepin-1-yl)acetic acid

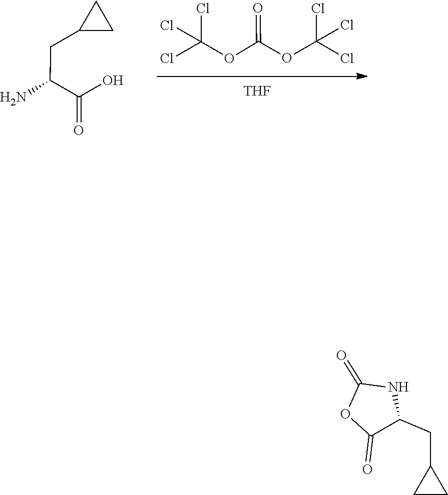 [SMILES: N[C@H](CC1CC1)C(=O)O ; O=C(OC(Cl)(Cl)Cl)OC(Cl)(Cl)Cl ; O=C1N[C@H](CC2CC2)C(=O)O1 ; "THF"]

Step 1: Synthesis of (R)-4-(cyclopropylmethyl)oxazolidine-2,5-dione (35): The title compound was prepared according to the procedure of (S)-4-cyclopropyloxazolidine-2,5-dione except that (S)-2-amino-2-cyclopropylacetic acid was replaced with (R)-2-amino-3-cyclopropylpropanoic acid to provide the title compound as a white solid.

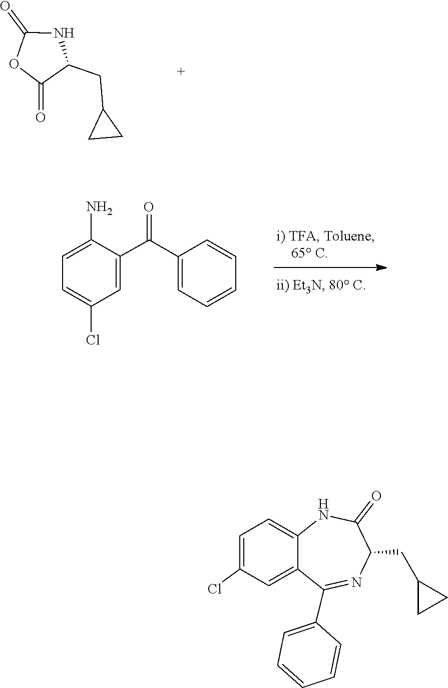 [SMILES: O=C1N[C@H](CC2CC2)C(=O)O1 ; Nc1ccc(Cl)cc1C(=O)c1ccccc1 ; O=C1Nc2ccc(Cl)cc2C(c2ccccc2)=N[C@H]1CC1CC1]

Step 2: Synthesis of (S)-7-chloro-3-(cyclopropylmethyl)-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (R)-4-(cyclopropylmethyl)oxazolidine-2,5-dione to provide the title compound as a white solid. Calculated for C₁₉H₁₇ClN₂O, 324.10; observed (M+H)⁺325.6

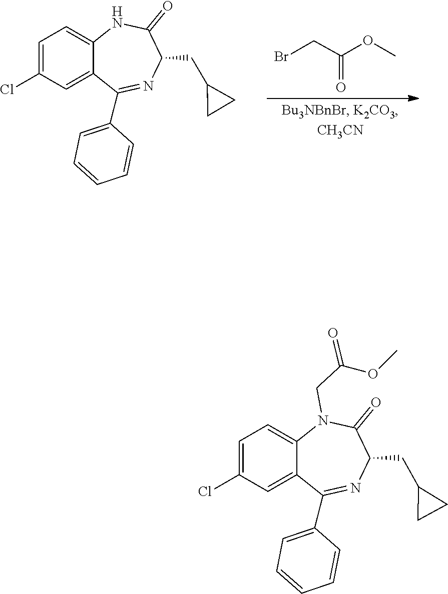

Step 3: Synthesis of (S)-methyl 2-(7-chloro-3-(cyclopropylmethyl)-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-(cyclopropylmethyl)-5-phenyl-1H-benzo[c][1,4]diazepin-2(3H)-one to provide the title compound as a white solid. Calculated for C₂₂H₂₁ClN₂O₃, 396.12; observed (M+H)⁺397.6

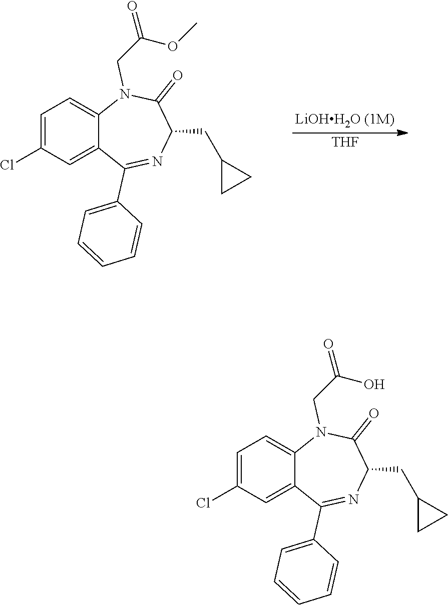 [SMILES: COC(=O)CN1C(=O)[C@H](CC2CC2)N=C(c2ccccc2)c2cc(Cl)ccc21 ; O=C(O)CN1C(=O)[C@H](CC2CC2)N=C(c2ccccc2)c2cc(Cl)ccc21 ; "THF"]

Step 4: Synthesis of (S)-2-(7-chloro-3-(cyclopropylmethyl)-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(7-chloro-3-(cyclopropylmethyl)-2-oxo-5-phenyl-2,3-dihydro-1H-benzo[e][1,4]diazepin-1-yl)acetate to provide the title compound as an Off-white solid. ¹H NMR (300 MHz, CDCl₃): δ (ppm) 7.70 (dd, J=5.86, 1.76 Hz, 1H), 7.57-7.63 (m, 3H), 7.50 (d, J=6.44 Hz, 2H), 7.43 (dd, J=5.86, 2.34 Hz, 1H), 7.32 (dd, J=6.15, 2.05 Hz, 1H), 4.63-4.76 (m, 1H), 4.42-4.52 (m, 1H), 3.86-3.93 (m, 1H), 2.16 (dd, J=12.89, 7.03 Hz, 2H), 0.82-0.93 (m, 1H), 0.45-0.54 (m, 2H), 0.19-0.27 (m, 1H), 0.10-0.17 (m, 1H); Calculated for C₂₁H₁₉ClN₂O₃, 382.11; observed (M+H)⁺383.6.

### Example 78: Synthesis of (S)-3-(7-chloro-3-(cyclopropylmethyl)-2-oxo-5-phenyl-2,3-dihydro-1H-benzo[e][1,4]diazepin-1-yl)propanoic acid

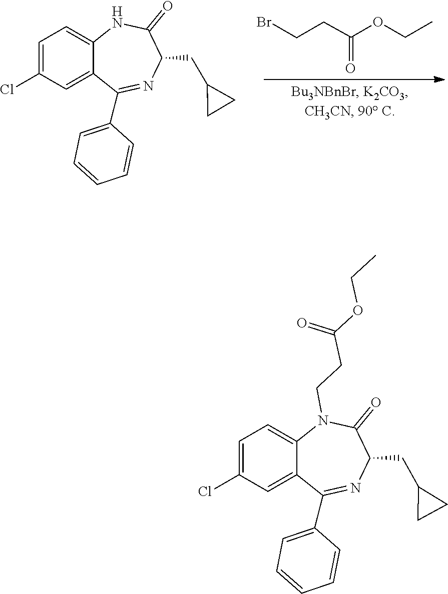

Step 1: Synthesis of (S)-ethyl 3-(7-chloro-3-(cyclopropylmethyl)-2-oxo-5-phenyl-2,3-dihydro-1H-benzo[e][1,4]diazepin-1-yl)propanoate: The title compound was prepared according to the procedure for (S)-2-(7-chloro-3-cyclopropyl-2-oxo-5-phenyl-2,3-dihydro-1H-benzo[c][1,4]diazepin-1-yl)acetic acid except that (S)-methyl 2-(7-chloro-3-cyclopropyl-2-oxo-5-phenyl-2,3-dihydro-1H-benzo[c][1,4]diazepin-1-yl)acetate was replaced with (S)-7-chloro-3-(cyclopropylmethyl)-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₄H₂₅ClN₂O₃, 424.16; observed (M+H)⁺425.6

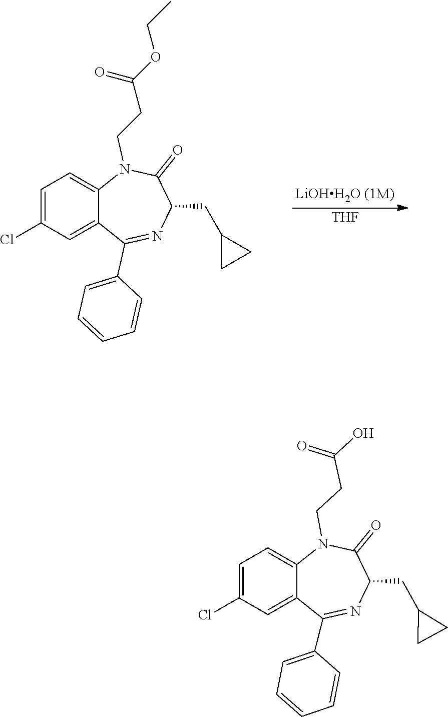 [SMILES: CCOC(=O)CCN1C(=O)[C@H](CC2CC2)N=C(c2ccccc2)c2cc(Cl)ccc21 ; O=C(O)CCN1C(=O)[C@H](CC2CC2)N=C(c2ccccc2)c2cc(Cl)ccc21 ; "THF"]

Step 2: Synthesis of (S)-3-(7-chloro-3-(cyclopropylmethyl)-2-oxo-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(7-chloro-3-(cyclopropylmethyl)-2-oxo-5-phenyl-2,3-dihydro-1H-benzo[e][1,4]diazepin-1-yl)propanoate to provide the title compound as an off-white solid. ¹H NMR (300 MHz, CDCl₃): δ (ppm) 7.74-7.78 (m, 1H), 7.61-7.71 (m, 4H), 7.50-7.56 (m, 2H), 7.27 (d, J=2.34 Hz, 1H), 4.54-4.63 (m, 1H), 4.01-4.10 (m, 1H), 3.83-3.88 (m, 1H), 2.56-2.67 (m, 1H), 2.43-2.54 (m, 1H), 2.03-2.17 (m, 2H), 0.83-0.93 (m, 1H), 0.40-0.55 (m, 2H), 0.08-0.26 (m, 2H); Calculated for C₂₂H₂₁ClN₂O₃, 396.12; observed (M+H)⁺397.6

### Example 79: Synthesis of (S)-2-(7-chloro-3-cyclobutyl-2-oxo-5-phenyl-2,3-dihydro-1H-benzo[e][1,4]diazepin-1-yl)acetic acid

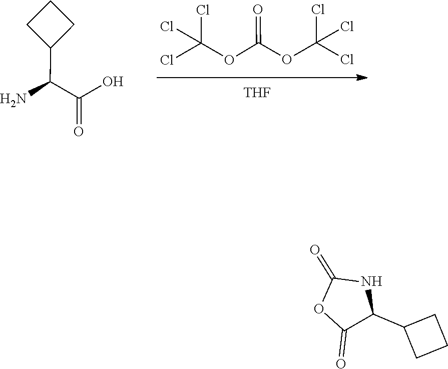 [SMILES: N[C@H](C(=O)O)C1CCC1 ; O=C(OC(Cl)(Cl)Cl)OC(Cl)(Cl)Cl ; O=C1N[C@@H](C2CCC2)C(=O)O1 ; "THF"]

Step 1: Synthesis of (S)-4-cyclobutyloxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-2-cyclobutylacetic acid to provide the title compound as a white solid.

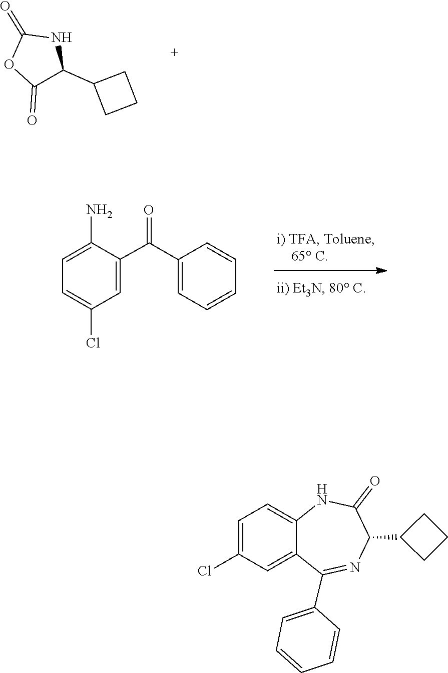 [SMILES: O=C1N[C@@H](C2CCC2)C(=O)O1 ; Nc1ccc(Cl)cc1C(=O)c1ccccc1 ; O=C1Nc2ccc(Cl)cc2C(c2ccccc2)=N[C@H]1C1CCC1]

Step 2: Synthesis of (S)-7-chloro-3-cyclobutyl-5-phenyl-1H-benzo[e][1,4]diazepin-2(3H)-one (41; 66025): The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-cyclobutyloxazolidine-2,5-dione to provide the title compound as an off-white solid. Calculated for C₁₉H₁₇ClN₂O, 324.10; observed (M+H)⁺325.6.

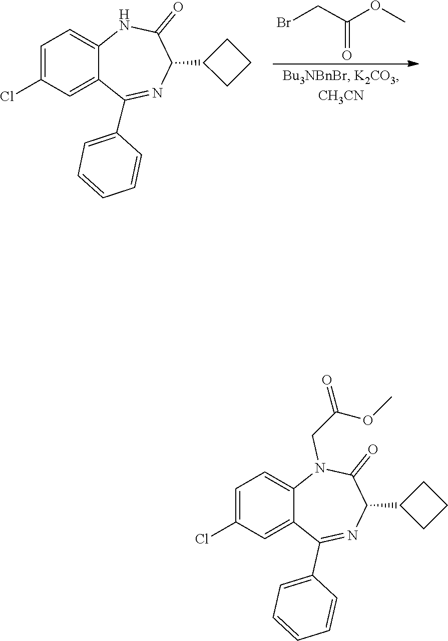

Step 3: Synthesis of (S)-methyl 2-(7-chloro-3-cyclobutyl-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-cyclobutyl-5-phenyl-1H-benzo[e][1,4]diazepin-2(3H)-one to provide the title compound as a white solid. Calculated for C₂₂H₂₁ClN₂O₃, 396.12; observed (M+H)⁺397.6.

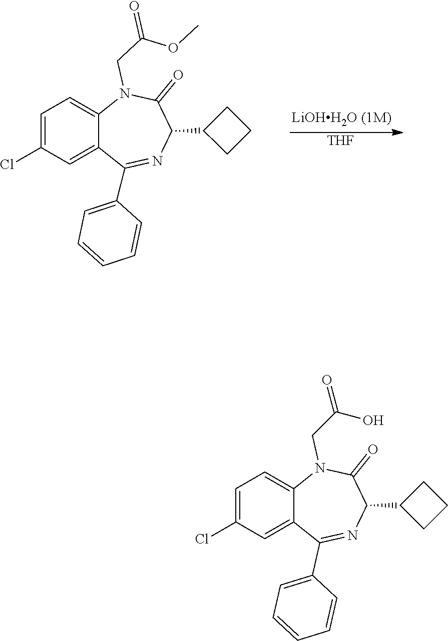 [SMILES: COC(=O)CN1C(=O)[C@H](C2CCC2)N=C(c2ccccc2)c2cc(Cl)ccc21 ; O=C(O)CN1C(=O)[C@H](C2CCC2)N=C(c2ccccc2)c2cc(Cl)ccc21 ; "THF"]

Step 4: Synthesis of (S)-2-(7-chloro-3-cyclobutyl-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1 H-benzo[e][1,4]diazepin-1-yl)acetate was replaced with (S)-methyl 2-(7-chloro-3-cyclobutyl-2-oxo-5-phenyl-2,3-dihydro-1H-benzo[e][1,4]diazepin-1-yl)acetate to provide the title compound as an off-white solid. 1H NMR (300 MHz, CDCl₃): δ (ppm) 7.72 (dd, J=9.08, 2.05 Hz, 1H), 7.65 (d, J=7.03 Hz, 1H), 7.58 (d, J=7.62 Hz, 2H), 7.41-7.52 (m, 3H), 7.30 (d, J=2.34 Hz, 1H), 4.61-4.71 (m, 1 H), 4.41-4.52 (m, 1H), 3.85 (d, J=10.54 Hz, 1H), 2.40-2.48 (m, 1H), 2.23-2.31 (m, 1H), 2.01-2.08 (m, 1H), 1.77-1.95 (m, 3H), 1.62-1.70 (m, 1H); Calculated for C₂₁H₁₉ClN₂O₃, 382.11; observed (M+H)⁺383.6

### Example 80: Synthesis of (S)-3-(7-chloro-3-cyclobutyl-2-oxo-5-phenyl-2,3-dihydro-1H-benzo[e][1,4]diazepin-1-yl)propanoic acid

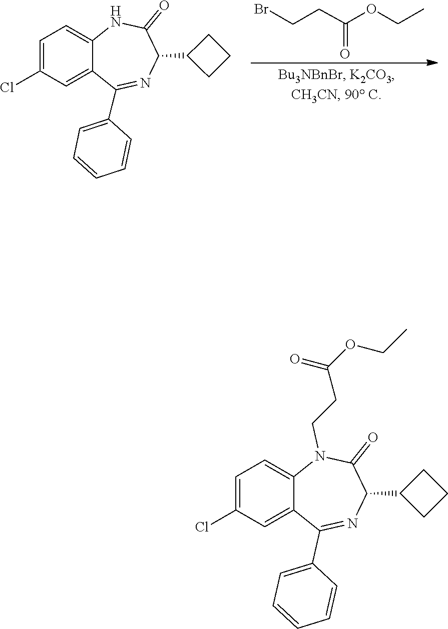

Step 1: Synthesis of (S)-ethyl 3-(7-chloro-3-cyclobutyl-2-oxo-5-phenyl-2,3-dihydro-1H-benzo[el[1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-cyclobutyl-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₄H₂₅ClN₂O₃, 424.16; observed (M+H)⁺425.6

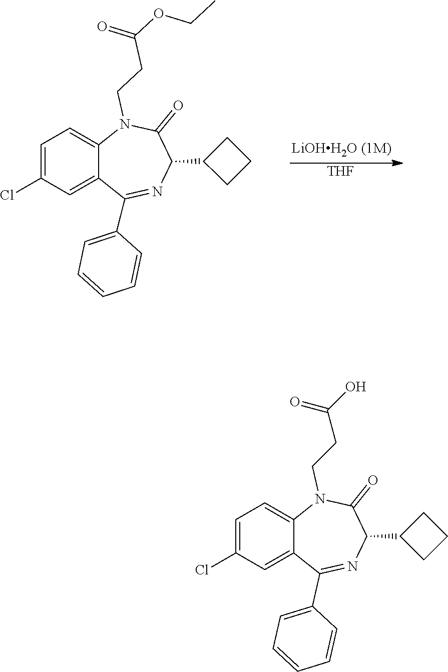 [SMILES: CCOC(=O)CCN1C(=O)[C@H](C2CCC2)N=C(c2ccccc2)c2cc(Cl)ccc21 ; O=C(O)CCN1C(=O)[C@H](C2CCC2)N=C(c2ccccc2)c2cc(Cl)ccc21 ; "THF"]

Step 2: Synthesis of (S)-3-(7-chloro-3-cyclobutyl-2-oxo-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[c][1,4]diazepin-1-yl)acetate was replaced with (S)-ethyl 3-(7-chloro-3-cyclobutyl-2-oxo-5-phenyl-2,3-dihydro-1H-benzo[e][1,4]diazepin-1-yl)propanoate to provide the title compound as an Off-white solid. ¹H NMR (300 MHz, CDCl₃): δ (ppm) 7.81 (dd, J=8.79, 2.34 Hz, 1H), 7.68-7.75 (m, 1H), 7.52-7.63 (m, 5H), 7.34 (d, J=2.34 Hz, 1H), 4.77-4.89 (m, 1H), 3.84-3.92 (m, 1H), 3.81 (d, J=10.54 Hz, 1H), 3.42-3.55 (m, 1H), 2.58-2.69 (m, 1H), 2.46-2.54 (m, 1H), 2.29-2.42 (m, 2H), 2.03-2.16 (m, 1H), 1.77-1.96 (m, 2H), 1.61-1.73 (m, 1H); Calculated for C₂₂H₂₁CN₂O₃, 396.12; observed (M+H)⁺397.6.

### Example 81: Synthesis of (S)-2-(7-chloro-3-cyclopentyl-2-oxo-5-phenyl-2,3-dihydro-1H-benzo[e][1,4]diazepin-1-yl)acetic acid

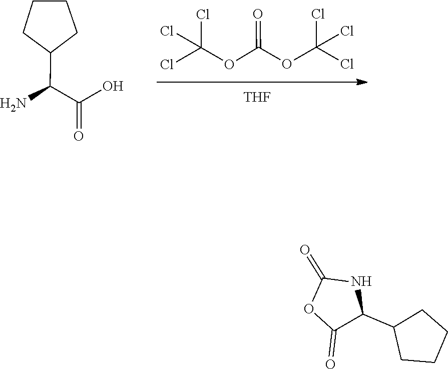 [SMILES: N[C@H](C(=O)O)C1CCCC1 ; O=C(OC(Cl)(Cl)Cl)OC(Cl)(Cl)Cl ; O=C1N[C@@H](C2CCCC2)C(=O)O1 ; "THF"]

Step 1: Synthesis of (S)-4-cyclopentyloxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-2-cyclopentylacetic acid to provide the title compound as a white solid.

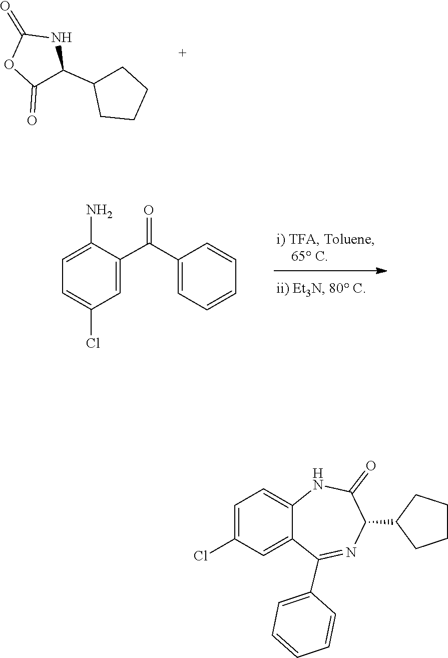 [SMILES: O=C1N[C@@H](C2CCCC2)C(=O)O1 ; Nc1ccc(Cl)cc1C(=O)c1ccccc1 ; O=C1Nc2ccc(Cl)cc2C(c2ccccc2)=N[C@H]1C1CCCC1]

Step 2: Synthesis of (S)-7-chloro-3-cyclopentyl-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-cyclopentyloxazolidine-2,5-dione to provide the title compound as a white solid. Calculated for C₂₀H₁₉ClN₂O, 338.12; observed (M+H)⁺339.6.

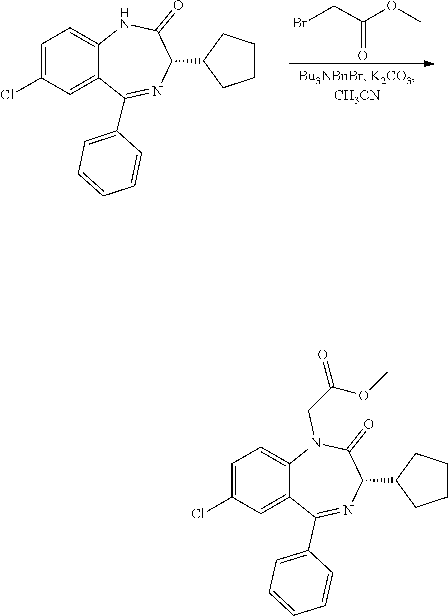

Step 3: Synthesis of (S)-methyl 2-(7-chloro-3-cyclopentyl-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-cyclopentyl-5-phenyl-1H-benzo[e][1,4]diazepin-2(3H)-one to provide the title as a colorless gum. Calculated for C₂₃H₂₃ClN₂O₃, 410.14; observed (M+H)⁺411.6

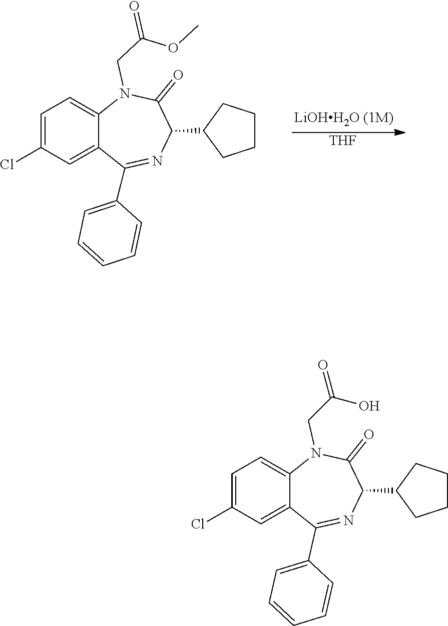 [SMILES: COC(=O)CN1C(=O)[C@H](C2CCCC2)N=C(c2ccccc2)c2cc(Cl)ccc21 ; O=C(O)CN1C(=O)[C@H](C2CCCC2)N=C(c2ccccc2)c2cc(Cl)ccc21 ; "THF"]

Step 4: Synthesis of (S)-2-(7-chloro-3-cyclopentyl-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(7-chloro-3-cyclopentyl-2-oxo-5-phenyl-2,3-dihydro-1H-benzo[e][1,4]diazepin-1-yl)acetate to provide the title compound as an off-white solid. ¹H NMR (300 MHz, CDCl₃): δ (ppm) 7.72-7.76 (m, 1H), 7.68 (d, J=7.62 Hz, 1H), 7.52-7.61 (m, 4H), 7.44 (d, J=8.79 Hz, 1H), 7.30 (d, J=2.34 Hz, 1H), 4.45-4.71 (m, 2H), 3.59 (d, J=10.54 Hz, 1H), 2.98-3.10 (m, 1H), 2.23-2.31 (m, 1H), 2.05-2.14 (m, 1H), 1.58-1.66 (m, 4H), 1.29-1.36 (m, 1H), 1.03-1.11 (m, 1H); Calculated for C₂₂H₂₁ClN₂O₃, 396.12; observed (M+H)⁺397.6.

### Example 82: Synthesis of (S)-3-(7-chloro-3-cyclopentyl-2-oxo-5-phenyl-2,3-dihydro-1H-benzo[e][1,4]diazepin-1-yl)propanoic acid

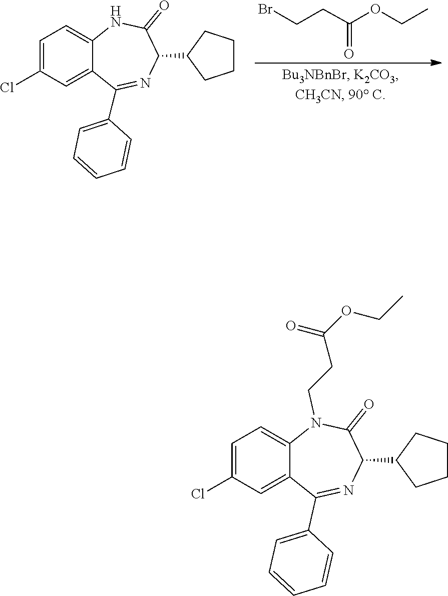

Step 1: Synthesis of (S)-ethyl 3-(7-chloro-3-cyclopentyl-2-oxo-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-cyclopentyl-5-phenyl-1H-benzo[e][1,4]diazepin-2(3H)-one and methyl 2-bromoacetate was replaced with ethyl 3-bromopropanoate to provide the title compound as a colorless gum. Calculated for C₂₅H₂₇CIN₂O₃, 438.17; observed (M+H)⁺439.6

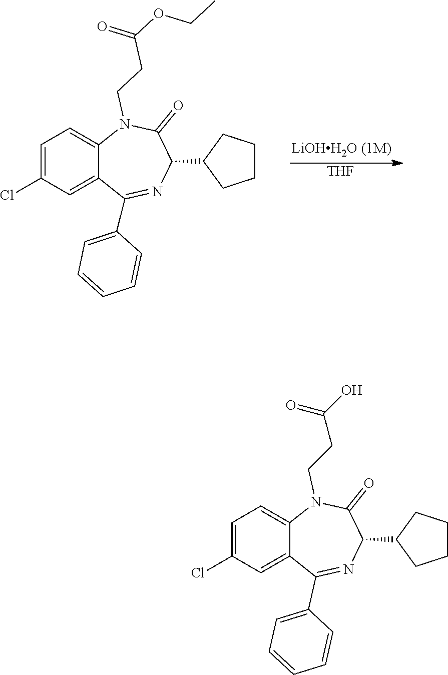 [SMILES: CCOC(=O)CCN1C(=O)[C@H](C2CCCC2)N=C(c2ccccc2)c2cc(Cl)ccc21 ; O=C(O)CCN1C(=O)[C@H](C2CCCC2)N=C(c2ccccc2)c2cc(Cl)ccc21 ; "THF"]

Step 2: Synthesis of (S)-3-(7-chloro-3-cyclopentyl-2-oxo-5-phenyl-2,3-dihydro-1H-benzo[el[1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with(S)-ethyl 3-(7-chloro-3-cyclopentyl-2-oxo-5-phenyl-2,3-dihydro-1H-benzo[e][1,4]diazepin-1-yl)propanoate to provide the title compound as an off-white solid. ¹H NMR (300 MHz, CDCl₃): δ (ppm) 7.78 (d, J=8.20 Hz, 1H), 7.71 (t, J=6.74 Hz, 1H), 7.52-7.61 (m, 5H), 7.26-7.34 (m, 1H), 4.81-4.92 (m, 1H), 3.83-3.94 (m, 1H), 3.48 (d, J=11.13 Hz, 1H), 3.01-3.14 (m, 1H), 2.57-2.68 (m, 1H), 2.28-2.38 (m, 2H), 2.14-2.24 (m, 1H), 1.54-1.70 (m, 4H), 1.25-1.35 (m, 1H), 0.99-1.12 (m, 1H); Calculated for C₂₃H₂₃ClN₂O₃, 410.14; observed (M+H)⁺411.6.

### Example 83: Synthesis of 2-(7-chloro-3-(2-fluoropropan-2-yl)-2-oxo-5-phenyl-2,3-dihydro-1H-benzo[e][1,4]diazepin-1-yl)acetic acid

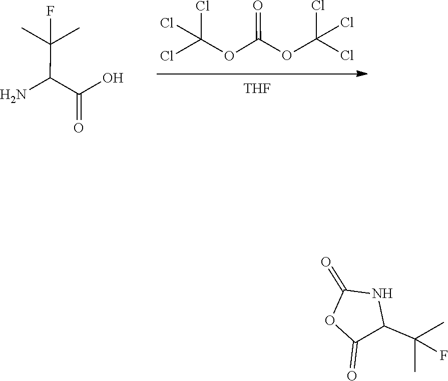

Step 1: Synthesis of 4-(2-fluoropropan-2-yl)oxazolidine-2,5-dione: The title compound was prepared according to the procedure of (S)-4-cyclopropyloxazolidine-2,5-dione except that (S)-2-amino-2-cyclopropylacetic acid was replaced with 2-amino-3-fluoro-3-methylbutanoic acid to provide the title compound as a white solid.

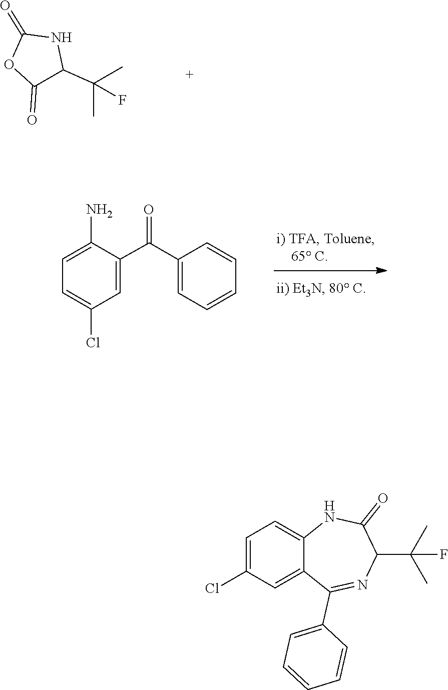 [SMILES: CC(C)(F)C1NC(=O)OC1=O ; Nc1ccc(Cl)cc1C(=O)c1ccccc1 ; CC(C)(F)C1N=C(c2ccccc2)c2cc(Cl)ccc2NC1=O]

Step 2: Synthesis of 7-chloro-3-(2-fluoropropan-2-yl)-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with 4-(2-fluoropropan-2-yl)oxazolidine-2,5-dione to provide the title compound as a white gummy solid. Calculated for C₁₈H₁₆ClFN₂O, 330.09; observed (M+H)⁺331.6

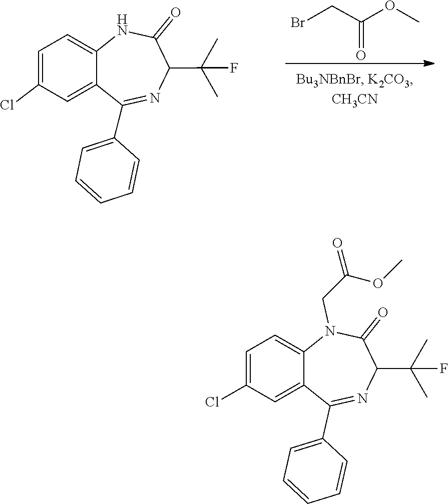

Step 3: Synthesis of methyl 2-(7-chloro-3-(2-fluoropropan-2-yl)-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with 7-chloro-3-(2-fluoropropan-2-yl)-5-phenyl-1H-benzo[e][1,4]diazepin-2(3H)-one to provide the title compound as a white solid. Calculated for C₂₁H₂₀ClFN₂O₃, 402.11; observed (M+H)⁺403.5

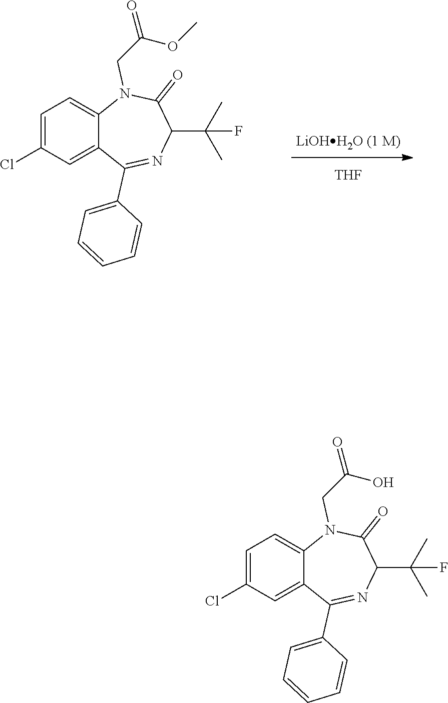 [SMILES: COC(=O)CN1C(=O)C(C(C)(C)F)N=C(c2ccccc2)c2cc(Cl)ccc21 ; CC(C)(F)C1N=C(c2ccccc2)c2cc(Cl)ccc2N(CC(=O)O)C1=O ; "THF"]

Step 4: Synthesis of 2-(7-chloro-3-(2-fluoropropan-2-yl)-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with methyl 2-(7-chloro-3-(2-fluoropropan-2-yl)-2-oxo-5-phenyl-2,3-dihydro-1H-benzo[e][1,4]diazepin-1-yl)acetate to provide the ttitle compound as an off-white solid; ¹H NMR (300 MHz, CDCl₃): δ (ppm) 7.62 (d, J=7.03 Hz, 2H), 7.48-7.57 (m, 2H), 7.44 (d, J=7.62 Hz, 2H), 7.26-7.34 (m, 2H), 4.55 (s, 2H), 3.79 (d, J=12.30 Hz, 1H), 1.60-1.81 (m, 6H); Calculated for C₂₀H₁₈ClFN₂O₃, 388.10; observed (M+H)⁺389.6

### Example 84: Synthesis of (S)-2-(7-chloro-3-(2-hydroxypropan-2-yl)-2-oxo-5-phenyl-2,3-dihydro-1H-benzo[e][1,4]diazepin-1-yl)acetic acid

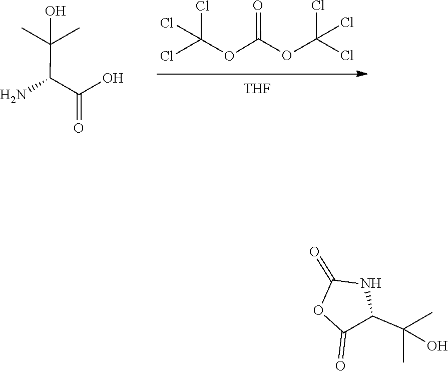

Step 1: Synthesis of (R)-4-(2-hydroxypropan-2-yl)oxazolidine-2,5-dione: The title compound was prepared according to the procedure of (S)-4-cyclopropyloxazolidine-2,5-dione except that (S)-2-amino-2-cyclopropylacetic acid was replaced with (R)-2-amino-3-hydroxy-3-methylbutanoic acid to provide the title compound as a white solid.

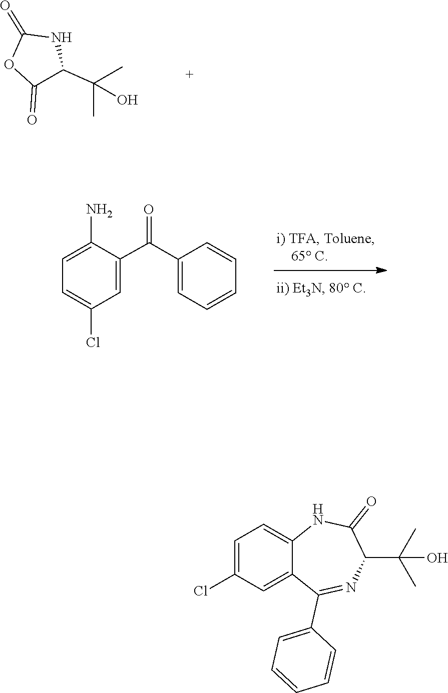 [SMILES: CC(C)(O)[C@H]1NC(=O)OC1=O ; Nc1ccc(Cl)cc1C(=O)c1ccccc1 ; CC(C)(O)[C@@H]1N=C(c2ccccc2)c2cc(Cl)ccc2NC1=O]

Step 2: Synthesis of (S)-7-chloro-3-(2-hydroxypropan-2-yl)-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (R)-4-(2-hydroxypropan-2-yl)oxazolidine-2,5-dione to provide the title compound as a colorless liquid. Calculated for C₁₈H₁₇ClN₂O₂, 328.10; observed (M+H)⁺329.5

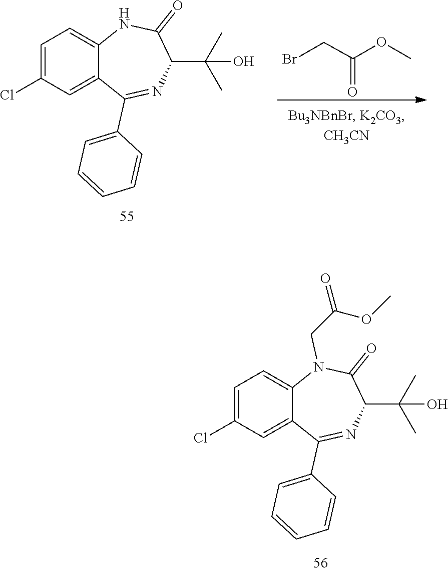

Step 3: Synthesis of (S)-methyl 2-(7-chloro-3-(2-hydroxypropan-2-yl)-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo|e111,41diazepin-1-yl)acetate except that (S)-7-chloro-3-cyclopropyl-5-phenyl-1H-benzo[e][1,4]diazepin-2(3H)-one was replaced with (S)-7-chloro-3-(2-hydroxypropan-2-yl)-5-phenyl-1H-benzo[e][1,4]diazepin-2(3H)-one to provide the title compound as a colorless liquid. Calculated for C₂₁H₂₁ClN₂O₄, 400.12; observed (M+H)⁺401.6

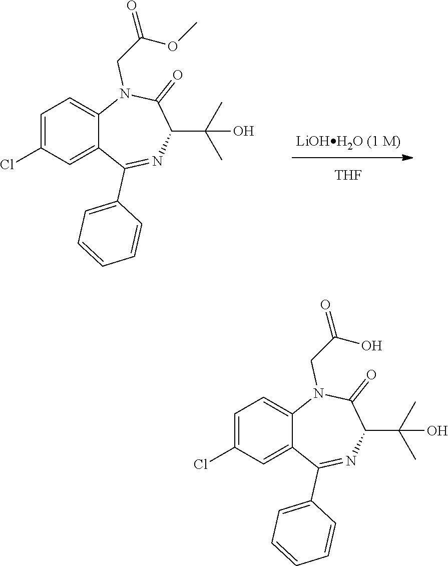 [SMILES: COC(=O)CN1C(=O)[C@H](C(C)(C)O)N=C(c2ccccc2)c2cc(Cl)ccc21 ; CC(C)(O)[C@@H]1N=C(c2ccccc2)c2cc(Cl)ccc2N(CC(=O)O)C1=O ; "THF"]

Step 4: Synthesis of (S)-2-(7-chloro-3-(2-hydroxypropan-2-yl)-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with(S)-methyl 2-(7-chloro-3-(2-hydroxypropan-2-yl)-2-oxo-5-phenyl-2,3-dihydro-1H-benzo[e][1,4]diazepin-1-yl)acetate to provide the title compound as an pale yellow solid. ¹H NMR (300 MHz, CDCl₃): δ (ppm) 7.53-7.61 (m, 4H), 7.42-7.48 (m, 2 H), 7.29-7.35 (m, 2H), 4.41-4.61 (m, 2H), 3.59 (s, 1H), 1.44 (s, 3H), 1.42 (s, 3H); Calculated for C₂₀H₁₉ClN₂O₄, 386.10; observed (M+H)⁺387.6.

### Example 85: Synthesis of (S)-3-(7-chloro-3-(2-hydroxypropan-2-yl)-2-oxo-5-phenyl-2,3-dihydro-1H-benzo[e][1,4]diazepin-1-yl)propanoic acid

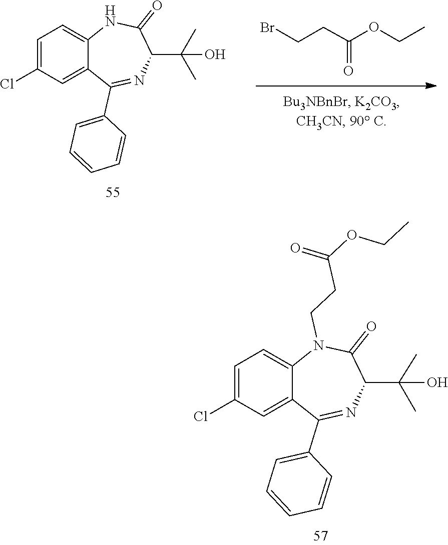

Step 1: Synthesis of (S)-ethyl 3-(7-chloro-3-(2-hydroxypropan-2-yl)-2-oxo-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-(2-hydroxypropan-2-yl)-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₃H₂₅ClN₂O₄, 428.15; observed (M+H)⁺429.7

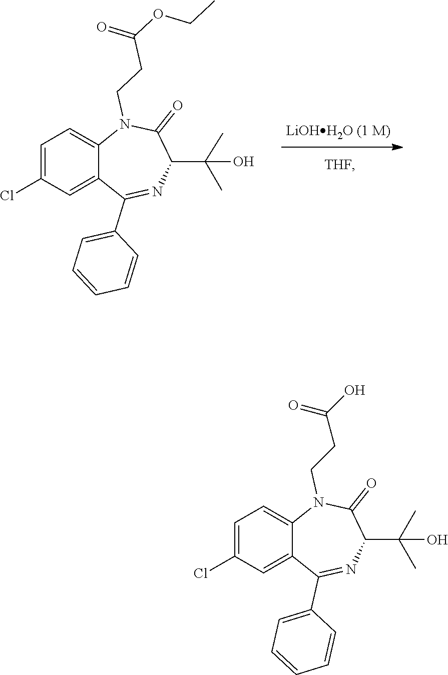 [SMILES: CCOC(=O)CCN1C(=O)[C@H](C(C)(C)O)N=C(c2ccccc2)c2cc(Cl)ccc21 ; "THF," ; CC(C)(O)[C@@H]1N=C(c2ccccc2)c2cc(Cl)ccc2N(CCC(=O)O)C1=O]

Step 2: Synthesis of (S)-3-(7-chloro-3-(2-hydroxypropan-2-yl)-2-oxo-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(7-chloro-3-(2-hydroxypropan-2-yl)-2-oxo-5-phenyl-2,3-dihydro-1H-benzo[e][1,4]diazepin-1-yl)propanoate to provide the title compound as a pale yellow solid. ¹H NMR (300 MHz, MeOD): δ (ppm) 7.59-7.64 (m, 4H), 7.37-7.48 (m, 3H), 7.18 (s, 1H), 4.43-4.54 (m, 1H), 3.90-3.99 (m, 1H), 3.39 (s, 1H), 2.40-2.50 (m, 2H), 1.39 (s, 3H), 1.37 (s, 3H); Calculated for C₂₁H₂₁ClN₂O₄, 400.12; observed (M+H)⁺401.6.

### Example 86: Synthesis of 2-(7-chloro-2-oxo-5-phenyl-3-(2,2,2-trifluoroethyl)-2,3-dihydro-1H-benzo[e][1,4]diazepin-1-yl)acetic acid

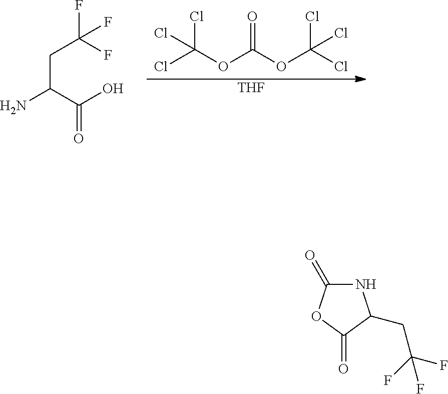 [SMILES: NC(CC(F)(F)F)C(=O)O ; O=C(OC(Cl)(Cl)Cl)OC(Cl)(Cl)Cl ; O=C1NC(CC(F)(F)F)C(=O)O1 ; "THF"]

Step 1: Synthesis of 4-(2,2,2-trifluoroethyl)oxazolidine-2,5-dione: The title compound was prepared according to the procedure of (S)-4-cyclopropyloxazolidine-2,5-dione except that (S)-2-amino-2-cyclopropylacetic acid was replaced with 2-amino-4,4,4-trifluorobutanoic acid to provide the title compound as a white solid.

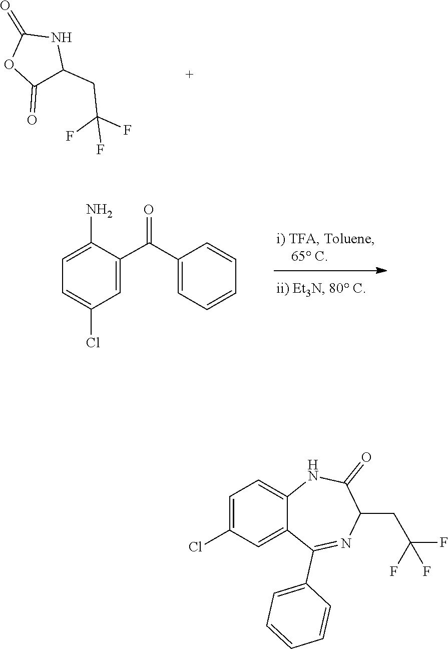 [SMILES: O=C1NC(CC(F)(F)F)C(=O)O1 ; Nc1ccc(Cl)cc1C(=O)c1ccccc1 ; O=C1Nc2ccc(Cl)cc2C(c2ccccc2)=NC1CC(F)(F)F]

Step 2: Synthesis of 7-chloro-5-phenyl-3-(2,2,2-trifluoroethyl)-1H-benzo[e][1,4]diazepin-2(3H)-one: The title compound was prepared according to the procedure of (S)-7-chloro-3-cyclopropyl-5-phenyl-1H-benzo[e][,4]diazepin-2(3H)-one except that (S)-4-cyclopropyloxazolidine-2,5-dione was replaced with 4-(2,2,2-trifluoroethyl)oxazolidine-2,5-dione to provide the title compound as a white solid. Calculated for C₁₇H₁₂ClF₃N₂O, 352.06; observed (M+H)⁺353.6

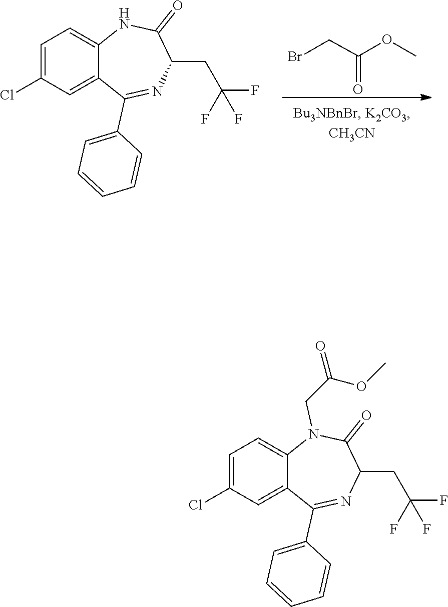

Step 3: Synthesis of methyl 2-(7-chloro-2-oxo-5-phenyl-3-(2,2,2-trifluoroethyl)-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with 7-chloro-5-phenyl-3-(2,2,2-trifluoroethyl)-1H-benzo[c][1,4]diazepin-2(3H)-one to provide the title compound as a white solid. Calculated for C₂₀H₁₆CIF₃N₂O₃, 424.08; observed (M+H)⁺425.6

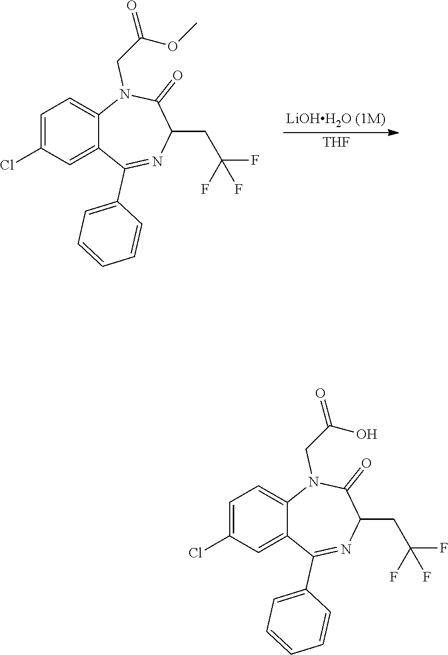 [SMILES: COC(=O)CN1C(=O)C(CC(F)(F)F)N=C(c2ccccc2)c2cc(Cl)ccc21 ; O=C(O)CN1C(=O)C(CC(F)(F)F)N=C(c2ccccc2)c2cc(Cl)ccc21 ; "THF"]

Step 4: Synthesis of 2-(7-chloro-2-oxo-5-phenyl-3-(2,2,2-trifluoroethyl)-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with methyl 2-(7-chloro-2-oxo-5-phenyl-3-(2,2,2-trifluoroethyl)-2,3-dihydro-1H-benzo[e][1,4]diazepin-1-yl)acetate to provide the title compound as an off-white solid. ¹H-NMR (300 MHz, CDCl₃) δ (ppm) 7.88-7.98 (m, 2H), 7.82 (d, J=1.76 Hz, 2H), 7.76 (d, J=7.03 Hz, 2H), 7.58-7.69 (m, 2H), 4.84 (q, J=18.16 Hz, 2H), 4.39-4.49 (m, 1H), 3.38-3.59 (m, 2H); Calculated for C₁₉H₁₄ClF₃N₂O₃, 410.06; observed (M+H)⁺411.6

### Example 87: Synthesis of 3-(7-chloro-2-oxo-5-phenyl-3-(2,2,2-trifluoroethyl)-2,3-dihydro-1H-benzo[e][1,4]diazepin-1-yl)propanoic acid

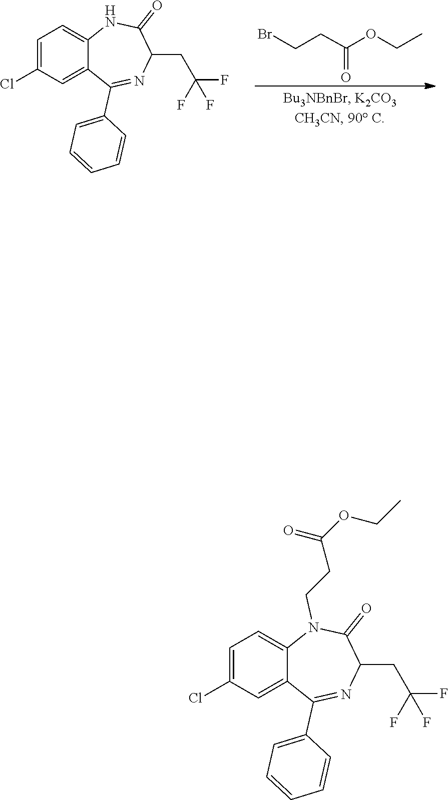

Step 1: Synthesis of ethyl 3-(7-chloro-2-oxo-5-phenyl-3-(2,2,2-trifluoroethyl)-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with 7-chloro-5-phenyl-3-(2,2,2-trifluoroethyl)-1H-benzo[e][1,4]diazepin-2(3H)-one and methyl 2-bromoacetate was replaced with ethyl 3 -bromopropanoate to provide the title compound as a colorless liquid. Calculated for C₂₂H₂₀ClF₃N₂O₃, 452.11; observed (M+H)⁺453.6

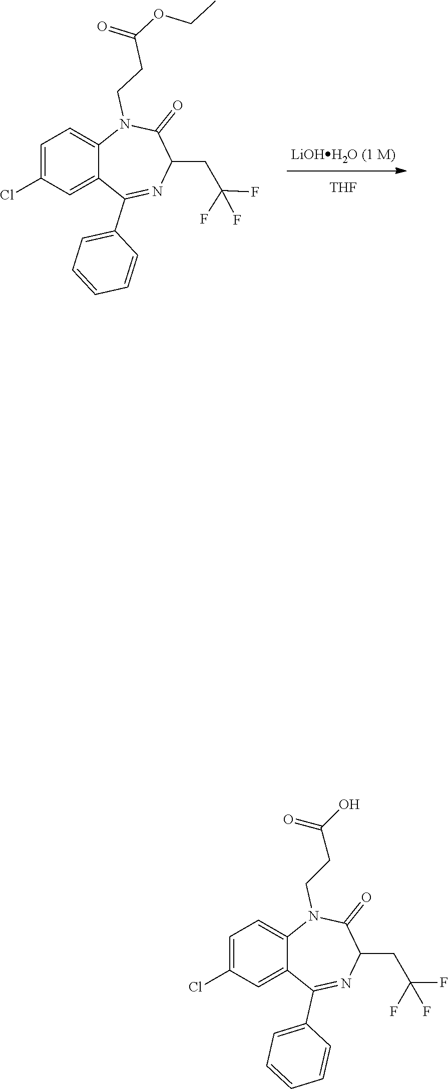 [SMILES: "THF" ; CCOC(=O)CCN1C(=O)C(CC(F)(F)F)N=C(c2ccccc2)c2cc(Cl)ccc21 ; O=C(O)CCN1C(=O)C(CC(F)(F)F)N=C(c2ccccc2)c2cc(Cl)ccc21]

Step 2: Synthesis of 3-(7-chloro-2-oxo-5-phenyl-3-(2,2,2-trifluoroethyl)-2,3-dihydro-1H-benzole 1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with ethyl 3-(7-chloro-2-oxo-5-phenyl-3-(2,2,2-trifluoroethyl)-2,3-dihydro-1H-benzo[e][1,4]diazepin-1-yl)propanoate to provide the title compound as a pale yellow solid. ¹H-NMR (300 MHz, MeOD) δ (ppm) 7.39-7.46 (m, 2H), 7.33-7.39 (m, 2H), 7.28-7.33 (m, 1H), 7.19-7.27 (m, 2H), 7.01 (d, J=2.34 Hz, 1H), 4.27-4.37 (m, 1H), 3.76-3.86 (m, 1H), 3.68-3.75 (m, 1H), 2.79-2.93 (m, 2H), 2.18-2.38 (m, 2H); Calculated for C₂₀H₁₆ClF₃N₂O₃, 424.08; observed (M+H)⁺425.6.

### Example 88: Synthesis of 2-((S)-7-bromo-3-((S)-sec-butyl)-2-oxo-5-phenyl-2,3-dihydro-1H-benzo[e][1,4]diazepin-1-yl)acetic acid

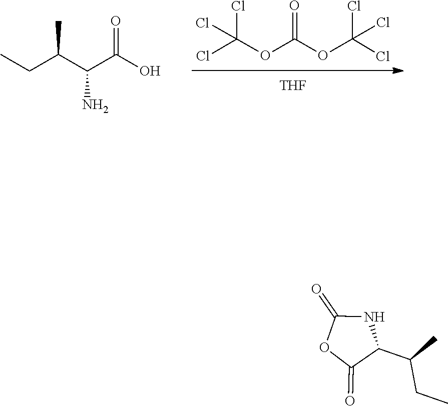

Step 1: Synthesis of (R)-4-((S)-sec-butyl)oxazolidine-2,5-dione: The title compound was prepared according to the procedure of (S)-4-cyclopropyloxazolidine-2,5-dione except that (S)-2-amino-2-cyclopropylacetic acid was replaced with D-isoleucine to provide the title compound as a white solid.

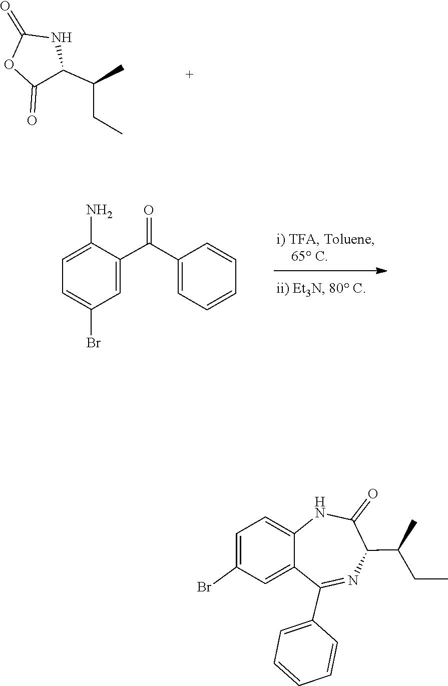 [SMILES: CC[C@H](C)[C@H]1NC(=O)OC1=O ; Nc1ccc(Br)cc1C(=O)c1ccccc1 ; CC[C@H](C)[C@@H]1N=C(c2ccccc2)c2cc(Br)ccc2NC1=O]

Step 2: (S)-7-bromo-3-((S)-sec-butyl)-5-phenyl-1H-benzo[c][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (R)-4-((S)-sec-butyl)oxazolidine-2,5-dione and 2-amino-5-chlorobenzophenone was replaced with 2-amino-5-bromobenzophenone to provide the title compound as a white solid. Calculated for C₁₉H₁₉BrN₂O, 370.07; observed (M+H)⁺371.6.

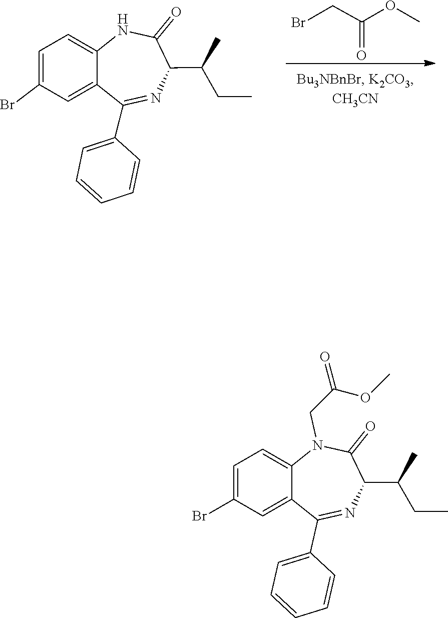

Step 3: Synthesis of methyl 2-((S)-7-bromo-3-((S)-sec-butyl)-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with(S)-7-bromo-3-((S)-sec-butyl)-5-phenyl-1H-benzo[e][1,4]diazepin-2(3H)-one to provide the title compound as a white solid. Calculated for C₂₂H₂₃BrN₂O₃, 442.09; observed (M+H)⁺443.7

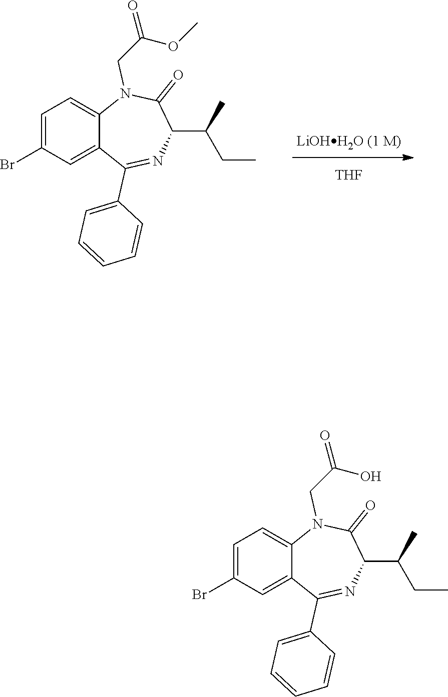 [SMILES: CC[C@H](C)[C@@H]1N=C(c2ccccc2)c2cc(Br)ccc2N(CC(=O)OC)C1=O ; CC[C@H](C)[C@@H]1N=C(c2ccccc2)c2cc(Br)ccc2N(CC(=O)O)C1=O ; "THF"]

Step 4: Synthesis of 2-((S)-7-bromo-3-((S)-sec-butyl)-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with methyl 2-((S)-7-bromo-3-((S)-sec-butyl)-2-oxo-5-phenyl-2,3-dihydro-1H-benzo[e][1,4]diazepin-1-yl)acetate to provide the title compound as an off-white solid. ¹H NMR (300 MHz, CDCl₃): δ (ppm) 7.77 (d, J=9.38 Hz, 1H), 7.42-7.62 (m, 7H), 4.43 -4.69 (m, 2H), 3.37 (d, J=9.96 Hz, 1H), 2.57-2.68 (m, 1H), 1.95-2.06 (m, 1H), 1.14-1.23 (m, 1 H), 0.94-1.03 (m, 6H); Calculated for C₂₁H₂₁BrN₂O₃, 428.07; observed (M+H)⁺429.6.

### Example 89: Synthesis of (S)-3-(7-iodo-2-oxo-3-(pentan-3-yl)-5-phenyl-2,3-dihydro-1H-benzo[e][1,4]diazepin-1-yl)propanoic acid

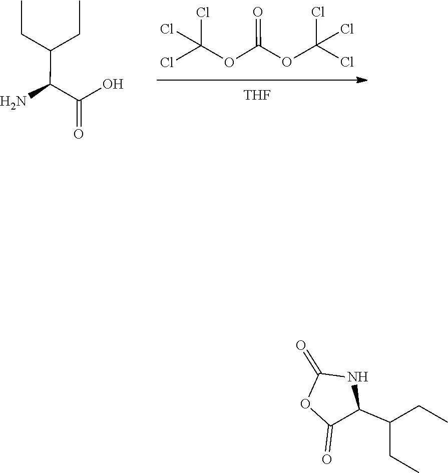

Step 1: Synthesis of (S)-4-(pentan-3-yl)oxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-3-ethylpentanoic acid to provide the title compound as a white solid.

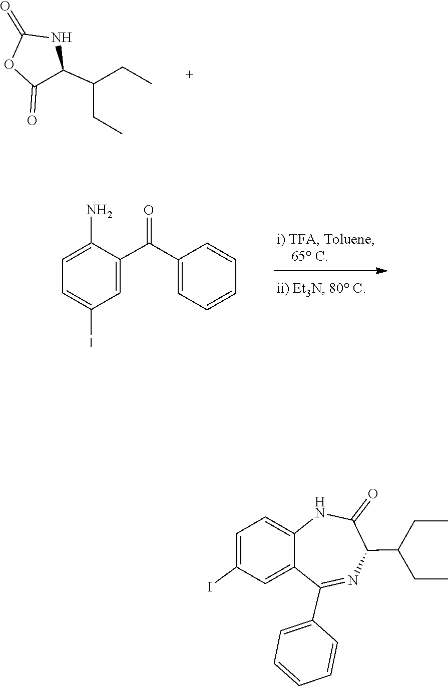 [SMILES: CCC(CC)[C@@H]1NC(=O)OC1=O ; Nc1ccc(I)cc1C(=O)c1ccccc1 ; CCC(CC)[C@@H]1N=C(c2ccccc2)c2cc(I)ccc2NC1=O]

Step 2: Synthesis of (S)-7-iodo-3-(pentan-3-yl)-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-(pentan-3-yl)oxazolidine-2,5-dione and 2-amino-5-chlorobenzophenone was replaced with 2-amino-5-iodobenzophenone to provide the title compound as an off white solid. Calculated for C₂₀H₂₁N₂O, 432.07; observed (M+H)⁺433.7

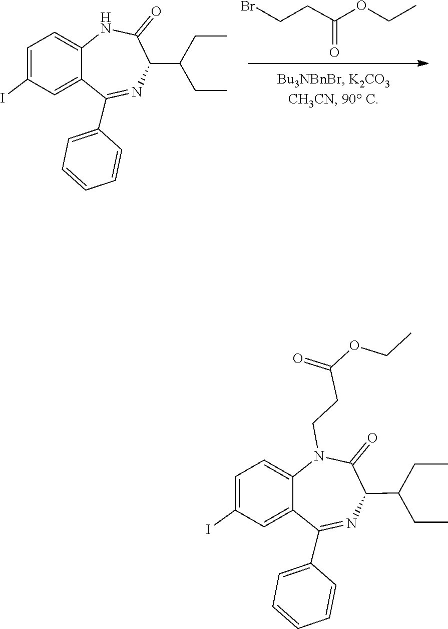

Step 3: Synthesis of (S)-ethyl 3-(7-iodo-2-oxo-3-(pentan-3-yl)-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[c][1,4]diazepin-1-yl)acetatc except that (S)-7-chloro-3-cyclopropyl-5-phenyl-1H-benzo[e][1,4]diazepin-2(3H)-one was replaced with(S)-7-iodo-3-(pentan-3-yl)-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₅H₂₉IN₂O₃, 532.12; observed (M+H)⁺533.9

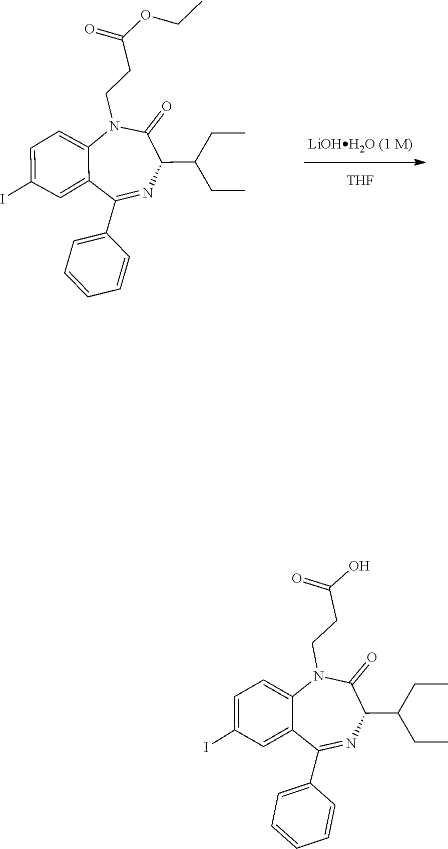 [SMILES: CCOC(=O)CCN1C(=O)[C@H](C(CC)CC)N=C(c2ccccc2)c2cc(I)ccc21 ; "THF" ; CCC(CC)[C@@H]1N=C(c2ccccc2)c2cc(I)ccc2N(CCC(=O)O)C1=O]

Step 4: Synthesis of (S)-3-(7-iodo-2-oxo-3-(pentan-3-yl)-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[el[1,4]diazepin-1-yl)acetate was replaced with (S)-ethyl 3-(7-iodo-2-oxo-3-(pentan-3-yl)-5-phenyl-2,3-dihydro-1H-benzo[e][1,4]diazepin-1-yl)propanoate to provide the title compound as an off-white solid. ¹H NMR (300 MHz, MeOD): δ (ppm) 8.02 (dd, J=8.79, 2.34 Hz, 1H), 7.54-7.60 (m, 4 H), 7.49-7.53 (m, 2H), 7.45-7.48 (m, 1H), 4.49-4.59 (m, 1H), 3.99-4.09 (m, 1H), 3.45 (d, J=10.54 Hz, 1H), 2.45-2.55 (m, 3H), 1.77-1.88 (m, 1H), 1.58-1.69 (m, 1H), 1.48-1.57 (m, 1H), 1.30-1.40 (m, 1H), 0.82-0.92 (m, 6H); Calculated for C₂₃H₂₅IN₂O₃, 504.09; observed (M+H)⁺505.9.

### Example 90: Synthesis of (S)-3-(7-methyl-2-oxo-3-(pentan-3-yl)-5-phenyl-2,3-dihydro-1H-benzo[e][1,4]diazepin-1-yl)propanoic acid

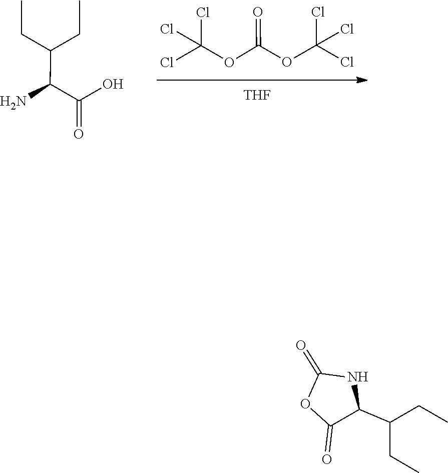

Step 1: Synthesis of (S)-4-(pentan-3-yl)oxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-3-ethylpentanoic acid to provide the title compound as a white solid.

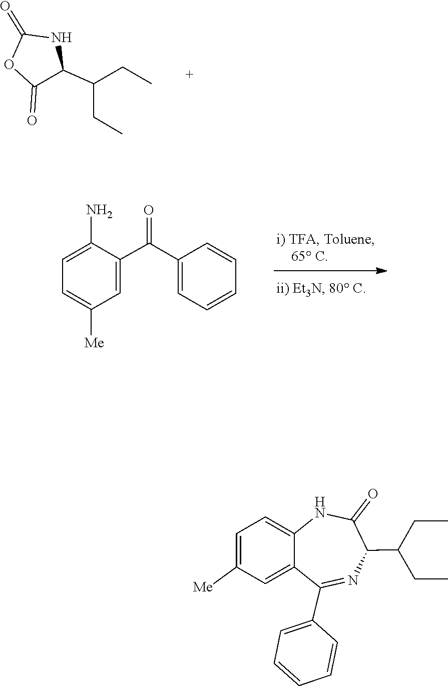 [SMILES: CCC(CC)[C@@H]1NC(=O)OC1=O ; Cc1ccc(N)c(C(=O)c2ccccc2)c1 ; CCC(CC)[C@@H]1N=C(c2ccccc2)c2cc(C)ccc2NC1=O]

Step 2: Synthesis of (S)-7-methyl-3-(pentan-3-yl)-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-(pentan-3-yl)oxazolidine-2,5-dione and 2-amino-5-chlorobenzophenone was replaced with 2-amino-5-methylbenzophenone to provide the title compound as a colorless liquid. Calculated for C₂₁H₂₄N₂O, 320.19; observed (M+H)⁺321.5

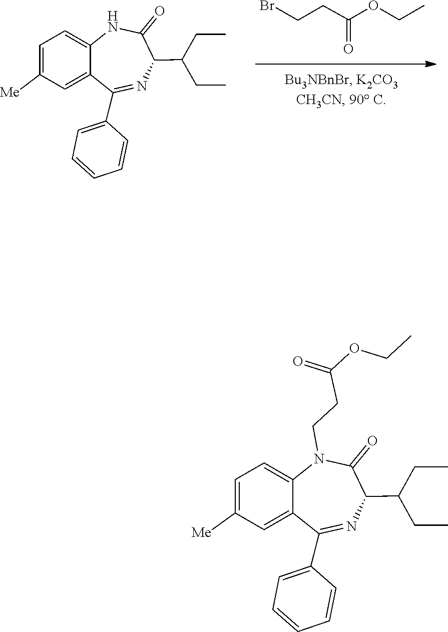

Step 3: Synthesis of (S)-ethyl 3-(7-methyl-2-oxo-3-(pentan-3-yl)-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[el[1,4]diazepin-2(3H)-one was replaced with (S)-7-methyl-3-(pentan-3-yl)-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₆H₃₂N₂O₃, 420.24; observed (M+H)⁺421.5

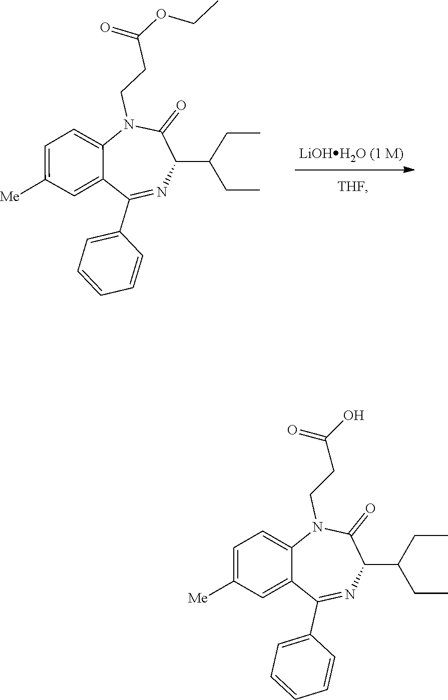 [SMILES: CCOC(=O)CCN1C(=O)[C@H](C(CC)CC)N=C(c2ccccc2)c2cc(C)ccc21 ; "THF," ; CCC(CC)[C@@H]1N=C(c2ccccc2)c2cc(C)ccc2N(CCC(=O)O)C1=O]

Step 4: Synthesis of (S)-3-(7-methyl-2-oxo-3-(pentan-3-yl)-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(7-methyl-2-oxo-3-(pentan-3-yl)-5-phenyl-2,3-dihydro-1H-benzo[e][1,4]diazepin-1-yl)propanoate to provide the title compound as an off-white solid. ¹H NMR (300 MHz, CDCl₃): δ (ppm) 7.64-7.72 (m, 2H), 7.47-7.60 (m, 5H), 7.16 (s, 1H), 4.89-5.00 (m, 1H), 3.82-3.94 (m, 1H), 3.57 (d, J=11.13 Hz, 1H), 2.65-2.76 (m, 1H), 2.48-2.60 (m, 1H), 2.39 (s, 3H), 2.19-2.31 (m, 1H), 1.83-1.98 (m, 1H), 1.64-1.75 (m, 1H), 1.49-1.63 (m, 1H), 1.28-1.40 (m, 1H), 0.80-0.93 (m, 6H); Calculated for C₂₄H₂₈N₂O₃, 392.21; observed (M+H)⁺393.8.

### Example 91: Synthesis of (S)-3-(2-oxo-3-(pentan-3-yl)-5-phenyl-7-(trifluoromethyl)-2,3-dihydro-1H-benzo[e][1,4]diazepin-1-yl)propanoic acid

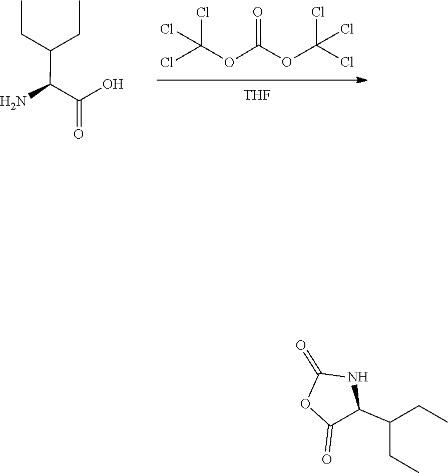

Step 1: Synthesis of (S)-4-(pentan-3-yl)oxazolidine-2,5-dione (72): The title compound was prepared according to the procedure of (S)-4-cyclopropyloxazolidine-2,5-dione except that (S)-2-amino-2-cyclopropylacetic acid was replaced with (S)-2-amino-3-ethylpentanoic acid to provide the title compound as a white solid.

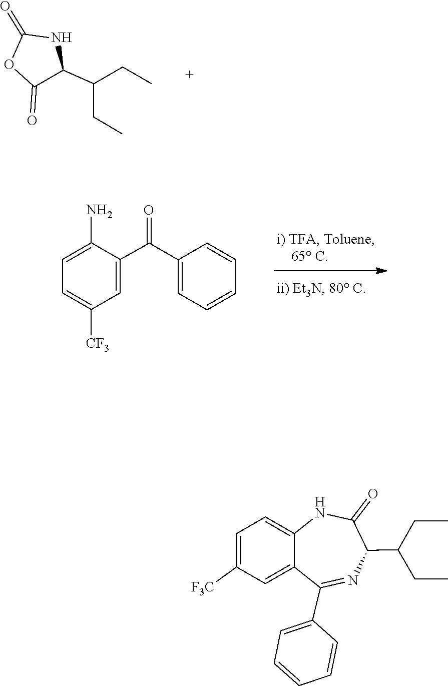 [SMILES: CCC(CC)[C@@H]1NC(=O)OC1=O ; Nc1ccc(C(F)(F)F)cc1C(=O)c1ccccc1 ; CCC(CC)[C@@H]1N=C(c2ccccc2)c2cc(C(F)(F)F)ccc2NC1=O]

Step 2: Synthesis of (S)-3-(pentan-3-yl)-5-phenyl-7-(trifluoromethyl)-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-(pentan-3-yl)oxazolidine-2,5-dione and 2-amino-5-chlorobenzophenone was replaced with 2-amino-5-trifluoromethylbenzophenone to provide the title compound as a colorless liquid. Calculated for C₂₁H₂₁F₃N₂O, 374.16; observed (M+H)⁺375.7

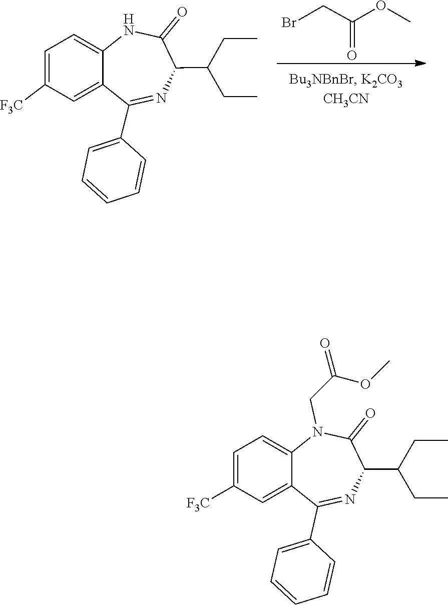

Step 3: Synthesis of (S)-methyl 2-(2-oxo-3-(pentan-3-yl)-5-phenyl-7-(trifluoromethyl)-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo|e111,41diazepin-1-yl)acetate except that (S)-7-chloro-3-cyclopropyl-5-phenyl-1H-benzo[e][1,4]diazepin-2(3H)-one was replaced with (S)-3-(pentan-3-yl)-5-phenyl-7-(trifluoromethyl)-1H-benzo[e][1,4]diazepin-2(3H)-one to provide the title compound as a colorless liquid. Calculated for C₂₄H₂₅F₃N₂O₃, 446.18; observed (M+H)⁺447.8

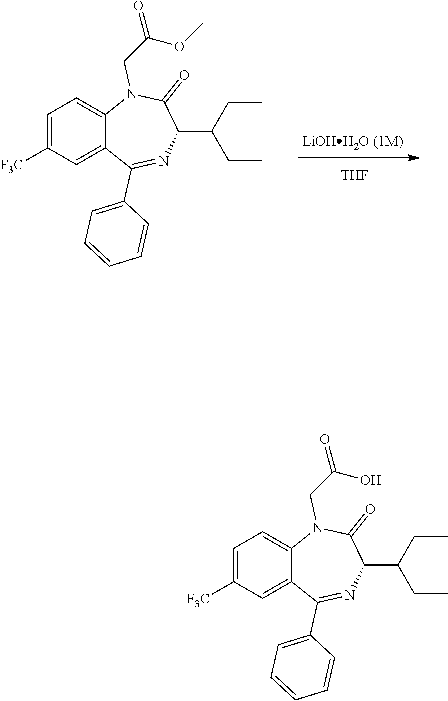 [SMILES: CCC(CC)[C@@H]1N=C(c2ccccc2)c2cc(C(F)(F)F)ccc2N(CC(=O)OC)C1=O ; CCC(CC)[C@@H]1N=C(c2ccccc2)c2cc(C(F)(F)F)ccc2N(CC(=O)O)C1=O ; "THF"]

Step 4: Synthesis of (S)-2-(2-oxo-3-(pentan-3-yl)-5-phenyl-7-(trifluoromethyl)-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(2-oxo-3-(pentan-3-yl)-5-phenyl-7-(trifluoromethyl)-2,3-dihydro-1H-benzo[e][1,4]diazepin-1-yl)acetate to provide the title compound as a white solid. ¹H NMR (300 MHz, CDCl₃): δ (ppm) 7.92 (dd, J=8.79 Hz, 1.76 Hz, 1H), 7.54-7.59 (m, 4H), 7.41-7.50 (m, 3H), 4.52-4.68 (m, 2H), 3.52 (d, J=10.55 Hz, I H), 2.54-2.63 (m, 1H), 1.81-1.94 (m, 1H), 1.59-1.71 (m, 1H), 1.47-1.58 (m, 1H), 1.32-1.41 (m, 1H), 0.83-0.89 (m, 6 H); Calculated for C₂₃H₂₃F₃N₂O₃, 432.17; observed (M+H)⁺433.8.

### Example 92: Synthesis of (S)-3-(2-oxo-3-(pentan-3-yl)-5-phenyl-7-(trifluoromethyl)-2,3-dihydro-1H-benzo[e][1,4]diazepin-1-yl)propanoic acid

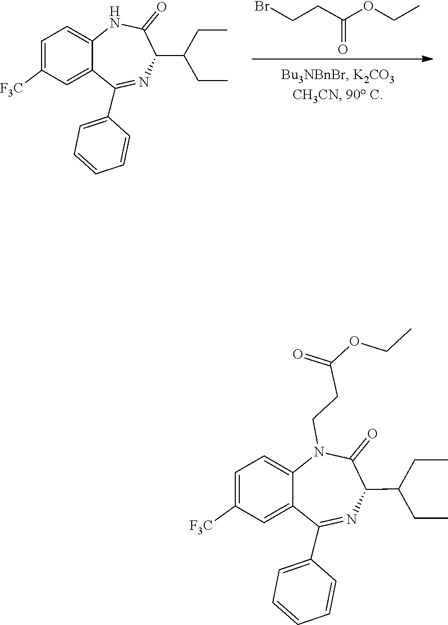

Step 1: Synthesis of (S)-ethyl 3-(2-oxo-3-(pentan-3-yl)-5-phenyl-7-(trifluoromethyl)-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-trifluoromethyl-3-(pentan-3-yl)-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₆H₂₉F₃N₂O₃, 474.21; observed (M+H)⁺475.3

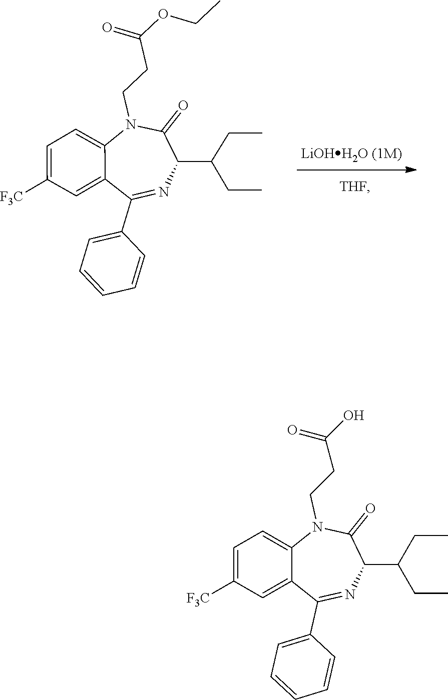 [SMILES: CCOC(=O)CCN1C(=O)[C@H](C(CC)CC)N=C(c2ccccc2)c2cc(C(F)(F)F)ccc21 ; "THF," ; CCC(CC)[C@@H]1N=C(c2ccccc2)c2cc(C(F)(F)F)ccc2N(CCC(=O)O)C1=O]

Step 2: Synthesis of (S)-3-(2-oxo-3-(pentan-3-yl)-5-phenyl-7-(trifluoromethyl)-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(2-oxo-3-(pentan-3-yl)-5-phenyl-7-(trifluoromethyl)-2,3-dihydro-1H-benzo[e][1,4]diazepin-1-yl)propanoate to provide the title compound as an white solid. 1H NMR (300 MHz, CDCl₃): δ (ppm) 7.80 (dd, J=8.50, 2.05 Hz, 1H), 7.50-7.58 (m, 4H), 7.42-7.47 (m, 1H), 7.35-7.41 (m, 2H), 4.53 (dt, J=13.92, 6.81 Hz, 1H), 3.96 (dt, J=13.63, 6.96 Hz, 1H), 3.27 (d, J=9.96 Hz, 1H), 2.46-2.60 (m, 3H), 1.77-1.91 (m, 1H), 1.58-1.70 (m, 1H), 1.44-1.55 (m, 1H), 1.25-1.36 (m, 1H), 0.85 (dt, J=12.31, 7.62 Hz, 6H); Calculated for C₂₄H₂₅F₃N₂O₃, 446.18; observed (M+H)⁺447.9.

### Example 93: Synthesis of (S)-2-(7-chloro-3-cyclohexyl-2-oxo-5-phenyl-2,3-dihydro-1H-benzo[e][1,4]diazepin-1-yl)acetic acid

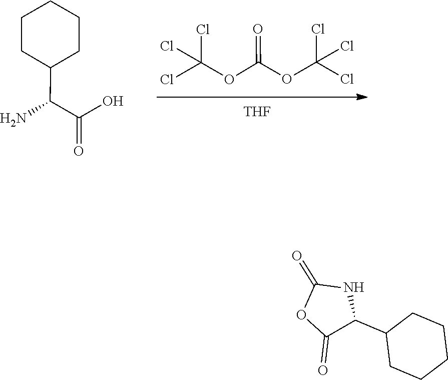 [SMILES: N[C@@H](C(=O)O)C1CCCCC1 ; O=C(OC(Cl)(Cl)Cl)OC(Cl)(Cl)Cl ; O=C1N[C@H](C2CCCCC2)C(=O)O1 ; "THF"]

Step 1: Synthesis of (R)-4-cyclohexyloxazolidine-2,5-dione: The title compound was prepared according to the procedure of (S)-4-cyclopropyloxazolidine-2,5-dione except that (S)-2-amino-2-cyclopropylacetic acid was replaced with (R)-2-amino-2-cyclohexylacetic acid to provide the title compound as a white solid.

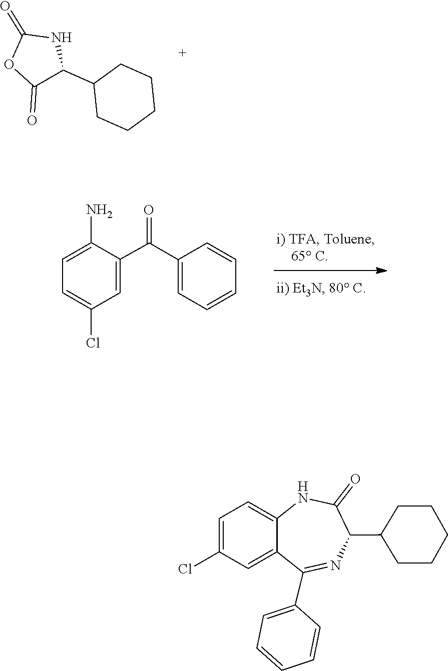 [SMILES: O=C1N[C@H](C2CCCCC2)C(=O)O1 ; Nc1ccc(Cl)cc1C(=O)c1ccccc1 ; O=C1Nc2ccc(Cl)cc2C(c2ccccc2)=N[C@H]1C1CCCCC1]

Step 2: Synthesis of (S)-7-chloro-3-cyclohexyl-5-phenyl-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (R)-4-cyclohexyloxazolidine-2,5-dione to provide the title compound as a white semi solid. Calculated for C₂₁H₂₁ClN₂O, 352.13; observed (M+H)⁺353.7.

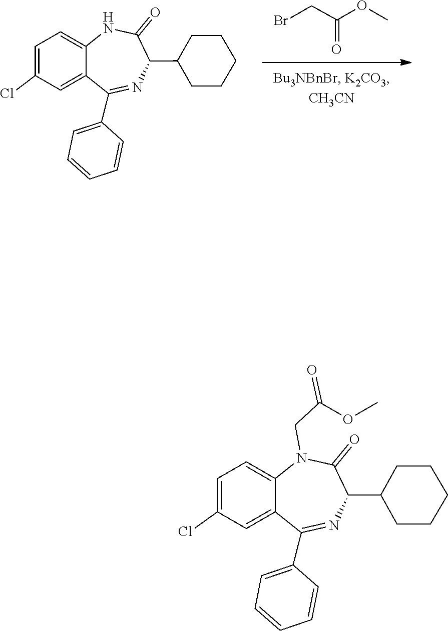

Step 3: Synthesis of (S)-methyl 2-(7-chloro-3-cyclohexyl-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-H-benzo[e][1,4]diazepin-2(3H)-one was replaced with (S)-7-chloro-3-cyclohexyl-5-phenyl-1H-benzo[e][1,4]diazepin-2(3H)-one to provide the title compound as a white semi solid. Calculated for C₂₄H₂₅ClN₂O₃, 424.16; observed (M+H)⁺425.8

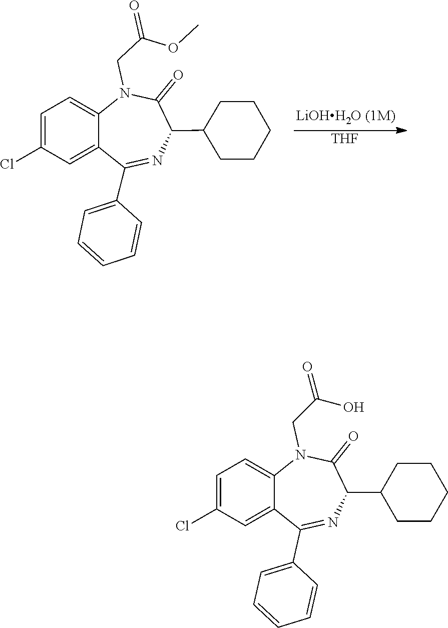 [SMILES: COC(=O)CN1C(=O)[C@H](C2CCCCC2)N=C(c2ccccc2)c2cc(Cl)ccc21 ; O=C(O)CN1C(=O)[C@H](C2CCCCC2)N=C(c2ccccc2)c2cc(Cl)ccc21 ; "THF"]

Step 4: Synthesis of (S)-2-(7-chloro-3-cyclohexyl-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(7-chloro-3-cyclohexyl-2-oxo-5-phenyl-2,3-dihydro-1H-benzo[e][1,4]diazepin-1-yl)acetate to provide the title compound as an off-white solid. ¹H NMR (300 MHz, CDCl₃): δ (ppm) 7.55-7.61 (m, 2H), 7.47-7.52 (m, 1H), 7.37-7.45 (m, 3H), 7.25-7.29 (m, 2H), 4.45-4.55 (m, 2H), 3.21 (d, J=10.55 Hz, 1H), 2.43-2.57 (m, 1 H), 2.30-2.38 (m, 1H), 1.89-1.98 (m, 1H), 1.67-1.81 (m, 3H), 1.37-1.48 (m, 2H), 1.13-1.23 (m, 1H), 0.78-0.93 (m, 2H); Calculated for C₂₃H₂₃ClN₂O₃, 410.14; observed (M+H)⁺411.8

### Example 94: Synthesis of (S)-3-(7-chloro-3-cyclohexyl-2-oxo-5-phenyl-2,3-dihydro-1H-benzo[e][1,4]diazepin-1-yl)propanoic acid

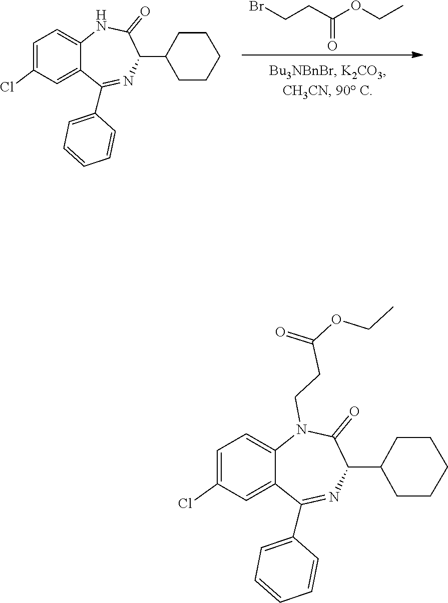

Step 1: Synthesis of (S)-ethyl 3-(7-chloro-3-cyclohexyl-2-oxo-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-cyclohexyl-5-phenyl-1H-benzo[e][1,4]diazepin-2(3H)-one and methyl 2-bromoacetate was replaced with ethyl 3 -bromopropanoate to provide the title compound as a colorless liquid. Calculated for C₂₆H₂₉ClN₂O₃, 452.19; observed (M+H)⁺453.9

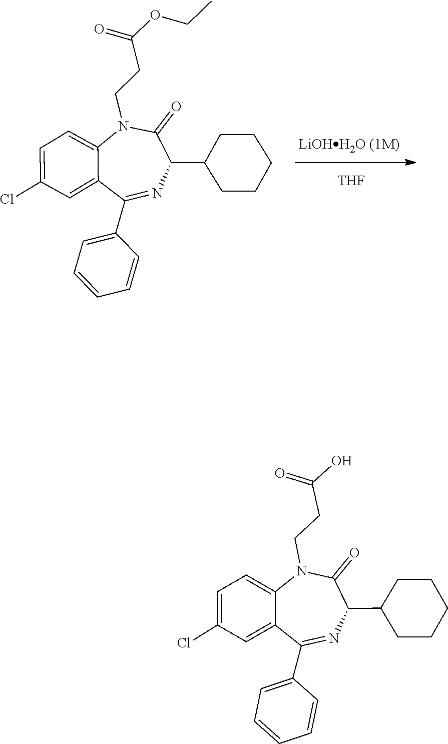 [SMILES: CCOC(=O)CCN1C(=O)[C@H](C2CCCCC2)N=C(c2ccccc2)c2cc(Cl)ccc21 ; "THF" ; O=C(O)CCN1C(=O)[C@H](C2CCCCC2)N=C(c2ccccc2)c2cc(Cl)ccc21]

Step 2: Synthesis of (S)-3-(7-chloro-3-cyclohexyl-2-oxo-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(7-chloro-3-cyclohexyl-2-oxo-5-phenyl-2,3-dihydro-1H-benzo[e][1,4]diazepin-1-yl)propanoate as a white solid. ¹H NMR (300 MHz, CDCl₃): δ (ppm) 7.49-7.58 (m, 3H), 7.34-7.45 (m, 4H), 7.26-7.27 (m, 1H), 4.47-4.57 (m, 1H), 3.85-3.93 (m, 1H), 3.10 (d, J=9.38 Hz, 1H), 2.47-2.56 (m, 3H), 2.28-2.36 (m, 1H), 1.94-2.03 (m, 1H), 1.67-1.81 (m, 3H), 1.37-1.49 (m, 2H), 1.13-1.26 (m, 1H), 0.75-0.91 (m, 2H); Calculated for C₂₄H₂₈ClN₂O₃, 424.16; observed (M+H)⁺425.8

### Example 95: Synthesis of (S)-2-(7-chloro-3-cycloheptyl-2-oxo-5-phenyl-2,3-dihydro-1H-benzo[e][1,4]diazepin-1-yl)acetic acid

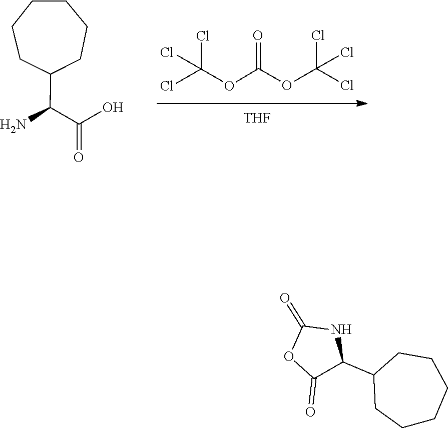 [SMILES: N[C@H](C(=O)O)C1CCCCCC1 ; O=C(OC(Cl)(Cl)Cl)OC(Cl)(Cl)Cl ; O=C1N[C@@H](C2CCCCCC2)C(=O)O1 ; "THF"]

Step 1: Synthesis of (S)-4-cycloheptyloxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-2-cycloheptylacetic acid to provide the title compound as a white solid.

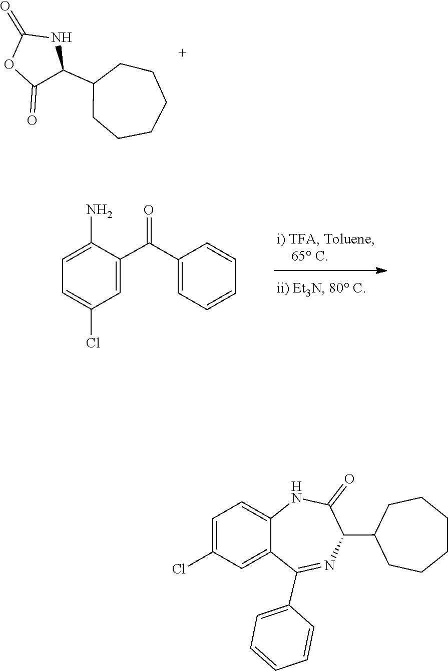 [SMILES: O=C1N[C@@H](C2CCCCCC2)C(=O)O1 ; Nc1ccc(Cl)cc1C(=O)c1ccccc1 ; O=C1Nc2ccc(Cl)cc2C(c2ccccc2)=N[C@H]1C1CCCCCC1]

Step 2: Synthesis of (S)-7-chloro-3-cycloheptyl-5-phenyl-1H-benzo[c][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-cycloheptyloxazolidine-2,5-dione to provide the title compound as a white solid. Calculated for C₂₂H₂₃ClN₂O, 366.15; observed (M+H)⁺367.7.

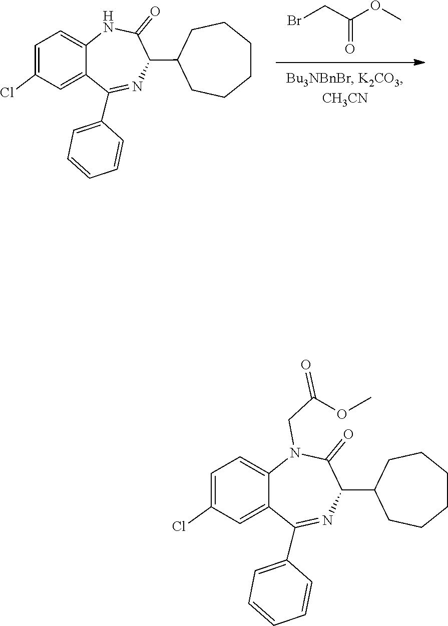

Step 3: Synthesis of (S)-methyl 2-(7-chloro-3-cycloheptyl-2-oxo-5-phenyl-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo|e111,41diazepin-1-yl)acetate except that (S)-7-chloro-3-cyclopropyl-5-phenyl-1H-benzo[e][1,4]diazepin-2(3H)-one was replaced with (S)-7-chloro-3-cycloheptyl-5-phenyl-1H-benzo[e][1,4]diazepin-2(3H)-one to provide the title compound as a colorless liquid. Calculated for C₂₅H₂₇ClN₂O₃, 438.17; observed (M+H)⁺439.8

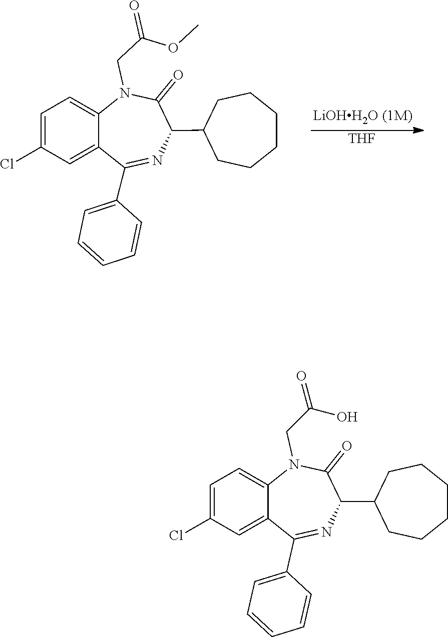 [SMILES: COC(=O)CN1C(=O)[C@H](C2CCCCCC2)N=C(c2ccccc2)c2cc(Cl)ccc21 ; O=C(O)CN1C(=O)[C@H](C2CCCCCC2)N=C(c2ccccc2)c2cc(Cl)ccc21 ; "THF"]

Step 4: Synthesis of (S)-2-(7-chloro-3-cycloheptyl-2-oxo-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(7-chloro-3-cycloheptyl-2-oxo-5-phenyl-2,3-dihydro-1H-benzo[e][1,4]diazepin-1-yl)acetate to provide the title compound as an off-white solid. ¹H NMR (300 MHz, CDCl₃): δ (ppm) 7.61-7.66 (m, 1H), 7.53-7.60 (m, 3H), 7.43-7.49 (m, 2H), 7.33-7.40 (m, 1H), 7.29 (d, J=2.34 Hz, 1H), 4.25-4.81 (m, 2H), 3.43-3.47 (m, 1H), 2.62-2.77 (m, 1H), 2.18-2.32 (m, 1H), 1.83-1.93 (m, 1H), 1.70-1.79 (m, 1H), 1.56-1.68 (m, 5 H), 1.42-1.54 (m, 2H), 1.19-1.30 (m, 1H), 1.04-1.18 (m, 1H); Calculated for C₂₄H₂₅ClN₂O₃, 424.16; observed (M+H)⁺425.8.

### Example 96: Synthesis of (S)-3-(7-chloro-3-cycloheptyl-2-oxo-5-phenyl-2,3-dihydro-1H-benzo[e][1,4]diazepin-1-yl)propanoic acid

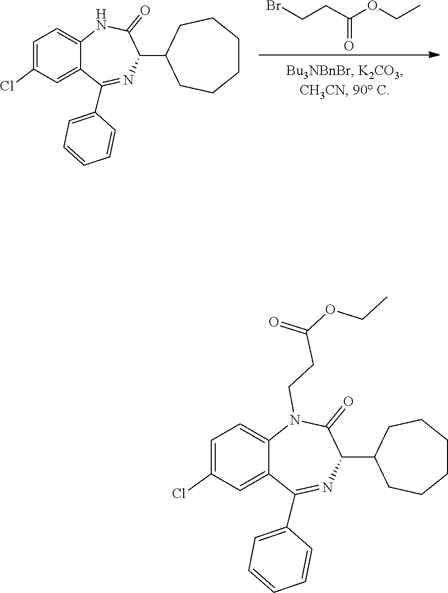

Step 1: Synthesis of (S)-ethyl 3-(7-chloro-3-cycloheptyl-2-oxo-5-phenyl-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with(S)-7-chloro-3-cycloheptyl-5-phenyl-1H-benzo[e][1,4]diazepin-2(3H)-one and methyl 2-bromoacetate was replaced with ethyl 3-bromopropanoate to provide the title compound as a colorless liquid. Calculated for C₂₂H₃,ClN₂O₃, 466.20; observed (M+H)⁺467.9.

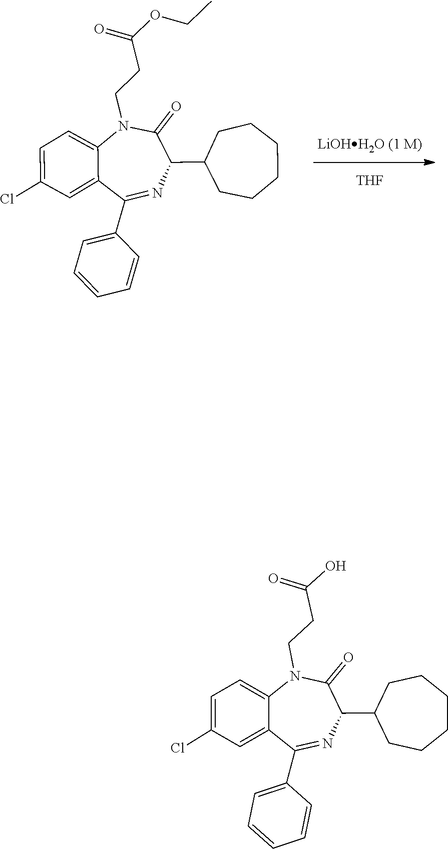 [SMILES: CCOC(=O)CCN1C(=O)[C@H](C2CCCCCC2)N=C(c2ccccc2)c2cc(Cl)ccc21 ; "THF" ; O=C(O)CCN1C(=O)[C@H](C2CCCCCC2)N=C(c2ccccc2)c2cc(Cl)ccc21]

Step 2: Synthesis of (S)-3-(7-chloro-3-cycloheptyl-2-oxo-5-phenyl-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-ethyl 3-(7-chloro-3-cycloheptyl-2-oxo-5-phenyl-2,3-dihydro-TH-benzo[e][1,4]diazepin-1-yl)propanoate to provide the title compound as an white solid. ¹H NMR (300 MHz, CDCl₃): δ (ppm) 7.51-7.60 (m, 3H), 7.34-7.46 (m, 4H), 7.27-7.29 (m, 1H), 4.45-4.57 (m, 1H), 3.83-3.90 (m, 1H), 3.13-3.22 (m, 1H), 2.63-2.71 (m, 1H), 2.46-2.56 (m, 2H), 2.22-2.31 (m, 1H), 1.83-1.91 (m, 1H), 1.60-1.73 (m, 6H), 1.49-1.56 (m, 2 H), 1.07-1.21 (m, 2H); Calculated for C₂₅H₂₇ClN₂O₃, 438.17; observed (M+H)⁺439.8

### Example 97: Synthesis of (S)-2-(7-bromo-2-oxo-3-(pentan-3-yl)-5-(pyridin-2-yl)-2,3-dihydro-1H-benzo[c][1,4]diazepin-1-yl)acetic acid

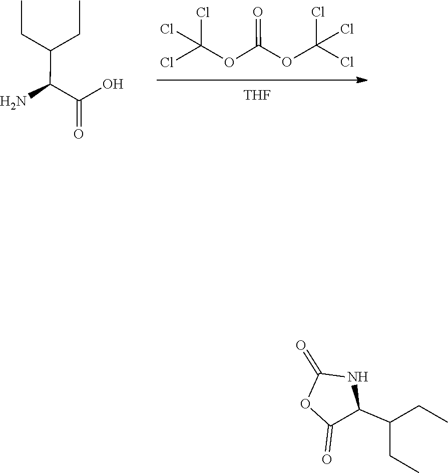

Step 1: Synthesis of (S)-4-(pentan-3-yl)oxazolidine-2,5-dione: The title compound was prepared according to the procedure of (S)-4-cyclopropyloxazolidine-2,5-dione except that (S)-2-amino-2-cyclopropylacetic acid was replaced with (S)-2-amino-3-ethylpentanoic acid to provide the title compound as a white solid.

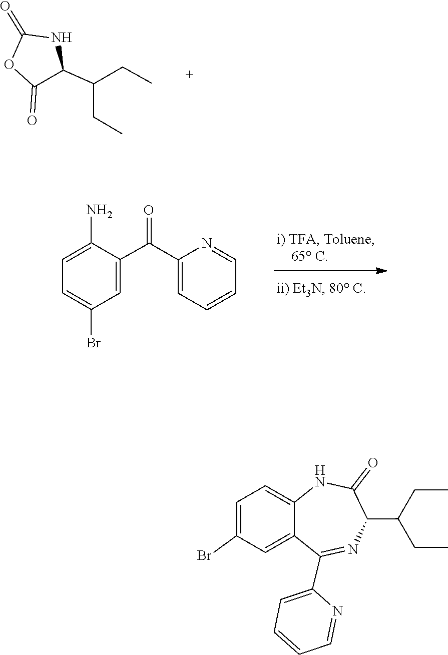 [SMILES: CCC(CC)[C@@H]1NC(=O)OC1=O ; Nc1ccc(Br)cc1C(=O)c1ccccn1 ; CCC(CC)[C@@H]1N=C(c2ccccn2)c2cc(Br)ccc2NC1=O]

Step 2: Synthesis of (S)-7-bromo-3-(pentan-3-yl)-5-(pyridin-2-yl)-1H-benzo[e][1,4]diazepin-2(3H)-one: The title compound was prepared according to the procedure of (S)-7-chloro-3-cyclopropyl-5-phenyl-1H-benzo[e][1,4]diazepin-2(3H)-one except that (S)-4-cyclopropyloxazolidine-2,5-dione was replaced with (S)-4-(pentan-3-yl)oxazolidine-2,5-dione and 2-amino-5-chlorobenzophenone was replaced with (2-amino-5-bromophenyl)(pyridin-2-yl)methanone to provide the title compound as a yellow semi solid. Calculated for C₁₉H₂₀BrN₃O, 385.08; observed (M+H)⁺386.7.

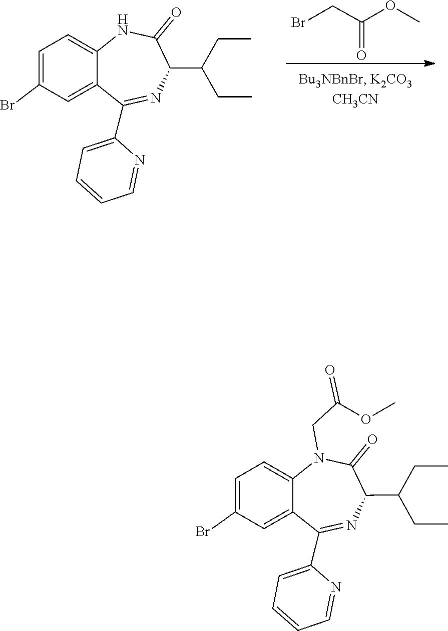

Step 3: Synthesis of (S)-methyl 2-(7-bromo-2-oxo-3-(pentan-3-yl)-5-(pyridin-2-yl)-2,3-dihydro-1H-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-bromo-3-(pentan-3-yl)-5-(pyridin-2-yl)-1H-benzo[e][1,4]diazepin-2(3H)-one to provide the title compounds as a colorless liquid. Calculated for C₂₂H₂₄BrN₃O₃, 457.10; observed (M+H)⁺458.8.

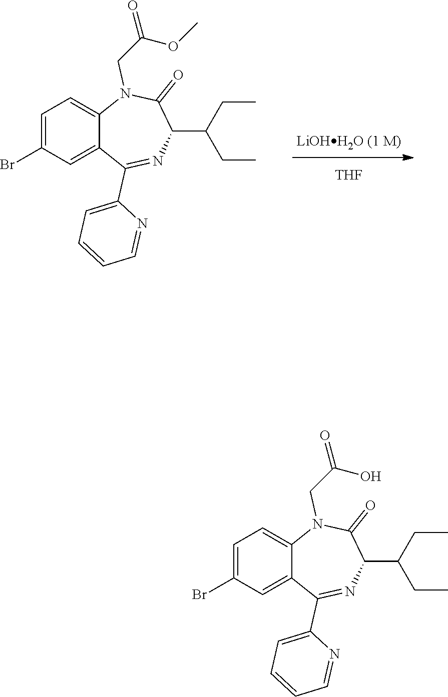 [SMILES: CCC(CC)[C@@H]1N=C(c2ccccn2)c2cc(Br)ccc2N(CC(=O)OC)C1=O ; CCC(CC)[C@@H]1N=C(c2ccccn2)c2cc(Br)ccc2N(CC(=O)O)C1=O ; "THF"]

Step 4: Synthesis of (S)-2-(7-bromo-2-oxo-3-(pentan-3-yl)-5-(pyridin-2-yl)-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(7-bromo-2-oxo-3-(pentan-3-yl)-5-(pyridin-2-yl)-2,3-dihydro-1H-benzo[e][1,4]diazepin-1-yl)acetate to provide the title compound as an off white solid. ¹H NMR (300 MHz, CDCl₃): δ (ppm) 8.71-8.77 (m, 1H), 8.19-8.27 (m, 1H), 7.91-8.00 (m, 1H), 7.71-7.81 (m, 2H), 7.39 (d, J=1.76 Hz, 1H), 7.26-7.29 (m, 1H), 4.61-4.77 (m, 1 H), 4.28 (d, J=17.58 Hz, 1H), 3.61 (d, J=9.96 Hz, 1H), 2.46-2.57 (m, 1H), 1.77-1.93 (m, 1H), 1.48-1.65 (m, 2H), 1.29-1.43 (m, 1H), 0.78-0.90 (m, 6H); Calculated for C₂₁H₂₂BrN₃O₃, 443.08; observed (M+H)⁺444.7.

### Example 98: Synthesis of (S)-3-(7-bromo-2-oxo-3-(pentan-3-yl)-5-(pyridin-2-yl)-2,3-dihydro-1H-benzo[e][1,4]diazepin-1-yl)propanoic acid

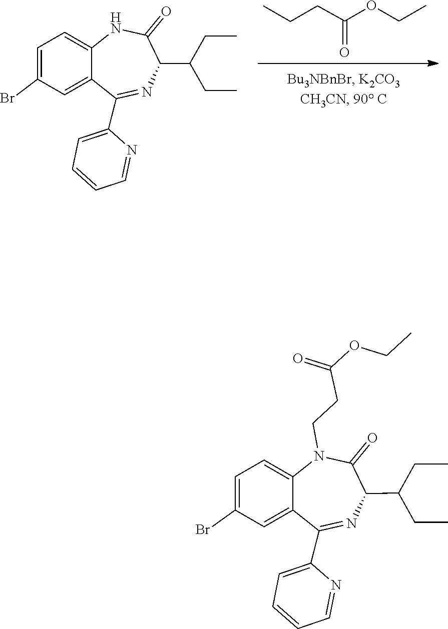

Step 1: Synthesis of (S)-ethyl 3-(7-bromo-2-oxo-3-(pentan-3-yl)-5-(pyridin-2-yl)-2,3-dihydro-1H-benzo[e][1,4]diazepin-1-yl)propanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-bromo-3-(pentan-3-yl)-5-(pyridin-2-yl)-1H-benzo[e][1,4]diazepin-2(3H)-one and methyl 2-bromoacetate was replaced with ethyl 3-bromopropanoate to provide the title compound as a colorless liquid. Calculated for C₂₄H₂₈BrN₃O₃, 485.13; observed (M+H)⁺486.9.

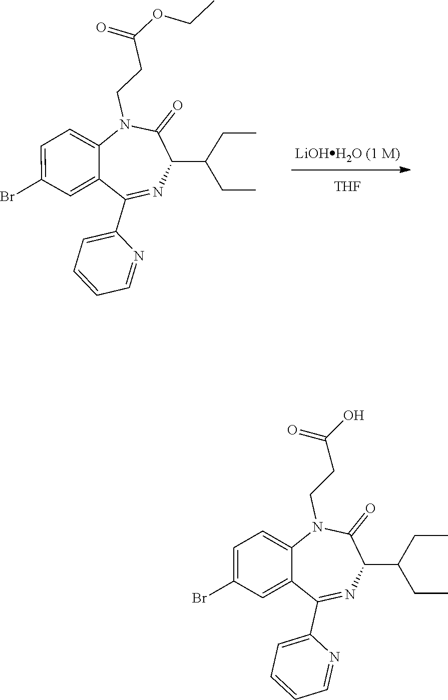 [SMILES: "THF" ; CCOC(=O)CCN1C(=O)[C@H](C(CC)CC)N=C(c2ccccn2)c2cc(Br)ccc21 ; CCC(CC)[C@@H]1N=C(c2ccccn2)c2cc(Br)ccc2N(CCC(=O)O)C1=O]

Step 2: Synthesis of (S)-3-(7-bromo-2-oxo-3-(pentan-3-yl)-5-(pyridin-2-yl)-2,3-dihydro-1H-benzo[e][1,4]diazepin-1-yl)propanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1 H-benzo[e][1,4]diazepin-1-yl)acetate was replaced with (S)-ethyl 3-(7-homo-2-oxo-3-(pentan-3-yl)-5-(pyridin-2-yl)-2,3-dihydro-1H-benzo[e][1,4]diazepin-1-yl)propanoate to provide the title compound as an off white solid. 1H NMR (300 MHz, CDCl₃): δ (ppm) 8.91 (d, J=5.27 Hz, 1H), 8.27-8.36 (m, 2H), 7.79-7.87 (m, 1H), 7.74 (dd, J=8.79, 2.34 Hz, 1H), 7.33-7.41 (m, 2H), 4.63 (dt, J=13.48, 5.57 Hz, 1H), 3.86-4.02 (m, 1H), 3.45 (d, J=9.38 Hz, 1H), 2.47-2.63 (m, 2H), 2.36-2.45 (m, 1H), 1.76-1.89 (m, 1H), 1.48-1.64 (m. 2 H), 1.29-1.42 (m, 1H), 0.80-0.93 (m, 6H); Calculated for C₂₂H₂₄BrN₃O₃, 457.10; observed (M+H)⁺458.8.

### Example 99: Synthesis of (S)-3-(7-chloro-2-oxo-3-(pentan-3-yl)-5-phenyl-2,3-dihydro-1H-benzo[e][1,4]diazepin-1-yl)propanoic acid

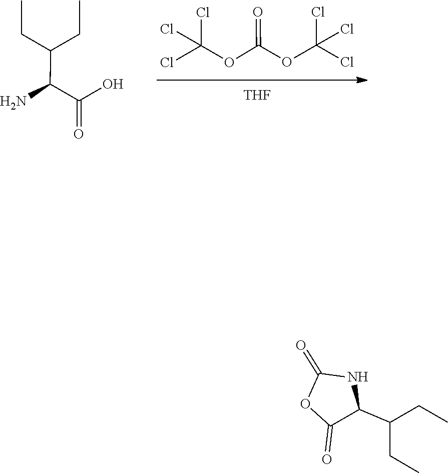

Step 1: Synthesis of (S)-4-(pentan-3-yl)oxazolidine-2,5-dione: A round bottom flask was equipped with magnetic stir bar, condenser, and N₂ inlet and (S)-2-Amino-3-ethylpentanoic acid (71, 220 mgr, 1.516 mmol) in dry tetrahydrofuran (3 mL) was added and heated at 50° C. The solution of Triphosgene (150 mgr, 0.505 mmol) in dry tetrahydrofuran (5 mL) was added into the reaction by dropping funnel and the reaction mixture was heated to reflux for 3 hours. The reaction mixture was cooled to 23° C., and the solvent was stripped under vacuum to provide the title compound as a white solid.

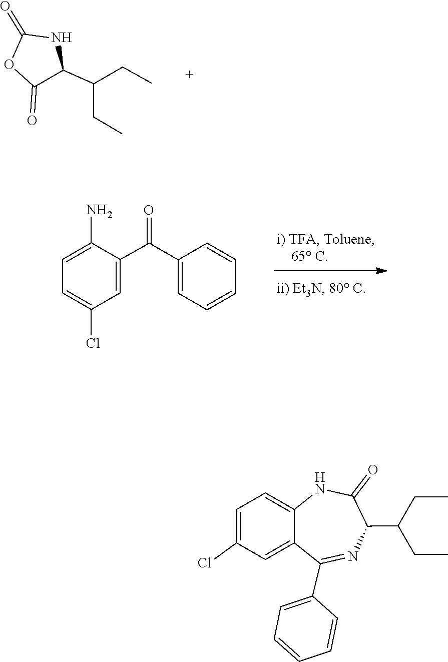 [SMILES: CCC(CC)[C@@H]1NC(=O)OC1=O ; Nc1ccc(Cl)cc1C(=O)c1ccccc1 ; CCC(CC)[C@@H]1N=C(c2ccccc2)c2cc(Cl)ccc2NC1=O]

Step 2: (S)-7-chloro-3-(pentan-3-yl)-5-phenyl-1H-benzo[e][1,4]diazepin-2(3H)-one: To a round bottom flask was equipped with magnetic stir bar, condenser, and N₂ inlet was added 4-(pentan-3-yl) oxazolidine-2,5-dione (72, 259 mgr, 1.512 mmol) and 2-amino-5-chlorobenzophenone (3, 292 mgr, 1.260 mmol) in dry toluene (8 mL). Trifluoroacetic acid (TFA) (0.194 mL, 2.52 mmol) was added to the reaction mixture and stirred at 65° C. for 18 hours. Then triethylamine (Et₃N) (0.35 mL, 2.52 mmol) was added to the reaction mixture and stirred at 80° C. for 3 hours. The reaction mixture was cooled to 23° C., the solvent was removed under vacuum, and the residue was partitioned between 1N HCl and ethyl acetate. The layers were separated, and the organic layer was washed with saturated NaHCO₃ solution and brine solution, dried over Na₂SO₄, filtered, and the solvent was removed under vacuum. The crude product was purified by normal phase chromatography (0-20% ethyl acetate in Hexanes) to provide the title compound as a colorless gum. ¹H NMR (300 MHz, CDCl3): δ (ppm) 7.49-7.53 (m, 2H), 7.37-7.48 (m, 4H), 7.32 (d, J=2.34 Hz, 1H), 7.14 (d, J=8.79 Hz, 1H), 3.38 (d, J=9.37 Hz, 1H), 2.40-2.51 (m, 1H), 1.86-1.99 (m, 1H), 1.62-1.76 (m, 2H), 1.36-1.49 (m, 1H), 0.90-0.96 (m, 6H); Calculated for C₂₀H₂₁ClN₂O, 340.13; observed (M+H)⁺341.6.

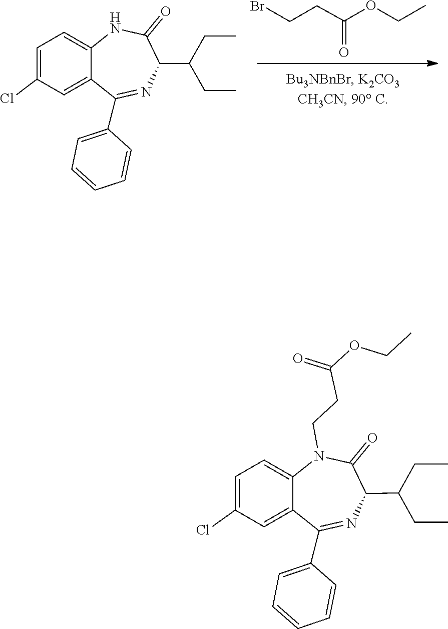

Step 3: Synthesis of (S)-ethyl 3-(7-chloro-2-oxo-3-(pentan-3-yl)-5-phenyl-2,3-dihydro-1H-benzo[e][1,4]diazepin-1-yl)propanoate. To a round bottom flask was equipped with magnetic stir bar, condenser, and N₂ inlet, was added (S)-7-chloro-3-(pentan-3-yl)-5-phenyl-TH-benzo[e][1,4]diazepin-2(3H)-one (97, 1 gr, 2.941 mmol) in dry acetonitrile (12 mL). Benzyltributylammonium bromide (0.155 gr, 0.441 mmol), potassium carbonate (0.801 gr, 5.882 mmol) and ethyl 3-bromopropanoate (0.403 mL, 3.235 mmol) was added to the reaction mixture and stirred at 90° C. for 16 hours. The reaction mixture was cooled, filtered, and the solvent was removed under vacuum. The crude product was purified by normal phase chromatography (0-40% ethyl acetate in dichloromethane) to provide the title compound as a colorless liquid. ¹H NMR (300 MHz, CDCl₃): δ (ppm) 7.59-7.64 (m, 2H), 7.49-7.55 (m, 1H), 7.36-7.48 (m, 4H), 7.27-7.30 (m, 1H), 4.44-4.57 (m, 1H), 3.83-3.99 (m, 3H), 3.31 (d, J=9.37 Hz, 1H), 2.46-2.64 (m, 3H), 1.78-1.92 (m, 1 H), 1.61-1.71 (m, 1H), 1.46-1.58 (m, 1H), 1.25-1.38 (m, 1H), 1.03-1.13 (m, 3H), 0.82-0.92 (m, 6H); Calculated for C₂₅H₂₉ClN₂O₃, 440.19; observed (M+H)⁺441.8.

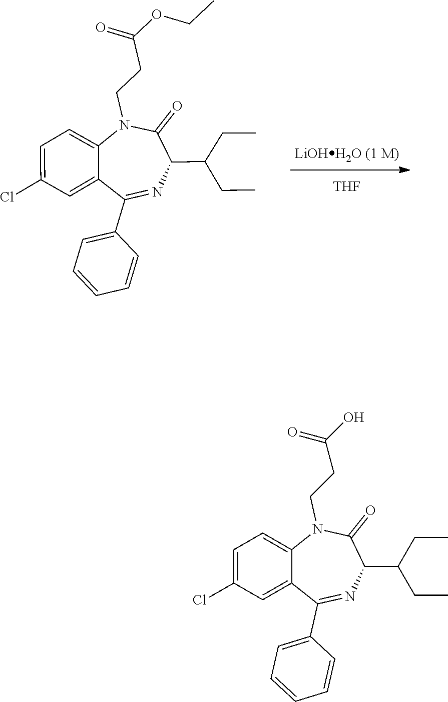 [SMILES: "THF" ; CCOC(=O)CCN1C(=O)[C@H](C(CC)CC)N=C(c2ccccc2)c2cc(Cl)ccc21 ; CCC(CC)[C@@H]1N=C(c2ccccc2)c2cc(Cl)ccc2N(CCC(=O)O)C1=O]

Step 4: Synthesis of (S)-3-(7-chloro-2-oxo-3-(pentan-3-yl)-5-phenyl-2,3-dihydro-1H-benzo[e][1,4]diazepin-1-yl)propanoic acid. To a round bottom flask was equipped with magnetic stir bar, was added (S)-ethyl 3-(7-chloro-2-oxo-3-(pentan-3-yl)-5-phenyl-2,3-dihydro-1H-benzo[e][1,4]diazepin-1-yl)propanoate (1.81 gr, 4.105 mmol) in tetrahydrofuran (15 mL). LiOH·H₂O (1 M) aqueous solution (9 mL, 9.031 mmol) was added to the reaction mixture and the reaction was stirred at 23° C. for 18 hours. The reaction mixture was concentrated under vacuum to half its original volume. The solution was cooled to 0° C. and the pH was adjusted to 3-4 by addition of 10% citric acid monohydrate aqueous solution and the resulting solution was extracted with ethyl acetate. The ethyl acetate was then dried over Na₂SO₄, filtered, and the solvent was removed under vacuum. The crude product was purified by normal phase chromatography (0-30% EtOAc/Hexanes) to provide the title compound as an off-white solid. ¹H NMR (300 MHz, CDCl₃): δ (ppm) 7.49-7.57 (m, 3H), 7.33-7.46 (m, 4H), 7.25-7.28 (m, 1H), 4.44-4.59 (m, 1H), 3.79-3.96 (m, 1H), 3.29 (d, J=9.96 Hz, 1 H), 2.47-2.55 (m, 3H), 1.78-1.90 (m, 1H), 1.59-1.72 (m, 1H), 1.43-1.55 (m, 1H), 1.26-1.33 (m, 1H), 0.79-0.90 (m, 6H); Calculated for C₂₃H₂₅ClN₂O₃, 412.16; observed (M+H)⁺413.8.

### Example 100: Synthesis of (S)-2-(7-chloro-2-oxo-3-(pentan-3-yl)-5-phenyl-2,3-dihydro-1H-benzo[e][1,4]diazepin-1-yl)acetic acid

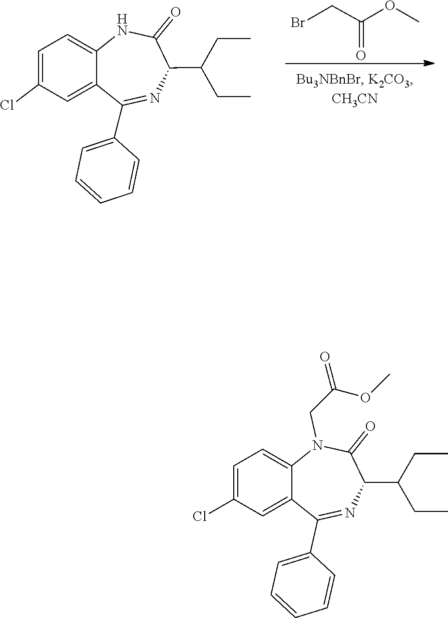

Step 1 Synthesis of (S)-methyl 2-(7-chloro-2-oxo-3-(pentan-3-yl)-5-phenyl-2,3-dihydro-TH-benzo[e][1,4]diazepin-1-yl)acetate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-(pentan-3-yl)-5-phenyl-1,3-dihydro-2H-benzo[e][1,4]diazepin-2-one to provide the title compound as a white solid. Calculated for C₂₃H₂₅CN₂O₃, 412.16; observed (M+H)⁺413.8

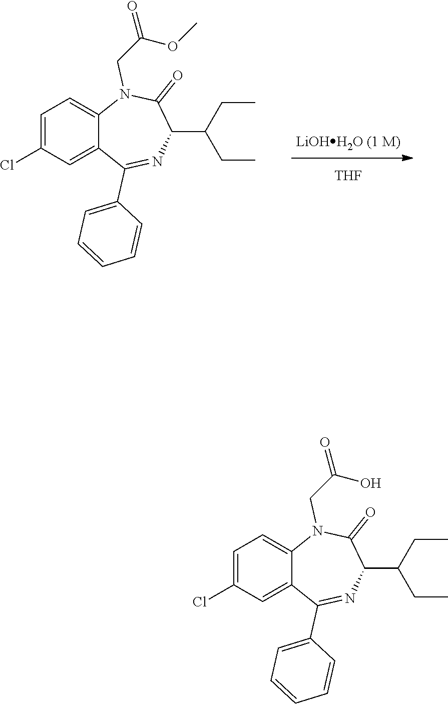 [SMILES: CCC(CC)[C@@H]1N=C(c2ccccc2)c2cc(Cl)ccc2N(CC(=O)OC)C1=O ; CCC(CC)[C@@H]1N=C(c2ccccc2)c2cc(Cl)ccc2N(CC(=O)O)C1=O ; "THF"]

Step 2: Synthesis of (S)-2-(7-chloro-2-oxo-3-(pentan-3-yl)-5-phenyl-2,3-dihydro-1H-benzo[e][1,4]diazepin-1-yl)acetic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 2-(7-chloro-2-oxo-3-(pentan-3-yl)-5 -phenyl-2,3-dihydro-1H-benzo[c][1,4]diazepin-1-yl)acetate to provide the title compound as an off-white solid. Calculated for C₂₂H₂₃ClN₂O₃, 398.14; observed (M+H)⁺399.7.

### Example 101: Synthesis of (S)-4-(7-chloro-2-oxo-3-(pentan-3-yl)-5-phenyl-2,3-dihydro-1H-benzo[e][1,4]diazepin-1-yl)butanoic acid

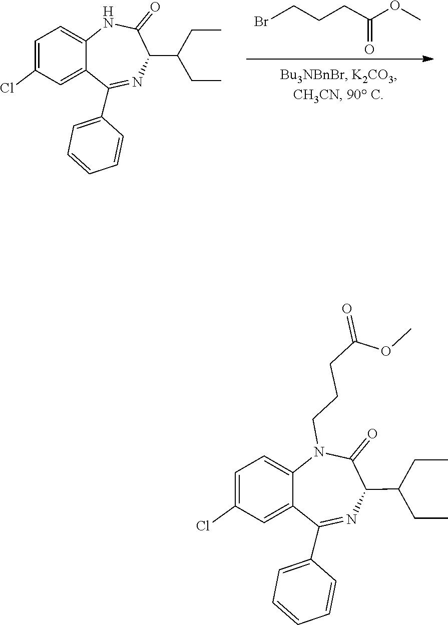

Step 1: Synthesis of (S)-methyl 4-(7-chloro-2-oxo-3-(pentan-3-yl)-5-phenyl-2,3-dihydro-1H-benzo[e][1,4]diazepin-1-yl)butanoate: The title compound was prepared according to the procedure for (S)-methyl 2-(7-chloro-3-cyclopropyl-2-oxo-5-phenyl-2,3-dihydro-1H-benzo[e][1,4]diazepin-1-yl)acetate except that (S)-7-chloro-3-cyclopropyl-5-phenyl-1H-benzo[e][1,4]diazepin-2(3H)-one was replaced with (S)-7-chloro-3-(pentan-3-yl)-5-phenyl-1,3-dihydro-2H-benzo[e][1,4]diazepin-2-one and methyl 2-bromoacetate was replaced with methyl 4-bromobutanoate to provide the title compound as a colorless liquid. Calculated for C₂₅H₂₉ClN₂O₃, 440.19; observed (M+H)⁺441.9

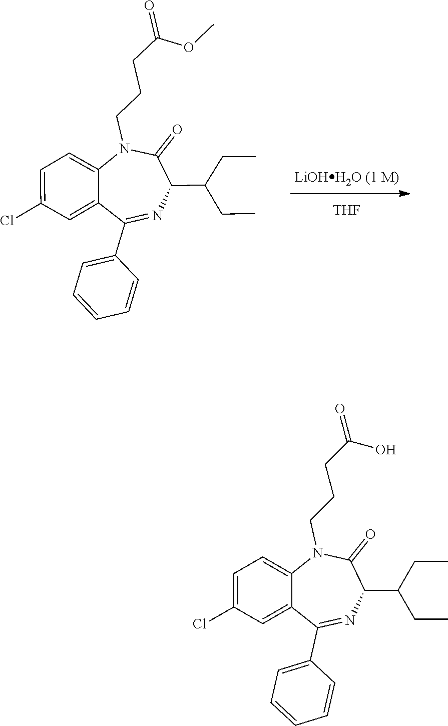 [SMILES: "THF" ; CCC(CC)[C@@H]1N=C(c2ccccc2)c2cc(Cl)ccc2N(CCCC(=O)OC)C1=O ; CCC(CC)[C@@H]1N=C(c2ccccc2)c2cc(Cl)ccc2N(CCCC(=O)O)C1=O]

Step 2: Synthesis of (S)-4-(7-chloro-2-oxo-3-(pentan-3-yl)-5-phenyl-2,3-dihydro-1H-benzo[el[1,4]diazepin-1-yl)butanoic acid: The title compound was prepared according to the procedure for (S)-2-(7-chloro-3-cyclopropyl-2-oxo-5-phenyl-2,3-dihydro-1H-benzo[e][1,4]diazepin-1-yl)acetic acid except that (S)-methyl 2-(7-chloro-3-cyclopropyl-2-oxo-5-phenyl-2,3-dihydro-1H-benzo[e][1,4]diazepin-1-yl)acetate was replaced with (S)-methyl 4-(7-chloro-2-oxo-3-(pentan-3-yl)-5-phenyl-2,3-dihydro-1H-benzo[e][1,4]diazepin-1-yl)butanoate to provide the title compound as an off white solid. ¹H NMR (300 MHz, CDCl₃): δ (ppm) 7.67-7.73 (m, 1H), 7.49-7.61 (m, 5H), 7.33-7.35 (m, 1H), 7.26-7.27 (m, 1H), 4.50-4.60 (m, 1H), 3.77-3.87 (m, 1H), 3.44 (d, J=11.14 Hz, 1 H), 2.61-2.71 (m, 1H), 2.19-2.27 (m, 2H), 1.84-1.95 (m, 2H), 1.65-1.75 (m, 2H), 1.53-1.61 (m, 1H), 1.31-1.39 (m, 1H), 0.82-0.90 (m, 6H); Calculated for C₂₄H₂₇ClN₂O₃, 426.17; observed (M+H)⁺427.8.

### Formulations

The present invention also relates to compositions or formulations which comprise the biological effect agents according to the present invention. In general, the compositions of the present invention comprise an effective amount of one or more diazepines of the present disclosure and salts thereof according to the present invention which are effective for treating or preventing Yellow Fever virus infection; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known biological effect agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more diazepines of the present disclosure according to the present invention and one or more excipients; from about 0.01 mg to about 100 mg of one or more diazepines of the present disclosure according to the present invention and one or more excipients; and from about 0.1 mg to about 10 mg of one or more diazepines of the present disclosure according to the present invention; and one or more excipients.

### Examples

The following procedures can be utilized in evaluating and selecting compounds as biological effect agents.

Determination of antiviral activity of compounds of the disclosure in human hepatoma-derived cell lines: Huh-7 cells were seeded into 96-well plates at a density of 5×10⁴ cells per well and cultured in Dulbecco's modified minimal essential medium (DMEM) media with 10% fetal bovine serum and (FBS), and 1% Penicillin-Streptomycin at 37° C. with 5% CO₂. On the second day of cell culture, cells were infected with YFV 17D strain at a multiplicity of infection (MOI) of 1 for 1 hour. After the virus-containing medium was removed, the cells were cultured in medium that contains serial dilution of compounds of the disclosure ranging from 10 □M to 0.004 □M. Cells mock-infected and infected with mock treatment (0.1% DMSO) served as controls. At 48 hours post-infection, cells were fixed with 3.5% paraformaldehyde (PFA) for 20 minutes away from light. The cells were then permeabilized by washing with mild detergent containing 0.5% Tris-100 for 15 min. After blocking cells with Intercept phosphate buffer saline protein-free blocking buffer (LI-COR) for 1 hour, the cells on plate were probed with Flavivirus Group Antigen antibody 4G2 at 1:1000 dilution (Novus biologicals) at room temperature for 3 hours to detect YFV envelope protein. After incubation with primary antibody, the cells were washed thrice with phosphate buffer saline with 0.1% tween 20, followed by incubation with the secondary antibody IRDye® 800CW Goat anti-mouse IgG (LI-COR) at 1:2000 dilution. Images and data quantification were obtained from duplicated experiments using LI-COR Odyssey. The antiviral efficacy of a compound was expressed as its concentration that reduce the amount of YFV envelope protein by 50% (EC₅₀) or 90% (EC₉₀). EC₅₀ are reported in the following ranges: +=>10 μM, ++=10 μM to 1 μM, +++=<1 μM to 500 nM, ++++<500 nM

Cell viability assay: Cell viability was determined simultaneously with antiviral activity, using the methods described to determine the antiviral activity of the compounds of the disclosure. DRAQ5 (Thermo Scientific) was added during the incubation with the secondary antibody to stain DNA present in the cellular nuclei. Images and data quantification were obtained from duplicated experiments using LI-COR Odyssey. The cell viability of a compound was expressed as its concentration that reduce the amount of nuclei staining by 50% (CC₅₀). CC₅₀ are reported in the following ranges: +=>10 μM, ++=10 μM to 1 μM, +++=<1 μM to 500 nM, ++++<500 nM

Exemplary embodiments include compounds having the formula (XXII) or a pharmaceutically acceptable salt form thereof:

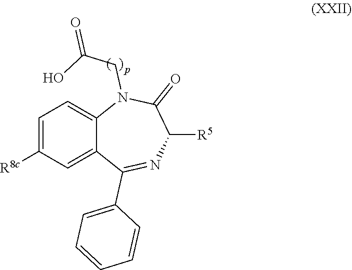

wherein non-limiting examples of R⁵, R^{8c} and p are defined herein below in Table 1.

TABLE 1

| Entry | R⁵ | R^{8c} | P | EC₅₀ | CC₅₀ |
|---|---|---|---|---|---|
| 1 | 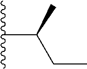 [SMILES: *[C@@H](C)CC] | Cl | 2 | ++++ | + |
| 2 | 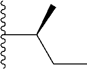 [SMILES: *[C@@H](C)CC] | Cl | 3 | ++++ | + |
| 3 | Me | Cl | 1 | + | + |
| 4 | 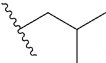 [SMILES: *CC(C)C] | Cl | 1 | ++ | + |
| 5 | phenyl | Cl | 1 | + | + |
| 6 | benzyl | Cl | 1 | + | + |
| 7 | isopropyl | Cl | 2 | ++++ | + |
| 8 | isopropyl | Cl | 1 | +++ | + |
| 9 | 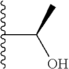 [SMILES: *[C@@H](C)O] | Cl | 1 | + | + |
| 10 | 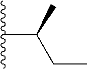 [SMILES: *[C@@H](C)CC] | H | 1 | ++ | + |
| 11 | 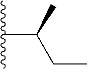 [SMILES: *[C@@H](C)CC] | NMe₂ | 1 | + | + |
| 12 | 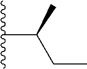 [SMILES: *[C@@H](C)CC] | F | 1 | ++ | + |
| 13 | 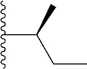 [SMILES: *[C@@H](C)CC] | Me | 1 | +++ | + |
| 14 | 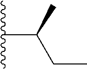 [SMILES: *[C@@H](C)CC] | OMc | 1 | ++ | + |
| 15 | 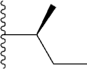 [SMILES: *[C@@H](C)CC] | CN | 1 | ++ | + |
| 16 | 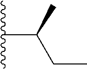 [SMILES: *[C@@H](C)CC] | Br | 1 | +++ | + |

Exemplary embodiments include compounds having the formula (XXIII) or a pharmaceutically acceptable salt form thereof:

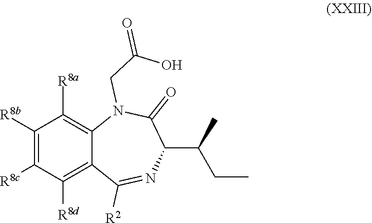

wherein non-limiting examples of R², R^{1a}, R^{5b}, R^{8c}, and R^{1d} are defined herein below in Table 2.

TABLE 2

| Entry | R² | R^{8a} | R^{8b} | R^{8c} | R^{8d} | EC₅₀ | CC₅₀ |
|---|---|---|---|---|---|---|---|
| 1 | Phenyl | H | H | Cl | H | ++++ | + |
| 2 | Cyclohexyl | H | H | Cl | H | + | + |
| 4 | 4-pyridyl | H | H | Cl | H | ++ | + |
| 5 | 2-pyridyl | H | H | Cl | H | ++ | + |
| 6 | Isopropyl | H | H | Cl | H | + | + |

Exemplary embodiments include compounds having the formula (XXIV) or a pharmaceutically acceptable salt form thereof:

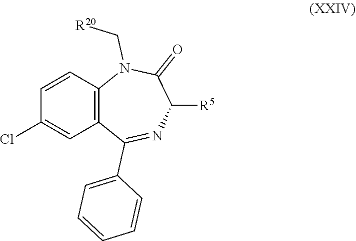

wherein non-limiting examples of R⁵ and R²⁰ are defined herein below in Table 3.

TABLE 3

| Entry | R²⁰ | R⁵ | EC₅₀ | CC₅₀ |
|---|---|---|---|---|
| 1 | 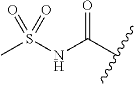 [SMILES: *C(=O)NS(C)(=O)=O] | 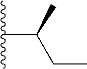 [SMILES: *[C@@H](C)CC] | ++++ | + |
| 2 | 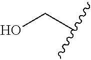 [SMILES: *CO] | 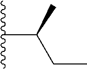 [SMILES: *[C@@H](C)CC] | ++ | + |
| 3 | 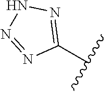 [SMILES: *c1nn[nH]n1] | 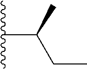 [SMILES: *[C@@H](C)CC] | ++++ | + |
| 4 | 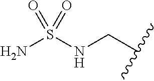 [SMILES: *CNS(N)(=O)=O] | 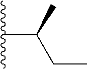 [SMILES: *[C@@H](C)CC] | ++ | + |
| 5 | 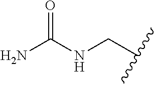 [SMILES: *CNC(N)=O] | 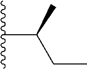 [SMILES: *[C@@H](C)CC] | ++ | + |
| 6 | 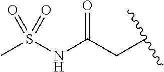 [SMILES: *CC(=O)NS(C)(=O)=O] | 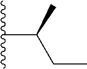 [SMILES: *[C@@H](C)CC] | ++++ | + |
| 7 | 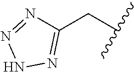 [SMILES: *Cc1nn[nH]n1] | 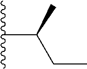 [SMILES: *[C@@H](C)CC] | ++++ | + |
| 8 | 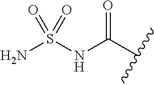 [SMILES: *C(=O)NS(N)(=O)=O] | 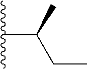 [SMILES: *[C@@H](C)CC] | ++++ | + |
| 9 | 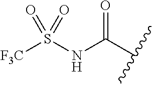 [SMILES: *C(=O)NS(=O)(=O)C(F)(F)F] | 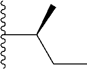 [SMILES: *[C@@H](C)CC] | ++ | + |
| 10 | 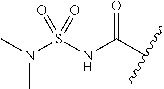 [SMILES: *C(=O)NS(=O)(=O)N(C)C] | 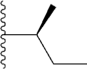 [SMILES: *[C@@H](C)CC] | ++++ | + |
| 11 | 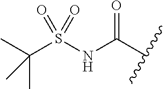 [SMILES: *C(=O)NS(=O)(=O)C(C)(C)C] | 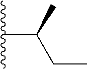 [SMILES: *[C@@H](C)CC] | ++ | + |
| 12 | 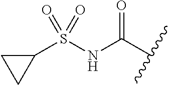 [SMILES: *C(=O)NS(=O)(=O)C1CC1] | 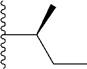 [SMILES: *[C@@H](C)CC] | +++ | + |
| 13 | 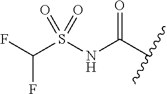 [SMILES: *C(=O)NS(=O)(=O)C(F)F] | 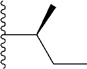 [SMILES: *[C@@H](C)CC] | ++ | + |
| 14 | 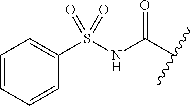 [SMILES: *C(=O)NS(=O)(=O)c1ccccc1] | 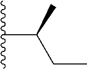 [SMILES: *[C@@H](C)CC] | ++++ | + |
| 15 | 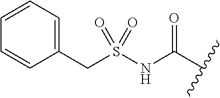 [SMILES: *C(=O)NS(=O)(=O)Cc1ccccc1] | 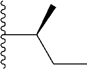 [SMILES: *[C@@H](C)CC] | +++ | + |
| 16 | 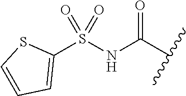 [SMILES: *C(=O)NS(=O)(=O)c1cccs1] | 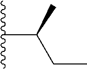 [SMILES: *[C@@H](C)CC] | ++++ | + |
| 17 | 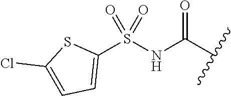 [SMILES: *C(=O)NS(=O)(=O)c1ccc(Cl)s1] | 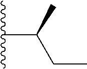 [SMILES: *[C@@H](C)CC] | +++ | + |
| 18 | 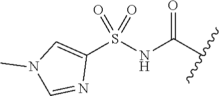 [SMILES: *C(=O)NS(=O)(=O)c1cn(C)cn1] | 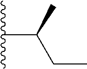 [SMILES: *[C@@H](C)CC] | + | + |
| 19 | 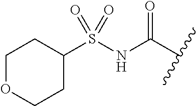 [SMILES: *C(=O)NS(=O)(=O)C1CCOCC1] | 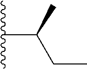 [SMILES: *[C@@H](C)CC] | ++ | + |
| 20 | 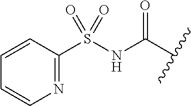 [SMILES: *C(=O)NS(=O)(=O)c1ccccn1] | 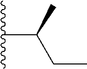 [SMILES: *[C@@H](C)CC] | ++++ | + |
| 21 | 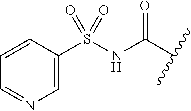 [SMILES: *C(=O)NS(=O)(=O)c1cccnc1] | 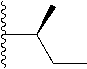 [SMILES: *[C@@H](C)CC] | +++ | + |
| 22 | 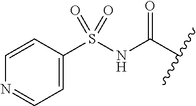 [SMILES: *C(=O)NS(=O)(=O)c1ccncc1] | 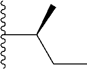 [SMILES: *[C@@H](C)CC] | ++ | + |
| 23 | 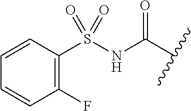 [SMILES: *C(=O)NS(=O)(=O)c1ccccc1F] | 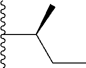 [SMILES: *[C@@H](C)CC] | ++++ | + |
| 24 | 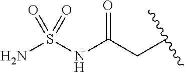 [SMILES: *CC(=O)NS(N)(=O)=O] | 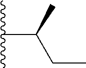 [SMILES: *[C@@H](C)CC] | ++ | + |
| 25 | 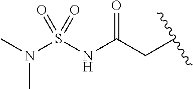 [SMILES: *CC(=O)NS(=O)(=O)N(C)C] | 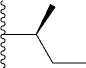 [SMILES: *[C@@H](C)CC] | ++ | + |
| 26 | 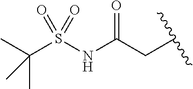 [SMILES: *CC(=O)NS(=O)(=O)C(C)(C)C] | 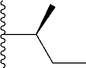 [SMILES: *[C@@H](C)CC] | ++ | + |
| 27 | 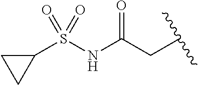 [SMILES: *CC(=O)NS(=O)(=O)C1CC1] | 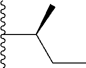 [SMILES: *[C@@H](C)CC] | ++ | + |
| 28 | 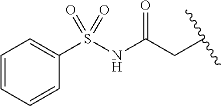 [SMILES: *CC(=O)NS(=O)(=O)c1ccccc1] | 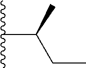 [SMILES: *[C@@H](C)CC] | ++ | + |
| 29 | 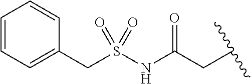 [SMILES: *CC(=O)NS(=O)(=O)Cc1ccccc1] | 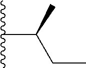 [SMILES: *[C@@H](C)CC] | +++ | + |
| 30 | 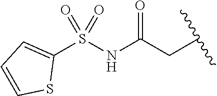 [SMILES: *CC(=O)NS(=O)(=O)c1cccs1] | 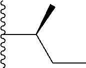 [SMILES: *[C@@H](C)CC] | ++ | + |
| 31 | 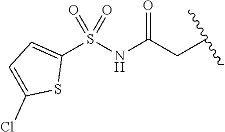 [SMILES: *CC(=O)NS(=O)(=O)c1ccc(Cl)s1] | 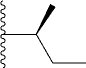 [SMILES: *[C@@H](C)CC] | ++ | + |
| 32 | 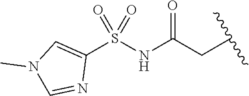 [SMILES: *CC(=O)NS(=O)(=O)c1cn(C)cn1] | 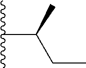 [SMILES: *[C@@H](C)CC] | ++ | + |
| 33 | 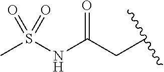 [SMILES: *CC(=O)NS(C)(=O)=O] | 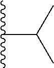 [SMILES: *C(C)C] | +++ | + |
| 34 | 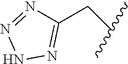 [SMILES: *Cc1nn[nH]n1] | 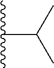 [SMILES: *C(C)C] | ++ | + |

Exemplary embodiments include compounds having the formula (XXV) or a pharmaceutically acceptable salt form thereof:

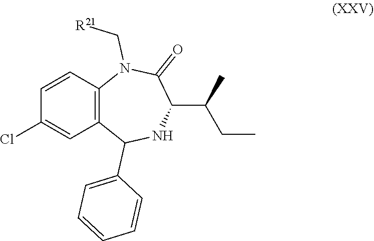

wherein non-limiting examples of R²¹ are defined herein below in Table 4.

TABLE 4

| Entry | R²¹ | EC₅₀ | CC₅₀ |
|---|---|---|---|
| 1 | 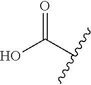 [SMILES: *C(=O)O] | ++ | + |
| 2 | 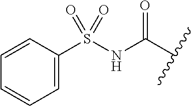 [SMILES: *C(=O)NS(=O)(=O)c1ccccc1] | ++ | + |
| 3 | 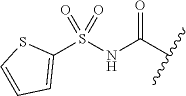 [SMILES: *C(=O)NS(=O)(=O)c1cccs1] | ++ | + |
| 4 | 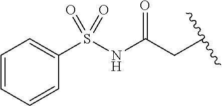 [SMILES: *CC(=O)NS(=O)(=O)c1ccccc1] | + | + |
| 5 | 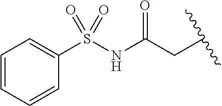 [SMILES: *CC(=O)NS(=O)(=O)c1ccccc1] | + | + |
| 6 | 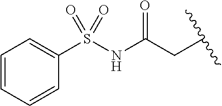 [SMILES: *CC(=O)NS(=O)(=O)c1ccccc1] | + | + |
| 7 | 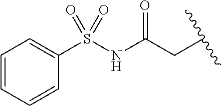 [SMILES: *CC(=O)NS(=O)(=O)c1ccccc1] | + | + |
| 8 | 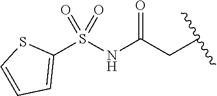 [SMILES: *CC(=O)NS(=O)(=O)c1cccs1] | + | + |
| 9 | 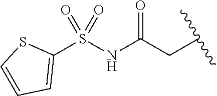 [SMILES: *CC(=O)NS(=O)(=O)c1cccs1] | + | + |
| 10 | 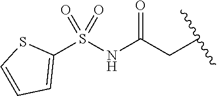 [SMILES: *CC(=O)NS(=O)(=O)c1cccs1] | + | + |
| 11 | 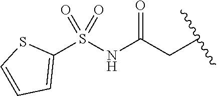 [SMILES: *CC(=O)NS(=O)(=O)c1cccs1] | + | + |

Exemplary embodiments include compounds having the formula (XXVI) or a pharmaceutically acceptable salt form thereof:

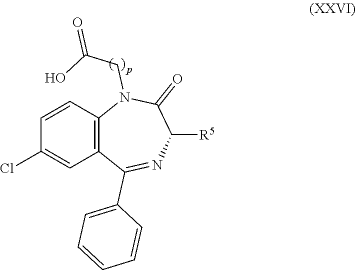

wherein non-limiting examples of R⁵ and p are defined herein below in Table 5.

TABLE 5

| Entry | R⁵ | P | EC₅₀ | CC₅₀ |
|---|---|---|---|---|
| 1 | Phenyl | 1 | + | + |
| 2 | 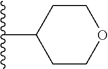 [SMILES: *C1CCOCC1] | 2 | + | + |
| | Racemic | | | |
| 3 | 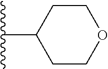 [SMILES: *C1CCOCC1] | 1 | + | + |
| | Racemic | | | |
| 4 | 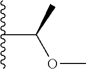 [SMILES: *[C@@H](C)OC] | 1 | + | + |
| 5 | 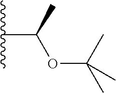 [SMILES: *[C@@H](C)OC(C)(C)C] | 1 | + | + |
| 6 | 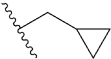 [SMILES: *CC1CC1] | 1 | + | + |
| 7 | 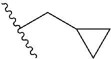 [SMILES: *CC1CC1] | 2 | ++ | + |
| 8 | 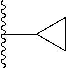 [SMILES: *C1CC1] | 1 | ++ | + |
| 9 | 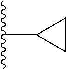 [SMILES: *C1CC1] | 2 | +++ | + |
| 10 | 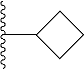 [SMILES: *C1CCC1] | 1 | + | + |
| 11 | 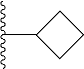 [SMILES: *C1CCC1] | 2 | ++ | + |
| 12 | 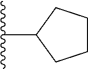 [SMILES: *C1CCCC1] | 1 | ++ | + |
| 13 | 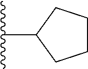 [SMILES: *C1CCCC1] | 2 | ++ | + |
| 14 | 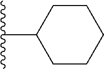 [SMILES: *C1CCCCC1] | 1 | + | + |
| 15 | 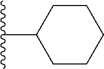 [SMILES: *C1CCCCC1] | 2 | + | + |
| 16 | 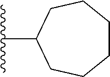 [SMILES: *C1CCCCCC1] | 2 | + | + |
| 17 | 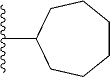 [SMILES: *C1CCCCCC1] | 1 | + | + |
| 18 | 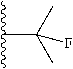 [SMILES: *C(C)(C)F] | 1 | ++ | + |
| 19 | 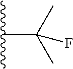 [SMILES: *C(C)(C)F] | 2 | ++ | + |
| 20 | 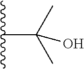 [SMILES: *C(C)(C)O] | 1 | ++ | + |
| 21 | 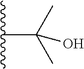 [SMILES: *C(C)(C)O] | 2 | ++ | + |
| 22 | 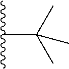 [SMILES: *C(C)(C)C] | 1 | ++ | + |
| 23 | 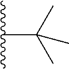 [SMILES: *C(C)(C)C] | 2 | ++++ | + |
| 24 | 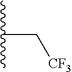 [SMILES: *CC(F)(F)F] | 1 | + | + |
| 25 | 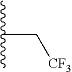 [SMILES: *CC(F)(F)F] | 2 | ++ | + |
| 26 | 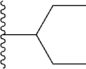 [SMILES: *C(CC)CC] | 1 | ++++ | + |
| 27 | 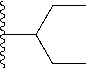 [SMILES: *C(CC)CC] | 2 | ++++ | + |
| 28 | 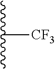 [SMILES: *C(F)(F)F] | 1 | + | + |
| 29 | 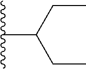 [SMILES: *C(CC)CC] | 3 | ++++ | + |

Exemplary embodiments include compounds having the formula (XXVII) or a pharmaceutically acceptable salt form thereof:

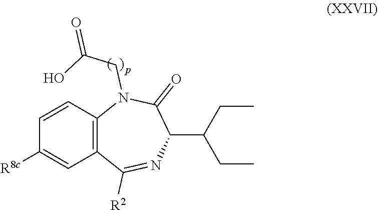

wherein non-limiting examples of R², R^{8c}, and p are defined herein below in Table 6.

TABLE 6

| Entry | R^{8c} | R² | n | EC₅₀ | CC₅₀ |
|---|---|---|---|---|---|
| 1 | Br | Phenyl | 2 | ++++ | + |
| 2 | I | Phenyl | 2 | ++++ | + |
| 3 | Me | Pheynl | 2 | ++++ | + |
| 4 | Phenyl | Phenyl | 2 | ++ | + |
| 5 | Cyclopropyl | Phenyl | 2 | ++ | + |
| 6 | 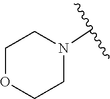 [SMILES: *N1CCOCC1] | Phenyl | 2 | + | + |
| 7 | CF₃ | Phenyl | 1 | ++++ | + |
| 8 | CF₃ | Phenyl | 2 | ++ + | + |
| 9 | Br | 2-pyridyl | 1 | +++ | + |
| 10 | Br | pyridyl | 2 | ++++ | + |

Exemplary embodiments include compounds having the formula (XXVIII) or a pharmaceutically acceptable salt form thereof:

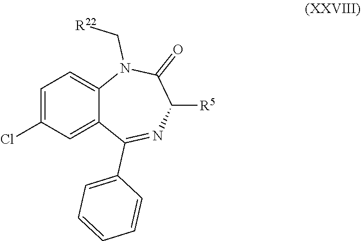

wherein non-limiting examples of R²², R⁵, and p are defined herein below in Table 7.

TABLE 7

| Entry | R²² | R⁵ | EC₅₀ | CC₅₀ |
|---|---|---|---|---|
| 1 | 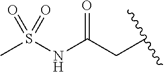 [SMILES: *CC(=O)NS(C)(=O)=O] | 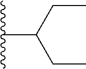 [SMILES: *C(CC)CC] | ++++ | + |
| 2 | 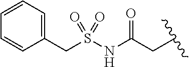 [SMILES: *CC(=O)NS(=O)(=O)Cc1ccccc1] | 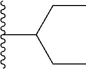 [SMILES: *C(CC)CC] | ++++ | + |
| 3 | 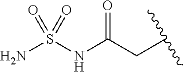 [SMILES: *CC(=O)NS(N)(=O)=O] | 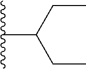 [SMILES: *C(CC)CC] | ++++ | + |
| 4 | 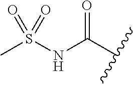 [SMILES: *C(=O)NS(C)(=O)=O] | 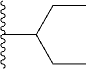 [SMILES: *C(CC)CC] | +++ | + |
| 5 | 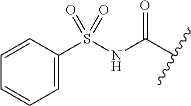 [SMILES: *C(=O)NS(=O)(=O)c1ccccc1] | 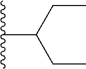 [SMILES: *C(CC)CC] | ++++ | + |
| 6 | 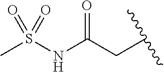 [SMILES: *CC(=O)NS(C)(=O)=O] | 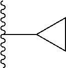 [SMILES: *C1CC1] | ++ | + |
| 7 | 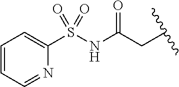 [SMILES: *CC(=O)NS(=O)(=O)c1ccccn1] | 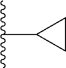 [SMILES: *C1CC1] | + | + |
| 8 | 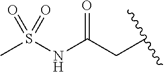 [SMILES: *CC(=O)NS(C)(=O)=O] | 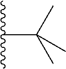 [SMILES: *C(C)(C)C] | +++ | + |
| 9 | 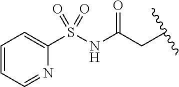 [SMILES: *CC(=O)NS(=O)(=O)c1ccccn1] | 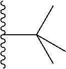 [SMILES: *C(C)(C)C] | +++ | + |

Exemplary embodiments include compounds having the formula (XXIX) or a pharmaceutically acceptable salt form thereof:

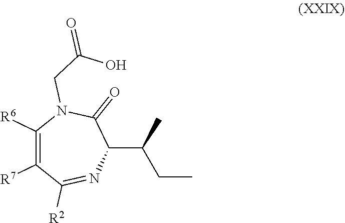

wherein non-limiting examples of R², R⁶, and R⁷ are defined herein below in Table 8.

TABLE 8

| | | | | | |
|---|---|---|---|---|---|
| Entry | R² | R⁶ | R⁷ | EC₅₀ | CC₅₀ |

| | | | | |
|---|---|---|---|---|
| 1 | Phenyl | 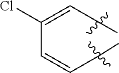 [SMILES: */C=C\C(Cl)=C/*] | ++ | + |
| 2 | Phenyl | 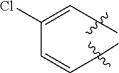 [SMILES: */C=C\C(Cl)=C/*] | ++ | + |

1. A compound having formula (I): 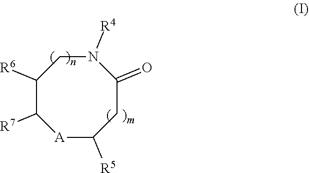
Including enantiomers, diasteromers, hydrates, solvates, pharmaceutically acceptable salts, isotopic analogs, prodrugs and complexes thereof, wherein:
A is selected from the groups consisting of 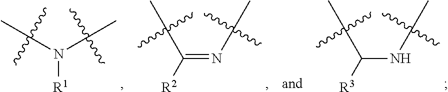
When A is 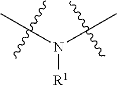
m is 0;
When A is 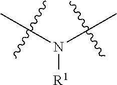
n is selected from 0 and 1;
R¹ is selected from the group consisting of C3-7 cycloalkyl, C3-C7 branched alkyls; C1-C6 haloalkyls, C3-C7 branched haloalkyl, optionally substituted aryl and optionally substituted heteroaryl;
When A is 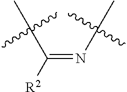
m is selected from 0 and 1;
When A is 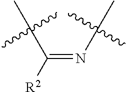
n is selected from 0 and 1;
R² is selected from consisting of C3-C8 cycloalkyl, C4-C10 bicyclic alkyl, C3-C7 branched alkyls, C1-C6 haloalkyls, C3-C7 branched haloalkyl, optionally substituted aryl, and optionally substituted heteroaryl;
When A is 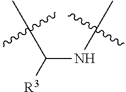
m is selected from 0 and 1;
When A is 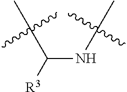
n is selected from 0 and 1;
R³ is selected from consisting of C3-C8 cycloalkyl; C3-C7 branched alkyls, C1-C6 haloalkyls, C3-C7 branched haloalkyl, optionally substituted aryl, and optionally substituted heteroaryl;
R⁴ is selected from the groups consisting of 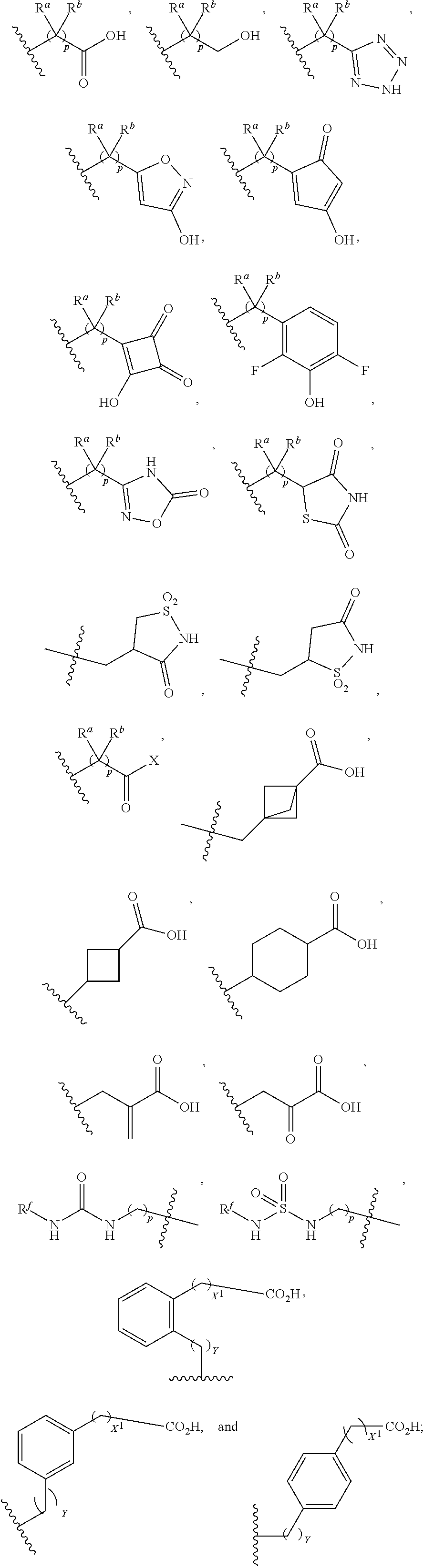
p is 1,2, 3,4, or 5;
When R⁴ is 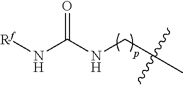
p is not 1;
When R⁴ is 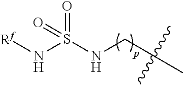
p is not 1;
X¹ is 0, 1, 2, or 3;
Y is 0, 1, 2, 3, or 4;
X is selected from the group consisting of 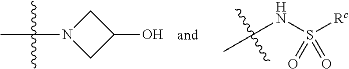
R^{a} is selected from the group consisting of H, C1-C6 alkyl, C3-C6 cycloalkyl, halogan;
R^{b} is selected from the group consisting of H, C1-C6 alkyl, C3-C6 cycloalkyl, halogen;
R^{c} is selected from the group consisting of C1-C12 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, NR^{d}R^{e}, optionally substituted benzyl, optionally substituted CH₂heteroaryl, optionally substituted aryl, optionally substituted heteroaryl, and C3-C6 cycloalkyl ring optionally containing an oxygen,
R^{a} and R^{b} can be taken together to form a C3-C6 cycloalkyl ring optionally containing an oxygen;
R^{d} is selected from the group consisting of hydrogen, C1-C6 alkyl, C3-C6 cycloalyl, optionally aromatic ring, and optionally substituted heteroaromatic ring;
R^{c} is selected from the group consisting of hydrogen, C1-C6 alkyl, C3-C6 cycloalyl, optionally aromatic ring, and optionally substituted heteroaromatic ring;
R^{f} is selected from the group consisting of hydrogen C1-C12 alkyl, C3-C11 branched alkyl, C3-C8 cycloalkyl, C5-C15 bicycloalkyls, CH₂F, CHF₂, CF₃, C2-C9 haloalkyls, C₃-C9 branched haloalkyl, optionally substituted benzyl, optionally substituted CH₂heteroaryl, optionally substituted aryl, optionally substituted heteroaryl, and C3-C6 cycloalkyl ring optionally containing an oxygen;
R⁵ is selected from the groups consisting of C2-C8 alkyl, C3-C11 branched alkyl, C3-C8 cycloalkyl, C5-C15 bicycloalkyls, CH₂F, CHF₂, CF₃, C2-C9 haloalkyls, C3-C9 branched haloalkyl, optionally substituted benzyl, optionally substituted CH₂heteroaryl, CH₂OR⁵a, CH(CH₃)OR^{5a}, C(R^{5e})₂OR^{5a}, C(R^{5e})₂OR^{5a}, CH₂SR^{5a}, CH₂CH₂SCH₃, CH₂CH₂SO₂CH₃, CH₂CH₂CH₂NR^{5c}R^{5d}, CH₂COR^{5b}, CH₂CH₂COR^{5b}, (CH₂)_{z}(C3-C7cycloakyl), and C3-C6 cycloalkyl ring optionally containing an oxygen;
z is 1, 2, 3, 4, 5, or 6;
R^{5a} is selected from the group consisting of hydrogen, C1-C6 alkyl, C3-C7 branched alkyl, optionally substituted phenyl, optionally substituted benzyl, and optionally substituted CH₂CH₂Ar;
R^{5b} is selected from the group consisting of OH, C1-C6 alkoxy, and NH₂;
R^{5c} and R^{5d} are each independently is selected from the group consisting of hydrogen and optionally substituted C1-C6 alkyl;
R^{5e} is C1-C4 alkyl;
R⁶ and R⁷ are hydrogen;
Or
R⁶ and R⁷ are taken together with the atoms to which they are bound to form a 6 membered aromatic ring that is substituted with at least one moiety that is selected from the group consisting of halogen, C1-C6 haloalkyl, cyano, C1-C6 alkyl, C1-C6 alkoxy, 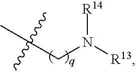
C3-C8 cycloalkyl, C3-C8 heterocycle, aryl, and heteroaryl;
Or
R⁶ and R⁷ are taken together with the atoms to which they are bound to form a substituted heteroaromatic ring containing 5 to 6 members containing 1 to 2 nitrogens that is substituted with at least one moiety that is selected from the group consisting of halogen, C₁-C₆ haloalkyl, cyano, C1-C6 alkoxy, 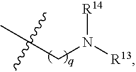
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1, 2, 3, 4, 5, or 6; wherein the compound of formula (I) does not include:

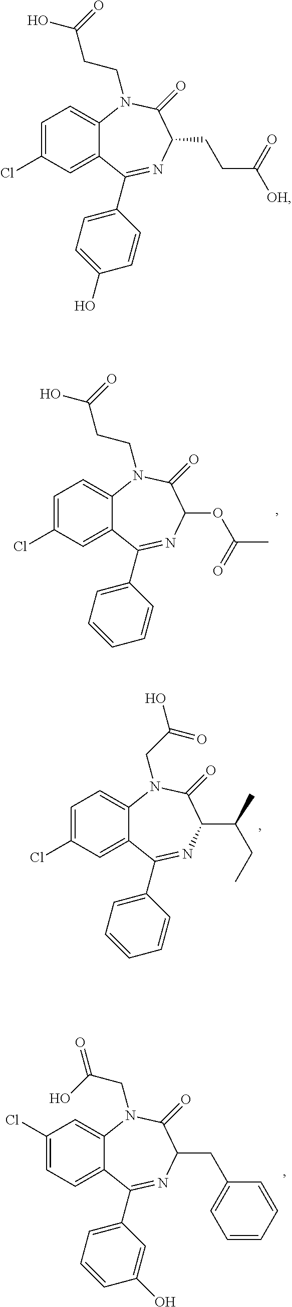 [SMILES: O=C(O)CC[C@@H]1N=C(c2ccc(O)cc2)c2cc(Cl)ccc2N(CCC(=O)O)C1=O ; CC(=O)OC1N=C(c2ccccc2)c2cc(Cl)ccc2N(CCC(=O)O)C1=O ; CC[C@H](C)[C@@H]1N=C(c2ccccc2)c2cc(Cl)ccc2N(CC(=O)O)C1=O ; O=C(O)CN1C(=O)C(Cc2ccccc2)N=C(c2cccc(O)c2)c2ccc(Cl)cc21]

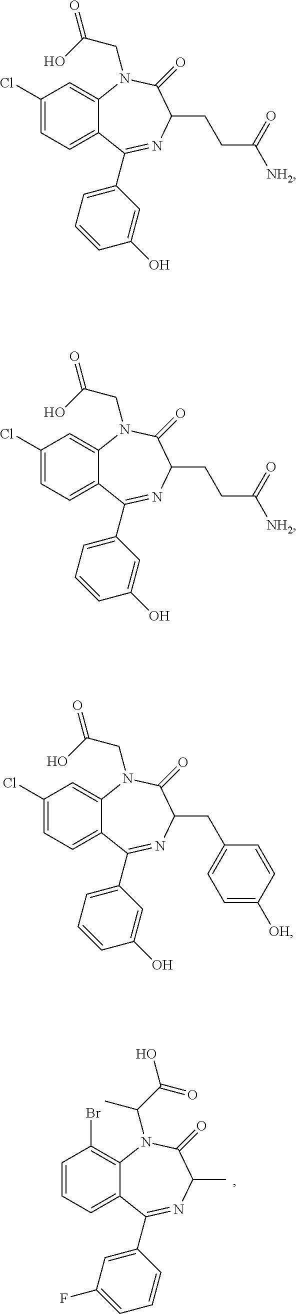 [SMILES: NC(=O)CCC1N=C(c2cccc(O)c2)c2ccc(Cl)cc2N(CC(=O)O)C1=O ; NC(=O)CCC1N=C(c2cccc(O)c2)c2ccc(Cl)cc2N(CC(=O)O)C1=O ; O=C(O)CN1C(=O)C(Cc2ccc(O)cc2)N=C(c2cccc(O)c2)c2ccc(Cl)cc21 ; CC1N=C(c2cccc(F)c2)c2cccc(Br)c2N(C(C)C(=O)O)C1=O]

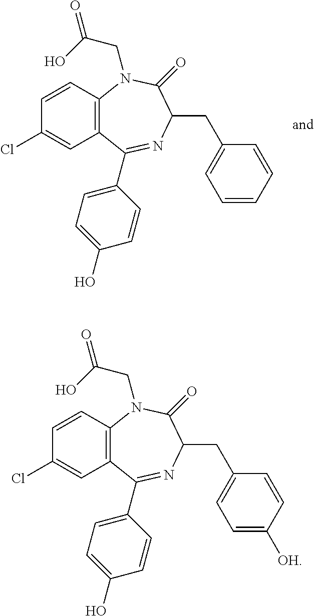 [SMILES: O=C(O)CN1C(=O)C(Cc2ccccc2)N=C(c2ccc(O)cc2)c2cc(Cl)ccc21 ; O=C(O)CN1C(=O)C(Cc2ccc(O)cc2)N=C(c2ccc(O)cc2)c2cc(Cl)ccc21]

2. The compound according to claim 1 having formula (II): 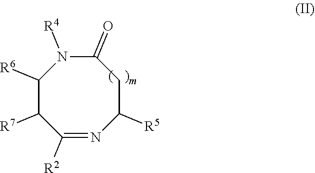
Including enantiomers, diasteromers, hydrates, solvates, pharmaceutically acceptable salts, prodrugs and complexes thereof.

3. The compound according to claim 1 having formula (III): 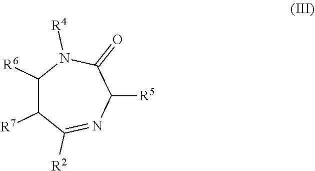
Including enantiomers, diasteromers, hydrates, solvates, pharmaceutically acceptable salts, prodrugs and complexes thereof.

4. The compound according to claim 1 having formula (IV): 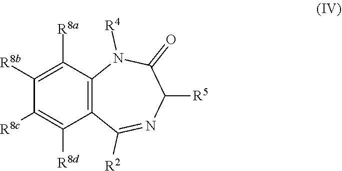
Including enantiomers, diasteromers, hydrates, solvates, pharmaceutically acceptable salts, prodrugs and complexes thereof wherein:
R^{8a} is selected from the group consisting of hydrogen halogen, C1-C6 haloalkyl, cyano, C1-C6 alkyl, C1-C6 alkoxy, 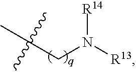
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{8b} is selected from the group consisting of hydrogen, halogen, C1-C6 haloalkyl, cyano, C1-C6 alkyl, C1-C6 alkoxy, 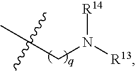
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{8c} is selected from the group consisting of halogen, C1-C6 haloalkyl, cyano, C₁-C₆ alkyl, C1-C6 alkoxy, 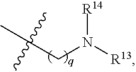
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{8d} is selected from the group consisting of hydrogen, halogen, C1-C6 haloalkyl, cyano, C1-C6 alkyl, C1-C6 alkoxy, 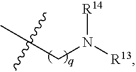
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0,1,2,3,4,5, or 6;
and at least two of R^{8a}, R^{8b}, and R^{8d} are hydrogen.

5. The compound according to claim 1 having formula (V): 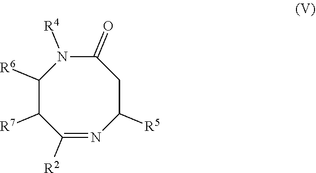
Including enantiomers, diasteromers, hydrates, solvates, pharmaceutically acceptable salts, prodrugs and complexes thereof.

6. The compound according to claim 1 having formula (VI): 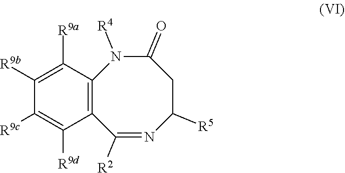
Including enantiomers, diasteromers, hydrates, solvates, pharmaceutically acceptable salts, prodrugs and complexes thereof wherein:
R^{9a} is selected from the group consisting of hydrogen, halogen, C1-C6 haloalkyl, cyano, C1-C6 alkyl, C1-C6 alkoxy, 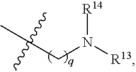
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{9b} is selected from the group consisting of hydrogen, halogen, C1-C6 haloalkyl, cyano, C1-C6 alkyl, C1-C6 alkoxy, 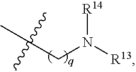
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{9c} is selected from the group consisting of halogen, C1-C6 haloalkyl, cyano, C₁-C₆ alkyl, C1-C6 alkoxy, 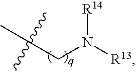
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{9d} is selected from the group consisting of hydrogen, halogen, C1-C6 14 haloalkyl, cyano, C1-C6 alkyl, C1-C6 alkoxy, 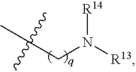
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6
At least two of R^{9a}, R^{9b}, and R^{9d} are hydrogen.

7. The compound according to claim 1 having formula (VII): 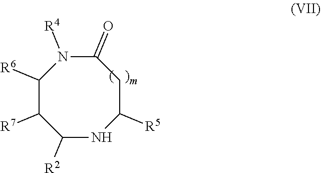
Including enantiomers, diasteromers, hydrates, solvates, pharmaceutically acceptable salts, prodrugs and complexes thereof.

8. The compound according to claim 1 having formula (VIII): 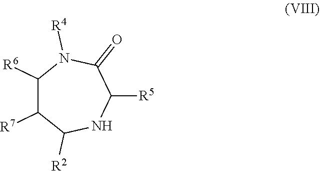
Including enantiomers, diasteromers, hydrates, solvates, pharmaceutically acceptable salts, prodrugs and complexes thereof.

9. The compound according to claim 1 having formula (IX): 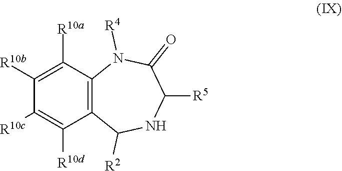
Including enantiomers, diasteromers, hydrates, solvates, pharmaceutically acceptable salts, prodrugs and complexes thereof wherein
R^{10a} is selected from the group consisting of hydrogen, halogen, C1-C6 haloalkyl, cyano, C1-C6 alkyl, C1-C6 alkoxy, 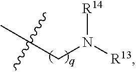
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{10b} is selected from the group consisting of hydrogen, halogen, C1-C6 haloalkyl, cyano, C1-C6 alkyl, C1-C6 alkoxy, 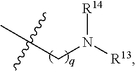
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{10c} is selected from the group consisting of halogen, C1-C6 haloalkyl, cyano, C₁-C₆ alkyl, C1-C6 alkoxy, 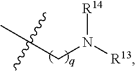
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{10d} is selected from the group consisting of hydrogen, halogen, C1-C6 haloalkyl, cyano, C1-C6 alkyl, C1-C6 alkoxy, 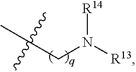
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
At least two of R^{10a}, R^{10b}, and R^{10d} are hydrogen.

10. The compound according to claim 1 having formula (X): 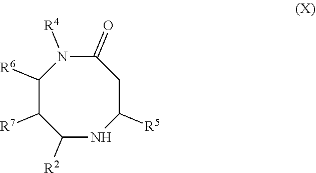
Including enantiomers, diasteromers, hydrates, solvates, pharmaceutically acceptable salts, prodrugs and complexes thereof.

11. The compound according to claim 1 having formula (XI): 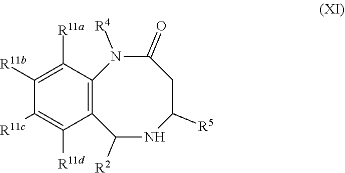
Including enantiomers, diasteromers, hydrates, solvates, pharmaceutically acceptable salts, prodrugs and complexes thereof wherein:
R^{1a} is selected from the group consisting of hydrogen, halogen, C1-C6 haloalkyl, cyano, C1-C6 alkyl, C1-C6 alkoxy, 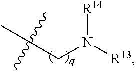
C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{11b} is selected from the group consisting of hydrogen, halogens, C1-C6 haloalkyl, cyano, C1-C6 alkyl, C1-C6 alkoxy, 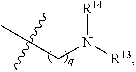
C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{11c} is selected from the group consisting of halogen, C1-C6 haloalkyl, cyano, C₁-C₆ alkyl, C1-C6 alkoxy, 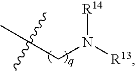
C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{11d} is selected from the group consisting of hydrogen, halogen, C1-C6 haloalkyl, cyano, C1-C6 alkyl, C1-C6 alkoxy, 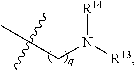
C3-C8 cycloalkyl, C3-C8 heterocycle, aryls, heteroaromatic rings;
R¹³ is selected from the group consisting of hydrogen and C1-C6 alkyl;
R¹⁴ is selected from the group consisting of hydrogen and C1-C6 alkyl;
q is 0,1,2,3,4,5, or 6;
At least two of R^{11a}, R^{11b}, and R^{11d} are hydrogen.

12. The compound according to claim 1 having formula (XII): 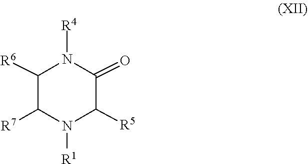
Including enantiomers, diasteromers, hydrates, solvates, pharmaceutically acceptable salts, prodrugs and complexes thereof.

13. The compound according to claim 1 having formula (XIII): 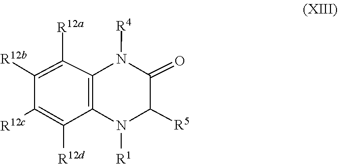
Including enantiomers, diasteromers, hydrates, solvates, pharmaceutically acceptable salts, prodrugs and complexes thereof wherein
R^{12a} is selected from the group consisting of hydrogen, halogen, C1-C6 haloalkyl, cyano, C1-C6 alkyl, C1-C6 alkoxy, 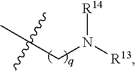
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{12b} is selected from the group consisting of hydrogen, halogen, C1-C6 haloalkyl, cyano, C1-C6 alkyl, C1-C6 alkoxy, 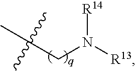
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{12c} is selected from the group consisting of halogens, C1-C6 haloalkyls, cyano, C1-C6 alkyl, C1-C6 alkoxy, 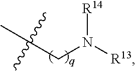
C3-C8 cycloalkyl, C₃-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
R^{12d} is selected from the group consisting of hydrogen, halogen, C1-C6 haloalkyl, cyano, C1-C6 alkyl, C1-C6 alkoxy, 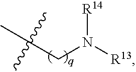
C3-C8 cycloalkyl, C3-C8 heterocycle, aryl, and heteroaryl;
R¹³ is selected from the group consisting of hydrogen and C1-C6 alkyl;
R¹⁴ is selected from the group consisting of hydrogen and C1-C6 alkyl;
q is 0, 1,2,3,4,5, or 6;
At least two of R^{12a}, R^{12b}, and R^{12d} are hydrogen.

14. The compound according to claim 1 having formula selected from: (XIV); (XV); (XVI); and (XVII): 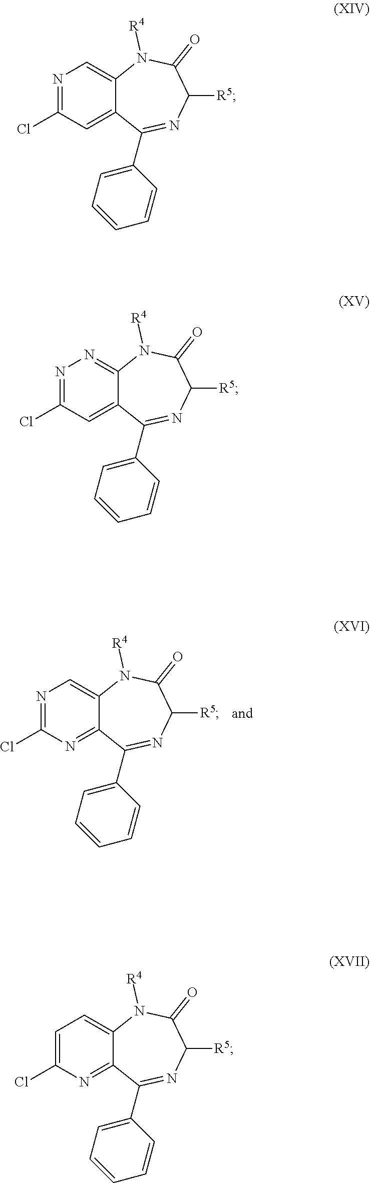
Including enantiomers, diasteromers, hydrates, solvates, pharmaceutically acceptable salts, prodrugs and complexes thereof.

15-17. (canceled)

18. The compound according to claim 1 having formula (XVII) 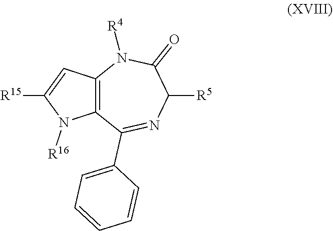
Including enantiomers, diasteromers, hydrates, solvates, pharmaceutically acceptable salts, prodrugs and complexes thereof wherein: R¹⁵ is selected from the group consisting of C1 -C₆ alkyl, C3-C6 cycloalkyl, and C1-C6 haloalkyl; R¹⁶ is selected from the group consisting of C₁-C₆ alkyl, C3-C6 cycloalkyl, and C1-C6 haloalkyl.

19. The compound according to claim 1 having formula (XIX): 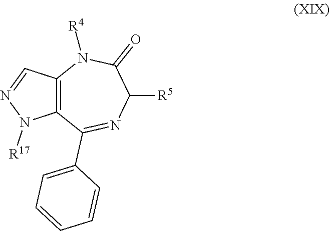
Including enantiomers, diasteromers, hydrates, solvates, pharmaceutically acceptable salts, prodrugs and complexes thereof wherein: R¹⁷ is selected from the group consisting of C1-C6 alkyl, C3-C6 cycloalkyl, and C1-C6 haloalkyl.

20. The compound according to claim 1 having formula (XX): 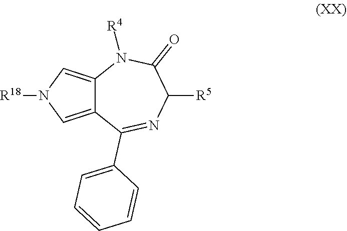
Including enantiomers, diasteromers, hydrates, solvates, pharmaceutically acceptable salts, prodrugs and complexes thereof wherein: R¹⁸ is selected from the group consisting of C1-C6 alkyl, C3-C6 cycloalkyl, and C1-C6 haloalkyl.

21. The compound according to claim 1 having formula (XXI): 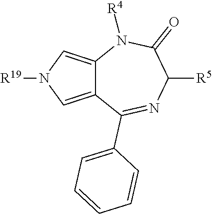
Including enantiomers, diasteromers, hydrates, solvates, pharmaceutically acceptable salts, prodrugs and complexes thereof wherein: R¹⁹ is selected from the group consisting of C1 -C6 alkyl, C3-C6 cycloalkyl, and C1-C6 haloalkyl.

22. A composition comprising an effective amount of at least one compound according to claim 1; and at least one excipient.

23. (canceled)

24. A method for treating or preventing Yellow Fever virus infection, said method comprising administering to a subject an effective amount of at least one compound according to the claim 1 to treat or prevent Yellow Fever virus infection.

25. (canceled)
